(19) United States
(12) Patent Application Publication
(10) Pub. No.: US 2022/0389454 A1
(43) Pub. Date: Dec. 8, 2022

(54) LENTIVIRAL VECTORS IN HEMATOPOIETIC STEM CELLS TO TREAT RECOMBINATION-ACTIVATING GENE 1 (RAG1) SEVERE COMBINED IMMUNODEFICIENCY (SCID)

(71) Applicant: The Regents of the University of California, Oakland, CA (US)

(72) Inventors: Donald B. Kohn, Tarzana, CA (US); Ryan L. Wong, Los Angeles, CA (US); Roger Paul Hollis, Los Angeles, CA (US); Richard A. Morgan, Santa Monica, CA (US)

(73) Assignee: The Regents of the University of California, Oakland, CA (US)

(21) Appl. No.: 17/775,859

(22) PCT Filed: Nov. 12, 2020
(86) PCT No.: PCT/US20/60279

§ 371 Date: May 10, 2022

Related U.S. Application Data
(60) Provisional application No. 62/935,022, filed on Nov. 13, 2019.

(51) Int. Cl. C12N 15/86

(52) U.S. Cl. CPC C12N 15/86; C12N 2740/16043

(57) ABSTRACT

In certain embodiments a lentiviral vector for the treatment of Recombination-Activating Gene 1 Severe Combined Immunodeficiency (RAG1 SCID) is provided. In certain embodiments the vector comprises an expression cassette comprising a nucleic acid construct comprising an effective fragment of the endogenous promoter of the RAG1 gene and a nucleic acid that encodes the RAG1 protein operably linked to the effective fragment of the endogenous promoter of the RAG1 gene.

### CROSS-REFERENCE TO RELATED APPLICATIONS

This application claims benefit of and priority to U.S. Ser. No. 62/935,022, filed on Nov. 13, 2019, which is incorporated herein by reference in its entirety for all purposes.

### STATEMENT OF GOVERNMENTAL SUPPORT

[Not Applicable]

### INCORPORATION BY REFERENCE OF SEQUENCE LISTING PROVIDED AS A TEXT FILE

A Sequence Listing is provided herewith as a text file, “UCLA-P219WO_ST25.txt” created on Nov. 12, 2020 and having a size of 165 KB. The contents of the text file are incorporated by reference herein in their entirety.

### BACKGROUND

RAG1 Severe Combined Immunodeficiency (RAG1 SCID) is a primary immune deficiency caused by mutations in Recombination-Activating Gene 1 (RAG1). RAG1 and RAG2 are two subunits of the RAG complex which is active in immune system cells (lymphocytes) called B cells and T cells. These cells have proteins on their surface that recognize foreign invaders and help protect the body from infection. These proteins need to be diverse to be able to recognize a wide variety of substances. The genes from which these proteins are made contain segments known as variable (V), diversity (D), and joining (J) segments. During protein production within lymphocytes, these gene segments are rearranged in different combinations to increase variability of the resulting proteins. The RAG complex is involved in this process, which is known as V(D)J recombination.

During V(D)J recombination, the RAG complex attaches (binds) to a section of DNA called a recombination signal sequence (RSS), which is next to a V, D, or J segment. The RAG complex makes small cuts in the DNA between the segment and the RSS so the segment can be separated and moved to a different area in the gene. This process of DNA rearrangement within B cells and T cells is repeated multiple times in different areas so that the V, D, and J segments are arranged in various combinations. The variety of proteins produced throughout life following V(D)J recombination provides greater recognition of foreign invaders and allows the body to fight infection efficiently.

A defective RAG complex results in severe combined immunodeficiency (SCID) due to the complete absence of mature T and B cells (T-B-NK+ phenotype). Patients are born without a functional adaptive immune system and are therefore extremely susceptible to life-threatening infections. The estimated incidence of RAG1 SCID is ˜1:1,000,000 live births (˜15-20% of all cases of SCID).

SCID is commonly known as “bubble baby disease” because early interventions involved isolating newborn patient in a sterile environment to avoid exposure to pathogens. Newborn patients with SCID usually develop a fatal infection within the first year of life. A common standard of care is an allogeneic hematopoietic stem cell transplantation from an HLA matched donor. However, this is not a viable option for many patients due to the unavailability of a suitable matched donor and risks of immunological complications.

An alternative curative therapy is an autologous hematopoietic stem cell transplantation with ex vivo gene therapy. Preclinical attempts at developing a RAG1 lentiviral vector include EFS-coRAG1-WPRE which failed to rescue block in T-cell development in the thymus, SFFV-RAG1-WPRE which fails to rescue block in T-cell development in the thymus, and UCOE-coRAG1-WPRE which provided partial reconstitution of T and B cells, but led to the development of autoreactive T-cells and thus to the progression of Omenn-like symptoms (severe autoimmunity) (see, e.g., Pike-Overzet et al. (2011) Leukemia, 25: 1471-1483; van Til et al. (2014) J. Allergy Clin. Immunol. 133(4): 1116-1123).

### SUMMARY

Described herein is the development of novel lentiviral vector(s) (LVs) for the treatment of Recombination-Activating Gene 1 Severe Combined Immunodeficiency (RAG1 SCID). In particular lentiviral vectors described herein are regulated by endogenous elements of the RAG1 locus (or effective fragments of such elements) for high-level, lineage and temporal specific expression.

Accordingly, various embodiments contemplated herein may include, but need not be limited to, one or more of the following:

Embodiment 1: A recombinant lentiviral vector (LV) for the treatment of Recombination-Activating Gene 1 (RAG1) Severe Combined Immunodeficiency, said vector comprising:
an expression cassette comprising:
   a nucleic acid encoding an effective fragment of the endogenous promoter of the RAG1 gene; and
   a nucleic acid that encodes the RAG1 protein operably linked to said effective fragment of the endogenous promoter of the RAG1 gene.

Embodiment 2: The vector of embodiment 1, wherein the sequence of said effective fragment of the endogenous promoter of the RAG1 gene comprises or consists of the sequence of RAGpro (SEQ ID NO:2).

Embodiment 3: The vector of embodiment 2, wherein the sequence of said effective fragment of the endogenous promoter of the RAG1 gene consists of the sequence of RAGpro (SEQ ID NO:2).

Embodiment 4: The vector according to any one of embodiments 1-3, wherein said expression cassette comprises a RAG1 enhancer element 1 (SEQ ID NO: 1) or an effective fragment thereof.

Embodiment 5: The vector of embodiment 4, wherein the sequence of said RAG1 enhancer element consists the sequence of SEQ ID NO: 1 or an effective fragment thereof.

Embodiment 6: The vector according to any one of embodiments 1-5, wherein said expression cassette comprises a RAG1 enhancer element 3 (SEQ ID NO:3) or an effective fragment thereof.

Embodiment 7: The vector of embodiment 6, wherein the sequence of said RAG1 enhancer element 3 consists of the sequence of SEQ ID NO:3 or an effective fragment thereof.

Embodiment 8: The vector according to any one of embodiments 1-7, wherein said expression cassette comprises a RAG1 enhancer element 4 (SEQ ID NO:4) or an effective fragment thereof.

Embodiment 9: The vector of embodiment 8, wherein the sequence of said RAG1 enhancer element 4 consists of the sequence of SEQ ID NO:4 or an effective fragment thereof.

Embodiment 10: The vector according to any one of embodiments 1-9, wherein said expression cassette comprises a RAG1 enhancer element 5 (SEQ ID NO:5) or an effective fragment thereof.

Embodiment 11: The vector of embodiment 10, wherein the sequence of said RAG1 enhancer element 5 consists of the sequence of SEQ ID NO:5 or an effective fragment thereof.

Embodiment 12: The vector of embodiment 10, wherein the sequence of said RAG1 enhancer element 5 consists of the sequence of SEQ ID NO:31 or an effective fragment thereof.

Embodiment 13: The vector according to any one of embodiments 1-12, wherein said expression cassette comprises a RAG1 enhancer element 6 (SEQ ID NO:6) or an effective fragment thereof.

Embodiment 14: The vector of embodiment 13, wherein the sequence of said RAG1 enhancer element 6 consists of the sequence of SEQ ID NO:6 or an effective fragment thereof.

Embodiment 15: The vector according to any one of embodiments 1-14, wherein said expression cassette comprises a RAG1 enhancer element 7 (SEQ ID NO:7) or an effective fragment thereof.

Embodiment 16: The vector of embodiment 15, wherein the sequence of said RAG1 enhancer element 7 consists of the sequence of SEQ ID NO:7 or an effective fragment thereof.

Embodiment 17: The vector according to any one of embodiments 1-16, wherein said expression cassette comprises a RAG1 enhancer element 8 (SEQ ID NO:8) or an effective fragment thereof.

Embodiment 18: The vector of embodiment 17, wherein the sequence of said RAG1 enhancer element 8 consists of the sequence of SEQ ID NO:8 or an effective fragment thereof.

Embodiment 19: The vector according to any one of embodiments 1-18, wherein said expression cassette comprises a RAG1 enhancer element 9.1 (SEQ ID NO:9) or an effective fragment thereof.

Embodiment 20: The vector of embodiment 19, wherein the sequence of said RAG1 enhancer element 9.1 consists of the sequence of SEQ ID NO:9 or an effective fragment thereof.

Embodiment 21: The vector of embodiment 19, wherein the sequence of said RAG1 enhancer element 9.1 consists of enhancer element 9.1 core sequence of SEQ ID NO:34 or an effective fragment thereof.

Embodiment 22: The vector according to any one of embodiments 1-21, wherein said expression cassette comprises a RAG1 enhancer element 9.2 (SEQ ID NO:10) or an effective fragment thereof.

Embodiment 23: The vector of embodiment 22, wherein the sequence of said RAG1 enhancer element 9.2 consists of the sequence of SEQ ID NO:10 or an effective fragment thereof.

Embodiment 24: The vector according to any one of embodiments 1-23, wherein said expression cassette comprises a RAG1 enhancer element 10 (SEQ ID NO:11) or an effective fragment thereof.

Embodiment 25: The vector of embodiment 24, wherein the sequence of said RAG1 enhancer element 10 consists of the sequence of SEQ ID NO:11 or an effective fragment thereof.

Embodiment 26: The vector according to any one of embodiments 1-25, wherein said expression cassette comprises a RAG1 enhancer element 11 extra (SEQ ID NO:33) or an effective fragment thereof.

Embodiment 27: The vector according to any one of embodiments 1-25, wherein said expression cassette comprises a RAG1 enhancer element 11 (SEQ ID NO:12) or an effective fragment thereof.

Embodiment 28: The vector of embodiment 27, wherein the sequence of said RAG1 enhancer element 11 consists of the sequence of SEQ ID NO:12 or an effective fragment thereof.

Embodiment 29: The vector according to any one of embodiments 1-28, wherein said expression cassette comprises a RAG1 enhancer element 12 (SEQ ID NO:13) or an effective fragment thereof.

Embodiment 30: The vector of embodiment 29, wherein the sequence of said RAG1 enhancer element 12 consists of the sequence of SEQ ID NO:13 or an effective fragment thereof.

Embodiment 31: The vector according to any one of embodiments 1-30, wherein said expression cassette comprises a RAG1 enhancer element 13 (SEQ ID NO:14) or an effective fragment thereof.

Embodiment 32: The vector of embodiment 31, wherein the sequence of said RAG1 enhancer element 13 consists of the sequence of SEQ ID NO:14 or an effective fragment thereof.

Embodiment 33: The vector of embodiment 31, wherein the sequence of said RAG1 enhancer element 13 consists of the sequence of SEQ ID NO:36 or an effective fragment thereof.

Embodiment 34: The vector according to any one of embodiments 1-33, wherein said expression cassette comprises a RAG1 enhancer element 14 (SEQ ID NO:15) or an effective fragment thereof.

Embodiment 35: The vector of embodiment 34, wherein said expression cassette comprises or consists of an enlarged RAG1 enhancer element 14 core (SEQ ID NO:32) or an effective fragment thereof.

Embodiment 36: The vector of embodiment 34, wherein the sequence of said RAG1 enhancer element 14 consists of the sequence of SEQ ID NO:15 or an effective fragment thereof.

Embodiment 37: The vector according to any one of embodiments 1-36, wherein said expression cassette comprises a RAG1 enhancer element 16 (SEQ ID NO:16) or an effective fragment thereof.

Embodiment 38: The vector of embodiment 37, wherein the sequence of said RAG1 enhancer element 16 consists of the sequence of SEQ ID NO:16 or an effective fragment thereof.

Embodiment 39: The vector according to any one of embodiments 1-38, wherein said expression cassette comprises a RAG1 enhancer element 17 (SEQ ID NO:17) or an effective fragment thereof.

Embodiment 40: The vector of embodiment 39, wherein the sequence of said RAG1 enhancer element 17 consists of the sequence of SEQ ID NO:17 or an effective fragment thereof.

Embodiment 41: The vector according to any one of embodiments 1-40, wherein said expression cassette comprises a RAG1 enhancer element 18 (SEQ ID NO:18) or an effective fragment thereof.

Embodiment 42: The vector of embodiment 41, wherein the sequence of said RAG1 enhancer element 18 consists of the sequence of SEQ ID NO:18 or an effective fragment thereof.

Embodiment 43: The vector of embodiment 41, wherein the sequence of said RAG1 enhancer element 18 consists of the sequence of SEQ ID NO:35 or an effective fragment thereof.

Embodiment 44: The vector according to any one of embodiments 1-43, wherein said nucleic acid that encodes a RAG1 Protein is a RAG1 cDNA or a codon-optimized RAG1 gene or cDNA.

Embodiment 45: The vector of embodiment 44, wherein said nucleic acid that encodes a nucleic acid that encodes RAG1 protein is a RAG1 cDNA (SEQ ID NO:19).

Embodiment 46: The vector of embodiment 44, wherein said nucleic acid that encodes a nucleic acid that encodes RAG1 protein is a codon optimized RAG1.

Embodiment 47: The vector of embodiment 46, wherein the sequence of said nucleic acid that encodes RAG1 protein is a codon optimized RAG1 selected from the group consisting of jCAT codon optimized RAG1, GeneArt optimized RAG1, and IDT optimized RAG1.

Embodiment 48: The vector according to any one of embodiments 1-47, wherein said vector comprises a ψ region vector genome packaging signal.

Embodiment 49: The vector according to any one of embodiments 1-48, wherein said vector comprise a 5′ LTR comprising a CMV enhancer/promoter.

Embodiment 50: The vector according to any one of embodiments 1-49, wherein said vector comprises a Rev Responsive Element (RRE).

Embodiment 51: The vector according to any one of embodiments 1-50, wherein said vector comprises a central polypurine tract.

Embodiment 52: The vector according to any one of embodiments 1-51, wherein said vector comprises a post-translational regulatory element.

Embodiment 53: The vector of embodiment 52, wherein the posttranscriptional regulatory element is modified Woodchuck Post-transcriptional Regulatory Element (WPRE).

Embodiment 54: The vector according to any one of embodiments 1-53, wherein said vector is incapable of reconstituting a wild-type lentivirus through recombination.

Embodiment 55: The vector according to any one of embodiments 1-54, wherein said vector shows high expression in T-cells.

Embodiment 56: The vector according to any one of embodiments 1-55, wherein said vector shows high expression in B-cells.

Embodiment 57: The vector of embodiment 1, wherein said vector comprises the vector elements selected from the group consisting of: 1) E5 Core, E14 Core, E9.1 Core, E12 OG—RAG1pro-RAG-WPRE; 2) E5 Core, E14 Core, E9.1 Core, E12 OG, E11 extra B-cell enhancer—RAG1pro-RAG-WPRE; 3) E5 Core, E14 Core, E9.1 OG large, E12 OG—RAG1pro-RAG-WPRE; 4) E5 Core, E14 Core, E9.1 OG large, E12 OG, E11 extra B-cell enhancer—RAG1pro-RAG-WPRE; 5) E5 Core, E14 Core, E9.1 Core, E12 OG+18 right, 11 whole, 13 right—RAG1pro-RAG-WPRE; 6) E5 Core, E14 Core, E9.1 Core, E12 OG+18 whole, 11 whole—RAG1pro-RAG-WPRE; 7) E5 Core, E14 Core, E9.1 Core, E12 OG+18 whole—RAG1pro-RAG-WPRE; 8) E5 Core, E14 Core, E9.1 Core, E12 OG, E11 extra B-cell enhancer+18 right, 11 whole, 13 right—RAG1pro-RAG-WPRE; 9) E5 Core, E14 Core, E9.1 Core, E12 OG, E11 extra B-cell enhancer+18 whole, 11 whole—RAG1pro-RAG-WPRE; and 10) E5 Core, E14 Core, E9.1 Core, E12 OG, E11 extra B-cell enhancer+18 whole—RAG1pro-RAG-WPRE.

Embodiment 58: The vector of embodiment 57, wherein said vector comprises the vector elements E5 Core, E14 Core, E9.1 Core, E12 OG—RAG1pro-RAG-WPRE;

Embodiment 59: The vector of embodiment 58, wherein said vector comprises the nucleotide sequence of SEQ ID NO:37.

Embodiment 60: The vector of embodiment 57, wherein said vector comprises the vector elements E5 Core, E14 Core, E9.1 Core, E12 OG, E11 extra B-cell enhancer—RAG1pro-RAG-WPRE.

Embodiment 61: The vector of embodiment 58, wherein said vector comprises the nucleotide sequence of SEQ ID NO:38.

Embodiment 62: The vector of embodiment 57, wherein said vector comprises the vector elements E5 Core, E14 Core, E9.1 OG large, E12 OG—RAG1pro-RAG-WPRE.

Embodiment 63: The vector of embodiment 58, wherein said vector comprises the nucleotide sequence of SEQ ID NO:39.

Embodiment 64: The vector of embodiment 57, wherein said vector comprises the vector elements E5 Core, E14 Core, E9.1 OG large, E12 OG, E11 extra B-cell enhancer—RAG1pro-RAG-WPRE.

Embodiment 65: The vector of embodiment 58, wherein said vector comprises the nucleotide sequence of SEQ ID NO:40.

Embodiment 66: The vector of embodiment 57, wherein said vector comprises the vector elements E5 Core, E14 Core, E9.1 Core, E12 OG+18 right, 11 whole, 13 right—RAG1pro-RAG-WPRE.

Embodiment 67: The vector of embodiment 58, wherein said vector comprises the nucleotide sequence of SEQ ID NO:41.

Embodiment 68: The vector of embodiment 57, wherein said vector comprises the vector elements E5 Core, E14 Core, E9.1 Core, E12 OG+18 whole, 11 whole—RAG1pro-RAG-WPRE.

Embodiment 69: The vector of embodiment 58, wherein said vector comprises the nucleotide sequence of SEQ ID NO:42.

Embodiment 70: The vector of embodiment 57, wherein said vector comprises the vector elements E5 Core, E14 Core, E9.1 Core, E12 OG+18 whole—RAG1pro-RAG-WPRE.

Embodiment 71: The vector of embodiment 58, wherein said vector comprises the nucleotide sequence of SEQ ID NO:43.

Embodiment 72: The vector of embodiment 57, wherein said vector comprises the vector elements E5 Core, E14 Core, E9.1 Core, E12 OG, E11 extra B-cell enhancer+18 right, 11 whole, 13 right—RAG1pro-RAG-WPRE.

Embodiment 73: The vector of embodiment 58, wherein said vector comprises the nucleotide sequence of SEQ ID NO:44.

Embodiment 74: The vector of embodiment 57, wherein said vector comprises the vector elements E5 Core, E14 Core, E9.1 Core, E12 OG, E11 extra B-cell enhancer+18 whole, 11 whole—RAG1pro-RAG-WPRE.

Embodiment 75: The vector of embodiment 58, wherein said vector comprises the nucleotide sequence of SEQ ID NO:45.

Embodiment 76: The vector of embodiment 57, wherein said vector comprises the vector elements E5 Core, E14 Core, E9.1 Core, E12 OG, E11 extra B-cell enhancer+18 whole—RAG1pro-RAG-WPRE.

Embodiment 77: The vector of embodiment 58, wherein said vector comprises the nucleotide sequence of SEQ ID NO:46.

Embodiment 78: A host cell transduced with a vector according to any one of embodiments 1-77.

Embodiment 79: The host cell of embodiment 78, wherein the cell is a stem cell.

Embodiment 80: The host cell of embodiment 79, wherein said cell is a stem cell derived from bone marrow, and/or from umbilical cord blood, and/or from peripheral blood.

Embodiment 81: The host cell of embodiment 78, wherein the cell is a human hematopoietic progenitor cell.

Embodiment 82: The host cell of embodiment 81, wherein the human hematopoietic progenitor cell is a CD34+ cell.

Embodiment 83: A method of treating Recombination-Activating Gene 1 (RAG1) Severe Combined Immunodeficiency (RAG1 SCID), in a subject, said method comprising:
transducing a stem cell and/or progenitor cell from said subject with a vector according to any one of embodiments 1-77; and
transplanting said transduced cell or cells derived therefrom into said subject where said cells or derivatives therefrom express said RAG1 protein.

Embodiment 84: The method of embodiment 83, wherein the cell is a stem cell.

Embodiment 85: The method of embodiment 83, wherein said cell is a stem cell derived from bone marrow.

Embodiment 86: The method of embodiment 83, wherein the cell is a human hematopoietic stem and progenitor cell.

Embodiment 87: The method of embodiment 86, wherein the human hematopoietic progenitor cell is a CD34⁺ cell.

Embodiment 88: A recombinant nucleic acid comprising one or more of the following: an effective fragment of the endogenous promoter of the RAG1 gene that comprises or consists of the sequence of RAGpro (SEQ ID NO:2); and/or
a RAG1 enhancer element 1 (SEQ ID NO: 1) or an effective fragment thereof; and/or
a RAG1 enhancer element 3 (SEQ ID NO:3) or an effective fragment thereof; and/or
a RAG1 enhancer element 4 (SEQ ID NO:4) or an effective fragment thereof; and/or
an enlarged RAG1 enhancer element 5 core (SEQ ID NO:31) or an effective fragment thereof a RAG1 enhancer element 5 (SEQ ID NO:5) or an effective fragment thereof; and/or
a RAG1 enhancer element 6 (SEQ ID NO:6) or an effective fragment thereof a RAG1 enhancer element 7 (SEQ ID NO:78) or an effective fragment thereof; and/or
a RAG1 enhancer element 8 (SEQ ID NO:8) or an effective fragment thereof; and/or
a RAG1 enhancer element 9.1 (SEQ ID NO:9) or an effective fragment thereof; and/or a RAG1 enhancer element 9.1 core sequence (SEQ ID NO:34) or an effective fragment thereof; and/or
a RAG1 enhancer element 9.2 (SEQ ID NO:10) or an effective fragment thereof; and/or a RAG1 enhancer element 10 (SEQ ID NO:11) or an effective fragment thereof; and/or
a RAG1 enhancer element 11 extra (SEQ ID NO:33) or an effective fragment thereof; and/or
a RAG1 enhancer element 11 (SEQ ID NO:12) or an effective fragment thereof; and/or
a RAG1 enhancer element 12 (SEQ ID NO:13) or an effective fragment thereof; and/or
a RAG1 enhancer element 13 (SEQ ID NO:14) or an effective fragment thereof; and/or
an enlarged RAG1 enhancer element 14 core (SEQ ID NO:32) or an effective fragment thereof; and/or
a RAG1 enhancer element 14 (SEQ ID NO:15) or an effective fragment thereof; and/or
a RAG1 enhancer element 16 (SEQ ID NO:16) or an effective fragment thereof; and/or
a RAG1 enhancer element 17 (SEQ ID NO:17) or an effective fragment thereof; and/or
a RAG1 enhancer element 18 (SEQ ID NO:18) or an effective fragment thereof.

Embodiment 89: The nucleic acid of embodiment 88, wherein said nucleic acid comprises a sequence consisting of an effective fragment of the endogenous promoter of the RAG1 gene (SEQ ID NO:2).

Embodiment 90: The nucleic acid according to any one of embodiments 88-89, wherein said nucleic acid comprises a sequence consisting of a RAG1 enhancer element 1 (SEQ ID NO: 1) or an effective fragment thereof.

Embodiment 91: The nucleic acid according to any one of embodiments 88-90, wherein said nucleic acid comprises a sequence consisting of a RAG1 enhancer element 3 (SEQ ID NO:3) or an effective fragment thereof.

Embodiment 92: The nucleic acid according to any one of embodiments 88-91, wherein said nucleic acid comprises a sequence consisting of a RAG1 enhancer element 4 (SEQ ID NO:4) or an effective fragment thereof.

Embodiment 93: The nucleic acid according to any one of embodiments 88-92, wherein said nucleic acid comprises a an enlarged RAG1 enhancer element 5 core (SEQ ID NO:31) or an effective fragment thereof.

Embodiment 94: The nucleic acid according to any one of embodiments 88-92, wherein said nucleic acid comprises a sequence consisting of a RAG1 enhancer element 5 (SEQ ID NO:5) or an effective fragment thereof.

Embodiment 95: The nucleic acid according to any one of embodiments 88-94, wherein said nucleic acid comprises a sequence consisting of a RAG1 enhancer element 6 (SEQ ID NO:6) or an effective fragment thereof.

Embodiment 96: The nucleic acid according to any one of embodiments 88-95, wherein said nucleic acid comprises a sequence consisting of a RAG1 enhancer element 7 (SEQ ID NO:78) or an effective fragment thereof.

Embodiment 97: The nucleic acid according to any one of embodiments 88-96, wherein said nucleic acid comprises a sequence consisting of a RAG1 enhancer element 8 (SEQ ID NO:8) or an effective fragment thereof.

Embodiment 98: The nucleic acid according to any one of embodiments 88-97, wherein said nucleic acid comprises a sequence consisting of a RAG1 enhancer element 9.1 (SEQ ID NO:9) or an effective fragment thereof.

Embodiment 99: The nucleic acid according to any one of embodiments 88-98, wherein said nucleic acid comprises a sequence consisting of a RAG1 enhancer element 9.2 (SEQ ID NO:10) or an effective fragment thereof.

Embodiment 100: The nucleic acid according to any one of embodiments 88-99, wherein said nucleic acid comprises a sequence consisting of a RAG1 enhancer element 10 (SEQ ID NO:11) or an effective fragment thereof.

Embodiment 101: The nucleic acid according to any one of embodiments 88-100, wherein said nucleic acid comprises a sequence consisting of a RAG1 enhancer element 11 (SEQ ID NO:12) or an effective fragment thereof.

Embodiment 102: The nucleic acid according to any one of embodiments 88-101, wherein said nucleic acid comprises a sequence consisting of a RAG1 enhancer element 12 (SEQ ID NO:13) or an effective fragment thereof.

Embodiment 103: The nucleic acid according to any one of embodiments 88-102, wherein said nucleic acid comprises a sequence consisting of a RAG1 enhancer element 13 (SEQ ID NO:14) or an effective fragment thereof.

Embodiment 104: The nucleic acid according to any one of embodiments 88-103, wherein said nucleic acid comprises a sequence consisting of an enlarged RAG1 enhancer element 14 core (SEQ ID NO:32) or an effective fragment thereof.

Embodiment 105: The nucleic acid according to any one of embodiments 88-103, wherein said nucleic acid comprises a sequence consisting of a RAG1 enhancer element 14 (SEQ ID NO:15) or an effective fragment thereof.

Embodiment 106: The nucleic acid according to any one of embodiments 88-105, wherein said nucleic acid comprises a sequence consisting of a RAG1 enhancer element 16 (SEQ ID NO:16) or an effective fragment thereof.

Embodiment 107: The nucleic acid according to any one of embodiments 88-106, wherein said nucleic acid comprises a sequence consisting of a RAG1 enhancer element 17 (SEQ ID NO:17) or an effective fragment thereof.

Embodiment 108: The nucleic acid according to any one of embodiments 88-107, wherein said nucleic acid comprises a sequence consisting of a RAG1 enhancer element 18 (SEQ ID NO:18) or an effective fragment thereof.

Embodiment 109: The nucleic acid according to any one of embodiments 88-108, wherein said nucleic acid comprises an expression cassette.

Embodiment 110: The nucleic acid of embodiment 109, wherein said expression cassette comprises a nucleic acid that encodes a RAG1 protein.

Embodiment 111: The nucleic acid of embodiment 110, wherein said nucleic acid that encodes a RAG1 protein comprises a RAG1 cDNA.

Embodiment 112: The nucleic acid of embodiment 110, wherein said nucleic acid that encodes a RAG1 protein comprise a codon-optimized RAG1 cDNA.

Embodiment 113: The nucleic acid according to any one of embodiments 110-112, wherein said nucleic acid that encodes a RAG1 protein is operably linked to an endogenous RAG1 promoter or an effective fragment thereof.

Embodiment 114: The nucleic acid according to any one of embodiments 110-113, wherein said nucleic acid comprises a lentiviral vector according to any one of embodiments 1-77.

Embodiment 115: The nucleic acid according to any one of embodiments 88-114, wherein said nucleic acid does not comprise the endogenous RAG1 gene.

### Definitions

A “promoter” refers to a regulatory sequence in a nucleic acid required to initiate transcription of a gene (e.g., a gene operably coupled to the promoter).

An “enhancer” refers to a regulatory DNA sequence that, when bound by specific proteins called transcription factors, enhance the transcription of an associated gene.

An “effective fragment” when used with respect to a promoter (e.g., an effective fragment of a RAG1 promoter) refers to a fragment of the full-length promoter that is sufficient to initiate transcription of a gene operably linked to that promoter (e.g., RAG1).

An “effective fragment” when used with respect to an enhancer (e.g., an effective fragment of a RAG1 enhancer) refers to a fragment of the full-length enhancer that is sufficient to provide regulate expression of an operably linked gene when bound by a transcription factor. In certain embodiments the regulation is comparable with respect to expression level and/or lineage offered by the full-length enhancer.

The term “operably linked” refers to a nucleic acid sequence placed into a functional relationship with another nucleic acid sequence. For example, a promoter is operably linked to a gene when that promoter is placed in a location that permits that promoter to initiate transcription of that gene. An enhancer is operably linked to a gene when that enhancer, when bound by an appropriate transcription factor, is able to regulate (e.g., to upregulate) expression of that gene.

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid.

As used herein, the term “recombinant lentiviral vector” or “recombinant LV) refers to an artificially created polynucleotide vector assembled from an LV and a plurality of additional segments as a result of human intervention and manipulation.

By “an effective amount” is meant the amount of a required agent or composition comprising the agent to ameliorate or eliminate symptoms of a disease relative to an untreated patient. The effective amount of composition(s) used to practice the methods described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

### BRIEF DESCRIPTION OF THE DRAWINGS

FIG. 1 illustrates vectors used to evaluate endogenous RAG1 enhancer elements.

FIG. 2 illustrates vectors containing bar codes (BC) used to evaluate endogenous RAG1 enhancer elements.

FIG. 3 shows expression levels in NALM6 cells (pre-B cell line) transduced with non-barcoded RAG1 vectors.

FIG. 4 shows expression levels in NALM6 cells (pre-B cell line) transduced with barcoded RAG1 vectors.

FIG. 5 illustrates the setup of the singleplex transduced barcoded vectors in NALM6 cells.

### DETAILED DESCRIPTION

In various embodiments, lentiviral vectors are provided for the treatment (or prophylaxis) of Recombination-Activating Gene 1 severe combined immunodeficiency (RAG1 SCID) are provided. In certain embodiments the vectors are optimized to reduce vector size, increase expression level and titer. Additionally, in various embodiments the vectors recapitulate the expression pattern of the native RAG1 gene, e.g., as described herein.

In particular, a bioinformatic analysis (using publicly available databases: Project Encode, Ensembl, FANTOM, VISTA Enhancer Browser, GeneHancer) was utilized to elucidate the endogenous regulatory elements of the native RAG1 gene. Eighteen putative enhancer elements were identified located within a 400,000 base pair window. It is believed that a subset of the 18 putative enhancer elements are each responsible for RAG1 expression at specific timepoints throughout T and B cell development and that various combinations of these elements can be incorporated into an expression cassette in, for example, a lentiviral vector, to provide vectors that can recapitulate the expression pattern of the native RAG1 gene at therapeutically effective levels.

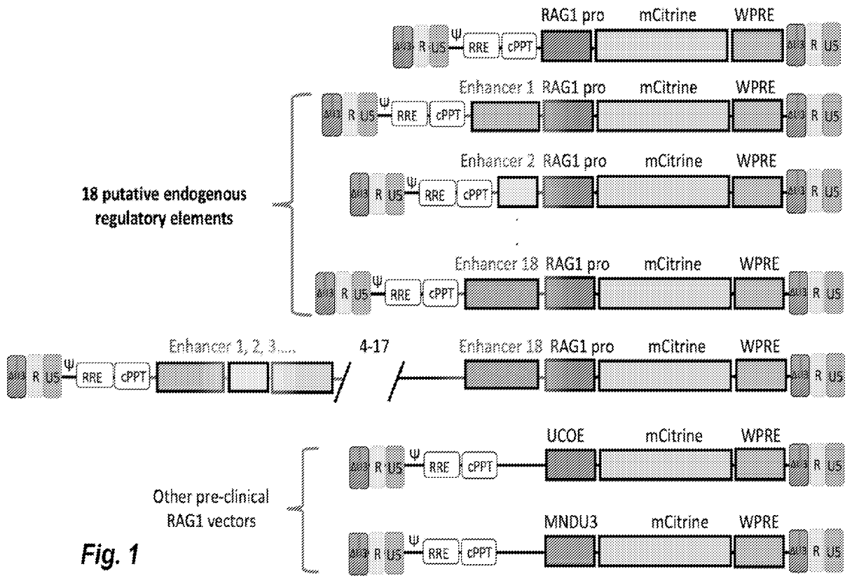

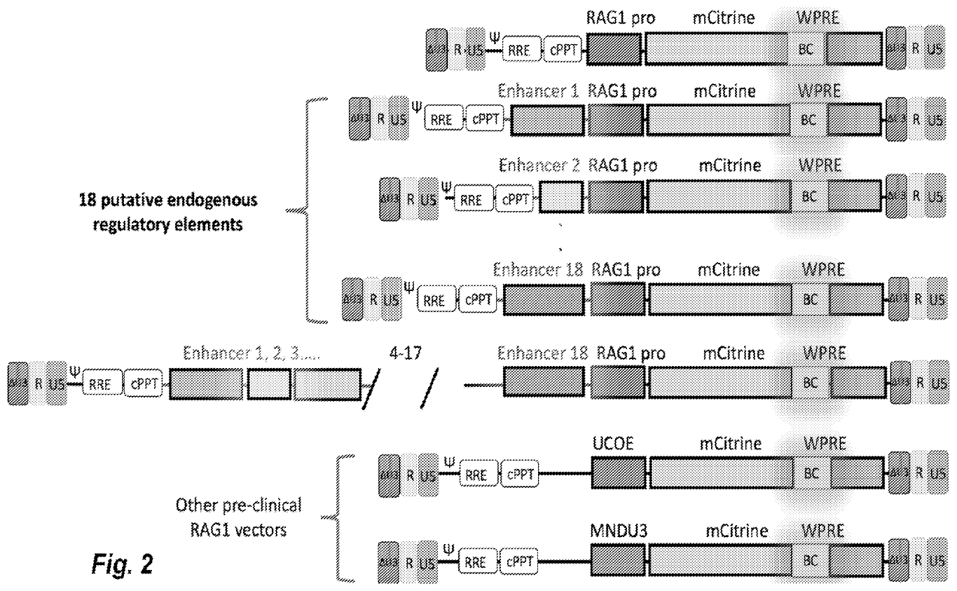

In order to identify the critical enhancer elements that regulate the RAG1 gene, each putative enhancer element was cloned upstream of the endogenous RAG1 promoter to drive expression of a reporter (mCitrine) (see, e.g., FIG. 1). A vector was also cloned containing all 18 elements (see, e.g., FIG. 1). Other pre-clinical RAG1 vectors were used as controls. A duplicate set of the vectors were re-cloned to contain a unique 15 nucleotide barcode in the 3′ untranslated region (3′UTR) to allow for multiplexing (see, e.g., FIG. 2). Each vector has been cloned to contain 2 unique barcodes for a total of 42 new vectors.

In various embodiments an expression cassette comprising various combinations of the identified regulatory elements (e.g., enhancers) and/or vectors comprising such expression cassettes are provided. Thus, in certain embodiments, a recombinant lentiviral vector (LV) for the treatment of Recombination-Activating Gene 1 (RAG1) Severe Combined Immunodeficiency is provided where the vector comprises an expression cassette comprising: 1) a nucleic acid encoding an effective fragment of the endogenous promoter of the RAG1 gene; and 2) a nucleic acid that encodes the RAG1 protein operably linked to the effective fragment of the endogenous promoter of the RAG1 gene.

In certain embodiments the expression cassette comprises an endogenous RAG1 promoter or an effective fragment thereof, and/or RAG1 Enhancer 1 or an effective fragment thereof, and/or RAG1 enhancer 2 or an effective fragment thereof, and/or RAG1 enhancer 3 or an effective fragment thereof, and/or RAG1 enhancer 4 or an effective fragment thereof, and/or RAG1 enhancer 5 or an effective fragment thereof, and/or RAG1 enhancer 6 or an effective fragment thereof, and/or RAG1 enhancer 7 or an effective fragment thereof, and/or RAG1 enhancer 8 or an effective fragment thereof, and/or RAG1 enhancer 9.1 or an effective fragment thereof, and/or RAG1 enhancer 9.2 or an effective fragment thereof, and/or RAG1 enhancer 10 or an effective fragment thereof, and/or RAG1 enhancer 11 or an effective fragment thereof, and/or RAG1 enhancer 12 or an effective fragment thereof, and/or RAG1 enhancer 13 or an effective fragment thereof, and/or RAG1 enhancer 14 or an effective fragment thereof, and/or RAG1 enhancer 16 or an effective fragment thereof, and/or RAG1 enhancer 17 or an effective fragment thereof, and/or RAG1 enhancer 18 or an effective fragment thereof (see, e.g., Table 1).

In certain embodiments the sequence of the effective fragment of the endogenous promoter of the RAG1 gene comprises or consists of the sequence of RAGpro (SEQ ID NO:2). In certain embodiments the sequence of the effective fragment of the endogenous promoter of the RAG1 gene consists of the sequence of RAGpro (SEQ ID NO:2).

In certain embodiments the expression cassette comprises a RAG1 enhancer element 1 (SEQ ID NO: 1) or an effective fragment thereof. In certain embodiments the RAG1 enhancer element consists the sequence of SEQ ID NO: 1 or an effective fragment thereof.

In certain embodiments the expression cassette comprises a RAG1 enhancer element 3 (SEQ ID NO:3) or an effective fragment thereof. In certain embodiments the sequence of the RAG1 enhancer element 3 consists of the sequence of SEQ ID NO:3 or an effective fragment thereof.

In certain embodiments the expression cassette comprises a RAG1 enhancer element 4 (SEQ ID NO:4) or an effective fragment thereof. In certain embodiments the sequence of the RAG1 enhancer element 4 consists of the sequence of SEQ ID NO:4 or an effective fragment thereof.

In certain embodiments the expression cassette comprises a RAG1 enhancer element 5 (SEQ ID NO:5) or an effective fragment thereof. In certain embodiments the sequence of the RAG1 enhancer element 5 consists of the sequence of SEQ ID NO:5 or an effective fragment thereof.

In certain embodiments the expression cassette comprises a RAG1 enhancer element 6 (SEQ ID NO:6) or an effective fragment thereof. In certain embodiments the sequence of the RAG1 enhancer element 6 consists of the sequence of SEQ ID NO:6 or an effective fragment thereof.

In certain embodiments the expression cassette comprises a RAG1 enhancer element 7 (SEQ ID NO:7) or an effective fragment thereof. In certain embodiments the sequence of the RAG1 enhancer element 7 consists of the sequence of SEQ ID NO:7 or an effective fragment thereof.

In certain embodiments the expression cassette comprises a RAG1 enhancer element 8 (SEQ ID NO: 8) or an effective fragment thereof. In certain embodiments the sequence of the RAG1 enhancer element 8 consists of the sequence of SEQ ID NO:8 or an effective fragment thereof.

In certain embodiments the expression cassette comprises a RAG1 enhancer element 9.1 (SEQ ID NO:9) or an effective fragment thereof. In certain embodiments the sequence of the RAG1 enhancer element 9.1 consists of the sequence of SEQ ID NO:9 or an effective fragment thereof.

In certain embodiments the expression cassette comprises a RAG1 enhancer element 9.2 (SEQ ID NO:10) or an effective fragment thereof. In certain embodiments the sequence of the RAG1 enhancer element 9.2 consists of the sequence of SEQ ID NO:10 or an effective fragment thereof.

In certain embodiments the expression cassette comprises a RAG1 enhancer element 10 (SEQ ID NO:11) or an effective fragment thereof. In certain embodiments the sequence of the RAG1 enhancer element 10 consists of the sequence of SEQ ID NO:11 or an effective fragment thereof.

In certain embodiments the expression cassette comprises a RAG1 enhancer element 11 (SEQ ID NO:12) or an effective fragment thereof. In certain embodiments the sequence of the RAG1 enhancer element 11 consists of the sequence of SEQ ID NO:12 or an effective fragment thereof.

In certain embodiments the expression cassette comprises a RAG1 enhancer element 12 (SEQ ID NO:13) or an effective fragment thereof. In certain embodiments the sequence of the RAG1 enhancer element 12 consists of the sequence of SEQ ID NO:13 or an effective fragment thereof.

In certain embodiments the expression cassette comprises a RAG1 enhancer element 13 (SEQ ID NO:14) or an effective fragment thereof. In certain embodiments the sequence of the RAG1 enhancer element 13 consists of the sequence of SEQ ID NO:14 or an effective fragment thereof.

In certain embodiments the expression cassette comprises a RAG1 enhancer element 14 (SEQ ID NO:15) or an effective fragment thereof. In certain embodiments the sequence of the RAG1 enhancer element 14 consists of the sequence of SEQ ID NO:15 or an effective fragment thereof.

In certain embodiments the expression cassette comprises a RAG1 enhancer element 16 (SEQ ID NO:16) or an effective fragment thereof. In certain embodiments the sequence of the RAG1 enhancer element 16 consists of the sequence of SEQ ID NO:16 or an effective fragment thereof.

In certain embodiments the expression cassette comprises a RAG1 enhancer element 17 (SEQ ID NO:17) or an effective fragment thereof. In certain embodiments the sequence of the RAG1 enhancer element 17 consists of the sequence of SEQ ID NO:17 or an effective fragment thereof.

In certain embodiments the expression cassette comprises a RAG1 enhancer element 18 (SEQ ID NO:18) or an effective fragment thereof. In certain embodiments the sequence of the RAG1 enhancer element 18 consists of the sequence of SEQ ID NO:18 or an effective fragment thereof.

In certain embodiments the nucleic acid that encodes RAG1 Protein is a RAG1 cDNA or a codon-optimized RAG1 gene or cDNA. In certain embodiments the nucleic acid that encodes RAG1 protein is a RAG1 cDNA (SEQ ID NO:19 in Table 1). In certain embodiments the nucleic acid that encodes a RAG1 protein is a codon optimized RAG1. In certain embodiments the sequence of the nucleic acid that encodes RAG1 protein is a codon optimized RAG1 selected from the group consisting of jCAT codon optimized RAG1, GeneArt optimized RAG1, and IDT optimized RAG1.

It will also be recognized that the expression cassettes described herein with respect to lentiviral vectors need not be limited to this use, and can be incorporated in essentially any other construct (e.g., a CRISPR construct) where expression of a RAG1 protein is desired. Thus, in certain embodiments, nucleic acid constructs comprising any of the expression cassette components described herein are contemplated.

TABLE 1
Nucleic acid sequences of various components of the lentiviral vectors for treatment
of RAG1 SCID as described herein.

| Element/ | |
|---|---|
| SEQ ID NO | Sequence |
| RAG1 Enhancer 1 | GGAGCAGAGGTCAGGGGACAGGGAACGGTGTCCCCGACCT |
| (TRAF6pro) | GTGGGAAGCGTGGGGACAGAGGCTGCGTCCCGACCTGCAG |
| (SEQ ID NO: 1) | GAAGCGAGGGGCAGGGAACGGTGTCCTCGACCAGCGGGAA |
| | GCGAGAGGACAGGGGCGGCGTCCCCGACCCGTGGGAAGCA |
| | AGCGGGTAGGGGCGGCGTCCCTGACCTGTGAAAAGCGAAG |
| | GGACAGGGGAGGCGTCCCCGACTGGTGGGAAGCGAGGGGA |
| | CAGGGACTGCGTCCCCGACCTGCCGAGAGCGAAGGGGCGG |
| | GGGGCGGCGTCCCTGACCGCTGGGAAGCGAGGCGGCGGCC |
| | GCCAGGAGGAGGCGCCTGAAGGAGACTCACCGTTCTAGTG |
| | CGCGGGGAGGCCGAACCAGGAGGGCAGGGCTCCCCCACCA |
| | ACCGCACGACTCCGCTCAGCCAAGGCGCTGGTAGAGGACG |
| | GACACAGACACTGCGCGCCGAGACGAGGCTGCTTGGACGG |
| | CAAACTCTGGATCCAGTGGGAGCCTTCGCCACCTTCGCTGG |
| | CCGCCCGCAGGCCAAGCCCCAGCTGCGGACGCCACTGCTTC |
| | CGCCTTCTCTGCTGGCTGCGGGGAGCGAGGGGCAGGGAGC |
| | GCGCCGGGGAGGAGACAGAGCGGCCGAGTTCGGAGAGAGT |
| | GCCCCCTGGCGGTGATCCTCCAGGCGGACTGTGGCGCGGGC |
| | CGGGCGGCGCCCCCAGCCCCGCAGCCTGGCTTTCTTCCTGC |
| | TCGGGTGTCCCACCTGGGAATTCGTTGCACCGCACAGCCGA |
| | ACCCGAGTGTGTCAATGGGTGTGTGTTGGAATGAGGATGGC |
| | GACGAGAAGATCCCTCCCGGAATAACTTAATTTGGTGAGCA |
| | GTGGCGACAGTAGTAATCCCAAACTGCGCTTTTATAGCACC |
| | CCTCTCCAAGCAGGAGAAAACCCCAGCTGGGCTAGATCGCT |
| | TGACCTTCAGGAATTCTTAAACCCCTCCCCAGCTCCACCCTC |
| | CTTGCCCAGGAAGTTTCTGCGATCCATAGAACCCTCTATTAT |
| | TACCTGCTCTTCCCCTTCATCCGGTCCAAGGGAGTCAGCCCT |
| | TGCCTTAA |
| RAG Enhancer 2 | CTAAAGAGCCAGGTGGCAGCTGGAGCTGGGGTCTCCTGGCC |
| Endogenous RAG | CATGATTGGCTGCCATCATTTGTGGTTAGCCCTCCATGGTGG |
| promoter | GGGAGGCTGGGAAGGACAGTGGAAGCTGATAAACAGCTCA |
| (SEQ ID NO: 2) | GCAGCATGTTCTGAGAAACAAGAGGGCAAGGAGAGAGCAG |
| | AGAACACACTTTGCCTTCTCTTTGGTATTGAGTAATATCAAC |
| | CAAATTGCAGACATCTCAACACTTTGGCCAGGCAGCCTGCT |
| | GAGCAAGGTACCTCAGCCAGC |
| RAG Enhancer 3 | CTCAGCCTGAGTGATTATGATCAGAGAGTTGTCCCATTTTG |
| RAG2-Int1 | GGTGGGGGGAACACCAAAGCCAAGGCCTGCTTGTTTCAAAC |
| (SEQ ID NO: 3) | ACCTGTGATGACTGTTATTGAGATACAAGGCAAGCCATACA |
| | GTGGTAATGGATTCCAGATCCAAAGGG |
| RAG Enhancer 4 | TTGAGGTTATGTTCTTGAGTCAGGATTCCTGGCCAGGGATT |
| (RAGI-Int1) | GTGATGATATATTTCTCTTTCTGAAGTGGTTCATGCAAGAGG |
| (SEQ ID NO: 4) | TTGTCTGAAGGAAGAGCAAGAATTGTAGTGTTATTTTGTGG |
| | ATACTTGAGACTTATAAAAAGGCTTTTT |
| RAG Enhancer 5 | AGGGACCTAGAGGCAGATGTAGACAAATAGCAGGTGAGGA |
| (25 kb upstream of | GGAAGGGTGGTGCTGAGCTGTAAACAACAGCTGCGGGCTG |
| RAG1) | TAAACAATGGAGCTATATTAAGAAAGTCCTGCATCTGCAGC |
| (SEQ ID NO: 5) | ATTCGGTTCAATCCTGCCACCCACTGGGCGTACTCCTGAATT |
| | GACTTTCCATTGTCAGCTGATCAGTGACAGATGTGAAATGA |
| | CTCGTTACACACACAGGTCACATTGGGAGGATTCTTAGACG |
| | TCATA |
| RAG Enhancer 6 | GGCACCAAACTGTTTTCTACTTGACAGGTTGTTATCATGCTG |
| (Full P. Gfl1ib B1 | TATTTGCACCCACCATCACAGAGATTGTTTAAAAAAAAAGT |
| (SEQ ID NO: 6) | TATCTAGGGCATGCCAAATTATATGTAAAAGTATAGTACAG |
| | ATTGCAGTGGAAAGATAACGTGATGATTCTGTGATATCTGG |
| | AGAGGAAACATAACCAGTTGGAGATAGTGCCTTCATTTTTG |
| | TTTTAAAAACAAAAGAGAACTCACTTGCGAACTCTTTTTTTT |
| | TTTTTCTAAATTTCTTCAGGCCACCATAGCTGAAGCTCCCTT |
| | TACTTCTCACCTGGGTTACTATGGTAGCTCCACACTTTCTTG |
| | TCTCTAGTACCTGAACCACTTTGATCCATTCTATCCATTTGT |
| | CTTCTTAGAGCACAGCTCTAATTGTGACTCCCCAGGATGTA |
| | GGTCAAGTACAGAGTTTTTGTCGGGCCCTCCAAATATACCC |
| | CGTCCATGTGTCCCCAAATCTTCCTGGAATAATCGGCGATTC |
| | TTACTCAGGCCTTGTGTGTTCCCACCTTGAGGGCTTTGCTCA |
| | TGGAATTTTCCCTTTGTAAAATGACCTCTTCACAGTGCTCAC |
| | CTGTTGAAATTGTAGCTATCTTTCAAAGCCCAGCTCAGATG |
| | CCTTAGTCCTATGTGAAGCCTTTTGTGATTCTGCCAAACCAA |
| | TTATGATTTTACCATTCTTTGTGTTTCTATCTCTCTTATATCA |
| | TCTGCCATCGGTGCCTTCTCTTTTGGTGGTTTGCATGTATGG |
| | CTTAACTTCCTAGTTTGTAAACTCTGTGAGGACAACAAGGA |
| | CTGAGTCTTAACTGTCTTTGTCACCTTGCATATAGTTGATG |
| RAG Enhancer 7 | TGTACTGTTAGCAAATTACTTCCCCTCTTGGAACCTCAATTT |
| (5′PRP5L) | CATCATCCGTAAAATGGGAACAATAATAAAAATACCTTTTG |
| (SEQ ID NO: 7) | GCCATTAGGGGGAGCTCAAGGATGACTTATACCCCAAAAG |
| | GGGAAGGATTGCTTATGTGGATTCTCAGTTCTAGTCATTGG |
| | CAACGCTAAAGGGCAGAGCTTTCCCCTGTTGTGGGCAAGGG |
| | TTTGCACGAGGGGGACTGGCCATCCCAGTT |
| RAG Enhancer 8 | CTGTGGCTGGGATTCTGCAAACCACATTGCTGCTTTGTCAGC |
| (3′ TRAF6) | TGGGTCCCTGGTAGGTTCTGCCAATGGAGGGCGCTAGAGGG |
| (SEQ ID NO: 8) | AGCCTGGGGGACTGGTGGAGGGAGAGGAGTTGGGCTCATC |
| | CTGTCTTTGTCCACATCACCTAGCATAGACAGCTGGGTCCA |
| | GCAGCAGTGTTATATCCCTTCCAGTAATTTTGGTGGTTGCTT |
| | CTCAGCATTCTTGAGTTTATAGGTGGAGCATACTTGATCCTT |
| | GTGTGAGTCCCCAAGAGACACTGGGTCCTATTTGTGGTTGC |
| | TTAGTCAATGACTTCATAGCTTAAAAGAAGTGACCTCGGAG |
| | CCCTTGTTCCTGTATCGTACAATTGCCTAGTTCTTCAGTTAC |
| | CTTCAGCAGTATGCATTTCTCACTAAGTCACTCATTAGATTG |
| | GCTTTTCATTTCC |
| RAG Enhancer 9.1 | GTGGGCTTTGCCTACAGCACCTGCTTCTGCTTTCCGCATTTA |
| (ASE-Full) | CTGATCCACTAAGTGTAAGTGTGACATATAGAGGTCTGTGT |
| (SEQ ID NO: 9) | TGGTTGTTGTTTTGTGTTCAAACAGCTGAACATCTGGCCTCT |
| | CATGTGCAGGTGCACAGAACTCAGGAGAAGGGGTTTTAATT |
| | TAAGATAAAAAAAATAAGGATATGTGCTAGTTGCAGATGA |
| | GGGTTTGGGTGCTGGACTTAAGATGCTTCTGTGTTCAAAAA |
| | CAAGGATATATGAGTCCCTTATATGTCAGGGACAATGTACA |
| | GTGCTGGTCATAATATTCATTTTCTGAAAATGTCTTCACTGA |
| | CATAATGGCTTCTGATTTGTCATCCTGAACAAGTCTATTATA |
| | TTTTATTTAGTTATTGTCCTCAATTTGCAAGGTTCATAAAGA |
| | GTTTCACACAGATGGCACCCAAAGTGCTTATTTCTCATTGTG |
| | CTGAAATTAAACTTGAATATGCTATATATCAAATAAATAAT |
| | CCATTCCATGCCATTAAAAGAGTTTAGAATGAGAGATGAAA |
| | TAAATTTTGCAAAAAGTGTGCCCACATGTTCATTTTCTCCAA |
| | ATAGCATAGTCATTACATTTAAAAACAATCATTGGTCAAAA |
| | TAGAAAGTATTAAGAAAATACTAAGTATCAATTGAGGTCTA |
| | CCACATGTTCAGCAACAACTTAGGTGAGATGTGGGAGTAGG |
| | AAAAAGGAATACTTTATAACATCACAGAGAGGCAGATTTTG |
| | GCACAGGGAAGAGAAGTCAATGCAATACCTCAAGGAACCC |
| | AGTGGTGATCTAAGAAAACACTAGTTTGACCCATTGCCTCA |
| | ATTAAGTATTTACCTTTCATAGCTAAGAAGAGAAGTAAATA |
| | GCCTTTCTTTATACTTTCTAGAAGTTGAGCTCAATGTTCTTT |
| | CGGAGAGAGCAGGCTCATCTCCATTATGTAGCGCCAATGAC |
| | AAATACCTGTATATGTCTGTATTTCCTTGGTCTGTCTTGCTC |
| | ACCTGTTAAGACACCTTGCTGCACAACAGATGGCACATGCT |
| | TATCTTGTGGCAGTGAAGAGTTGACATCAAAGGCCAAGAAA |
| | ACCTTGAAACTCATCCTTTTCAGCCCACAAGAGCAGCTTCTC |
| | TCTACCACAGACGATGAAAGCTGGAGATAACAGGGGCATT |
| | CCCACCGCTGCACTCTCTCTTGTGGCCAGCTGACAGTTAAC |
| | ACAGGTGGGCGTGAGGTGCTGCCTACATTTTGAAGGTTGTC |
| | TATTGATGGGGAACACCACCTCTCTGAGCTCAGGCCTCTGC |
| | TTTGGCCCGTGGAGCTGCTGCTTCTGAGAAGTAACCAGGTG |
| | GTGAATACCTGCCTGCTTTTCAGAAAATCCGAGGTGGCTGT |
| | CAAAACACCCACTATCATTCAAAGGCTGTCAGGCAAGGTGG |
| | CCGTTTGAGGGGCCACTTTAGTTCTTGGTTTTTTTCAGAAGT |
| | CGAATTGGATTGCCAAACAGCTTGCTCAACTGCCTCTTCCC |
| | ATAAGCTAGATGGGAGGAAGGCTGGGTCTGTGTAACTTGGG |
| | GTGCGCACAACTAAAGTGCATAAAAAGAGAAGGA |
| RAG Enhancer 9.2 | GGCTTTGCCTACAGCACCTGCTTCTGCTTTCCGCATTTACTG |
| (ASE-core) | ATCCACTAAGTGTAAGTGTGACATATAGAGGTCTGTGTTGG |
| (SEQ ID NO: 10) | TTGTTGTTTTGTGTTCAAACAGCTGAACATCTGGCCTCTCAT |
| | GTGCAGGTGCACAGAACTCAGGAGAATCTTGCTCACCTGTT |
| | AAGACACCTTGCTGCACAACAGATGGCACATGCTTATCTTG |
| | TGGCAGTGAAGAGTTGACATCAAAGGCCAAGAAAACCTTG |
| | AAACTCATCCTTTTCAGCCCACAAGAGCAGCTTCTCTCTACC |
| | ACAGACGATGAAAGCTGGAGATAACAGGGGCATTCCCACC |
| | GCTGCACTCTCTCTTGTGGCCAGCTGACAGTTAACACAGGT |
| | GGGCGTGAGGTGCTGTTTGAGGGGCCACTTTAGTTCTTGGT |
| | TTTTTTCAGAAGTCGAATTGGATTGCCAAACAGCTTGCTCA |
| | ACTGCCTCTTCCCATAAGCTAGATGGGAGGAAGGCTGGGTC |
| | TGTGTAACTTGGGGTGCGCACAACTAAAGTGCATAAAAAGA |
| | G |
| RAG Enhancer 10 | ATTACCCACCTCACTATTATGAAGTATAACATACAGCGTGC |
| (PRR5L Int1) | ACAGCACAGTGTCTGGCCCATTGTAGTTGCTCAATAGCTTTT |
| (SEQ ID NO: 11) | AGTGTTTGCTTTTATGAGTTTGAGTCATCTTTGTGGTGAGGA |
| | TTACAGACCCTGACCCCTCTACTTGAGGTAGCTAAAGTTTCC |
| | CTCTTGATGATTTAATCTTGGTTGGGGTGAGAAAAAAGCAG |
| | GTATCAGCTGTGCTATTGGGGATTTCCTAACCCTTTATATTA |
| | TTACATGTTTCATCCTGCCTTGAGGAAGAAGTTTAAGCTAC |
| | GCCAATATTCGGGGCT |
| RAG Enhancer 11 | TTTTTTAGGTCTACTACTCTTGTGTCTGAAAGTTGCATTTTGT |
| (7.4kb 5′ RAG2) | TTGAGTAAGTTCATTAAGCAGGGACCTGAAAGTAAAGATCA |
| (SEQ ID NO: 12) | AAGGTGTGCTGAATCAGTGGTGACAATTTCATGTATCATAT |
| | TTCATAATAATGTTCTTTCAGAGAGCACATAACATTAACTA |
| | GCATGGAGCAGTGCATGCTCCCTGTATTTCCTTCTGGGGGC |
| | CATGATTGTGAAAGTGTTGCTTGTGATTTACCAGCAGATATT |
| | TATACACATTTACTTATCACAAGACGTTTTGAGTCTTTCATG |
| | TGGAGCTCAAAGCCTTAAAACATAAAAACCAA |
| RAG Enhancer 12 | TTGACAATTATTTATTCATTGTAAAATAATCACAGGAACAG |
| (TRAF6 3′UTR) | CAGCAGTGTAGGTTTCCCTACCTAGAGGGTGGTATGCAGTG |
| (SEQ ID NO: 13) | ATTCTCAGGCGCTGGTTGGAAGGCACAGCTGAGGGACACA |
| | AACTGCCAGGAAGTAATGTGGTAACTAGCCATGAGCTTGTG |
| | GTACTAATGGTGGCACGGGAAACAAGGTCTCTGCTTGACTT |
| | TTATTTTCACTCCATAACAAACTCACCAGATCAGGAGCCTA |
| | AGGGTGGGTGGGGAGGGAGAAGAGAGAAAAAAGCAAAGG |
| | GAAAGTTCAAAGTGACACTCGCTGGGGCTGAAAACCACACT |
| | CCCCTGCAGATGAGGTCCTTGGCT |
| RAG Enhancer 13 | CTGAAACCGTTTCACCTTTTATTAGAATAGAGCACTGAGGC |
| (P. Gfi1b b2) | ATGGCAGTAGCCACAGGAAGTCTGCTTTCCTTTGAAGATTG |
| | ATGAAGTTTGAGGAACCCTTTCATCCTTCAGTTAACTCCTAT |
| (SEQ ID NO: 14) | TTCCAAGTGTCTCCATGCCTATCTTTGGCTTGATTTCTGCCA |
| | CTTGACTTTTTTTGAATTCCTTTATCACTCTTTTACTCTTTTA |
| | GTGATCATTGACGTAGGTTCCTGTACTTGCTGTTGTCAGAAG |
| | AAGCAAGTTTCAGTTTTGGAATGTACCCTGCACGCTTAGTTC |
| | TTCTTTGACTATTGTAAGCCATTCTTCTTTGTCTCCTTTCTGG |
| | CTCCCTGCCCCCACCTTTCTGCTCCTTTTTTCTTCTATTCTCT |
| | AAAGACCTTTTCTTTTCATTTACTGCATTAATGCAGGAAGCA |
| | AGGCACAGTGGTTAGGAGGATAGGTTCTAGAGTCAGAAGG |
| | CAGTGTTCAGATCCCCACTCTGCTACTTAACCATCTGTAT |
| RAG Enhancer 14 | AATCCTTGCTGTGTAGCCTTTTGCAGTTATTTTCTGTCATGT |
| (eRAG) | GTTAGATAGAGACCAGAGGGCTTAACATTGTTTTGATACCC |
| (SEQ ID NO: 15) | TTGCATCTGGCAGATTGCCTGTCAGAGGAAGTTGGAAACGA |
| | ACTGACCAGCTTTATGCTTCCAGGTGGCACTGAAGCCACAT |
| | TTTCAGTGCCGTTGTTATCAGTCATTTAATTCCCTACTGAAT |
| | ATTGAGTGGTGTTTTTAGTTCACAGGCCATTTGCAGTGCCTT |
| RAG Enhancer 16 | TATCATTATTTACTGTCTAGCTACTGCAAACACAGTTTATTC |
| (8.4 kb 5′RAG2 | AGCTTTGCATTGCTAAAGCCTGGTGGTGTTACAAGTAAATG |
| (SEQ ID NO: 16) | TGCATAAGCCATAACCAGTGCTTTTTCGTGTATCTAATTTTT |
| | TCAGGTACAAATAGCTTGAAACAGTT |
| RAG Enhancer 17 | ATGATTTCTTCTTAGCCAGTTCATGGATTCAACAACTTCTGA |
| (HS1) | GACTTTTTCTCATGGCTTTTTCTGATAGTCAAGAATATAGGC |
| (SEQ ID NO: 17) | TTTTCTTTGCCCACACGGTGGCACTAGCTGCTTAGAAAACA |
| | GTGTAGCTAAAATCAGAAAGCCCGCAGCAGATCTCCTGAAA |
| | GCATGGAGAGTGAGCTGCAGCCGACGTT |
| RAG Enhancer 18 | AAAAGTCTATTTTATCTAATATAAGCGTAGTTACTCCTGCTT |
| (5′ of HS1) | ACTTTTGGTTTCCATTTGCATGGAGTATCTTTTTCCATCCTTT |
| (SEQ ID NO: 18) | CACTTTTCATCTATGTGTGTCTTTGTAGTTGAAGTGAGTTTC |
| | TTGTAGATAGCACATGGTTATGTCCTGTTTGTTTTTGTCCAT |
| | TCCTTCTGTCATTTTATTGTTTTCTGGTTGTTTTGCATTTCTCT |
| | TCCTCGTTTCTTTATCTCTTACTGTTTATCTTTGTGTGGTTTG |
| | CTTGGTGACAAATTTTAGTTACTTTCTCTTTCGCATCTGTGT |
| | ATCTGCTCTACTAGTGGGTTCTATACTTTTGTTTGTTCTCCTG |
| | ATGGTAGTTATTGCCCTTTGGCTTTCAGATATAGGGCTCC |
| RAG1 cDNA | ATGGCAGCCTCTTTCCCACCCACCTTGGGACTCAGTTCT |
| (SEQ ID NO: 19) | GCCCCAGATGAAATTCAGCACCCACATATTAAATTTTCA |
| | GAATGGAAATTTAAGCTGTTCCGGGTGAGATCCTTTGAA |
| | AAGACACCTGAAGAAGCTCAAAAGGAAAAGAAGGATTC |
| | CTTTGAGGGGAAACCCTCTCTGGAGCAATCTCCAGCAGT |
| | CCTGGACAAGGCTGATGGTCAGAAGCCAGTCCCAACTC |
| | AGCCATTGTTAAAAGCCCACCCTAAGTTTTCAAAGAAAT |
| | TTCACGACAACGAGAAAGCAAGAGGCAAAGCGATCCAT |
| | CAAGCCAACCTTCGACATCTCTGCCGCATCTGTGGGAAT |
| | TCTTTTAGAGCTGATGAGCACAACAGGAGATATCCAGTC |
| | CATGGTCCTGTGGATGGTAAAACCCTAGGCCTTTTACGA |
| | AAGAAGGAAAAGAGAGCTACTTCCTGGCCGGACCTCAT |
| | TGCCAAGGTTTTCCGGATCGATGTGAAGGCAGATGTTGA |
| | CTCGATCCACCCCACTGAGTTCTGCCATAACTGCTGGAG |
| | CATCATGCACAGGAAGTTTAGCAGTGCCCCATGTGAGGT |
| | TTACTTCCCGAGGAACGTGACCATGGAGTGGCACCCCC |
| | ACACACCATCCTGTGACATCTGCAACACTGCCCGTCGGG |
| | GACTCAAGAGGAAGAGTCTTCAGCCAAACTTGCAGCTCA |
| | GCAAAAAACTCAAAACTGTGCTTGACCAAGCAAGACAAG |
| | CCCGTCAGCACAAGAGAAGAGCTCAGGCAAGGATCAGC |
| | AGCAAGGATGTCATGAAGAAGATCGCCAACTGCAGTAA |
| | GATACATCTTAGTACCAAGCTCCTTGCAGTGGACTTCCC |
| | AGAGCACTTTGTGAAATCCATCTCCTGCCAGATCTGTGA |
| | ACACATTCTGGCTGACCCTGTGGAGACCAACTGTAAGCA |
| | TGTCTTTTGCCGGGTCTGCATTCTCAGATGCCTCAAAGT |
| | CATGGGCAGCTATTGTCCCTCTTGCCGATATCCATGCTT |
| | CCCTACTGACCTGGAGAGTCCAGTGAAGTCCTTTCTGAG |
| | CGTCTTGAATTCCCTGATGGTGAAATGTCCAGCAAAAGA |
| | GTGCAATGAGGAGGTCAGTTTGGAAAAATATAATCACCA |
| | CATCTCAAGTCACAAGGAATCAAAAGAGATTTTTGTGCA |
| | CATTAATAAAGGGGGCCGGCCCCGCCAACATCTTCTGTC |
| | GCTGACTCGGAGAGCTCAGAAGCACCGGCTGAGGGAGC |
| | TCAAGCTGCAAGTCAAAGCCTTTGCTGACAAAGAAGAAG |
| | GTGGAGATGTGAAGTCCGTGTGCATGACCTTGTTCCTGC |
| | TGGCTCTGAGGGCGAGGAATGAGCACAGGCAAGCTGAT |
| | GAGCTGGAGGCCATCATGCAGGGAAAGGGCTCTGGCCT |
| | GCAGCCAGCTGTTTGCTTGGCCATCCGTGTCAACACCTT |
| | CCTCAGCTGCAGTCAGTACCACAAGATGTACAGGACTGT |
| | GAAAGCCatcacagggagacagatttttcagcctttgcatGCCCTTCGGAAT |
| | GCTGAGAAGGTACTTCTGCCAGGCTACCACCACTTTGAG |
| | TGGCAGCCACCTCTGAAGAATGTGTCTTCCAGCACTGAT |
| | GTTGGCATTATTGATGGGCTGTCTGGACTATCATCCTCT |
| | GTGGATGATTACCCAGTGGACACCATTGCAAAGAGGTTC |
| | CGCTATGATTCAGCTTTGGTGTCTGCTTTGATGGACATG |
| | GAAGAAGACATCTTGGAAGGCATGAGATCCCAAGACCT |
| | TGATGATTACCTGAATGGCCCCTTCACTGTGGTGGTGAA |
| | GGAGTCTTGTGATGGAATGGGAGACGTGAGTGAGAAGC |
| | ATGGGAGTGGGCCTGTAGTTCCAGAAAAGGCAGTCCGT |
| | TTTTCATTCACAATCATGAAAATTACTATTGCCCACAGCT |
| | CTCAGAATGTGAAAGTATTTGAAGAAGCCAAACCTAACT |
| | CTGAACTGTGTTGCAAGCCATTGTGCCTTATGCTGGCAG |
| | ATGAGTCTGACCACGAGACGCTGACTGCCATCCTGAGTC |
| | CTCTCATTGCTGAGAGGGAGGCCATGAAGAGCAGTGAA |
| | TTAATGCTTGAGCTGGGAGGCATTCTCCGGACTTTCAAG |
| | TTCATCTTCAGGGGCACCGGCTATGATGAAAAACTTGTG |
| | CGGGAAGTGGAAGGCCTCGAGGCTTCTGGCTCAGTCTA |
| | CATTTGTACTCTTTGTGATGCCACCCGTCTGGAAGCCTC |
| | TCAAAATCTTGTCTTCCACTCTATAACCAGAAGCCATGC |
| | TGAGAACCTGGAACGTTATGAGGTCTGGCGTTCCAACCC |
| | TTACCATGAGTCTGTGGAAGAACTGCGGGATCGGGTGA |
| | AAGGGGTCTCAGCTAAACCTTTCATTGAGACAGTCCCTT |
| | CCATAGATGCACTCCACTGTGACATTGGCAATGCAGCTG |
| | AGTTCTACAAGATCTTCCAGCTAGAGATAGGGGAAGTGT |
| | ATAAGAATCCCAATGCTTCCAAAGAGGAAAGGAAAAGG |
| | TGGCAGGCCACACTGGACAAGCATCTCCGGAAGAAGAT |
| | GAACCTCAAACCAATCATGAGGATGAATGGCAACTTTGC |
| | CAGGAAGCTCATGACCAAAGAGACTGTGGATGCAGTTT |
| | GTGAGTTAATTCCTTCCGAGGAGAGGCACGAGGCTCTG |
| | AGGGAGCTGATGGATCTTTACCTGAAGATGAAACCAGTA |
| | TGGCGATCATCATGCCCTGCTAAAGAGTGCCCAGAATCC |
| | CTCTGCCAGTACAGTTTCAATTCACAGCGTTTTGCTGAG |
| | CTCCTTTCTACGAAGTTCAAGTATAGGTATGAGGGAAAA |
| | ATCACCAATTATTTTCACAAAACCCTGGCCCATGTTCCT |
| | GAAATTATTGAGAGGGATGGCTCCATTGGGGCATGGGC |
| | AAGTGAGGGAAATGAGTCTGGTAACAAACTGTTTAGGC |
| | GCTTCCGGAAAATGAATGCCAGGCAGTCCAAATGCTATG |
| | AGATGGAAGATGTCCTGAAACACCACTGGTTGTACACCT |
| | CCAAATACCTCCAGAAGTTTATGAATGCTCATAATGCAT |
| | TAAAAACCTCTGGGTTTACCATGAACCCTCAGGCAAGCT |
| | TAGGGGACCCATTAGGCATAGAGGACTCTCTGGAAAGC |
| | CAAGATTCAATGGAATTTTAA |
| Codon Optimized | ATGGCCGCCAGCTTCCCCCCCACCCTGGGCCTGAGCAG |
| RAG1 | CGCCCCCGACGAGATCCAGCACCCCCACATCAAGTTCA |
| (SEQ ID NO: 20) | GCGAGTGGAAGTTCAAGCTGTTCCGCGTGCGCAGCTTC |
| | GAGAAGACCCCCGAGGAGGCCCAGAAGGAGAAGAAGGA |
| | CAGCTTCGAGGGCAAGCCCAGCCTGGAGCAGAGCCCCG |
| | CCGTGCTGGACAAGGCCGACGGCCAGAAGCCCGTGCCC |
| | ACCCAGCCCCTGCTGAAGGCCCACCCCAAGTTCAGCAA |
| | GAAGTTCCACGACAACGAGAAGGCCCGCGGCAAGGCCA |
| | TCCACCAGGCCAACCTGCGCCACCTGTGCCGCATCTGC |
| | GGCAACAGCTTCCGCGCCGACGAGCACAACCGCCGCTA |
| | CCCCGTGCACGGCCCCGTGGACGGCAAGACCCTGGGCC |
| | TGCTGCGCAAGAAGGAGAAGCGCGCCACCAGCTGGCCC |
| | GACCTGATCGCCAAGGTGTTCCGCATCGACGTGAAGGC |
| | CGACGTGGACAGCATCCACCCCACCGAGTTCTGCCACA |
| | ACTGCTGGAGCATCATGCACCGCAAGTTCAGCAGCGCC |
| | CCCTGCGAGGTGTACTTCCCCCGCAACGTGACCATGGA |
| | GTGGCACCCCCACACCCCCAGCTGCGACATCTGCAACA |
| | CCGCCCGCCGCGGCCTGAAGCGCAAGAGCCTGCAGCCC |
| | AACCTGCAGCTGAGCAAGAAGCTGAAGACCGTGCTGGA |
| | CCAGGCCCGCCAGGCCCGCCAGCACAAGCGCCGCGCCC |
| | AGGCCCGCATCAGCAGCAAGGACGTGATGAAGAAGATC |
| | GCCAACTGCAGCAAGATCCACCTGAGCACCAAGCTGCT |
| | GGCCGTGGACTTCCCCGAGCACTTCGTGAAGAGCATCA |
| | GCTGCCAGATCTGCGAGCACATCCTGGCCGACCCCGTG |
| | GAGACCAACTGCAAGCACGTGTTCTGCCGCGTGTGCAT |
| | CCTGCGCTGCCTGAAGGTGATGGGCAGCTACTGCCCCA |
| | GCTGCCGCTACCCCTGCTTCCCCACCGACCTGGAGAGC |
| | CCCGTGAAGAGCTTCCTGAGCGTGCTGAACAGCCTGAT |
| | GGTGAAGTGCCCCGCCAAGGAGTGCAACGAGGAGGTGA |
| | GCCTGGAGAAGTACAACCACCACATCAGCAGCCACAAG |
| | GAGAGCAAGGAGATCTTCGTGCACATCAACAAGGGCGG |
| | CCGCCCCCGCCAGCACCTGCTGAGCCTGACCCGCCGCG |
| | CCCAGAAGCACCGCCTGCGCGAGCTGAAGCTGCAGGTG |
| | AAGGCCTTCGCCGACAAGGAGGAGGGCGGCGACGTGAA |
| | GAGCGTGTGCATGACCCTGTTCCTGCTGGCCCTGCGCG |
| | CCCGCAACGAGCACCGCCAGGCCGACGAGCTGGAGGCC |
| | ATCATGCAGGGCAAGGGCAGCGGCCTGCAGCCCGCCGT |
| | GTGCCTGGCCATCCGCGTGAACACCTTCCTGAGCTGCA |
| | GCCAGTACCACAAGATGTACCGCACCGTGAAGGCCATC |
| | ACCGGCCGCCAGATCTTCCAGCCCCTGCACGCCCTGCG |
| | CAACGCCGAGAAGGTGCTGCTGCCCGGCTACCACCACT |
| | TCGAGTGGCAGCCCCCCCTGAAGAACGTGAGCAGCAGC |
| | ACCGACGTGGGCATCATCGACGGCCTGAGCGGCCTGAG |
| | CAGCAGCGTGGACGACTACCCCGTGGACACCATCGCCA |
| | AGCGCTTCCGCTACGACAGCGCCCTGGTGAGCGCCCTG |
| | ATGGACATGGAGGAGGACATCCTGGAGGGCATGCGCAG |
| | CCAGGACCTGGACGACTACCTGAACGGCCCCTTCACCG |
| | TGGTGGTGAAGGAGAGCTGCGACGGCATGGGCGACGTG |
| | AGCGAGAAGCACGGCAGCGGCCCCGTGGTGCCCGAGAA |
| | GGCCGTGCGCTTCAGCTTCACCATCATGAAGATCACCAT |
| | CGCCCACAGCAGCCAGAACGTGAAGGTGTTCGAGGAGG |
| | CCAAGCCCAACAGCGAGCTGTGCTGCAAGCCCCTGTGC |
| | CTGATGCTGGCCGACGAGAGCGACCACGAGACCCTGAC |
| | CGCCATCCTGAGCCCCCTGATCGCCGAGCGCGAGGCCA |
| | TGAAGAGCAGCGAGCTGATGCTGGAGCTGGGCGGCATC |
| | CTGCGCACCTTCAAGTTCATCTTCCGCGGCACCGGCTAC |
| | GACGAGAAGCTGGTGCGCGAGGTGGAGGGCCTGGAGG |
| | CCAGCGGCAGCGTGTACATCTGCACCCTGTGCGACGCC |
| | ACCCGCCTGGAGGCCAGCCAGAACCTGGTGTTCCACAG |
| | CATCACCCGCAGCCACGCCGAGAACCTGGAGCGCTACG |
| | AGGTGTGGCGCAGCAACCCCTACCACGAGAGCGTGGAG |
| | GAGCTGCGCGACCGCGTGAAGGGCGTGAGCGCCAAGCC |
| | CTTCATCGAGACCGTGCCCAGCATCGACGCCCTGCACTG |
| | CGACATCGGCAACGCCGCCGAGTTCTACAAGATCTTCCA |
| | GCTGGAGATCGGCGAGGTGTACAAGAACCCCAACGCCA |
| | GCAAGGAGGAGCGCAAGCGCTGGCAGGCCACCCTGGAC |
| | AAGCACCTGCGCAAGAAGATGAACCTGAAGCCCATCAT |
| | GCGCATGAACGGCAACTTCGCCCGCAAGCTGATGACCA |
| | AGGAGACCGTGGACGCCGTGTGCGAGCTGATCCCCAGC |
| | GAGGAGCGCCACGAGGCCCTGCGCGAGCTGATGGACCT |
| | GTACCTGAAGATGAAGCCCGTGTGGCGCAGCAGCTGCC |
| | CCGCCAAGGAGTGCCCCGAGAGCCTGTGCCAGTACAGC |
| | TTCAACAGCCAGCGCTTCGCCGAGCTGCTGAGCACCAA |
| | GTTCAAGTACCGCTACGAGGGCAAGATCACCAACTACTT |
| | CCACAAGACCCTGGCCCACGTGCCCGAGATCATCGAGC |
| | GCGACGGCAGCATCGGCGCCTGGGCCAGCGAGGGCAAC |
| | GAGAGCGGCAACAAGCTGTTCCGCCGCTTCCGCAAGAT |
| | GAACGCCCGCCAGAGCAAGTGCTACGAGATGGAGGACG |
| | TGCTGAAGCACCACTGGCTGTACACCAGCAAGTACCTGC |
| | AGAAGTTCATGAACGCCCACAACGCCCTGAAGACCAGC |
| | GGCTTCACCATGAACCCCCAGGCCAGCCTGGGCGACCC |
| | CCTGGGCATCGAGGACAGCCTGGAGAGCCAGGACAGCA |
| | TGGAGTTCTAA |
| Sequence of the | AGAAAAGAGGGATTGGGGCCCTCTCCCAACAGACATGA |
| larger element 5 B- | GTATTTAGGAGTCCCCAGTTCCGGTTTCCACATGACATG |
| cell enhancer- | ATATGATGGTCTGTTGTTTTCCTCAGGGACCTAGAGGCA |
| “Element 5 Core | GATGTAGACAAATAGCAGGTGAGGAGGAAGGGTGGTGC |
| (SEQ ID NO: 31) | TGAGCTGTAAACAACAGCTGCGGGCTGTAAACAATGGA |
| The underlined | GCTATATTAAGAAAGTCCTGCATCTGCAGCATTCGGTTC |
| sequence in is SEQ | AATCCTGCCACCCACTGGGCGTACTCCTGAATTGACTTT |
| ID NO: 5. We have | CCATTGTCAGCTGATCAGTGACAGATGTGAAATGACTCG |
| expanded the | TTACACACACAGGTCACATTGGGAGGATTCTTAGACGTC |
| boundaries of the | ATATTCTTTGGTGCTTCACAAAGTCTTACTGTGGCACAT |
| element to include | GAAATGATTTCAGATGGTGCACAAGATGATTTCACATG |
| extra sequence here | |
| to add to our lead | |
| candidate vector | |
| Sequence of the | TAAATTTCCATGAGGGTGCAAAGAGAGGCTTTTCCCAAT |
| larger element 14 B- | GAGAGAAAAATATAGAAAGCAGCTGTCCCTCTTACCAAT |
| cell Enhancer- | TTCAGAGCAGGAAAGGAATTTCAGTAGAGCATAAAAACA |
| “Element 14 Core” | GCTTTCTCTCATAAAAGAAAACACAACAAGAAAAATAAT |
| (SEQ ID NO: 32) | CCTTGCTGTGTAGCCTTTTGCAGTTATTTTCTGTCATGT |
| The underlined | CTGAAGAATGAATTTAAAATCTGCTGTAGAAAAATACCA |
| sequence in yellow is | GTTAGATAGAGACCAGAGGGCTTAACATTGTTTTGATAC |
| SEQ ID NO: 14. | CCTTGCATCTGGCAGATTGCCTGTCAGAGGAAGTTGGAA |
| Wwe have expanded | ACGAACTGACCAGCTTTATGCTTCCAGGTGGCACTGAAG |
| the boundaries of the | CCACATTTTCAGTGCCGTTGTTATCAGTCATTTAATTCCC |
| element to include | TACTGAATATTGAGTGGTGTTTTTAGTTCACAGGCCATT |
| extra sequence here | TGCAGTGCCTTTCTGTTTCCTTTTTAACGTTAGCCACATT |
| to add to our lead | AAGAAAAAAA |
| candidate vector | |
| Sequence of “E11 | TCTAGGCAAGTGTCTCTCTCTCTCTCCCCCTCTTTTTCTT |
| extra B-cell | TTAGAAAAATACTTCGTGATAAAAACACAAAAGTATGTG |
| Enhancer” | AATGGTAATTGAAAATGCATTGCTTCCTTTTAGAAACCC |
| (SEQ ID NO: 33) | ACTAGGATTTGCATAAACCACTTGGCTGTCTGATAAGTC |
| | ATTATCTGTTCTGTCAGTCCCTGGGGACTTTTAATATTA |
| | GGTTTATATTTTTATAGAAAAGGCAAGTTCTAAATTTGA |
| | AGATGGGTATTGTTAAACTCTTCAATAATGACAGTTTGT |
| | GAAACTGCATGTTCCAAAAGTCCTTTAAAAAGTCAATAA |
| | GGC |
| Element 9.1 core | GTGGGCTTTGCCTACAGCACCTGCTTCTGCTTTCCGCAT |
| (SEQ ID NO: 34) | TTACTGATCCACTAAGTGTAAGTGTGACATATAGAGGTC |
| | TGTGTTGGTTGTTGTTTTGTGTTCAAACAGCTGAACATC |
| | TGGCCTCTCATGTGCAGGTGCACAGAACTCAGGAGAAG |
| | GGGTTTTAATTTAAGATAAAAAAAATAAGGATATGTGCT |
| | AGTTGCAGATGAGGGTTTGGGTGCTGGACTTAAGATGC |
| | TTCTGTGTTCAAAAACAAGGAGTCCTCAATTTGCAAGGT |
| | TCATAAAGAGTTTCACACAGATGGCACCCAAAGTGCTTA |
| | TTTCTCATTGTGCTGAAATTAAACTTGAATATGCTATATA |
| | TCAAATAAATAGAAGTAAATAGCCTTTCTTTATACTTTCT |
| | AGAAGTTGAGCTCAATGTTCTTTCGGAGAGAGCAGGCTC |
| | ATCTCCATTATGTAGCGCCAATGACAAATACCTGTATAT |
| | GTCTGTATTTCCTTGGTCTGTCTTGCTCACCTGTTAAGA |
| | CACCTTGCTGCACAACAGATGGCACATGCTTATCTTGTG |
| | GCAGTGAAGAGTTGACATCAAAGGCCAAGAAAACCTTG |
| | AAACTCATCCTTTTCAGCCCACAAGAGCAGCTTCTCTCT |
| | ACCACAGACGATGAAAGCTGGAGATAACAGGGGCATTC |
| | CCACCGCTGCACTCTCTCTTGTGGCCAGCTGACAGTTAA |
| | CACAGGTGGGCGTGAGGTGCTGCCTACATTTTGAAGGT |
| | TGTCTATTGATGGGGAACACCACCTCTCTGAGCTCAGGC |
| | CTCTGCTTTGGCCCGTGGAGCTGCTGCTTCTGAGAAGTA |
| | ACCAGGTGGTGAATACCTGCCTGCTTTTCAGAAAATCCG |
| | AGGTGGCTGTCAA |
| Element 18 right | CTTCTGTCATTTTATTGTTTTCTGGTTGTTTTGCATTTCT |
| fragment | CTTCCTCGTTTCTTTATCTCTTACTGTTTATCTTTGTGTG |
| (SEQ ID NO: 35) | GTTTGCTTGGTGACAAATTTTAGTTACTTTCTCTTTCGCA |
| | TCTGTGTATCTGCTCTACTAGTGGGTTCTATACTTTTGTT |
| | TGTTCTCCTGATGGTAGTTATTGCCCTTTGGCTTTCAGA |
| | TATAGGGCTCC |
| Element 13 right | TGGCTTGATTTCTGCCACTTGACTTTTTTTGAATTCCTTT |
| fragment | ATCACTCTTTTACTCTTTTAGTGATCATTGACGTAGGTTC |
| (SEQ ID NO: 36) | CTGTACTTGCTGTTGTCAGAAGAAGCAAGTTTCAGTTTT |
| | GGAATGTACCCTGCACGCTTAGTTCTTCTTTGACTATTG |
| | TAAGCCATTCTTCTTTGTCTCCTTTCTGGCTCCCTGCCCC |
| | CACCTTTCTGCTCCTTTTTTCTTCTATTCTCTAAAGACCT |
| | TTTCTTTTCATTTACTGCATTAATGCAGGAAGCAAGGCA |
| | CAGTGGTTAGGAGGATAGGTTCTAGAGTCAGAAGGCAG |
| | TGTTCAGATCCCCACTCTGCTACTTAACCATCTGTAT |

10 Lead candidates:

| | |
|---|---|
| 1) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG-RAG1pro- | CCTGGCTGACCGCCCAACGACCCCCGCCCATTGACGTC |
| RAG-WPRE | AATAATGACGTATGTTCCCATAGTAACGCCAATAGGGAC |
| (SEQ ID NO: 37) | TTTCCATTGACGTCAATGGGTGGAGTATTTACGGTAAAC |
| | TGCCCACTTGGCAGTACATCAAGTGTATCATATGCCAAG |
| | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTctcgagCTAA |
| | AGAGCCAGGTGGCAGCTGGAGCTGGGGTCTCCTGGCCC |
| | ATGATTGGCTGCCATCATTTGTGGTTAGCCCTCCATGGT |
| | GGGGGAGGCTGGGAAGGACAGTGGAAGCTGATAAACAG |
| | CTCAGCAGCATGTTCTGAGAAACAAGAGGGCAAGGAGA |
| | GAGCAGAGAACACACTTTGCCTTCTCTTTGGTATTGAGT |
| | AATATCAACCAAATTGCAGACATCTCAACACTTTGGCCA |
| | GGCAGCCTGCTGAGCAAGGTACCTCAGCCAGCATGgtgag |
| | caagggcgaggagctgttcaccggggtggtgcccatcctggtcgagctggacggcgacgta |
| | aacggccacaagttcagcgtgtccggcgagggcgagggcgatgccacctacggcaagctga |
| | ccctgaagttcatctgcaccaccggcaagctgcccgtgccctggcccaccctcgtgaccacct |
| | tcggctacggcctgatgtgcttcgcccgctaccccgaccacatgaagcagcacgacttcttca |
| | agtccgccatgcccgaaggctacgtccaggagcgcaccatcttcttcaaggacgacggcaac |
| | tacaagacccgcgccgaggtgaagttcgagggcgacaccctggtgaaccgcatcgagctga |
| | agggcatcgacttcaaggaggacggcaacatcctggggcacaagctggagtacaactacaa |
| | cagccacaacgtctatatcatggccgacaagcagaagaacggcatcaaggtgaacttcaag |
| | atccgccacaacatcgaggacggcagcgtgcagctcgccgaccactaccagcagaacaccc |
| | ccatcggcgacggccccgtgctgctgcccgacaaccactacctgagctaccagtccgccctg |
| | agcaaagaccccaacgagaagcgcgatcacatggtcctgctggagttcgtgaccgccgccg |
| | ggatcactctcggcatggacgagctgtacaagtgactgcaggaattcgagcatcttaccgcc |
| | atttattcccatatttgttctgtttttcttgatttgggtatacatttaaatgttaataaaacaaaat |
| | ggtggggcaatcatttacatttttagggatatgtaattactagttcaggtgtattgccacaagaca |
| | aacatgttaagaaactttcccgttatttacgctctgttcctgttaatcaacctctggattacaaaa |
| | tttgtgaaagattgactgatattcttaactatgttgctccttttacgctgtgtggatatgctgcttt |
| | aatgcctctgtatcatgctattgcttcccgtacggctttcgttttctcctccttgtataaatcctgg |
| | ttgctgtctctttatgaggagttgtggcccgttgtccgtcaacgtggcgtggtgtgctctgtgtttg |
| | ctgacgcaacccccactggctggggcattgccaccacctgtcaactcctttctgggactttcgc |
| | tttccccctcccgatcgccacggcagaactcatcgccgcctgccttgcccgctgctggacagg |
| | ggctaggttgctgggcactgataattccgtggtgttgtcggggaagggcctgctgccggctct |
| | gcggcctcttccgcgtcttcgccttcgccctcagacgagtcggatctccctttgggccgcctccc |
| | cgcctggaattcgagctcggtacctttaagaccaatgacttacaaggcagctgtagatcttag |
| | ccactttttaaaagaaaaggggggactggaagggctaattcactcccaacgaagacaagat |
| | ctgctttttgcttgtactgggtctctctggttagaccagatctgagcctgggagctctctggctaa |
| | ctagggaacctactgcttaagcctcaataaagcttgccttgagtgcttCAAGTAGTGT |
| | GTGCCCGTCTGTTGTGTGACTCTGGTAACTAGAGATCCC |
| | TCAGACCCTTTTAGTCAGTGTGGAAAATCTCTAGCagtagta |
| | gttcatgtcatcttattattcagtatttataacttgcaaagaaatgaatatcagagagtgagag |
| | gaacttgtttattgcagcttataatggttacaaataaagcaatagcatcacaaatttcacaaat |
| | aaagcatttttttcactgcattctagttgtggtttgtccaaactcatcaatgtatcttatcatgtct |
| | ggctctagctatcccgcccctaactccgcccatcccgcccctaactccgcccagttccgcccat |
| | tctccgccccatggctgactaattttttttatttatgcagaggccgaggccgcctcggcctctga |
| | gctattccagaagtagtgaggaggcttttttggaggcctagggacgtacccaattcgccctat |
| | agtgagtcgtattacgcgcgctcactggccgtcgttttacaacgtcgtgactgggaaaaccct |
| | ggcgttacccaacttaatcgccttgcagcacatccccctttcgccagctggcgtaatagcgaa |
| | gaggcccgcaccgatcgcccttcccaacagttgcgcagcctgaatggcgaatgggacgcgc |
| | cctgtagcggcgcattaagcgcggcgggtgtggtggttacgcgcagcgtgaccgctacactt |
| | gccagcgccctagcgcccgctcctttcgctttcttcccttcctttctcgccacgttcgccggctttc |
| | cccgtcaagctctaaatcgggggctccctttagggttccgatttagtgctttacggcacctcga |
| | ccccaaaaaacttgattagggtgatggttcacgtagtgggccatcgccctgatagacggttttt |
| | cgccctttgacgttggagtccacgttctttaatagtggactcttgttccaaactggaacaacact |
| | caaccctatctcggtctattcttttgatttataagggattttgccgatttcggcctattggttaaaa |
| | aatgagctgatttaacaaaaatttaacgcgaattttaacaaaatattaacgcttacaatttagg |
| | tggcacttttcggggaaatgtgcgcggaacccctatttgtttatttttctaaatacattcaaatat |
| | gtatccgctcatgagacaataaccctgataaatgcttcaataatagcacctagatcaagagac |
| | aggatgaggatcgtttcgcatgattgaacaagatggattgcacgcaggttctccggccgcttg |
| | ggtggagaggctattcggctatgactgggcacaacagacaatcggctgctctgatgccgccg |
| | tgttccggctgtcagcgcaggggcgcccggttctttttgtcaagaccgacctgtccggtgccct |
| | gaatgaactgcaagacgaggcagcgcggctatcgtggctggccacgacgggcgttccttgc |
| | gcagctgtgctcgacgttgtcactgaagcgggaagggactggctgctattgggcgaagtgcc |
| | ggggcaggatctcctgtcatctcaccttgctcctgccgagaaagtatccatcatggctgatgca |
| | atgcggcggctgcatacgcttgatccggctacctgcccattcgaccaccaagcgaaacatcg |
| | catcgagcgagcacgtactcggatggaagccggtcttgtcgatcaggatgatctggacgaag |
| | agcatcaggggctcgcgccagccgaactgttcgccaggctcaaggcgagcatgcccgacgg |
| | cgaggatctcgtcgtgacccatggcgatgcctgcttgccgaatatcatggtggaaaatggccg |
| | cttttctggattcatcgactgtggccggctgggtgtggcggaccgctatcaggacatagcgttg |
| | gctacccgtgatattgctgaagagcttggcggcgaatgggctgaccgcttcctcgtgctttacg |
| | gtatcgccgctcccgattcgcagcgcatcgccttctatcgccttcttgacgagttcttctgaatta |
| | ttaacgcttacaatttcctgatgcggtattttctccttacgcatctgtgcggtatttcacaccgcat |
| | caggtggcacttttcggggaaatgtgcgcggaacccctatttgtttatttttctaaatacattca |
| | aatatgtatccgctcatgaccaaaatcccttaacgtgagttttcgttccactgagcgtcagacc |
| | ccgtagaaaagatcaaaggatcttcttgagatcctttttttctgcgcgtaatctgctgcttgcaa |
| | acaaaaaaaccaccgctaccagcggtggtttgtttgccggatcaagagctaccaactctttttc |
| | cgaaggtaactggcttcagcagagcgcagataccaaatactgttcttctagtgtagccgtagt |
| | taggccaccacttcaagaactctgtagcaccgcctacatacctcgctctgctaatcctgttacc |
| | agtggctgctgccagtggcgataagtcgtgtcttaccgggttggactcaagacgatagttacc |
| | ggataaggcgcagcggtcgggctgaacggggggttcgtgcacacagcccagcttggagcga |
| | acgacctacaccgaactgagatacctacagcgtgagctatgagaaagcgccacgcttcccg |
| | aagggagaaaggcggacaggtatccggtaagcggcagggtcggaacaggagagcgcacg |
| | agggagcttccagggggaaacgcctggtatctttatagtcctgtcgggtttcgccacctctgac |
| | ttgagcgtcgatttttgtgatgctcgtcaggggggcggagcctatggaaaaacgccagcaac |
| | gcggcctttttacggttcctggccttttgctggccttttgctcacatgttctttcctgcgttatccc |
| | ctgattctgtggataaccgtattaccgcctttgagtgagctgataccgctcgccgcagccgaacg |
| | accgagcgcagcgagtcagtgagcgaggaagcggaagagcgcccaatacgcaaaccgcct |
| | ctccccgcgcgttggccgattcattaatgcagctggcacgacaggtttcccgactggaaagcg |
| | ggcagtgagcgcaacgcaattaatgtgagttagctcactcattaggcaccccaggctttacac |
| | tttatgcttccggctcgtatgttgtgtggaattgtgagcggataacaatttcacacaggaaaca |
| | gctatgaccatgattacgccaagcgcgcaattaaccctcactaaagggaacaaaagctgga |
| | gctgcaagcttggccattgcatacgttgtatccatatcataatatgtacatttatattggctcatg |
| | tccaacattaccgccat |
| 2) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG, E11 extra B-cell | CCTGGCTGACCGCCCAACGACCCCCGCCCATTGACGTC |
| enhancer- | AATAATGACGTATGTTCCCATAGTAACGCCAATAGGGAC |
| RAG1pro-RAG- | TTTCCATTGACGTCAATGGGTGGAGTATTTACGGTAAAC |
| WPRE | TGCCCACTTGGCAGTACATCAAGTGTATCATATGCCAAG |
| (SEQ ID NO: 38) | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTcTCTAGGC |
| | AAGTGTCTCTCTCTCTCTCCCCCTCTTTTTCTTTTAGAAA |
| | AATACTTCGTGATAAAAACACAAAAGTATGTGAATGGTA |
| | ATTGAAAATGCATTGCTTCCTTTTAGAAACCCACTAGGA |
| | TTTGCATAAACCACTTGGCTGTCTGATAAGTCATTATCT |
| | GTTCTGTCAGTCCCTGGGGACTTTTAATATTAGGTTTAT |
| | ATTTTTATAGAAAAGGCAAGTTCTAAATTTGAAGATGGG |
| | TATTGTTAAACTCTTCAATAATGACAGTTTGTGAAACTG |
| | CATGTTCCAAAAGTCCTTTAAAAAGTCAATAAGGCtcgagC |
| | TAAAGAGCCAGGTGGCAGCTGGAGCTGGGGTCTCCTGG |
| | CCCATGATTGGCTGCCATCATTTGTGGTTAGCCCTCCAT |
| | GGTGGGGGAGGCTGGGAAGGACAGTGGAAGCTGATAAA |
| | CAGCTCAGCAGCATGTTCTGAGAAACAAGAGGGCAAGG |
| | AGAGAGCAGAGAACACACTTTGCCTTCTCTTTGGTATTG |
| | AGTAATATCAACCAAATTGCAGACATCTCAACACTTTGG |
| | CCAGGCAGCCTGCTGAGCAAGGTACCTCAGCCAGCATGg |
| | tgagcaagggcgaggagctgttcaccggggtggtgcccatcctggtcgagctggacggcga |
| | cgtaaacggccacaagttcagcgtgtccggcgagggcgagggcgatgccacctacggcaag |
| | ctgaccctgaagttcatctgcaccaccggcaagctgcccgtgccctggcccaccctcgtgacc |
| | accttcggctacggcctgatgtgcttcgcccgctaccccgaccacatgaagcagcacgacttc |
| | ttcaagtccgccatgcccgaaggctacgtccaggagcgcaccatcttcttcaaggacgacgg |
| | caactacaagacccgcgccgaggtgaagttcgagggcgacaccctggtgaaccgcatcgag |
| | ctgaagggcatcgacttcaaggaggacggcaacatcctggggcacaagctggagtacaact |
| | acaacagccacaacgtctatatcatggccgacaagcagaagaacggcatcaaggtgaactt |
| | caagatccgccacaacatcgaggacggcagcgtgcagctcgccgaccactaccagcagaa |
| | cacccccatcggcgacggccccgtgctgctgcccgacaaccactacctgagctaccagtccg |
| | ccctgagcaaagaccccaacgagaagcgcgatcacatggtcctgctggagttcgtgaccgcc |
| | gccgggatcactctcggcatggacgagctgtacaagtgactgcaggaattcgagcatcttac |
| | cgccatttattcccatatttgttctgtttttcttgatttgggtatacatttaaatgttaataaaaca |
| | aaatggtggggcaatcatttacatttttagggatatgtaattactagttcaggtgtattgccacaa |
| | gacaaacatgttaagaaactttcccgttatttacgctctgttcctgttaatcaacctctggattac |
| | aaaatttgtgaaagattgactgatattcttaactatgttgctccttttacgctgtgtggatatgct |
| | gctttaatgcctctgtatcatgctattgcttcccgtacggctttcgttttctcctccttgtataaat |
| | cctggttgctgtctctttatgaggagttgtggcccgttgtccgtcaacgtggcgtggtgtgctctgt |
| | gtttgctgacgcaacccccactggctggggcattgccaccacctgtcaactcctttctgggact |
| | ttcgctttccccctcccgatcgccacggcagaactcatcgccgcctgccttgcccgctgctgga |
| | caggggctaggttgctgggcactgataattccgtggtgttgtcggggaagggcctgctgccgg |
| | ctctgcggcctcttccgcgtcttcgccttcgccctcagacgagtcggatctccctttgggccgcc |
| | tccccgcctggaattcgagctcggtacctttaagaccaatgacttacaaggcagctgtagatct |
| | tagccactttttaaaagaaaaggggggactggaagggctaattcactcccaacgaagacaa |
| | gatctgctttttgcttgtactgggtctctctggttagaccagatctgagcctgggagctctctggc |
| | taactagggaacctactgcttaagcctcaataaagcttgccttgagtgcttCAAGTAGT |
| | GTGTGCCCGTCTGTTGTGTGACTCTGGTAACTAGAGATC |
| | CCTCAGACCCTTTTAGTCAGTGTGGAAAATCTCTAGCagt |
| | agtagttcatgtcatcttattattcagtatttataacttgcaaagaaatgaatatcagagagtga |
| | gaggaacttgtttattgcagcttataatggttacaaataaagcaatagcatcacaaatttcaca |
| | aataaagcatttttttcactgcattctagttgtggtttgtccaaactcatcaatgtatcttatcatg |
| | tctggctctagctatcccgcccctaactccgcccatcccgcccctaactccgcccagttccgcc |
| | cattctccgccccatggctgactaattttttttatttatgcagaggccgaggccgcctcggcctct |
| | gagctattccagaagtagtgaggaggcttttttggaggcctagggacgtacccaattcgccct |
| | atagtgagtcgtattacgcgcgctcactggccgtcgttttacaacgtcgtgactgggaaaacc |
| | ctggcgttacccaacttaatcgccttgcagcacatccccctttcgccagctggcgtaatagcga |
| | agaggcccgcaccgatcgcccttcccaacagttgcgcagcctgaatggcgaatgggacgcg |
| | ccctgtagcggcgcattaagcgcggcgggtgtggtggttacgcgcagcgtgaccgctacactt |
| | gccagcgccctagcgcccgctcctttcgctttcttcccttcctttctcgccacgttcgccggctttc |
| | cccgtcaagctctaaatcgggggctccctttagggttccgatttagtgctttacggcacctcga |
| | ccccaaaaaacttgattagggtgatggttcacgtagtgggccatcgccctgatagacggttttt |
| | cgccctttgacgttggagtccacgttctttaatagtggactcttgttccaaactggaacaacact |
| | caaccctatctcggtctattcttttgatttataagggattttgccgatttcggcctattggttaaaa |
| | aatgagctgatttaacaaaaatttaacgcgaattttaacaaaatattaacgcttacaatttagg |
| | tggcacttttcggggaaatgtgcgcggaacccctatttgtttatttttctaaatacattcaaatat |
| | gtatccgctcatgagacaataaccctgataaatgcttcaataatagcacctagatcaagagac |
| | aggatgaggatcgtttcgcatgattgaacaagatggattgcacgcaggttctccggccgcttg |
| | ggtggagaggctattcggctatgactgggcacaacagacaatcggctgctctgatgccgccg |
| | tgttccggctgtcagcgcaggggcgcccggttctttttgtcaagaccgacctgtccggtgccct |
| | gaatgaactgcaagacgaggcagcgcggctatcgtggctggccacgacgggcgttccttgc |
| | gcagctgtgctcgacgttgtcactgaagcgggaagggactggctgctattgggcgaagtgcc |
| | ggggcaggatctcctgtcatctcaccttgctcctgccgagaaagtatccatcatggctgatgca |
| | atgcggcggctgcatacgcttgatccggctacctgcccattcgaccaccaagcgaaacatcg |
| | catcgagcgagcacgtactcggatggaagccggtcttgtcgatcaggatgatctggacgaag |
| | agcatcaggggctcgcgccagccgaactgttcgccaggctcaaggcgagcatgcccgacgg |
| | cgaggatctcgtcgtgacccatggcgatgcctgcttgccgaatatcatggtggaaaatggccg |
| | cttttctggattcatcgactgtggccggctgggtgtggcggaccgctatcaggacatagcgttg |
| | gctacccgtgatattgctgaagagcttggcggcgaatgggctgaccgcttcctcgtgctttacg |
| | gtatcgccgctcccgattcgcagcgcatcgccttctatcgccttcttgacgagttcttctgaatta |
| | ttaacgcttacaatttcctgatgcggtattttctccttacgcatctgtgcggtatttcacaccgcat |
| | caggtggcacttttcggggaaatgtgcgcggaacccctatttgtttatttttctaaatacattca |
| | aatatgtatccgctcatgaccaaaatcccttaacgtgagttttcgttccactgagcgtcagacc |
| | ccgtagaaaagatcaaaggatcttcttgagatcctttttttctgcgcgtaatctgctgcttgcaa |
| | acaaaaaaaccaccgctaccagcggtggtttgtttgccggatcaagagctaccaactctttttc |
| | cgaaggtaactggcttcagcagagcgcagataccaaatactgttcttctagtgtagccgtagt |
| | taggccaccacttcaagaactctgtagcaccgcctacatacctcgctctgctaatcctgttacc |
| | agtggctgctgccagtggcgataagtcgtgtcttaccgggttggactcaagacgatagttacc |
| | ggataaggcgcagcggtcgggctgaacggggggttcgtgcacacagcccagcttggagcga |
| | acgacctacaccgaactgagatacctacagcgtgagctatgagaaagcgccacgcttcccg |
| | aagggagaaaggcggacaggtatccggtaagcggcagggtcggaacaggagagcgcacg |
| | agggagcttccagggggaaacgcctggtatctttatagtcctgtcgggtttcgccacctctgac |
| | ttgagcgtcgatttttgtgatgctcgtcaggggggcggagcctatggaaaaacgccagcaac |
| | gcggcctttttacggttcctggccttttgctggccttttgctcacatgttctttcctgcgttatccc |
| | ctgattctgtggataaccgtattaccgcctttgagtgagctgataccgctcgccgcagccgaacg |
| | accgagcgcagcgagtcagtgagcgaggaagcggaagagcgcccaatacgcaaaccgcct |
| | ctccccgcgcgttggccgattcattaatgcagctggcacgacaggtttcccgactggaaagcg |
| | ggcagtgagcgcaacgcaattaatgtgagttagctcactcattaggcaccccaggctttacac |
| | tttatgcttccggctcgtatgttgtgtggaattgtgagcggataacaatttcacacaggaaaca |
| | gctatgaccatgattacgccaagcgcgcaattaaccctcactaaagggaacaaaagctgga |
| | gctgcaagcttggccattgcatacgttgtatccatatcataatatgtacatttatattggctcatg |
| | tccaacattaccgccat |
| 3) E5 Core, E14 | Cttgacattgattattgactagttattaatagtaatcaattacggggtcattagttcatagccca |
| Core, E9.1 OG large, | tatatggAGTTCCGCGTTACATAACTTACGGTAAATGGCCCG |
| E12 OG-RAG1pro- | CCTGGCTGACCGCCCAACGACCCCCGCCCATTGACGTC |
| RAG-WPRE | AATAATGACGTATGTTCCCATAGTAACGCCAATAGGGAC |
| (SEQ ID NO: 39) | TTTCCATTGACGTCAATGGGTGGAGTATTTACGGTAAAC |
| | TGCCCACTTGGCAGTACATCAAGTGTATCATATGCCAAG |
| | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGATAT |
| | ATGAGTCCCTTATATGTCAGGGACAATGTACAGTGCTGG |
| | TCATAATATTCATTTTCTGAAAATGTCTTCACTGACATAA |
| | TGGCTTCTGATTTGTCATCCTGAACAAGTCTATTATATTT |
| | TATTTAGTTATTGTCCTCAATTTGCAAGGTTCATAAAGA |
| | GTTTCACACAGATGGCACCCAAAGTGCTTATTTCTCATT |
| | GTGCTGAAATTAAACTTGAATATGCTATATATCAAATAA |
| | ATAATCCATTCCATGCCATTAAAAGAGTTTAGAATGAGA |
| | GATGAAATAAATTTTGCAAAAAGTGTGCCCACATGTTCA |
| | TTTTCTCCAAATAGCATAGTCATTACATTTAAAAACAATC |
| | ATTGGTCAAAATAGAAAGTATTAAGAAAATACTAAGTAT |
| | CAATTGAGGTCTACCACATGTTCAGCAACAACTTAGGTG |
| | AGATGTGGGAGTAGGAAAAAGGAATACTTTATAACATCA |
| | CAGAGAGGCAGATTTTGGCACAGGGAAGAGAAGTCAAT |
| | GCAATACCTCAAGGAACCCAGTGGTGATCTAAGAAAACA |
| | CTAGTTTGACCCATTGCCTCAATTAAGTATTTACCTTTCA |
| | TAGCTAAGAAGAGAAGTAAATAGCCTTTCTTTATACTTT |
| | CTAGAAGTTGAGCTCAATGTTCTTTCGGAGAGAGCAGGC |
| | TCATCTCCATTATGTAGCGCCAATGACAAATACCTGTAT |
| | ATGTCTGTATTTCCTTGGTCTGTCTTGCTCACCTGTTAA |
| | GACACCTTGCTGCACAACAGATGGCACATGCTTATCTTG |
| | TGGCAGTGAAGAGTTGACATCAAAGGCCAAGAAAACCT |
| | TGAAACTCATCCTTTTCAGCCCACAAGAGCAGCTTCTCT |
| | CTACCACAGACGATGAAAGCTGGAGATAACAGGGGCAT |
| | TCCCACCGCTGCACTCTCTCTTGTGGCCAGCTGACAGTT |
| | AACACAGGTGGGCGTGAGGTGCTGCCTACATTTTGAAG |
| | GTTGTCTATTGATGGGGAACACCACCTCTCTGAGCTCAG |
| | GCCTCTGCTTTGGCCCGTGGAGCTGCTGCTTCTGAGAAG |
| | TAACCAGGTGGTGAATACCTGCCTGCTTTTCAGAAAATC |
| | CGAGGTGGCTGTCAAAACACCCACTATCATTCAAAGGCT |
| | GTCAGGCAAGGTGGCCGTTTGAGGGGCCACTTTAGTTC |
| | TTGGTTTTTTTCAGAAGTCGAATTGGATTGCCAAACAGC |
| | TTGCTCAACTGCCTCTTCCCATAAGCTAGATGGGAGGAA |
| | GGCTGGGTCTGTGTAACTTGGGGTGCGCACAACTAAAG |
| | TGCATAAAAAGAGAAGGATTGACAATTATTTATTCATTG |
| | TAAAATAATCACAGGAACAGCAGCAGTGTAGGTTTCCCT |
| | ACCTAGAGGGTGGTATGCAGTGATTCTCAGGCGCTGGT |
| | TGGAAGGCACAGCTGAGGGACACAAACTGCCAGGAAGT |
| | AATGTGGTAACTAGCCATGAGCTTGTGGTACTAATGGTG |
| | GCACGGGAAACAAGGTCTCTGCTTGACTTTTATTTTCAC |
| | TCCATAACAAACTCACCAGATCAGGAGCCTAAGGGTGG |
| | GTGGGGAGGGAGAAGAGAGAAAAAAGCAAAGGGAAAGT |
| | TCAAAGTGACACTCGCTGGGGCTGAAAACCACACTCCCC |
| | TGCAGATGAGGTCCTTGGCTctcgagCTAAAGAGCCAGGTG |
| | GCAGCTGGAGCTGGGGTCTCCTGGCCCATGATTGGCTG |
| | CCATCATTTGTGGTTAGCCCTCCATGGTGGGGGAGGCT |
| | GGGAAGGACAGTGGAAGCTGATAAACAGCTCAGCAGCA |
| | TGTTCTGAGAAACAAGAGGGCAAGGAGAGAGCAGAGAA |
| | CACACTTTGCCTTCTCTTTGGTATTGAGTAATATCAACC |
| | AAATTGCAGACATCTCAACACTTTGGCCAGGCAGCCTGC |
| | TGAGCAAGGTACCTCAGCCAGCATGgtgagcaagggcgaggagctg |
| | ttcaccggggtggtgcccatcctggtcgagctggacggcgacgtaaacggccacaagttcag |
| | cgtgtccggcgagggcgagggcgatgccacctacggcaagctgaccctgaagttcatctgca |
| | ccaccggcaagctgcccgtgccctggcccaccctcgtgaccaccttcggctacggcctgatgt |
| | gcttcgcccgctaccccgaccacatgaagcagcacgacttcttcaagtccgccatgcccgaa |
| | ggctacgtccaggagcgcaccatcttcttcaaggacgacggcaactacaagacccgcgccg |
| | aggtgaagttcgagggcgacaccctggtgaaccgcatcgagctgaagggcatcgacttcaa |
| | ggaggacggcaacatcctggggcacaagctggagtacaactacaacagccacaacgtctat |
| | atcatggccgacaagcagaagaacggcatcaaggtgaacttcaagatccgccacaacatcg |
| | aggacggcagcgtgcagctcgccgaccactaccagcagaacacccccatcggcgacggcc |
| | ccgtgctgctgcccgacaaccactacctgagctaccagtccgccctgagcaaagaccccaac |
| | gagaagcgcgatcacatggtcctgctggagttcgtgaccgccgccgggatcactctcggcat |
| | ggacgagctgtacaagtgactgcaggaattcgagcatcttaccgccatttattcccatatttgtt |
| | ctgtttttcttgatttgggtatacatttaaatgttaataaaacaaaatggtggggcaatcatttac |
| | atttttagggatatgtaattactagttcaggtgtattgccacaagacaaacatgttaagaaact |
| | ttcccgttatttacgctctgttcctgttaatcaacctctggattacaaaatttgtgaaagattgac |
| | tgatattcttaactatgttgctccttttacgctgtgtggatatgctgctttaatgcctctgtatcat |
| | gctattgcttcccgtacggctttcgttttctcctccttgtataaatcctggttgctgtctctttatg |
| | aggagttgtggcccgttgtccgtcaacgtggcgtggtgtgctctgtgtttgctgacgcaaccccca |
| | ctggctggggcattgccaccacctgtcaactcctttctgggactttcgctttccccctcccgatc |
| | gccacggcagaactcatcgccgcctgccttgcccgctgctggacaggggctaggttgctggg |
| | cactgataattccgtggtgttgtcggggaagggcctgctgccggctctgcggcctcttccgcgt |
| | cttcgccttcgccctcagacgagtcggatctccctttgggccgcctccccgcctggaattcgag |
| | ctcggtacctttaagaccaatgacttacaaggcagctgtagatcttagccactttttaaaagaa |
| | aaggggggactggaagggctaattcactcccaacgaagacaagatctgctttttgcttgtact |
| | gggtctctctggttagaccagatctgagcctgggagctctctggctaactagggaacctactg |
| | cttaagcctcaataaagcttgccttgagtgcttCAAGTAGTGTGTGCCCGTC |
| | TGTTGTGTGACTCTGGTAACTAGAGATCCCTCAGACCCT |
| | TTTAGTCAGTGTGGAAAATCTCTAGCagtagtagttcatgtcatcttat |
| | tattcagtatttataacttgcaaagaaatgaatatcagagagtgagaggaacttgtttattgca |
| | gcttataatggttacaaataaagcaatagcatcacaaatttcacaaataaagcatttttttcac |
| | tgcattctagttgtggtttgtccaaactcatcaatgtatcttatcatgtctggctctagctatcccg |
| | cccctaactccgcccatcccgcccctaactccgcccagttccgcccattctccgccccatggct |
| | gactaattttttttatttatgcagaggccgaggccgcctcggcctctgagctattccagaagtag |
| | tgaggaggcttttttggaggcctagggacgtacccaattcgccctatagtgagtcgtattacgc |
| | gcgctcactggccgtcgttttacaacgtcgtgactgggaaaaccctggcgttacccaacttaat |
| | cgccttgcagcacatccccctttcgccagctggcgtaatagcgaagaggcccgcaccgatcg |
| | cccttcccaacagttgcgcagcctgaatggcgaatgggacgcgccctgtagcggcgcattaa |
| | gcgcggcgggtgtggtggttacgcgcagcgtgaccgctacacttgccagcgccctagcgccc |
| | gctcctttcgctttcttcccttcctttctcgccacgttcgccggctttccccgtcaagctctaaatc |
| | gggggctccctttagggttccgatttagtgctttacggcacctcgaccccaaaaaacttgatta |
| | gggtgatggttcacgtagtgggccatcgccctgatagacggtttttcgccctttgacgttggag |
| | tccacgttctttaatagtggactcttgttccaaactggaacaacactcaaccctatctcggtcta |
| | ttcttttgatttataagggattttgccgatttcggcctattggttaaaaaatgagctgatttaaca |
| | aaaatttaacgcgaattttaacaaaatattaacgcttacaatttaggtggcacttttcggggaa |
| | atgtgcgcggaacccctatttgtttatttttctaaatacattcaaatatgtatccgctcatgagac |
| | aataaccctgataaatgcttcaataatagcacctagatcaagagacaggatgaggatcgtttc |
| | gcatgattgaacaagatggattgcacgcaggttctccggccgcttgggtggagaggctattcg |
| | gctatgactgggcacaacagacaatcggctgctctgatgccgccgtgttccggctgtcagcgc |
| | aggggcgcccggttctttttgtcaagaccgacctgtccggtgccctgaatgaactgcaagacg |
| | aggcagcgcggctatcgtggctggccacgacgggcgttccttgcgcagctgtgctcgacgttg |
| | tcactgaagcgggaagggactggctgctattgggcgaagtgccggggcaggatctcctgtca |
| | tctcaccttgctcctgccgagaaagtatccatcatggctgatgcaatgcggcggctgcatacg |
| | cttgatccggctacctgcccattcgaccaccaagcgaaacatcgcatcgagcgagcacgtac |
| | tcggatggaagccggtcttgtcgatcaggatgatctggacgaagagcatcaggggctcgcgc |
| | cagccgaactgttcgccaggctcaaggcgagcatgcccgacggcgaggatctcgtcgtgacc |
| | catggcgatgcctgcttgccgaatatcatggtggaaaatggccgcttttctggattcatcgact |
| | gtggccggctgggtgtggcggaccgctatcaggacatagcgttggctacccgtgatattgctg |
| | aagagcttggcggcgaatgggctgaccgcttcctcgtgctttacggtatcgccgctcccgattc |
| | gcagcgcatcgccttctatcgccttcttgacgagttcttctgaattattaacgcttacaatttcct |
| | gatgcggtattttctccttacgcatctgtgcggtatttcacaccgcatcaggtggcacttttcgg |
| | ggaaatgtgcgcggaacccctatttgtttatttttctaaatacattcaaatatgtatccgctcatg |
| | accaaaatcccttaacgtgagttttcgttccactgagcgtcagaccccgtagaaaagatcaaa |
| | ggatcttcttgagatcctttttttctgcgcgtaatctgctgcttgcaaacaaaaaaaccaccgct |
| | accagcggtggtttgtttgccggatcaagagctaccaactctttttccgaaggtaactggcttc |
| | agcagagcgcagataccaaatactgttcttctagtgtagccgtagttaggccaccacttcaag |
| | aactctgtagcaccgcctacatacctcgctctgctaatcctgttaccagtggctgctgccagtg |
| | gcgataagtcgtgtcttaccgggttggactcaagacgatagttaccggataaggcgcagcgg |
| | tcgggctgaacggggggttcgtgcacacagcccagcttggagcgaacgacctacaccgaac |
| | tgagatacctacagcgtgagctatgagaaagcgccacgcttcccgaagggagaaaggcgg |
| | acaggtatccggtaagcggcagggtcggaacaggagagcgcacgagggagcttccagggg |
| | gaaacgcctggtatctttatagtcctgtcgggtttcgccacctctgacttgagcgtcgatttttgt |
| | gatgctcgtcaggggggcggagcctatggaaaaacgccagcaacgcggcctttttacggttc |
| | ctggccttttgctggccttttgctcacatgttctttcctgcgttatcccctgattctgtggataacc |
| | gtattaccgcctttgagtgagctgataccgctcgccgcagccgaacgaccgagcgcagcgag |
| | tcagtgagcgaggaagcggaagagcgcccaatacgcaaaccgcctctccccgcgcgttggc |
| | cgattcattaatgcagctggcacgacaggtttcccgactggaaagcgggcagtgagcgcaac |
| | gcaattaatgtgagttagctcactcattaggcaccccaggctttacactttatgcttccggctcg |
| | tatgttgtgtggaattgtgagcggataacaatttcacacaggaaacagctatgaccatgatta |
| | cgccaagcgcgcaattaaccctcactaaagggaacaaaagctggagctgcaagcttggcca |
| | ttgcatacgttgtatccatatcataatatgtacatttatattggctcatgtccaacattaccgcca |
| | t |
| 4) E5 Core, E14 | Cttgacattgattattgactagttattaatagtaatcaattacggggtcattagttcatagccca |
| Core, E9.1 OG large, | tatatggAGTTCCGCGTTACATAACTTACGGTAAATGGCCCG |
| E12 OG, E11 extra | CCTGGCTGACCGCCCAACGACCCCCGCCCATTGACGTC |
| B-cell enhancer - | AATAATGACGTATGTTCCCATAGTAACGCCAATAGGGAC |
| RAG1pro-RAG- | TTTCCATTGACGTCAATGGGTGGAGTATTTACGGTAAAC |
| WPRE | TGCCCACTTGGCAGTACATCAAGTGTATCATATGCCAAG |
| (SEQ ID NO: 40) | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGATAT |
| | ATGAGTCCCTTATATGTCAGGGACAATGTACAGTGCTGG |
| | TCATAATATTCATTTTCTGAAAATGTCTTCACTGACATAA |
| | TGGCTTCTGATTTGTCATCCTGAACAAGTCTATTATATTT |
| | TATTTAGTTATTGTCCTCAATTTGCAAGGTTCATAAAGA |
| | GTTTCACACAGATGGCACCCAAAGTGCTTATTTCTCATT |
| | GTGCTGAAATTAAACTTGAATATGCTATATATCAAATAA |
| | ATAATCCATTCCATGCCATTAAAAGAGTTTAGAATGAGA |
| | GATGAAATAAATTTTGCAAAAAGTGTGCCCACATGTTCA |
| | TTTTCTCCAAATAGCATAGTCATTACATTTAAAAACAATC |
| | ATTGGTCAAAATAGAAAGTATTAAGAAAATACTAAGTAT |
| | CAATTGAGGTCTACCACATGTTCAGCAACAACTTAGGTG |
| | AGATGTGGGAGTAGGAAAAAGGAATACTTTATAACATCA |
| | CAGAGAGGCAGATTTTGGCACAGGGAAGAGAAGTCAAT |
| | GCAATACCTCAAGGAACCCAGTGGTGATCTAAGAAAACA |
| | CTAGTTTGACCCATTGCCTCAATTAAGTATTTACCTTTCA |
| | TAGCTAAGAAGAGAAGTAAATAGCCTTTCTTTATACTTT |
| | CTAGAAGTTGAGCTCAATGTTCTTTCGGAGAGAGCAGGC |
| | TCATCTCCATTATGTAGCGCCAATGACAAATACCTGTAT |
| | ATGTCTGTATTTCCTTGGTCTGTCTTGCTCACCTGTTAA |
| | GACACCTTGCTGCACAACAGATGGCACATGCTTATCTTG |
| | TGGCAGTGAAGAGTTGACATCAAAGGCCAAGAAAACCT |
| | TGAAACTCATCCTTTTCAGCCCACAAGAGCAGCTTCTCT |
| | CTACCACAGACGATGAAAGCTGGAGATAACAGGGGCAT |
| | TCCCACCGCTGCACTCTCTCTTGTGGCCAGCTGACAGTT |
| | AACACAGGTGGGCGTGAGGTGCTGCCTACATTTTGAAG |
| | GTTGTCTATTGATGGGGAACACCACCTCTCTGAGCTCAG |
| | GCCTCTGCTTTGGCCCGTGGAGCTGCTGCTTCTGAGAAG |
| | TAACCAGGTGGTGAATACCTGCCTGCTTTTCAGAAAATC |
| | CGAGGTGGCTGTCAAAACACCCACTATCATTCAAAGGCT |
| | GTCAGGCAAGGTGGCCGTTTGAGGGGCCACTTTAGTTC |
| | TTGGTTTTTTTCAGAAGTCGAATTGGATTGCCAAACAGC |
| | TTGCTCAACTGCCTCTTCCCATAAGCTAGATGGGAGGAA |
| | GGCTGGGTCTGTGTAACTTGGGGTGCGCACAACTAAAG |
| | TGCATAAAAAGAGAAGGATTGACAATTATTTATTCATTG |
| | TAAAATAATCACAGGAACAGCAGCAGTGTAGGTTTCCCT |
| | ACCTAGAGGGTGGTATGCAGTGATTCTCAGGCGCTGGT |
| | TGGAAGGCACAGCTGAGGGACACAAACTGCCAGGAAGT |
| | AATGTGGTAACTAGCCATGAGCTTGTGGTACTAATGGTG |
| | GCACGGGAAACAAGGTCTCTGCTTGACTTTTATTTTCAC |
| | TCCATAACAAACTCACCAGATCAGGAGCCTAAGGGTGG |
| | GTGGGGAGGGAGAAGAGAGAAAAAAGCAAAGGGAAAGT |
| | TCAAAGTGACACTCGCTGGGGCTGAAAACCACACTCCCC |
| | TGCAGATGAGGTCCTTGGCTcTCTAGGCAAGTGTCTCTC |
| | TCTCTCTCCCCCTCTTTTTCTTTTAGAAAAATACTTCGTG |
| | ATAAAAACACAAAAGTATGTGAATGGTAATTGAAAATGC |
| | ATTGCTTCCTTTTAGAAACCCACTAGGATTTGCATAAAC |
| | CACTTGGCTGTCTGATAAGTCATTATCTGTTCTGTCAGT |
| | CCCTGGGGACTTTTAATATTAGGTTTATATTTTTATAGAA |
| | AAGGCAAGTTCTAAATTTGAAGATGGGTATTGTTAAACT |
| | CTTCAATAATGACAGTTTGTGAAACTGCATGTTCCAAAA |
| | GTCCTTTAAAAAGTCAATAAGGCtcgagCTAAAGAGCCAG |
| | GTGGCAGCTGGAGCTGGGGTCTCCTGGCCCATGATTGG |
| | CTGCCATCATTTGTGGTTAGCCCTCCATGGTGGGGGAG |
| | GCTGGGAAGGACAGTGGAAGCTGATAAACAGCTCAGCA |
| | GCATGTTCTGAGAAACAAGAGGGCAAGGAGAGAGCAGA |
| | GAACACACTTTGCCTTCTCTTTGGTATTGAGTAATATCA |
| | ACCAAATTGCAGACATCTCAACACTTTGGCCAGGCAGCC |
| | TGCTGAGCAAGGTACCTCAGCCAGCATGgtgagcaagggcgagg |
| | agctgttcaccggggtggtgcccatcctggtcgagctggacggcgacgtaaacggccacaag |
| | ttcagcgtgtccggcgagggcgagggcgatgccacctacggcaagctgaccctgaagttcat |
| | ctgcaccaccggcaagctgcccgtgccctggcccaccctcgtgaccaccttcggctacggcct |
| | gatgtgcttcgcccgctaccccgaccacatgaagcagcacgacttcttcaagtccgccatgcc |
| | cgaaggctacgtccaggagcgcaccatcttcttcaaggacgacggcaactacaagacccgc |
| | gccgaggtgaagttcgagggcgacaccctggtgaaccgcatcgagctgaagggcatcgact |
| | tcaaggaggacggcaacatcctggggcacaagctggagtacaactacaacagccacaacg |
| | tctatatcatggccgacaagcagaagaacggcatcaaggtgaacttcaagatccgccacaa |
| | catcgaggacggcagcgtgcagctcgccgaccactaccagcagaacacccccatcggcgac |
| | ggccccgtgctgctgcccgacaaccactacctgagctaccagtccgccctgagcaaagaccc |
| | caacgagaagcgcgatcacatggtcctgctggagttcgtgaccgccgccgggatcactctcg |
| | gcatggacgagctgtacaagtgactgcaggaattcgagcatcttaccgccatttattcccatat |
| | ttgttctgtttttcttgatttgggtatacatttaaatgttaataaaacaaaatggtggggcaatca |
| | tttacatttttagggatatgtaattactagttcaggtgtattgccacaagacaaacatgttaaga |
| | aactttcccgttatttacgctctgttcctgttaatcaacctctggattacaaaatttgtgaaagat |
| | tgactgatattcttaactatgttgctccttttacgctgtgtggatatgctgctttaatgcctctgta |
| | tcatgctattgcttcccgtacggctttcgttttctcctccttgtataaatcctggttgctgtctctt |
| | tatgaggagttgtggcccgttgtccgtcaacgtggcgtggtgtgctctgtgtttgctgacgcaaccc |
| | ccactggctggggcattgccaccacctgtcaactcctttctgggactttcgctttccccctcccg |
| | atcgccacggcagaactcatcgccgcctgccttgcccgctgctggacaggggctaggttgctg |
| | ggcactgataattccgtggtgttgtcggggaagggcctgctgccggctctgcggcctcttccgc |
| | gtcttcgccttcgccctcagacgagtcggatctccctttgggccgcctccccgcctggaattcg |
| | agctcggtacctttaagaccaatgacttacaaggcagctgtagatcttagccactttttaaaag |
| | aaaaggggggactggaagggctaattcactcccaacgaagacaagatctgctttttgcttgta |
| | ctgggtctctctggttagaccagatctgagcctgggagctctctggctaactagggaacctact |
| | gcttaagcctcaataaagcttgccttgagtgcttCAAGTAGTGTGTGCCCGT |
| | CTGTTGTGTGACTCTGGTAACTAGAGATCCCTCAGACCC |
| | TTTTAGTCAGTGTGGAAAATCTCTAGCagtagtagttcatgtcatctt |
| | attattcagtatttataacttgcaaagaaatgaatatcagagagtgagaggaacttgtttattg |
| | cagcttataatggttacaaataaagcaatagcatcacaaatttcacaaataaagcatttttttc |
| | actgcattctagttgtggtttgtccaaactcatcaatgtatcttatcatgtctggctctagctatcc |
| | cgcccctaactccgcccatcccgcccctaactccgcccagttccgcccattctccgccccatgg |
| | ctgactaattttttttatttatgcagaggccgaggccgcctcggcctctgagctattccagaagt |
| | agtgaggaggcttttttggaggcctagggacgtacccaattcgccctatagtgagtcgtattac |
| | gcgcgctcactggccgtcgttttacaacgtcgtgactgggaaaaccctggcgttacccaactt |
| | aatcgccttgcagcacatccccctttcgccagctggcgtaatagcgaagaggcccgcaccga |
| | tcgcccttcccaacagttgcgcagcctgaatggcgaatgggacgcgccctgtagcggcgcatt |
| | aagcgcggcgggtgtggtggttacgcgcagcgtgaccgctacacttgccagcgccctagcgc |
| | ccgctcctttcgctttcttcccttcctttctcgccacgttcgccggctttccccgtcaagctctaaa |
| | tcgggggctccctttagggttccgatttagtgctttacggcacctcgaccccaaaaaacttgatt |
| | agggtgatggttcacgtagtgggccatcgccctgatagacggtttttcgccctttgacgttgga |
| | gtccacgttctttaatagtggactcttgttccaaactggaacaacactcaaccctatctcggtct |
| | attcttttgatttataagggattttgccgatttcggcctattggttaaaaaatgagctgatttaac |
| | aaaaatttaacgcgaattttaacaaaatattaacgcttacaatttaggtggcacttttcgggga |
| | aatgtgcgcggaacccctatttgtttatttttctaaatacattcaaatatgtatccgctcatgaga |
| | caataaccctgataaatgcttcaataatagcacctagatcaagagacaggatgaggatcgttt |
| | cgcatgattgaacaagatggattgcacgcaggttctccggccgcttgggtggagaggctattc |
| | ggctatgactgggcacaacagacaatcggctgctctgatgccgccgtgttccggctgtcagcg |
| | caggggcgcccggttctttttgtcaagaccgacctgtccggtgccctgaatgaactgcaagac |
| | gaggcagcgcggctatcgtggctggccacgacgggcgttccttgcgcagctgtgctcgacgtt |
| | gtcactgaagcgggaagggactggctgctattgggcgaagtgccggggcaggatctcctgtc |
| | atctcaccttgctcctgccgagaaagtatccatcatggctgatgcaatgcggcggctgcatac |
| | gcttgatccggctacctgcccattcgaccaccaagcgaaacatcgcatcgagcgagcacgta |
| | ctcggatggaagccggtcttgtcgatcaggatgatctggacgaagagcatcaggggctcgcg |
| | ccagccgaactgttcgccaggctcaaggcgagcatgcccgacggcgaggatctcgtcgtgac |
| | ccatggcgatgcctgcttgccgaatatcatggtggaaaatggccgcttttctggattcatcgac |
| | tgtggccggctgggtgtggcggaccgctatcaggacatagcgttggctacccgtgatattgct |
| | gaagagcttggcggcgaatgggctgaccgcttcctcgtgctttacggtatcgccgctcccgatt |
| | cgcagcgcatcgccttctatcgccttcttgacgagttcttctgaattattaacgcttacaatttcc |
| | tgatgcggtattttctccttacgcatctgtgcggtatttcacaccgcatcaggtggcacttttcgg |
| | ggaaatgtgcgcggaacccctatttgtttatttttctaaatacattcaaatatgtatccgctcatg |
| | accaaaatcccttaacgtgagttttcgttccactgagcgtcagaccccgtagaaaagatcaaa |
| | ggatcttcttgagatcctttttttctgcgcgtaatctgctgcttgcaaacaaaaaaaccaccgct |
| | accagcggtggtttgtttgccggatcaagagctaccaactctttttccgaaggtaactggcttc |
| | agcagagcgcagataccaaatactgttcttctagtgtagccgtagttaggccaccacttcaag |
| | aactctgtagcaccgcctacatacctcgctctgctaatcctgttaccagtggctgctgccagtg |
| | gcgataagtcgtgtcttaccgggttggactcaagacgatagttaccggataaggcgcagcgg |
| | tcgggctgaacggggggttcgtgcacacagcccagcttggagcgaacgacctacaccgaac |
| | tgagatacctacagcgtgagctatgagaaagcgccacgcttcccgaagggagaaaggcgg |
| | acaggtatccggtaagcggcagggtcggaacaggagagcgcacgagggagcttccagggg |
| | gaaacgcctggtatctttatagtcctgtcgggtttcgccacctctgacttgagcgtcgatttttgt |
| | gatgctcgtcaggggggcggagcctatggaaaaacgccagcaacgcggcctttttacggttc |
| | ctggccttttgctggccttttgctcacatgttctttcctgcgttatcccctgattctgtggataacc |
| | gtattaccgcctttgagtgagctgataccgctcgccgcagccgaacgaccgagcgcagcgag |
| | tcagtgagcgaggaagcggaagagcgcccaatacgcaaaccgcctctccccgcgcgttggc |
| | cgattcattaatgcagctggcacgacaggtttcccgactggaaagcgggcagtgagcgcaac |
| | gcaattaatgtgagttagctcactcattaggcaccccaggctttacactttatgcttccggctcg |
| | tatgttgtgtggaattgtgagcggataacaatttcacacaggaaacagctatgaccatgatta |
| | cgccaagcgcgcaattaaccctcactaaagggaacaaaagctggagctgcaagcttggcca |
| | ttgcatacgttgtatccatatcataatatgtacatttatattggctcatgtccaacattaccgcca |
| | t |
| 5) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG + 18 right, 11 | CCTGGCTGACCGCCCAACGACCCCCGCCCATTGACGTC |
| whole, 13 right- | AATAATGACGTATGTTCCCATAGTAACGCCAATAGGGAC |
| RAG1pro-RAG- | TTTCCATTGACGTCAATGGGTGGAGTATTTACGGTAAAC |
| WPRE | TGCCCACTTGGCAGTACATCAAGTGTATCATATGCCAAG |
| (SEQ ID NO: 41) | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTctcgagCTTC |
| | TGTCATTTTATTGTTTTCTGGTTGTTTTGCATTTCTCTTC |
| | CTCGTTTCTTTATCTCTTACTGTTTATCTTTGTGTGGTTT |
| | GCTTGGTGACAAATTTTAGTTACTTTCTCTTTCGCATCTG |
| | TGTATCTGCTCTACTAGTGGGTTCTATACTTTTGTTTGTT |
| | CTCCTGATGGTAGTTATTGCCCTTTGGCTTTCAGATATA |
| | GGGCTCCTTTTTTAGGTCTACTACTCTTGTGTCTGAAAG |
| | TTGCATTTTGTTTGAGTAAGTTCATTAAGCAGGGACCTG |
| | AAAGTAAACATCAAAGGTGTGCTGAATCAGTGGTGACAA |
| | TTTCATGTATCATATTTCATAATAATGTTCTTTCAGAGAG |
| | CACATAACATTAACTAGCATGGAGCAGTGCATGCTCCCT |
| | GTATTTCCTTCTGGGGGCCATGATTGTGAAAGTGTTGCT |
| | TGTGATTTACCAGCAGATATTTATACACATTTACTTATCA |
| | CAAGACGTTTTGAGTCTTTCATGTGGAGCTCAAAGCCTT |
| | AAAACATAAAAACCAATGGCTTGATTTCTGCCACTTGAC |
| | TTTTTTTGAATTCCTTTATCACTCTTTTACTCTTTTAGTG |
| | ATCATTGACGTAGGTTCCTGTACTTGCTGTTGTCAGAAG |
| | AAGCAAGTTTCAGTTTTGGAATGTACCCTGCACGCTTAG |
| | TTCTTCTTTGACTATTGTAAGCCATTCTTCTTTGTCTCCT |
| | TTCTGGCTCCCTGCCCCCACCTTTCTGCTCCTTTTTTCTT |
| | CTATTCTCTAAAGACCTTTTCTTTTCATTTACTGCATTAA |
| | TGCAGGAAGCAAGGCACAGTGGTTAGGAGGATAGGTTC |
| | TAGAGTCAGAAGGCAGTGTTCAGATCCCCACTCTGCTAC |
| | TTAACCATCTGTATgCTAAAGAGCCAGGTGGCAGCTGGA |
| | GCTGGGGTCTCCTGGCCCATGATTGGCTGCCATCATTTG |
| | TGGTTAGCCCTCCATGGTGGGGGAGGCTGGGAAGGACA |
| | GTGGAAGCTGATAAACAGCTCAGCAGCATGTTCTGAGA |
| | AACAAGAGGGCAAGGAGAGAGCAGAGAACACACTTTGC |
| | CTTCTCTTTGGTATTGAGTAATATCAACCAAATTGCAGA |
| | CATCTCAACACTTTGGCCAGGCAGCCTGCTGAGCAAGGT |
| | ACCTCAGCCAGCATGgtgagcaagggcgaggagctgttcaccggggtggtgc |
| | ccatcctggtcgagctggacggcgacgtaaacggccacaagttcagcgtgtccggcgaggg |
| | cgagggcgatgccacctacggcaagctgaccctgaagttcatctgcaccaccggcaagctgc |
| | ccgtgccctggcccaccctcgtgaccaccttcggctacggcctgatgtgcttcgcccgctaccc |
| | cgaccacatgaagcagcacgacttcttcaagtccgccatgcccgaaggctacgtccaggagc |
| | gcaccatcttcttcaaggacgacggcaactacaagacccgcgccgaggtgaagttcgaggg |
| | cgacaccctggtgaaccgcatcgagctgaagggcatcgacttcaaggaggacggcaacatc |
| | ctggggcacaagctggagtacaactacaacagccacaacgtctatatcatggccgacaagc |
| | agaagaacggcatcaaggtgaacttcaagatccgccacaacatcgaggacggcagcgtgc |
| | agctcgccgaccactaccagcagaacacccccatcggcgacggccccgtgctgctgcccga |
| | caaccactacctgagctaccagtccgccctgagcaaagaccccaacgagaagcgcgatcac |
| | atggtcctgctggagttcgtgaccgccgccgggatcactctcggcatggacgagctgtacaag |
| | tgactgcaggaattcgagcatcttaccgccatttattcccatatttgttctgtttttcttgatttgg |
| | gtatacatttaaatgttaataaaacaaaatggtggggcaatcatttacatttttagggatatgt |
| | aattactagttcaggtgtattgccacaagacaaacatgttaagaaactttcccgttatttacgct |
| | ctgttcctgttaatcaacctctggattacaaaatttgtgaaagattgactgatattcttaactatg |
| | ttgctccttttacgctgtgtggatatgctgctttaatgcctctgtatcatgctattgcttcccgtac |
| | ggctttcgttttctcctccttgtataaatcctggttgctgtctctttatgaggagttgtggcccgtt |
| | gtccgtcaacgtggcgtggtgtgctctgtgtttgctgacgcaacccccactggctggggcattgc |
| | caccacctgtcaactcctttctgggactttcgctttccccctcccgatcgccacggcagaactca |
| | tcgccgcctgccttgcccgctgctggacaggggctaggttgctgggcactgataattccgtggt |
| | gttgtcggggaagggcctgctgccggctctgcggcctcttccgcgtcttcgccttcgccctcag |
| | acgagtcggatctccctttgggccgcctccccgcctggaattcgagctcggtacctttaagacc |
| | aatgacttacaaggcagctgtagatcttagccactttttaaaagaaaaggggggactggaag |
| | ggctaattcactcccaacgaagacaagatctgctttttgcttgtactgggtctctctggttagac |
| | cagatctgagcctgggagctctctggctaactagggaacctactgcttaagcctcaataaagc |
| | ttgccttgagtgcttCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtagtagttcatgtcatcttattattcagtatttataactt |
| | gcaaagaaatgaatatcagagagtgagaggaacttgtttattgcagcttataatggttacaaa |
| | taaagcaatagcatcacaaatttcacaaataaagcatttttttcactgcattctagttgtggttt |
| | gtccaaactcatcaatgtatcttatcatgtctggctctagctatcccgcccctaactccgcccat |
| | cccgcccctaactccgcccagttccgcccattctccgccccatggctgactaattttttttatttat |
| | gcagaggccgaggccgcctcggcctctgagctattccagaagtagtgaggaggcttttttgga |
| | ggcctagggacgtacccaattcgccctatagtgagtcgtattacgcgcgctcactggccgtcg |
| | ttttacaacgtcgtgactgggaaaaccctggcgttacccaacttaatcgccttgcagcacatcc |
| | ccctttcgccagctggcgtaatagcgaagaggcccgcaccgatcgcccttcccaacagttgcg |
| | cagcctgaatggcgaatgggacgcgccctgtagcggcgcattaagcgcggcgggtgtggtg |
| | gttacgcgcagcgtgaccgctacacttgccagcgccctagcgcccgctcctttcgctttcttccc |
| | ttcctttctcgccacgttcgccggctttccccgtcaagctctaaatcgggggctccctttagggtt |
| | ccgatttagtgctttacggcacctcgaccccaaaaaacttgattagggtgatggttcacgtagt |
| | gggccatcgccctgatagacggtttttcgccctttgacgttggagtccacgttctttaatagtgg |
| | actcttgttccaaactggaacaacactcaaccctatctcggtctattcttttgatttataagggat |
| | tttgccgatttcggcctattggttaaaaaatgagctgatttaacaaaaatttaacgcgaatttta |
| | acaaaatattaacgcttacaatttaggtggcacttttcggggaaatgtgcgcggaacccctatt |
| | tgtttatttttctaaatacattcaaatatgtatccgctcatgagacaataaccctgataaatgctt |
| | caataatagcacctagatcaagagacaggatgaggatcgtttcgcatgattgaacaagatgg |
| | attgcacgcaggttctccggccgcttgggtggagaggctattcggctatgactgggcacaaca |
| | gacaatcggctgctctgatgccgccgtgttccggctgtcagcgcaggggcgcccggttcttttt |
| | gtcaagaccgacctgtccggtgccctgaatgaactgcaagacgaggcagcgcggctatcgtg |
| | gctggccacgacgggcgttccttgcgcagctgtgctcgacgttgtcactgaagcgggaaggg |
| | actggctgctattgggcgaagtgccggggcaggatctcctgtcatctcaccttgctcctgccga |
| | gaaagtatccatcatggctgatgcaatgcggcggctgcatacgcttgatccggctacctgccc |
| | attcgaccaccaagcgaaacatcgcatcgagcgagcacgtactcggatggaagccggtcttg |
| | tcgatcaggatgatctggacgaagagcatcaggggctcgcgccagccgaactgttcgccag |
| | gctcaaggcgagcatgcccgacggcgaggatctcgtcgtgacccatggcgatgcctgcttgc |
| | cgaatatcatggtggaaaatggccgcttttctggattcatcgactgtggccggctgggtgtggc |
| | ggaccgctatcaggacatagcgttggctacccgtgatattgctgaagagcttggcggcgaat |
| | gggctgaccgcttcctcgtgctttacggtatcgccgctcccgattcgcagcgcatcgccttctat |
| | cgccttcttgacgagttcttctgaattattaacgcttacaatttcctgatgcggtattttctcctta |
| | cgcatctgtgcggtatttcacaccgcatcaggtggcacttttcggggaaatgtgcgcggaacc |
| | cctatttgtttatttttctaaatacattcaaatatgtatccgctcatgaccaaaatcccttaacgtg |
| | agttttcgttccactgagcgtcagaccccgtagaaaagatcaaaggatcttcttgagatcctttt |
| | tttctgcgcgtaatctgctgcttgcaaacaaaaaaaccaccgctaccagcggtggtttgtttgc |
| | cggatcaagagctaccaactctttttccgaaggtaactggcttcagcagagcgcagatacca |
| | aatactgttcttctagtgtagccgtagttaggccaccacttcaagaactctgtagcaccgccta |
| | catacctcgctctgctaatcctgttaccagtggctgctgccagtggcgataagtcgtgtcttacc |
| | gggttggactcaagacgatagttaccggataaggcgcagcggtcgggctgaacggggggtt |
| | cgtgcacacagcccagcttggagcgaacgacctacaccgaactgagatacctacagcgtga |
| | gctatgagaaagcgccacgcttcccgaagggagaaaggcggacaggtatccggtaagcgg |
| | cagggtcggaacaggagagcgcacgagggagcttccagggggaaacgcctggtatctttat |
| | agtcctgtcgggtttcgccacctctgacttgagcgtcgatttttgtgatgctcgtcaggggggcg |
| | gagcctatggaaaaacgccagcaacgcggcctttttacggttcctggccttttgctggccttttg |
| | ctcacatgttctttcctgcgttatcccctgattctgtggataaccgtattaccgcctttgagtgag |
| | ctgataccgctcgccgcagccgaacgaccgagcgcagcgagtcagtgagcgaggaagcgg |
| | aagagcgcccaatacgcaaaccgcctctccccgcgcgttggccgattcattaatgcagctgg |
| | cacgacaggtttcccgactggaaagcgggcagtgagcgcaacgcaattaatgtgagttagct |
| | cactcattaggcaccccaggctttacactttatgcttccggctcgtatgttgtgtggaattgtga |
| | gcggataacaatttcacacaggaaacagctatgaccatgattacgccaagcgcgcaattaac |
| | cctcactaaagggaacaaaagctggagctgcaagcttggccattgcatacgttgtatccatat |
| | cataatatgtacatttatattggctcatgtccaacattaccgccat |
| 6) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG + 18 whole, 11 | CCTGGCTGACCGCCCAACGACCCCCGCCCATTGACGTC |
| whole-RAG1pro- | AATAATGACGTATGTTCCCATAGTAACGCCAATAGGGAC |
| RAG-WPRE | TTTCCATTGACGTCAATGGGTGGAGTATTTACGGTAAAC |
| (SEQ ID NO: 42) | TGCCCACTTGGCAGTACATCAAGTGTATCATATGCCAAG |
| | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTctcgagAAAA |
| | GTCTATTTTATCTAATATAAGCGTAGTTACTCCTGCTTAC |
| | TTTTGGTTTCCATTTGCATGGAGTATCTTTTTCCATCCTT |
| | TCACTTTTCATCTATGTGTGTCTTTGTAGTTGAAGTGAG |
| | TTTCTTGTAGATAGCACATGGTTATGTCCTGTTTGTTTTT |
| | GTCCATTCCTTCTGTCATTTTATTGTTTTCTGGTTGTTTT |
| | GCATTTCTCTTCCTCGTTTCTTTATCTCTTACTGTTTATC |
| | TTTGTGTGGTTTGCTTGGTGACAAATTTTAGTTACTTTCT |
| | CTTTCGCATCTGTGTATCTGCTCTACTAGTGGGTTCTAT |
| | ACTTTTGTTTGTTCTCCTGATGGTAGTTATTGCCCTTTGG |
| | CTTTCAGATATAGGGCTCCTTTTTTAGGTCTACTACTCTT |
| | GTGTCTGAAAGTTGCATTTTGTTTGAGTAAGTTCATTAA |
| | GCAGGGACCTGAAAGTAAACATCAAAGGTGTGCTGAAT |
| | CAGTGGTGACAATTTCATGTATCATATTTCATAATAATG |
| | TTCTTTCAGAGAGCACATAACATTAACTAGCATGGAGCA |
| | GTGCATGCTCCCTGTATTTCCTTCTGGGGGCCATGATTG |
| | TGAAAGTGTTGCTTGTGATTTACCAGCAGATATTTATAC |
| | ACATTTACTTATCACAAGACGTTTTGAGTCTTTCATGTG |
| | GAGCTCAAAGCCTTAAAACATAAAAACCAAgCTAAAGAG |
| | CCAGGTGGCAGCTGGAGCTGGGGTCTCCTGGCCCATGA |
| | TTGGCTGCCATCATTTGTGGTTAGCCCTCCATGGTGGGG |
| | GAGGCTGGGAAGGACAGTGGAAGCTGATAAACAGCTCA |
| | GCAGCATGTTCTGAGAAACAAGAGGGCAAGGAGAGAGC |
| | AGAGAACACACTTTGCCTTCTCTTTGGTATTGAGTAATA |
| | TCAACCAAATTGCAGACATCTCAACACTTTGGCCAGGCA |
| | GCCTGCTGAGCAAGGTACCTCAGCCAGCATGgtgagcaaggg |
| | cgaggagctgttcaccggggtggtgcccatcctggtcgagctggacggcgacgtaaacggcc |
| | acaagttcagcgtgtccggcgagggcgagggcgatgccacctacggcaagctgaccctgaa |
| | gttcatctgcaccaccggcaagctgcccgtgccctggcccaccctcgtgaccaccttcggcta |
| | cggcctgatgtgcttcgcccgctaccccgaccacatgaagcagcacgacttcttcaagtccgc |
| | catgcccgaaggctacgtccaggagcgcaccatcttcttcaaggacgacggcaactacaag |
| | acccgcgccgaggtgaagttcgagggcgacaccctggtgaaccgcatcgagctgaagggca |
| | tcgacttcaaggaggacggcaacatcctggggcacaagctggagtacaactacaacagcca |
| | caacgtctatatcatggccgacaagcagaagaacggcatcaaggtgaacttcaagatccgc |
| | cacaacatcgaggacggcagcgtgcagctcgccgaccactaccagcagaacacccccatcg |
| | gcgacggccccgtgctgctgcccgacaaccactacctgagctaccagtccgccctgagcaaa |
| | gaccccaacgagaagcgcgatcacatggtcctgctggagttcgtgaccgccgccgggatcac |
| | tctcggcatggacgagctgtacaagtgactgcaggaattcgagcatcttaccgccatttattcc |
| | catatttgttctgtttttcttgatttgggtatacatttaaatgttaataaaacaaaatggtggggc |
| | aatcatttacatttttagggatatgtaattactagttcaggtgtattgccacaagacaaacatgt |
| | taagaaactttcccgttatttacgctctgttcctgttaatcaacctctggattacaaaatttgtga |
| | aagattgactgatattcttaactatgttgctccttttacgctgtgtggatatgctgctttaatgcct |
| | ctgtatcatgctattgcttcccgtacggctttcgttttctcctccttgtataaatcctggttgctgt |
| | ctctttatgaggagttgtggcccgttgtccgtcaacgtggcgtggtgtgctctgtgtttgctgacg |
| | caacccccactggctggggcattgccaccacctgtcaactcctttctgggactttcgctttcccc |
| | ctcccgatcgccacggcagaactcatcgccgcctgccttgcccgctgctggacaggggctag |
| | gttgctgggcactgataattccgtggtgttgtcggggaagggcctgctgccggctctgcggcct |
| | cttccgcgtcttcgccttcgccctcagacgagtcggatctccctttgggccgcctccccgcctgg |
| | aattcgagctcggtacctttaagaccaatgacttacaaggcagctgtagatcttagccacttttt |
| | aaaagaaaaggggggactggaagggctaattcactcccaacgaagacaagatctgcttttt |
| | gcttgtactgggtctctctggttagaccagatctgagcctgggagctctctggctaactaggga |
| | acctactgcttaagcctcaataaagcttgccttgagtgcttCAAGTAGTGTGTGC |
| | CCGTCTGTTGTGTGACTCTGGTAACTAGAGATCCCTCAG |
| | ACCCTTTTAGTCAGTGTGGAAAATCTCTAGCagtagtagttcatg |
| | tcatcttattattcagtatttataacttgcaaagaaatgaatatcagagagtgagaggaacttg |
| | tttattgcagcttataatggttacaaataaagcaatagcatcacaaatttcacaaataaagcat |
| | ttttttcactgcattctagttgtggtttgtccaaactcatcaatgtatcttatcatgtctggctcta |
| | gctatcccgcccctaactccgcccatcccgcccctaactccgcccagttccgcccattctccgcc |
| | ccatggctgactaattttttttatttatgcagaggccgaggccgcctcggcctctgagctattcc |
| | agaagtagtgaggaggcttttttggaggcctagggacgtacccaattcgccctatagtgagtc |
| | gtattacgcgcgctcactggccgtcgttttacaacgtcgtgactgggaaaaccctggcgttacc |
| | caacttaatcgccttgcagcacatccccctttcgccagctggcgtaatagcgaagaggcccgc |
| | accgatcgcccttcccaacagttgcgcagcctgaatggcgaatgggacgcgccctgtagcgg |
| | cgcattaagcgcggcgggtgtggtggttacgcgcagcgtgaccgctacacttgccagcgccc |
| | tagcgcccgctcctttcgctttcttcccttcctttctcgccacgttcgccggctttccccgtcaagc |
| | tctaaatcgggggctccctttagggttccgatttagtgctttacggcacctcgaccccaaaaaa |
| | cttgattagggtgatggttcacgtagtgggccatcgccctgatagacggtttttcgccctttgac |
| | gttggagtccacgttctttaatagtggactcttgttccaaactggaacaacactcaaccctatct |
| | cggtctattcttttgatttataagggattttgccgatttcggcctattggttaaaaaatgagctga |
| | tttaacaaaaatttaacgcgaattttaacaaaatattaacgcttacaatttaggtggcacttttc |
| | ggggaaatgtgcgcggaacccctatttgtttatttttctaaatacattcaaatatgtatccgctc |
| | atgagacaataaccctgataaatgcttcaataatagcacctagatcaagagacaggatgag |
| | gatcgtttcgcatgattgaacaagatggattgcacgcaggttctccggccgcttgggtggaga |
| | ggctattcggctatgactgggcacaacagacaatcggctgctctgatgccgccgtgttccggc |
| | tgtcagcgcaggggcgcccggttctttttgtcaagaccgacctgtccggtgccctgaatgaact |
| | gcaagacgaggcagcgcggctatcgtggctggccacgacgggcgttccttgcgcagctgtgc |
| | tcgacgttgtcactgaagcgggaagggactggctgctattgggcgaagtgccggggcaggat |
| | ctcctgtcatctcaccttgctcctgccgagaaagtatccatcatggctgatgcaatgcggcggc |
| | tgcatacgcttgatccggctacctgcccattcgaccaccaagcgaaacatcgcatcgagcga |
| | gcacgtactcggatggaagccggtcttgtcgatcaggatgatctggacgaagagcatcaggg |
| | gctcgcgccagccgaactgttcgccaggctcaaggcgagcatgcccgacggcgaggatctc |
| | gtcgtgacccatggcgatgcctgcttgccgaatatcatggtggaaaatggccgcttttctggat |
| | tcatcgactgtggccggctgggtgtggcggaccgctatcaggacatagcgttggctacccgtg |
| | atattgctgaagagcttggcggcgaatgggctgaccgcttcctcgtgctttacggtatcgccgc |
| | tcccgattcgcagcgcatcgccttctatcgccttcttgacgagttcttctgaattattaacgctta |
| | caatttcctgatgcggtattttctccttacgcatctgtgcggtatttcacaccgcatcaggtggca |
| | cttttcggggaaatgtgcgcggaacccctatttgtttatttttctaaatacattcaaatatgtatc |
| | cgctcatgaccaaaatcccttaacgtgagttttcgttccactgagcgtcagaccccgtagaaa |
| | agatcaaaggatcttcttgagatcctttttttctgcgcgtaatctgctgcttgcaaacaaaaaaa |
| | ccaccgctaccagcggtggtttgtttgccggatcaagagctaccaactctttttccgaaggtaa |
| | ctggcttcagcagagcgcagataccaaatactgttcttctagtgtagccgtagttaggccacc |
| | acttcaagaactctgtagcaccgcctacatacctcgctctgctaatcctgttaccagtggctgct |
| | gccagtggcgataagtcgtgtcttaccgggttggactcaagacgatagttaccggataaggc |
| | gcagcggtcgggctgaacggggggttcgtgcacacagcccagcttggagcgaacgacctac |
| | accgaactgagatacctacagcgtgagctatgagaaagcgccacgcttcccgaagggagaa |
| | aggcggacaggtatccggtaagcggcagggtcggaacaggagagcgcacgagggagcttc |
| | cagggggaaacgcctggtatctttatagtcctgtcgggtttcgccacctctgacttgagcgtcg |
| | atttttgtgatgctcgtcaggggggcggagcctatggaaaaacgccagcaacgcggccttttt |
| | acggttcctggccttttgctggccttttgctcacatgttctttcctgcgttatcccctgattctgtg |
| | gataaccgtattaccgcctttgagtgagctgataccgctcgccgcagccgaacgaccgagcgc |
| | agcgagtcagtgagcgaggaagcggaagagcgcccaatacgcaaaccgcctctccccgcg |
| | cgttggccgattcattaatgcagctggcacgacaggtttcccgactggaaagcgggcagtga |
| | gcgcaacgcaattaatgtgagttagctcactcattaggcaccccaggctttacactttatgcttc |
| | cggctcgtatgttgtgtggaattgtgagcggataacaatttcacacaggaaacagctatgacc |
| | atgattacgccaagcgcgcaattaaccctcactaaagggaacaaaagctggagctgcaagc |
| | ttggccattgcatacgttgtatccatatcataatatgtacatttatattggctcatgtccaacatt |
| | accgccat |
| 7) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG + 18 whole- | CCTGGCTGACCGCCCAACGACCCCCGCCCATTGACGTC |
| RAG1pro-RAG- | AATAATGACGTATGTTCCCATAGTAACGCCAATAGGGAC |
| WPRE | TTTCCATTGACGTCAATGGGTGGAGTATTTACGGTAAAC |
| (SEQ ID NO: 43) | TGCCCACTTGGCAGTACATCAAGTGTATCATATGCCAAG |
| | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTctcgagAAAA |
| | GTCTATTTTATCTAATATAAGCGTAGTTACTCCTGCTTAC |
| | TTTTGGTTTCCATTTGCATGGAGTATCTTTTTCCATCCTT |
| | TCACTTTTCATCTATGTGTGTCTTTGTAGTTGAAGTGAG |
| | TTTCTTGTAGATAGCACATGGTTATGTCCTGTTTGTTTTT |
| | GTCCATTCCTTCTGTCATTTTATTGTTTTCTGGTTGTTTT |
| | GCATTTCTCTTCCTCGTTTCTTTATCTCTTACTGTTTATC |
| | TTTGTGTGGTTTGCTTGGTGACAAATTTTAGTTACTTTCT |
| | CTTTCGCATCTGTGTATCTGCTCTACTAGTGGGTTCTAT |
| | ACTTTTGTTTGTTCTCCTGATGGTAGTTATTGCCCTTTGG |
| | CTTTCAGATATAGGGCTCCgCTAAAGAGCCAGGTGGCAG |
| | CTGGAGCTGGGGTCTCCTGGCCCATGATTGGCTGCCAT |
| | CATTTGTGGTTAGCCCTCCATGGTGGGGGAGGCTGGGA |
| | AGGACAGTGGAAGCTGATAAACAGCTCAGCAGCATGTT |
| | CTGAGAAACAAGAGGGCAAGGAGAGAGCAGAGAACACA |
| | CTTTGCCTTCTCTTTGGTATTGAGTAATATCAACCAAATT |
| | GCAGACATCTCAACACTTTGGCCAGGCAGCCTGCTGAG |
| | CAAGGTACCTCAGCCAGCATGgtgagcaagggcgaggagctgttcaccg |
| | gggtggtgcccatcctggtcgagctggacggcgacgtaaacggccacaagttcagcgtgtcc |
| | ggcgagggcgagggcgatgccacctacggcaagctgaccctgaagttcatctgcaccaccg |
| | gcaagctgcccgtgccctggcccaccctcgtgaccaccttcggctacggcctgatgtgcttcgc |
| | ccgctaccccgaccacatgaagcagcacgacttcttcaagtccgccatgcccgaaggctacg |
| | tccaggagcgcaccatcttcttcaaggacgacggcaactacaagacccgcgccgaggtgaa |
| | gttcgagggcgacaccctggtgaaccgcatcgagctgaagggcatcgacttcaaggaggac |
| | ggcaacatcctggggcacaagctggagtacaactacaacagccacaacgtctatatcatggc |
| | cgacaagcagaagaacggcatcaaggtgaacttcaagatccgccacaacatcgaggacgg |
| | cagcgtgcagctcgccgaccactaccagcagaacacccccatcggcgacggccccgtgctg |
| | ctgcccgacaaccactacctgagctaccagtccgccctgagcaaagaccccaacgagaagc |
| | gcgatcacatggtcctgctggagttcgtgaccgccgccgggatcactctcggcatggacgag |
| | ctgtacaagtgactgcaggaattcgagcatcttaccgccatttattcccatatttgttctgtttttc |
| | ttgatttgggtatacatttaaatgttaataaaacaaaatggtggggcaatcatttacatttttag |
| | ggatatgtaattactagttcaggtgtattgccacaagacaaacatgttaagaaactttcccgtt |
| | atttacgctctgttcctgttaatcaacctctggattacaaaatttgtgaaagattgactgatattc |
| | ttaactatgttgctccttttacgctgtgtggatatgctgctttaatgcctctgtatcatgctattgc |
| | ttcccgtacggctttcgttttctcctccttgtataaatcctggttgctgtctctttatgaggagttg |
| | tggcccgttgtccgtcaacgtggcgtggtgtgctctgtgtttgctgacgcaacccccactggctgg |
| | ggcattgccaccacctgtcaactcctttctgggactttcgctttccccctcccgatcgccacggc |
| | agaactcatcgccgcctgccttgcccgctgctggacaggggctaggttgctgggcactgataa |
| | ttccgtggtgttgtcggggaagggcctgctgccggctctgcggcctcttccgcgtcttcgccttc |
| | gccctcagacgagtcggatctccctttgggccgcctccccgcctggaattcgagctcggtacct |
| | ttaagaccaatgacttacaaggcagctgtagatcttagccactttttaaaagaaaagggggg |
| | actggaagggctaattcactcccaacgaagacaagatctgctttttgcttgtactgggtctctct |
| | ggttagaccagatctgagcctgggagctctctggctaactagggaacctactgcttaagcctc |
| | aataaagcttgccttgagtgcttCAAGTAGTGTGTGCCCGTCTGTTGT |
| | GTGACTCTGGTAACTAGAGATCCCTCAGACCCTTTTAGT |
| | CAGTGTGGAAAATCTCTAGCagtagtagttcatgtcatcttattattcagtat |
| | ttataacttgcaaagaaatgaatatcagagagtgagaggaacttgtttattgcagcttataatg |
| | gttacaaataaagcaatagcatcacaaatttcacaaataaagcatttttttcactgcattctagt |
| | tgtggtttgtccaaactcatcaatgtatcttatcatgtctggctctagctatcccgcccctaactc |
| | cgcccatcccgcccctaactccgcccagttccgcccattctccgccccatggctgactaattttt |
| | tttatttatgcagaggccgaggccgcctcggcctctgagctattccagaagtagtgaggaggc |
| | ttttttggaggcctagggacgtacccaattcgccctatagtgagtcgtattacgcgcgctcact |
| | ggccgtcgttttacaacgtcgtgactgggaaaaccctggcgttacccaacttaatcgccttgca |
| | gcacatccccctttcgccagctggcgtaatagcgaagaggcccgcaccgatcgcccttccca |
| | acagttgcgcagcctgaatggcgaatgggacgcgccctgtagcggcgcattaagcgcggcg |
| | ggtgtggtggttacgcgcagcgtgaccgctacacttgccagcgccctagcgcccgctcctttc |
| | gctttcttcccttcctttctcgccacgttcgccggctttccccgtcaagctctaaatcgggggctc |
| | cctttagggttccgatttagtgctttacggcacctcgaccccaaaaaacttgattagggtgatg |
| | gttcacgtagtgggccatcgccctgatagacggtttttcgccctttgacgttggagtccacgttc |
| | tttaatagtggactcttgttccaaactggaacaacactcaaccctatctcggtctattcttttgat |
| | ttataagggattttgccgatttcggcctattggttaaaaaatgagctgatttaacaaaaatttaa |
| | cgcgaattttaacaaaatattaacgcttacaatttaggtggcacttttcggggaaatgtgcgcg |
| | gaacccctatttgtttatttttctaaatacattcaaatatgtatccgctcatgagacaataaccct |
| | gataaatgcttcaataatagcacctagatcaagagacaggatgaggatcgtttcgcatgattg |
| | aacaagatggattgcacgcaggttctccggccgcttgggtggagaggctattcggctatgact |
| | gggcacaacagacaatcggctgctctgatgccgccgtgttccggctgtcagcgcaggggcgc |
| | ccggttctttttgtcaagaccgacctgtccggtgccctgaatgaactgcaagacgaggcagcg |
| | cggctatcgtggctggccacgacgggcgttccttgcgcagctgtgctcgacgttgtcactgaa |
| | gcgggaagggactggctgctattgggcgaagtgccggggcaggatctcctgtcatctcacctt |
| | gctcctgccgagaaagtatccatcatggctgatgcaatgcggcggctgcatacgcttgatccg |
| | gctacctgcccattcgaccaccaagcgaaacatcgcatcgagcgagcacgtactcggatgga |
| | agccggtcttgtcgatcaggatgatctggacgaagagcatcaggggctcgcgccagccgaa |
| | ctgttcgccaggctcaaggcgagcatgcccgacggcgaggatctcgtcgtgacccatggcga |
| | tgcctgcttgccgaatatcatggtggaaaatggccgcttttctggattcatcgactgtggccgg |
| | ctgggtgtggcggaccgctatcaggacatagcgttggctacccgtgatattgctgaagagctt |
| | ggcggcgaatgggctgaccgcttcctcgtgctttacggtatcgccgctcccgattcgcagcgc |
| | atcgccttctatcgccttcttgacgagttcttctgaattattaacgcttacaatttcctgatgcggt |
| | attttctccttacgcatctgtgcggtatttcacaccgcatcaggtggcacttttcggggaaatgt |
| | gcgcggaacccctatttgtttatttttctaaatacattcaaatatgtatccgctcatgaccaaaat |
| | cccttaacgtgagttttcgttccactgagcgtcagaccccgtagaaaagatcaaaggatcttct |
| | tgagatcctttttttctgcgcgtaatctgctgcttgcaaacaaaaaaaccaccgctaccagcgg |
| | tggtttgtttgccggatcaagagctaccaactctttttccgaaggtaactggcttcagcagagc |
| | gcagataccaaatactgttcttctagtgtagccgtagttaggccaccacttcaagaactctgta |
| | gcaccgcctacatacctcgctctgctaatcctgttaccagtggctgctgccagtggcgataagt |
| | cgtgtcttaccgggttggactcaagacgatagttaccggataaggcgcagcggtcgggctga |
| | acggggggttcgtgcacacagcccagcttggagcgaacgacctacaccgaactgagatacc |
| | tacagcgtgagctatgagaaagcgccacgcttcccgaagggagaaaggcggacaggtatcc |
| | ggtaagcggcagggtcggaacaggagagcgcacgagggagcttccagggggaaacgcct |
| | ggtatctttatagtcctgtcgggtttcgccacctctgacttgagcgtcgatttttgtgatgctcgtc |
| | aggggggcggagcctatggaaaaacgccagcaacgcggcctttttacggttcctggccttttg |
| | ctggccttttgctcacatgttctttcctgcgttatcccctgattctgtggataaccgtattaccgcc |
| | tttgagtgagctgataccgctcgccgcagccgaacgaccgagcgcagcgagtcagtgagcg |
| | aggaagcggaagagcgcccaatacgcaaaccgcctctccccgcgcgttggccgattcattaa |
| | tgcagctggcacgacaggtttcccgactggaaagcgggcagtgagcgcaacgcaattaatgt |
| | gagttagctcactcattaggcaccccaggctttacactttatgcttccggctcgtatgttgtgtg |
| | gaattgtgagcggataacaatttcacacaggaaacagctatgaccatgattacgccaagcgc |
| | gcaattaaccctcactaaagggaacaaaagctggagctgcaagcttggccattgcatacgtt |
| | gtatccatatcataatatgtacatttatattggctcatgtccaacattaccgccat |
| 8) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG, E11 extra B-cell | CCTGGCTGACCGCCCAACGACCCCCGCCCATTGACGTC |
| enhancer + 18 right, | AATAATGACGTATGTTCCCATAGTAACGCCAATAGGGAC |
| 11 whole, 13 right- | TTTCCATTGACGTCAATGGGTGGAGTATTTACGGTAAAC |
| RAG1pro-RAG- | TGCCCACTTGGCAGTACATCAAGTGTATCATATGCCAAG |
| WPRE | TACGCCCCCTATTGACGTCAATGACGGTAAATGGCCCGC |
| (SEQ ID NO: 44) | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTcTCTAGGC |
| | AAGTGTCTCTCTCTCTCTCCCCCTCTTTTTCTTTTAGAAA |
| | AATACTTCGTGATAAAAACACAAAAGTATGTGAATGGTA |
| | ATTGAAAATGCATTGCTTCCTTTTAGAAACCCACTAGGA |
| | TTTGCATAAACCACTTGGCTGTCTGATAAGTCATTATCT |
| | GTTCTGTCAGTCCCTGGGGACTTTTAATATTAGGTTTAT |
| | ATTTTTATAGAAAAGGCAAGTTCTAAATTTGAAGATGGG |
| | TATTGTTAAACTCTTCAATAATGACAGTTTGTGAAACTG |
| | CATGTTCCAAAAGTCCTTTAAAAAGTCAATAAGGCgCTT |
| | CTGTCATTTTATTGTTTTCTGGTTGTTTTGCATTTCTCTT |
| | CCTCGTTTCTTTATCTCTTACTGTTTATCTTTGTGTGGTT |
| | TGCTTGGTGACAAATTTTAGTTACTTTCTCTTTCGCATCT |
| | GTGTATCTGCTCTACTAGTGGGTTCTATACTTTTGTTTGT |
| | TCTCCTGATGGTAGTTATTGCCCTTTGGCTTTCAGATAT |
| | AGGGCTCCTTTTTTAGGTCTACTACTCTTGTGTCTGAAA |
| | GTTGCATTTTGTTTGAGTAAGTTCATTAAGCAGGGACCT |
| | GAAAGTAAACATCAAAGGTGTGCTGAATCAGTGGTGAC |
| | AATTTCATGTATCATATTTCATAATAATGTTCTTTCAGAG |
| | AGCACATAACATTAACTAGCATGGAGCAGTGCATGCTCC |
| | CTGTATTTCCTTCTGGGGGCCATGATTGTGAAAGTGTTG |
| | CTTGTGATTTACCAGCAGATATTTATACACATTTACTTAT |
| | CACAAGACGTTTTGAGTCTTTCATGTGGAGCTCAAAGCC |
| | TTAAAACATAAAAACCAATGGCTTGATTTCTGCCACTTG |
| | ActttttttGAAttcctttAtcActcttttActcttttAG |
| | TGATCATTGACGTAGGTTCCTGTACTTGCTGTTGTCAGA |
| | AGAAGCAAGTTTCAGTTTTGGAATGTACCCTGCACGCTT |
| | AGTTCTTCTTTGACTATTGTAAGCCATTCTTCTTTGTCTC |
| | CTTTCTGGCTCCCTGCCCCCACCTTTCTGCTCCTTTTTTC |
| | TTCTATTCTCTAAAGACCTTTTCTTTTCATTTACTGCATT |
| | AATGCAGGAAGCAAGGCACAGTGGTTAGGAGGATAGGT |
| | TCTAGAGTCAGAAGGCAGTGTTCAGATCCCCACTCTGCT |
| | ACTTAACCATCTGTATgCTAAAGAGCCAGGTGGCAGCTG |
| | GAGCTGGGGTCTCCTGGCCCATGATTGGCTGCCATCATT |
| | TGTGGTTAGCCCTCCATGGTGGGGGAGGCTGGGAAGGA |
| | CAGTGGAAGCTGATAAACAGCTCAGCAGCATGTTCTGA |
| | GAAACAAGAGGGCAAGGAGAGAGCAGAGAACACACTTT |
| | GCCTTCTCTTTGGTATTGAGTAATATCAACCAAATTGCA |
| | GACATCTCAACACTTTGGCCAGGCAGCCTGCTGAGCAA |
| | GGTACCTCAGCCAGCATGgtgagcaagggcgaggagctgttcaccggggt |
| | ggtgcccatcctggtcgagctggacggcgacgtaaacggccacaagttcagcgtgtccggcg |
| | agggcgagggcgatgccacctacggcaagctgaccctgaagttcatctgcaccaccggcaa |
| | gctgcccgtgccctggcccaccctcgtgaccaccttcggctacggcctgatgtgcttcgcccgc |
| | taccccgaccacatgaagcagcacgacttcttcaagtccgccatgcccgaaggctacgtcca |
| | ggagcgcaccatcttcttcaaggacgacggcaactacaagacccgcgccgaggtgaagttc |
| | gagggcgacaccctggtgaaccgcatcgagctgaagggcatcgacttcaaggaggacggc |
| | aacatcctggggcacaagctggagtacaactacaacagccacaacgtctatatcatggccga |
| | caagcagaagaacggcatcaaggtgaacttcaagatccgccacaacatcgaggacggcag |
| | cgtgcagctcgccgaccactaccagcagaacacccccatcggcgacggccccgtgctgctgc |
| | ccgacaaccactacctgagctaccagtccgccctgagcaaagaccccaacgagaagcgcga |
| | tcacatggtcctgctggagttcgtgaccgccgccgggatcactctcggcatggacgagctgta |
| | caagtgactgcaggaattcgagcatcttaccgccatttattcccatatttgttctgtttttcttgat |
| | ttgggtatacatttaaatgttaataaaacaaaatggtggggcaatcatttacatttttagggat |
| | atgtaattactagttcaggtgtattgccacaagacaaacatgttaagaaactttcccgttattta |
| | cgctctgttcctgttaatcaacctctggattacaaaatttgtgaaagattgactgatattcttaac |
| | tatgttgctccttttacgctgtgtggatatgctgctttaatgcctctgtatcatgctattgcttccc |
| | gtacggctttcgttttctcctccttgtataaatcctggttgctgtctctttatgaggagttgtggcc |
| | cgttgtccgtcaacgtggcgtggtgtgctctgtgtttgctgacgcaacccccactggctggggca |
| | ttgccaccacctgtcaactcctttctgggactttcgctttccccctcccgatcgccacggcagaa |
| | ctcatcgccgcctgccttgcccgctgctggacaggggctaggttgctgggcactgataattccg |
| | tggtgttgtcggggaagggcctgctgccggctctgcggcctcttccgcgtcttcgccttcgccct |
| | cagacgagtcggatctccctttgggccgcctccccgcctggaattcgagctcggtacctttaag |
| | accaatgacttacaaggcagctgtagatcttagccactttttaaaagaaaaggggggactgg |
| | aagggctaattcactcccaacgaagacaagatctgctttttgcttgtactgggtctctctggtta |
| | gaccagatctgagcctgggagctctctggctaactagggaacctactgcttaagcctcaataa |
| | agcttgccttgagtgcttCAAGTAGTGTGTGCCCGTCTGTTGTGTG |
| | ACTCTGGTAACTAGAGATCCCTCAGACCCTTTTAGTCAG |
| | TGTGGAAAATCTCTAGCagtagtagttcatgtcatcttattattcagtatttata |
| | acttgcaaagaaatgaatatcagagagtgagaggaacttgtttattgcagcttataatggtta |
| | caaataaagcaatagcatcacaaatttcacaaataaagcatttttttcactgcattctagttgtg |
| | gtttgtccaaactcatcaatgtatcttatcatgtctggctctagctatcccgcccctaactccgcc |
| | catcccgcccctaactccgcccagttccgcccattctccgccccatggctgactaattttttttat |
| | ttatgcagaggccgaggccgcctcggcctctgagctattccagaagtagtgaggaggctttttt |
| | ggaggcctagggacgtacccaattcgccctatagtgagtcgtattacgcgcgctcactggccg |
| | tcgttttacaacgtcgtgactgggaaaaccctggcgttacccaacttaatcgccttgcagcaca |
| | tccccctttcgccagctggcgtaatagcgaagaggcccgcaccgatcgcccttcccaacagtt |
| | gcgcagcctgaatggcgaatgggacgcgccctgtagcggcgcattaagcgcggcgggtgtg |
| | gtggttacgcgcagcgtgaccgctacacttgccagcgccctagcgcccgctcctttcgctttctt |
| | cccttcctttctcgccacgttcgccggctttccccgtcaagctctaaatcgggggctccctttag |
| | ggttccgatttagtgctttacggcacctcgaccccaaaaaacttgattagggtgatggttcacg |
| | tagtgggccatcgccctgatagacggtttttcgccctttgacgttggagtccacgttctttaata |
| | gtggactcttgttccaaactggaacaacactcaaccctatctcggtctattcttttgatttataag |
| | ggattttgccgatttcggcctattggttaaaaaatgagctgatttaacaaaaatttaacgcgaa |
| | ttttaacaaaatattaacgcttacaatttaggtggcacttttcggggaaatgtgcgcggaaccc |
| | ctatttgtttatttttctaaatacattcaaatatgtatccgctcatgagacaataaccctgataaa |
| | tgcttcaataatagcacctagatcaagagacaggatgaggatcgtttcgcatgattgaacaa |
| | gatggattgcacgcaggttctccggccgcttgggtggagaggctattcggctatgactgggca |
| | caacagacaatcggctgctctgatgccgccgtgttccggctgtcagcgcaggggcgcccggtt |
| | ctttttgtcaagaccgacctgtccggtgccctgaatgaactgcaagacgaggcagcgcggcta |
| | tcgtggctggccacgacgggcgttccttgcgcagctgtgctcgacgttgtcactgaagcggga |
| | agggactggctgctattgggcgaagtgccggggcaggatctcctgtcatctcaccttgctcctg |
| | ccgagaaagtatccatcatggctgatgcaatgcggcggctgcatacgcttgatccggctacct |
| | gcccattcgaccaccaagcgaaacatcgcatcgagcgagcacgtactcggatggaagccgg |
| | tcttgtcgatcaggatgatctggacgaagagcatcaggggctcgcgccagccgaactgttcg |
| | ccaggctcaaggcgagcatgcccgacggcgaggatctcgtcgtgacccatggcgatgcctgc |
| | ttgccgaatatcatggtggaaaatggccgcttttctggattcatcgactgtggccggctgggtg |
| | tggcggaccgctatcaggacatagcgttggctacccgtgatattgctgaagagcttggcggcg |
| | aatgggctgaccgcttcctcgtgctttacggtatcgccgctcccgattcgcagcgcatcgccttc |
| | tatcgccttcttgacgagttcttctgaattattaacgcttacaatttcctgatgcggtattttctcc |
| | ttacgcatctgtgcggtatttcacaccgcatcaggtggcacttttcggggaaatgtgcgcggaa |
| | cccctatttgtttatttttctaaatacattcaaatatgtatccgctcatgaccaaaatcccttaac |
| | gtgagttttcgttccactgagcgtcagaccccgtagaaaagatcaaaggatcttcttgagatcc |
| | tttttttctgcgcgtaatctgctgcttgcaaacaaaaaaaccaccgctaccagcggtggtttgtt |
| | tgccggatcaagagctaccaactctttttccgaaggtaactggcttcagcagagcgcagatac |
| | caaatactgttcttctagtgtagccgtagttaggccaccacttcaagaactctgtagcaccgcc |
| | tacatacctcgctctgctaatcctgttaccagtggctgctgccagtggcgataagtcgtgtctta |
| | ccgggttggactcaagacgatagttaccggataaggcgcagcggtcgggctgaacgggggg |
| | ttcgtgcacacagcccagcttggagcgaacgacctacaccgaactgagatacctacagcgtg |
| | agctatgagaaagcgccacgcttcccgaagggagaaaggcggacaggtatccggtaagcg |
| | gcagggtcggaacaggagagcgcacgagggagcttccagggggaaacgcctggtatcttta |
| | tagtcctgtcgggtttcgccacctctgacttgagcgtcgatttttgtgatgctcgtcaggggggc |
| | ggagcctatggaaaaacgccagcaacgcggcctttttacggttcctggccttttgctggcctttt |
| | gctcacatgttctttcctgcgttatcccctgattctgtggataaccgtattaccgcctttgagtga |
| | gctgataccgctcgccgcagccgaacgaccgagcgcagcgagtcagtgagcgaggaagcg |
| | gaagagcgcccaatacgcaaaccgcctctccccgcgcgttggccgattcattaatgcagctg |
| | gcacgacaggtttcccgactggaaagcgggcagtgagcgcaacgcaattaatgtgagttag |
| | ctcactcattaggcaccccaggctttacactttatgcttccggctcgtatgttgtgtggaattgtg |
| | agcggataacaatttcacacaggaaacagctatgaccatgattacgccaagcgcgcaattaa |
| | ccctcactaaagggaacaaaagctggagctgcaagcttggccattgcatacgttgtatccata |
| | tcataatatgtacatttatattggctcatgtccaacattaccgccat |
| 9) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG, E11 extra B-cell | CCTGGCTGACCGCCCAACGACCCCCGCCCATTGACGTC |
| enhancer + 18 whole, | AATAATGACGTATGTTCCCATAGTAACGCCAATAGGGAC |
| 11 whole- | TTTCCATTGACGTCAATGGGTGGAGTATTTACGGTAAAC |
| RAG1pro-RAG- | TGCCCACTTGGCAGTACATCAAGTGTATCATATGCCAAG |
| WPRE | TACGCCCCCTATTGACGTCAATGACGGTAAATGGCCCGC |
| (SEQ ID NO 45) | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTctcgagTCTA |
| | GGCAAGTGTCTCTCTCTCTCTCCCCCTCTTTTTCTTTTAG |
| | AAAAATACTTCGTGATAAAAACACAAAAGTATGTGAATG |
| | GTAATTGAAAATGCATTGCTTCCTTTTAGAAACCCACTA |
| | GGATTTGCATAAACCACTTGGCTGTCTGATAAGTCATTA |
| | TCTGTTCTGTCAGTCCCTGGGGACTTTTAATATTAGGTT |
| | TATATTTTTATAGAAAAGGCAAGTTCTAAATTTGAAGAT |
| | GGGTATTGTTAAACTCTTCAATAATGACAGTTTGTGAAA |
| | CTGCATGTTCCAAAAGTCCTTTAAAAAGTCAATAAGGCg |
| | AAAAGTCTATTTTATCTAATATAAGCGTAGTTACTCCTG |
| | CTTACTTTTGGTTTCCATTTGCATGGAGTATCTTTTTCCA |
| | TCCTTTCACTTTTCATCTATGTGTGTCTTTGTAGTTGAAG |
| | TGAGTTTCTTGTAGATAGCACATGGTTATGTCCTGTTTG |
| | TTTTTGTCCATTCCTTCTGTCATTTTATTGTTTTCTGGTT |
| | GTTTTGCATTTCTCTTCCTCGTTTCTTTATCTCTTACTGT |
| | TTATCTTTGTGTGGTTTGCTTGGTGACAAATTTTAGTTAC |
| | TTTCTCTTTCGCATCTGTGTATCTGCTCTACTAGTGGGTT |
| | CTATACTTTTGTTTGTTCTCCTGATGGTAGTTATTGCCCT |
| | TTGGCTTTCAGATATAGGGCTCCTTTTTTAGGTCTACTA |
| | CTCTTGTGTCTGAAAGTTGCATTTTGTTTGAGTAAGTTC |
| | ATTAAGCAGGGACCTGAAAGTAAACATCAAAGGTGTGCT |
| | GAATCAGTGGTGACAATTTCATGTATCATATTTCATAAT |
| | AATGTTCTTTCAGAGAGCACATAACATTAACTAGCATGG |
| | AGCAGTGCATGCTCCCTGTATTTCCTTCTGGGGGCCATG |
| | ATTGTGAAAGTGTTGCTTGTGATTTACCAGCAGATATTT |
| | ATACACATTTACTTATCACAAGACGTTTTGAGTCTTTCAT |
| | GTGGAGCTCAAAGCCTTAAAACATAAAAACCAAgCTAAA |
| | GAGCCAGGTGGCAGCTGGAGCTGGGGTCTCCTGGCCCA |
| | TGATTGGCTGCCATCATTTGTGGTTAGCCCTCCATGGTG |
| | GGGGAGGCTGGGAAGGACAGTGGAAGCTGATAAACAGC |
| | TCAGCAGCATGTTCTGAGAAACAAGAGGGCAAGGAGAG |
| | AGCAGAGAACACACTTTGCCTTCTCTTTGGTATTGAGTA |
| | ATATCAACCAAATTGCAGACATCTCAACACTTTGGCCAG |
| | GCAGCCTGCTGAGCAAGGTACCTCAGCCAGCATGgtgagca |
| | agggcgaggagctgttcaccggggtggtgcccatcctggtcgagctggacggcgacgtaaa |
| | cggccacaagttcagcgtgtccggcgagggcgagggcgatgccacctacggcaagctgacc |
| | ctgaagttcatctgcaccaccggcaagctgcccgtgccctggcccaccctcgtgaccaccttc |
| | ggctacggcctgatgtgcttcgcccgctaccccgaccacatgaagcagcacgacttcttcaag |
| | tccgccatgcccgaaggctacgtccaggagcgcaccatcttcttcaaggacgacggcaacta |
| | caagacccgcgccgaggtgaagttcgagggcgacaccctggtgaaccgcatcgagctgaag |
| | ggcatcgacttcaaggaggacggcaacatcctggggcacaagctggagtacaactacaaca |
| | gccacaacgtctatatcatggccgacaagcagaagaacggcatcaaggtgaacttcaagat |
| | ccgccacaacatcgaggacggcagcgtgcagctcgccgaccactaccagcagaacaccccc |
| | atcggcgacggccccgtgctgctgcccgacaaccactacctgagctaccagtccgccctgag |
| | caaagaccccaacgagaagcgcgatcacatggtcctgctggagttcgtgaccgccgccggg |
| | atcactctcggcatggacgagctgtacaagtgactgcaggaattcgagcatcttaccgccatt |
| | tattcccatatttgttctgtttttcttgatttgggtatacatttaaatgttaataaaacaaaatggt |
| | ggggcaatcatttacatttttagggatatgtaattactagttcaggtgtattgccacaagacaa |
| | acatgttaagaaactttcccgttatttacgctctgttcctgttaatcaacctctggattacaaaat |
| | ttgtgaaagattgactgatattcttaactatgttgctccttttacgctgtgtggatatgctgcttta |
| | atgcctctgtatcatgctattgcttcccgtacggctttcgttttctcctccttgtataaatcctggt |
| | tgctgtctctttatgaggagttgtggcccgttgtccgtcaacgtggcgtggtgtgctctgtgtttgc |
| | tgacgcaacccccactggctggggcattgccaccacctgtcaactcctttctgggactttcgctt |
| | tccccctcccgatcgccacggcagaactcatcgccgcctgccttgcccgctgctggacagggg |
| | ctaggttgctgggcactgataattccgtggtgttgtcggggaagggcctgctgccggctctgcg |
| | gcctcttccgcgtcttcgccttcgccctcagacgagtcggatctccctttgggccgcctccccgc |
| | ctggaattcgagctcggtacctttaagaccaatgacttacaaggcagctgtagatcttagcca |
| | ctttttaaaagaaaaggggggactggaagggctaattcactcccaacgaagacaagatctgc |
| | tttttgcttgtactgggtctctctggttagaccagatctgagcctgggagctctctggctaactag |
| | ggaacctactgcttaagcctcaataaagcttgccttgagtgcttCAAGTAGTGTGT |
| | GCCCGTCTGTTGTGTGACTCTGGTAACTAGAGATCCCTC |
| | AGACCCTTTTAGTCAGTGTGGAAAATCTCTAGCagtagtagtt |
| | catgtcatcttattattcagtatttataacttgcaaagaaatgaatatcagagagtgagaggaa |
| | cttgtttattgcagcttataatggttacaaataaagcaatagcatcacaaatttcacaaataaa |
| | gcatttttttcactgcattctagttgtggtttgtccaaactcatcaatgtatcttatcatgtctggc |
| | tctagctatcccgcccctaactccgcccatcccgcccctaactccgcccagttccgcccattctcc |
| | gccccatggctgactaattttttttatttatgcagaggccgaggccgcctcggcctctgagctat |
| | tccagaagtagtgaggaggcttttttggaggcctagggacgtacccaattcgccctatagtga |
| | gtcgtattacgcgcgctcactggccgtcgttttacaacgtcgtgactgggaaaaccctggcgtt |
| | acccaacttaatcgccttgcagcacatccccctttcgccagctggcgtaatagcgaagaggcc |
| | cgcaccgatcgcccttcccaacagttgcgcagcctgaatggcgaatgggacgcgccctgtag |
| | cggcgcattaagcgcggcgggtgtggtggttacgcgcagcgtgaccgctacacttgccagcg |
| | ccctagcgcccgctcctttcgctttcttcccttcctttctcgccacgttcgccggctttccccgtca |
| | agctctaaatcgggggctccctttagggttccgatttagtgctttacggcacctcgaccccaaa |
| | aaacttgattagggtgatggttcacgtagtgggccatcgccctgatagacggtttttcgcccttt |
| | gacgttggagtccacgttctttaatagtggactcttgttccaaactggaacaacactcaaccct |
| | atctcggtctattcttttgatttataagggattttgccgatttcggcctattggttaaaaaatgag |
| | ctgatttaacaaaaatttaacgcgaattttaacaaaatattaacgcttacaatttaggtggcac |
| | ttttcggggaaatgtgcgcggaacccctatttgtttatttttctaaatacattcaaatatgtatcc |
| | gctcatgagacaataaccctgataaatgcttcaataatagcacctagatcaagagacaggat |
| | gaggatcgtttcgcatgattgaacaagatggattgcacgcaggttctccggccgcttgggtgg |
| | agaggctattcggctatgactgggcacaacagacaatcggctgctctgatgccgccgtgttcc |
| | ggctgtcagcgcaggggcgcccggttctttttgtcaagaccgacctgtccggtgccctgaatg |
| | aactgcaagacgaggcagcgcggctatcgtggctggccacgacgggcgttccttgcgcagct |
| | gtgctcgacgttgtcactgaagcgggaagggactggctgctattgggcgaagtgccggggca |
| | ggatctcctgtcatctcaccttgctcctgccgagaaagtatccatcatggctgatgcaatgcgg |
| | cggctgcatacgcttgatccggctacctgcccattcgaccaccaagcgaaacatcgcatcga |
| | gcgagcacgtactcggatggaagccggtcttgtcgatcaggatgatctggacgaagagcatc |
| | aggggctcgcgccagccgaactgttcgccaggctcaaggcgagcatgcccgacggcgagg |
| | atctcgtcgtgacccatggcgatgcctgcttgccgaatatcatggtggaaaatggccgcttttc |
| | tggattcatcgactgtggccggctgggtgtggcggaccgctatcaggacatagcgttggctac |
| | ccgtgatattgctgaagagcttggcggcgaatgggctgaccgcttcctcgtgctttacggtatc |
| | gccgctcccgattcgcagcgcatcgccttctatcgccttcttgacgagttcttctgaattattaac |
| | gcttacaatttcctgatgcggtattttctccttacgcatctgtgcggtatttcacaccgcatcagg |
| | tggcacttttcggggaaatgtgcgcggaacccctatttgtttatttttctaaatacattcaaatat |
| | gtatccgctcatgaccaaaatcccttaacgtgagttttcgttccactgagcgtcagaccccgta |
| | gaaaagatcaaaggatcttcttgagatcctttttttctgcgcgtaatctgctgcttgcaaacaaa |
| | aaaaccaccgctaccagcggtggtttgtttgccggatcaagagctaccaactctttttccgaag |
| | gtaactggcttcagcagagcgcagataccaaatactgttcttctagtgtagccgtagttaggcc |
| | accacttcaagaactctgtagcaccgcctacatacctcgctctgctaatcctgttaccagtggc |
| | tgctgccagtggcgataagtcgtgtcttaccgggttggactcaagacgatagttaccggataa |
| | ggcgcagcggtcgggctgaacggggggttcgtgcacacagcccagcttggagcgaacgacc |
| | tacaccgaactgagatacctacagcgtgagctatgagaaagcgccacgcttcccgaaggga |
| | gaaaggcggacaggtatccggtaagcggcagggtcggaacaggagagcgcacgagggag |
| | cttccagggggaaacgcctggtatctttatagtcctgtcgggtttcgccacctctgacttgagcg |
| | tcgatttttgtgatgctcgtcaggggggcggagcctatggaaaaacgccagcaacgcggcctt |
| | tttacggttcctggccttttgctggccttttgctcacatgttctttcctgcgttatcccctgattct |
| | gtggataaccgtattaccgcctttgagtgagctgataccgctcgccgcagccgaacgaccgagc |
| | gcagcgagtcagtgagcgaggaagcggaagagcgcccaatacgcaaaccgcctctccccg |
| | cgcgttggccgattcattaatgcagctggcacgacaggtttcccgactggaaagcgggcagt |
| | gagcgcaacgcaattaatgtgagttagctcactcattaggcaccccaggctttacactttatgc |
| | ttccggctcgtatgttgtgtggaattgtgagcggataacaatttcacacaggaaacagctatg |
| | accatgattacgccaagcgcgcaattaaccctcactaaagggaacaaaagctggagctgca |
| | agcttggccattgcatacgttgtatccatatcataatatgtacatttatattggctcatgtccaac |
| | attaccgccat |
| 10) E5 Core, E14 | Cttgacattgattattgactagttattaatagtaatcaattacggggtcattagttcatagccca |
| Core, E9.1 Core, E12 | tatatggAGTTCCGCGTTACATAACTTACGGTAAATGGCCCG |
| OG, E11 extra B-cell | CCTGGCTGACCGCCCAACGACCCCCGCCCATTGACGTC |
| enhancer + 18 whole- | AATAATGACGTATGTTCCCATAGTAACGCCAATAGGGAC |
| RAG1pro-RAG- | TTTCCATTGACGTCAATGGGTGGAGTATTTACGGTAAAC |
| WPRE | TGCCCACTTGGCAGTACATCAAGTGTATCATATGCCAAG |
| (SEQ ID NO: 46) | TACGCCCCCTATTGACGTCAATGACGGTAAATGGCCCGC |
| | CTGGCATTATGCCCAGTACATGACCTTATGGGACTTTCC |
| | TACTTGGCAGTACATCTACGTATTAGTCATCGCTATTAC |
| | CATGGTGATGCGGTTTTGGCAGTACATCAATGGGCGTG |
| | GATAGCGGTTTGACTCACGGGGATTTCCAAGTCTCCACC |
| | CCATTGACGTCAATGGGAGTTTGTTTTGGCACCAAAATC |
| | AACGGGACTTTCCAAAATGTCGTAACAACTCCGCCCCAT |
| | TGACGCAAATGGGCGGTAGGCGTGTACGGTGGGAGGTC |
| | TATATAAGCAGAGCTCGTTTAGTGAACCGGGGTCTCTCT |
| | GGTTAGACCAGATCTGAGCCTGGGAGCTCTCTGGCTAA |
| | CTAGGGAACCCACTGCTTAAGCCTCAATAAAGCTTGCCT |
| | TGAGTGCTTCAAGTAGTGTGTGCCCGTCTGTTGTGTGAC |
| | TCTGGTAACTAGAGATCCCTCAGACCCTTTTAGTCAGTG |
| | TGGAAAATCTCTAGCagtggcgcccgaacagggacttgaaagcgaaaggga |
| | aaccagaggagctctctcgacgcaggactcggcttgctgaagcgcgcacggcaagaggcga |
| | ggggcggcgactggtgagtacgccaaaaattttgactagcggaggctagaaggagagagat |
| | gggtgcgagagcgtcagtattaagcgggggagaattagatcgcgatgggaaaaaattcggt |
| | taaggccagggggaaagaaaaaatataaattaaaacatatagtatgggcaagcagggagc |
| | tagaacgattcgcagttaatcctggcctgttagaaacatcagaaggctgtagacaaatactgg |
| | gacagctacaaccatcccttcagacaggatcagaagaacttagatcattatataatacagta |
| | gcaaccctctattgtgtgcatcaaaggatagagataaaagacaccaaggaagctttagacaa |
| | gatagaggaagagcaaaacaaaagtaagaccaccgcacagcaagcggccgctgatcttca |
| | gacctggaggaggagatatgagggacaattggagaagtgaattatataaatataaagtagt |
| | aaaaattgaaccattaggagtagcacccaccaaggcaaagagaagagtggtgcagagaga |
| | aaaaagagcagtgggaataggagctttgttccttgggttcttgggagcagcaggaagcacta |
| | tgggcgcagcgtcaatgacgctgacggtacaggccagacaattattgtctggtatagtgcag |
| | cagcagaacaatttgctgagggctattgaggcgcaacagcatctgttgcaactcacagtctgg |
| | ggcatcaagcagctccaggcaagaatcctggctgtggaaagatacctaaaggatcaacagc |
| | tcctggggatttggggttgctctggaaaactcatttgcaccactgctgtgccttggaatgctagt |
| | tggagtaataaatctctggaacagatttggaatcacacgacctggatggagtgggacagaga |
| | aattaacaattacacaagcttaatacactccttaattgaagaatcgcaaaaccagcaagaaa |
| | agaatgaacaagaattattggaattagataaatgggcaagtttgtggaattggtttaacataa |
| | caaattggctgtggtatataaaattattcataatgatagtaggaggcttggtaggtttaagaat |
| | agtttttgctgtactttctatagtgaatagagttaggcagggatattcaccattatcgtttcagac |
| | ccacctcccaaccccgaggggacccgacaggcccgaaggaatagaagaagaaggtggag |
| | agagagacagagacagatccattcgattagtgaacggatctcgacggtatcggttaactttta |
| | aaagaaaaggggggattggggggtacagtgcaggggaaagaatagtagacataatagca |
| | acagacatacaaactaaagaattacaaaaacaaattacaaaaattcaaaattttatcgatca |
| | cgagactagccAGAAAAGAGGGATTGGGGCCCTCTCCCAACA |
| | GACATGAGTATTTAGGAGTCCCCAGTTCCGGTTTCCACA |
| | TGACATGATATGATGGTCTGTTGTTTTCCTCAGGGACCT |
| | AGAGGCAGATGTAGACAAATAGCAGGTGAGGAGGAAGG |
| | GTGGTGCTGAGCTGTAAACAACAGCTGCGGGCTGTAAA |
| | CAATGGAGCTATATTAAGAAAGTCCTGCATCTGCAGCAT |
| | TCGGTTCAATCCTGCCACCCACTGGGCGTACTCCTGAAT |
| | TGACTTTCCATTGTCAGCTGATCAGTGACAGATGTGAAA |
| | TGACTCGTTACACACACAGGTCACATTGGGAGGATTCTT |
| | AGACGTCATATTCTTTGGTGCTTCACAAAGTCTTACTGT |
| | GGCACATGAAATGATTTCAGATGGTGCACAAGATGATTT |
| | CACATGTAAATTTCCATGAGGGTGCAAAGAGAGGCTTTT |
| | CCCAATCTGAAGAATGAATTTAAAATCTGCTGTAGAAAA |
| | ATACCAGAGAGAAAAATATAGAAAGCAGCTGTCCCTCTT |
| | ACCAATTTCAGAGCAGGAAAGGAATTTCAGTAGAGCATA |
| | AAAACAGCTTTCTCTCATAAAAGAAAACACAACAAGAAA |
| | AATAATCCTTGCTGTGTAGCCTTTTGCAGTTATTTTCTGT |
| | CATGTGTTAGATAGAGACCAGAGGGCTTAACATTGTTTT |
| | GATACCCTTGCATCTGGCAGATTGCCTGTCAGAGGAAGT |
| | TGGAAACGAACTGACCAGCTTTATGCTTCCAGGTGGCAC |
| | TGAAGCCACATTTTCAGTGCCGTTGTTATCAGTCATTTA |
| | ATTCCCTACTGAATATTGAGTGGTGTTTTTAGTTCACAG |
| | GCCATTTGCAGTGCCTTTCTGTTTCCTTTTTAACGTTAGC |
| | CACATTAAGAAAAAAAGTGGGCTTTGCCTACAGCACCTG |
| | CTTCTGCTTTCCGCATTTACTGATCCACTAAGTGTAAGT |
| | GTGACATATAGAGGTCTGTGTTGGTTGTTGTTTTGTGTT |
| | CAAACAGCTGAACATCTGGCCTCTCATGTGCAGGTGCAC |
| | AGAACTCAGGAGAAGGGGTTTTAATTTAAGATAAAAAAA |
| | ATAAGGATATGTGCTAGTTGCAGATGAGGGTTTGGGTG |
| | CTGGACTTAAGATGCTTCTGTGTTCAAAAACAAGGAGTC |
| | CTCAATTTGCAAGGTTCATAAAGAGTTTCACACAGATGG |
| | CACCCAAAGTGCTTATTTCTCATTGTGCTGAAATTAAAC |
| | TTGAATATGCTATATATCAAATAAATAGAAGTAAATAGC |
| | CTTTCTTTATACTTTCTAGAAGTTGAGCTCAATGTTCTTT |
| | CGGAGAGAGCAGGCTCATCTCCATTATGTAGCGCCAAT |
| | GACAAATACCTGTATATGTCTGTATTTCCTTGGTCTGTC |
| | TTGCTCACCTGTTAAGACACCTTGCTGCACAACAGATGG |
| | CACATGCTTATCTTGTGGCAGTGAAGAGTTGACATCAAA |
| | GGCCAAGAAAACCTTGAAACTCATCCTTTTCAGCCCACA |
| | AGAGCAGCTTCTCTCTACCACAGACGATGAAAGCTGGA |
| | GATAACAGGGGCATTCCCACCGCTGCACTCTCTCTTGTG |
| | GCCAGCTGACAGTTAACACAGGTGGGCGTGAGGTGCTG |
| | CCTACATTTTGAAGGTTGTCTATTGATGGGGAACACCAC |
| | CTCTCTGAGCTCAGGCCTCTGCTTTGGCCCGTGGAGCTG |
| | CTGCTTCTGAGAAGTAACCAGGTGGTGAATACCTGCCTG |
| | CTTTTCAGAAAATCCGAGGTGGCTGTCAATTGACAATTA |
| | TTTATTCATTGTAAAATAATCACAGGAACAGCAGCAGTG |
| | TAGGTTTCCCTACCTAGAGGGTGGTATGCAGTGATTCTC |
| | AGGCGCTGGTTGGAAGGCACAGCTGAGGGACACAAACT |
| | GCCAGGAAGTAATGTGGTAACTAGCCATGAGCTTGTGG |
| | TACTAATGGTGGCACGGGAAACAAGGTCTCTGCTTGACT |
| | TTTATTTTCACTCCATAACAAACTCACCAGATCAGGAGC |
| | CTAAGGGTGGGTGGGGAGGGAGAAGAGAGAAAAAAGCA |
| | AAGGGAAAGTTCAAAGTGACACTCGCTGGGGCTGAAAA |
| | CCACACTCCCCTGCAGATGAGGTCCTTGGCTctcgagTCTA |
| | GGCAAGTGTCTCTCTCTCTCTCCCCCTCTTTTTCTTTTAG |
| | AAAAATACTTCGTGATAAAAACACAAAAGTATGTGAATG |
| | GTAATTGAAAATGCATTGCTTCCTTTTAGAAACCCACTA |
| | GGATTTGCATAAACCACTTGGCTGTCTGATAAGTCATTA |
| | TCTGTTCTGTCAGTCCCTGGGGACTTTTAATATTAGGTT |
| | TATATTTTTATAGAAAAGGCAAGTTCTAAATTTGAAGAT |
| | GGGTATTGTTAAACTCTTCAATAATGACAGTTTGTGAAA |
| | CTGCATGTTCCAAAAGTCCTTTAAAAAGTCAATAAGGCg |
| | AAAAGTCTATTTTATCTAATATAAGCGTAGTTACTCCTG |
| | CTTACTTTTGGTTTCCATTTGCATGGAGTATCTTTTTCCA |
| | TCCTTTCACTTTTCATCTATGTGTGTCTTTGTAGTTGAAG |
| | TGAGTTTCTTGTAGATAGCACATGGTTATGTCCTGTTTG |
| | tttttgtccattccttctgtcattttattgttttctggtt |
| | GTTTTGCATTTCTCTTCCTCGTTTCTTTATCTCTTACTGT |
| | TTATCTTTGTGTGGTTTGCTTGGTGACAAATTTTAGTTAC |
| | TTTCTCTTTCGCATCTGTGTATCTGCTCTACTAGTGGGTT |
| | CTATACTTTTGTTTGTTCTCCTGATGGTAGTTATTGCCCT |
| | TTGGCTTTCAGATATAGGGCTCCTTTTTTAGGTCTACTA |
| | CTCTTGTGTCTGAAAGTTGCATTTTGTTTGAGTAAGTTC |
| | ATTAAGCAGGGACCTGAAAGTAAACATCAAAGGTGTGCT |
| | GAATCAGTGGTGACAATTTCATGTATCATATTTCATAAT |
| | AATGTTCTTTCAGAGAGCACATAACATTAACTAGCATGG |
| | AGCAGTGCATGCTCCCTGTATTTCCTTCTGGGGGCCATG |
| | ATTGTGAAAGTGTTGCTTGTGATTTACCAGCAGATATTT |
| | ATACACATTTACTTATCACAAGACGTTTTGAGTCTTTCAT |
| | GTGGAGCTCAAAGCCTTAAAACATAAAAACCAAgCTAAA |
| | GAGCCAGGTGGCAGCTGGAGCTGGGGTCTCCTGGCCCA |
| | TGATTGGCTGCCATCATTTGTGGTTAGCCCTCCATGGTG |
| | GGGGAGGCTGGGAAGGACAGTGGAAGCTGATAAACAGC |
| | TCAGCAGCATGTTCTGAGAAACAAGAGGGCAAGGAGAG |
| | AGCAGAGAACACACTTTGCCTTCTCTTTGGTATTGAGTA |
| | ATATCAACCAAATTGCAGACATCTCAACACTTTGGCCAG |
| | GCAGCCTGCTGAGCAAGGTACCTCAGCCAGCATGgtgagca |
| | agggcgaggagctgttcaccggggtggtgcccatcctggtcgagctggacggcgacgtaaa |
| | cggccacaagttcagcgtgtccggcgagggcgagggcgatgccacctacggcaagctgacc |
| | ctgaagttcatctgcaccaccggcaagctgcccgtgccctggcccaccctcgtgaccaccttc |
| | ggctacggcctgatgtgcttcgcccgctaccccgaccacatgaagcagcacgacttcttcaag |
| | tccgccatgcccgaaggctacgtccaggagcgcaccatcttcttcaaggacgacggcaacta |
| | caagacccgcgccgaggtgaagttcgagggcgacaccctggtgaaccgcatcgagctgaag |
| | ggcatcgacttcaaggaggacggcaacatcctggggcacaagctggagtacaactacaaca |
| | gccacaacgtctatatcatggccgacaagcagaagaacggcatcaaggtgaacttcaagat |
| | ccgccacaacatcgaggacggcagcgtgcagctcgccgaccactaccagcagaacaccccc |
| | atcggcgacggccccgtgctgctgcccgacaaccactacctgagctaccagtccgccctgag |
| | caaagaccccaacgagaagcgcgatcacatggtcctgctggagttcgtgaccgccgccggg |
| | atcactctcggcatggacgagctgtacaagtgactgcaggaattcgagcatcttaccgccatt |
| | tattcccatatttgttctgtttttcttgatttgggtatacatttaaatgttaataaaacaaaatggt |
| | ggggcaatcatttacatttttagggatatgtaattactagttcaggtgtattgccacaagacaa |
| | acatgttaagaaactttcccgttatttacgctctgttcctgttaatcaacctctggattacaaaat |
| | ttgtgaaagattgactgatattcttaactatgttgctccttttacgctgtgtggatatgctgcttta |
| | atgcctctgtatcatgctattgcttcccgtacggctttcgttttctcctccttgtataaatcctggt |
| | tgctgtctctttatgaggagttgtggcccgttgtccgtcaacgtggcgtggtgtgctctgtgtttgc |
| | tgacgcaacccccactggctggggcattgccaccacctgtcaactcctttctgggactttcgctt |
| | tccccctcccgatcgccacggcagaactcatcgccgcctgccttgcccgctgctggacagggg |
| | ctaggttgctgggcactgataattccgtggtgttgtcggggaagggcctgctgccggctctgcg |
| | gcctcttccgcgtcttcgccttcgccctcagacgagtcggatctccctttgggccgcctccccgc |
| | ctggaattcgagctcggtacctttaagaccaatgacttacaaggcagctgtagatcttagcca |
| | ctttttaaaagaaaaggggggactggaagggctaattcactcccaacgaagacaagatctgc |
| | tttttgcttgtactgggtctctctggttagaccagatctgagcctgggagctctctggctaactag |
| | ggaacctactgcttaagcctcaataaagcttgccttgagtgcttCAAGTAGTGTGT |
| | GCCCGTCTGTTGTGTGACTCTGGTAACTAGAGATCCCTC |
| | AGACCCTTTTAGTCAGTGTGGAAAATCTCTAGCagtagtagtt |
| | catgtcatcttattattcagtatttataacttgcaaagaaatgaatatcagagagtgagaggaa |
| | cttgtttattgcagcttataatggttacaaataaagcaatagcatcacaaatttcacaaataaa |
| | gcatttttttcactgcattctagttgtggtttgtccaaactcatcaatgtatcttatcatgtctggc |
| | tctagctatcccgcccctaactccgcccatcccgcccctaactccgcccagttccgcccattctcc |
| | gccccatggctgactaattttttttatttatgcagaggccgaggccgcctcggcctctgagctat |
| | tccagaagtagtgaggaggcttttttggaggcctagggacgtacccaattcgccctatagtga |
| | gtcgtattacgcgcgctcactggccgtcgttttacaacgtcgtgactgggaaaaccctggcgtt |
| | acccaacttaatcgccttgcagcacatccccctttcgccagctggcgtaatagcgaagaggcc |
| | cgcaccgatcgcccttcccaacagttgcgcagcctgaatggcgaatgggacgcgccctgtag |
| | cggcgcattaagcgcggcgggtgtggtggttacgcgcagcgtgaccgctacacttgccagcg |
| | ccctagcgcccgctcctttcgctttcttcccttcctttctcgccacgttcgccggctttccccgtca |
| | agctctaaatcgggggctccctttagggttccgatttagtgctttacggcacctcgaccccaaa |
| | aaacttgattagggtgatggttcacgtagtgggccatcgccctgatagacggtttttcgcccttt |
| | gacgttggagtccacgttctttaatagtggactcttgttccaaactggaacaacactcaaccct |
| | atctcggtctattcttttgatttataagggattttgccgatttcggcctattggttaaaaaatgag |
| | ctgatttaacaaaaatttaacgcgaattttaacaaaatattaacgcttacaatttaggtggcac |
| | ttttcggggaaatgtgcgcggaacccctatttgtttatttttctaaatacattcaaatatgtatcc |
| | gctcatgagacaataaccctgataaatgcttcaataatagcacctagatcaagagacaggat |
| | gaggatcgtttcgcatgattgaacaagatggattgcacgcaggttctccggccgcttgggtgg |
| | agaggctattcggctatgactgggcacaacagacaatcggctgctctgatgccgccgtgttcc |
| | ggctgtcagcgcaggggcgcccggttctttttgtcaagaccgacctgtccggtgccctgaatg |
| | aactgcaagacgaggcagcgcggctatcgtggctggccacgacgggcgttccttgcgcagct |
| | gtgctcgacgttgtcactgaagcgggaagggactggctgctattgggcgaagtgccggggca |
| | ggatctcctgtcatctcaccttgctcctgccgagaaagtatccatcatggctgatgcaatgcgg |
| | cggctgcatacgcttgatccggctacctgcccattcgaccaccaagcgaaacatcgcatcga |
| | gcgagcacgtactcggatggaagccggtcttgtcgatcaggatgatctggacgaagagcatc |
| | aggggctcgcgccagccgaactgttcgccaggctcaaggcgagcatgcccgacggcgagg |
| | atctcgtcgtgacccatggcgatgcctgcttgccgaatatcatggtggaaaatggccgcttttc |
| | tggattcatcgactgtggccggctgggtgtggcggaccgctatcaggacatagcgttggctac |
| | ccgtgatattgctgaagagcttggcggcgaatgggctgaccgcttcctcgtgctttacggtatc |
| | gccgctcccgattcgcagcgcatcgccttctatcgccttcttgacgagttcttctgaattattaac |
| | gcttacaatttcctgatgcggtattttctccttacgcatctgtgcggtatttcacaccgcatcagg |
| | tggcacttttcggggaaatgtgcgcggaacccctatttgtttatttttctaaatacattcaaatat |
| | gtatccgctcatgaccaaaatcccttaacgtgagttttcgttccactgagcgtcagaccccgta |
| | gaaaagatcaaaggatcttcttgagatcctttttttctgcgcgtaatctgctgcttgcaaacaaa |
| | aaaaccaccgctaccagcggtggtttgtttgccggatcaagagctaccaactctttttccgaag |
| | gtaactggcttcagcagagcgcagataccaaatactgttcttctagtgtagccgtagttaggcc |
| | accacttcaagaactctgtagcaccgcctacatacctcgctctgctaatcctgttaccagtggc |
| | tgctgccagtggcgataagtcgtgtcttaccgggttggactcaagacgatagttaccggataa |
| | ggcgcagcggtcgggctgaacggggggttcgtgcacacagcccagcttggagcgaacgacc |
| | tacaccgaactgagatacctacagcgtgagctatgagaaagcgccacgcttcccgaaggga |
| | gaaaggcggacaggtatccggtaagcggcagggtcggaacaggagagcgcacgagggag |
| | cttccagggggaaacgcctggtatctttatagtcctgtcgggtttcgccacctctgacttgagcg |
| | tcgatttttgtgatgctcgtcaggggggcggagcctatggaaaaacgccagcaacgcggcctt |
| | tttacggttcctggccttttgctggccttttgctcacatgttctttcctgcgttatcccctgattct |
| | gtggataaccgtattaccgcctttgagtgagctgataccgctcgccgcagccgaacgaccgagc |
| | gcagcgagtcagtgagcgaggaagcggaagagcgcccaatacgcaaaccgcctctccccg |
| | cgcgttggccgattcattaatgcagctggcacgacaggtttcccgactggaaagcgggcagt |
| | gagcgcaacgcaattaatgtgagttagctcactcattaggcaccccaggctttacactttatgc |
| | ttccggctcgtatgttgtgtggaattgtgagcggataacaatttcacacaggaaacagctatg |
| | accatgattacgccaagcgcgcaattaaccctcactaaagggaacaaaagctggagctgca |
| | agcttggccattgcatacgttgtatccatatcataatatgtacatttatattggctcatgtccaac |
| | attaccgccat |

In various embodiments, the lentiviral vectors (LVs) described herein can have various “safety” features that can include, for example, the presence of an insulator (e.g., an FB insulator in the 3′LTR). Additionally, or alternatively, in certain embodiments, the HIV LTR has been substituted with an alternative promoter (e.g., a CMV promoter, see, e.g., SEQ ID NO:21) to yield a higher titer vector without the inclusion of the HIV TAT protein during packaging. Other strong promoters (e.g., RSV, and the like can also be used).

As noted above, in various embodiments the lentiviral vectors described herein contain any one or more of the elements typically found in lentiviral vectors. Such elements include, but need not be limited to a ψ region vector genome packaging signal (see, e.g., SEQ ID NO:23), a Rev Responsive Element (RRE) (see, e.g., SEQ ID NO:24), a polypurine tract (e.g., a central polypurine tract, a 3′ polypurine tract (see, e.g., SEQ ID NO:26), etc.), a post-translational regulatory element (e.g., a modified Woodchuck Post-transcriptional Regulatory Element (WPRE) (see, e.g., SEQ ID NO:25), an insulator, and the like, e.g., as described below.

In various embodiments the vector is a SIN vector substantially incapable of reconstituting a wild-type lentivirus through recombination.

As shown above, in Example 1, the vectors described herein are believed to be effective to transduce cells at high titer and to also provide high levels of expression of a nucleic acid encoding RAG1 protein.

In view of these results, it is believed that LVs described herein, e.g., recombinant TAT-independent, SIN LVs that express a nucleic acid encoding a RAG1 protein can be used to effectively treat RAG1 SCID in subjects (e.g., human and non-human mammals) It is believed these vectors can be used for the modification of stem cells (e.g., hematopoietic stem and progenitor cells) that can be introduced into a subject in need thereof for the treatment of, e.g., subjects identified as having RAG1 SCID. Moreover, it is believed that the resulting cells will produce enough of the transgenic RAG1 protein to demonstrate significant improvement in subject health. It is also believed the vectors can be directly administered to a subject to achieve in vivo transduction of the target (e.g., hematopoietic stem or progenitor cells) and thereby also effect a treatment of subjects in need thereof.

As noted above, in various embodiments the LVs described herein can comprise various safety features. For example, the HIV LTR has been substituted with a CMV promoter to yield higher titer vector without the inclusion of the HIV TAT protein during packaging. In certain embodiments an insulator (e.g., the FB insulator) can be introduced into the 3′LTR for safety. The LVs are also constructed to provide efficient transduction and high titer.

It will be appreciated that the foregoing elements are illustrative and need not be limiting. In view of the teachings provided herein, suitable substitutions for these elements will be recognized by one of skill in the art and are contemplated within the scope of the teachings provided herein.

### RAG1 Codon Optimization.

As noted above, in various embodiments the lentiviral vector can comprise a RAG1 gene or cDNA. However, in certain embodiments the nucleic acid encoding RAG1 protein is codon optimized. Numerous methods of codon optimization are known to those of skill in the art. One illustrative method is JCat (Java Codon Adaptation Tool). The jCAT tool adapts gene codon usage to most sequenced prokaryotes and various eukaryotic gene expression hosts. In contrast to many tools, JCat does not require the manual definition of highly expressed genes and is, therefore, a very rapid and easy method. Further options of JCat for codon adaptation include the avoidance of unwanted cleavage sites for restriction enzymes and Rho-independent transcription terminators. The output of JCat is both graphically and as Codon Adaptation Index (CAI) values given for the input sequence and the newly adapted sequence. JCat optimization is described by Grote et al. (2005) Nucleic Acids Res. 33 (suppl 2): W526-W531) and a JCat tool is available online at www.jcat.de.

Another codon optimization tool is provided by GeneArt (from ThermoFisher Scientific®.

Still another codon optimization tool is IDT. The IDT codon optimization tool was developed to optimize a DNA or protein sequence from one organism for expression in another by reassigning codon usage based on the frequencies of each codon's usage in the new organism. For example, valine is encoded by 4 different codons (GUG, GUU, GUC, and GUA). In human cell lines, however, the GUG codon is preferentially used (46% use vs. 18, 24, and 12%, respectively). The codon optimization tool takes this information into account and assigns valine codons with those same frequencies. In addition, the tool algorithm eliminates codons with less than 10% frequency and re-normalizes the remaining frequencies to 100%. Moreover, the optimization tool reduces complexities that can interfere with manufacturing and downstream expression, such as repeats, hairpins, and extreme GC content. The IDT optimization tool is available from IDT (Integrated DNA Technologies, Coralville, Iowa) and can be found at ww.idtdna.com/CodonOpt.

Other codon optimization tools include, but are not limited to CodonW an open source software program that can be found at codonw.sourceforge.net, and the OptimumGene™ algorithm from GenScript.

These codon optimizations are illustrative and non-limiting. Using the teaching provided here and in Example 1, the RAG1 codon usage can readily be optimized for particular applications.

### TAT-Independent and Self Inactivating Lentiviral Vectors.

To further improve safety, in various embodiments, the lentiviral vectors described herein comprise a TAT-independent, self-inactivating (SIN) configuration. Thus, in various embodiments it is desirable to employ in the LVs described herein an LTR region that has reduced promoter activity relative to wild-type LTR. Such constructs can be provided that are effectively “self-inactivating” (SIN) which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed.

Furthermore, a SIN design reduces the possibility of interference between the LTR and the promoter that is driving the expression of the transgene. SIN LVs can often permit full activity of the internal promoter.

The SIN design increases the biosafety of the LVs. The majority of the HIV LTR is comprised of the U3 sequences. The U3 region contains the enhancer and promoter elements that modulate basal and induced expression of the HIV genome in infected cells and in response to cell activation. Several of these promoter elements are essential for viral replication. Some of the enhancer elements are highly conserved among viral isolates and have been implicated as critical virulence factors in viral pathogenesis. The enhancer elements may act to influence replication rates in the different cellular target of the virus

As viral transcription starts at the 3′ end of the U3 region of the 5′ LTR, those sequences are not part of the viral mRNA and a copy thereof from the 3′ LTR acts as template for the generation of both LTR's in the integrated provirus. If the 3′ copy of the U3 region is altered in a retroviral vector construct, the vector RNA is still produced from the intact 5′ LTR in producer cells, but cannot be regenerated in target cells. Transduction of such a vector results in the inactivation of both LTR's in the progeny virus. Thus, the retrovirus is self-inactivating (SIN) and those vectors are known as SIN transfer vectors.

In certain embodiments self-inactivation is achieved through the introduction of a deletion in the U3 region of the 3′ LTR of the vector DNA, i.e., the DNA used to produce the vector RNA. During RT, this deletion is transferred to the 5′ LTR of the proviral DNA. Typically, it is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function typically spread out over U3, R and U5. Accordingly, in certain embodiments, it is desirable to eliminate as many of the transcriptionally important motifs from the LTR as possible while sparing the polyadenylation determinants.

The SIN design is described in detail in Zufferey et al. (1998) J Virol. 72(12): 9873-9880, and in U.S. Pat. No. 5,994,136. As described therein, there are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′LTR may decrease polyadenylation of vector transcripts with adverse consequences both on the titer of the vector in producer cells and the transgene expression in target cells.

Additional SIN designs are described in U.S. Patent Publication No: 2003/0039636. As described therein, in certain embodiments, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

Accordingly, in certain embodiments, the SIN configuration provides a retroviral LTR comprising a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In a particular embodiment, the retroviral LTR comprises a hybrid R region, wherein the hybrid R region comprises a portion of the HIV R region (e.g., a portion comprising or consisting of the nucleotide sequence shown in SEQ ID NO: 10 in US 2003/0039636) lacking the TAR sequence, and a portion of the MoMSV R region (e.g., a portion comprising or consisting of the nucleotide sequence shown in SEQ ID NO: 9 in 2003/0039636) comparable to the TAR sequence lacking from the HIV R region. In another particular embodiment, the entire hybrid R region comprises or consists of the nucleotide sequence shown in SEQ ID NO: 11 in 2003/0039636.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruses. In one illustrative embodiment, the lentivirus is HIV and the non-lentiviral retrovirus is MoMSV.

In another embodiment described in US 2003/0039636, the LTR comprising a hybrid R region is a left (5′) LTR and further comprises a promoter sequence upstream from the hybrid R region. Preferred promoters are non-lentiviral in origin and include, for example, the U3 region from a non-lentiviral retrovirus (e.g., the MoMSV U3 region). In one particular embodiment, the U3 region comprises the nucleotide sequence shown in SEQ ID NO: 12 in US 2003/0039636. In another embodiment, the left (5′) LTR further comprises a lentiviral U5 region downstream from the hybrid R region. In one embodiment, the U5 region is the HIV U5 region including the HIV att site necessary for genomic integration. In another embodiment, the U5 region comprises the nucleotide sequence shown in SEQ ID NO: 13 in US 2003/0039636. In yet another embodiment, the entire left (5′) hybrid LTR comprises the nucleotide sequence shown in SEQ ID NO: 1 in US 2003/0039636.

In another illustrative embodiment, the LTR comprising a hybrid R region is a right (3′) LTR and further comprises a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region consists of the 3′ end of a lentiviral (e.g., HIV) U3 region up to and including the lentiviral U3 att site. In one embodiment, the U3 region comprises the nucleotide sequence shown in SEQ ID NO: 15 in US 2003/0039636. In another embodiment, the right (3′) LTR further comprises a polyadenylation sequence downstream from the hybrid R region. In another embodiment, the polyadenylation sequence comprises the nucleotide sequence shown in SEQ ID NO: 16 in US 2003/0039636. In yet another embodiment, the entire right (5′) LTR comprises the nucleotide sequence shown in SEQ ID NO: 2 or 17 of US 2003/0039636.

Thus, in the case of HIV based LV, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower.

It has also been demonstrated that the trans-acting function of Tat becomes dispensable if part of the upstream LTR in the transfer vector construct is replaced by constitutively active promoter sequences (see, e.g., Dull et al. (1998) J Virol. 72(11): 8463-8471. Furthermore, we show that the expression of rev in trans allows the production of high-titer HIV-derived vector stocks from a packaging construct which contains only gag and pol. This design makes the expression of the packaging functions conditional on complementation available only in producer cells. The resulting gene delivery system, conserves only three of the nine genes of HIV-1 and relies on four separate transcriptional units for the production of transducing particles.

In one embodiments illustrated in Example 1, the cassette expressing a nucleic acid encoding RAG1 protein is a SIN vector with the CMV enhancer/promoter substituted in the 5′ LTR.

It will be recognized that the CMV promoter typically provides a high level of non-tissue specific expression. Other promoters with similar constitutive activity include, but are not limited to the RSV promoter, and the SV40 promoter. Mammalian promoters such as the beta-actin promoter, ubiquitin C promoter, elongation factor 1αpromoter, tubulin promoter, etc., may also be used.

The foregoing SIN configurations are illustrative and non-limiting. Numerous SIN configurations are known to those of skill in the art. As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95% at least about 96%, at least about 97%, at least about 98%, or at least about 99% transcriptionally inactive.

### Insulator Element

In certain embodiments, to further enhance biosafety, insulators are inserted into the lentiviral vectors described herein. Insulators are DNA sequence elements present throughout the genome. They bind proteins that modify chromatin and alter regional gene expression. The placement of insulators in the vectors described herein offer various potential benefits including, inter alia: 1) Shielding of the vector from positional effect variegation of expression by flanking chromosomes (i.e., barrier activity); and 2) Shielding flanking chromosomes from insertional trans-activation of gene expression by the vector (enhancer blocking). Thus, insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Natl. Acad. Sci. USA, 99: 16433; and Zhan et al. (2001) Hum. Genet., 109: 471). In the present context insulators may contribute to protecting lentivirus-expressed sequences from integration site effects, which may be mediated by cis-acting elements present in genomic DNA and lead to deregulated expression of transferred sequences. In various embodiments LVs are provided in which an insulator sequence is inserted into one or both LTRs or elsewhere in the region of the vector that integrates into the cellular genome.

The first and best characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb fragment containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp fragment. Within this stretch is a 49-bp cHS4 core (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

One illustrative and suitable insulator is FB (FII/BEAD-A), a 77 bp insulator element, that contains the minimal CTCF binding site enhancer-blocking components of the chicken β-globin 5′ HS4 insulators and a homologous region from the human T-cell receptor alpha/delta blocking element alpha/delta I (BEAD-I) insulator described by Ramezani et al. (2008) Stem Cell 26: 3257-3266. The FB “synthetic” insulator has full enhancer blocking activity. This insulator is illustrative and non-limiting. Other suitable insulators may be used including, for example, the full-length chicken beta-globin HS4 or insulator sub-fragments thereof, the ankyrin gene insulator, and other synthetic insulator elements.

### Packaging Signal.

In various embodiments the vectors described herein further comprise a packaging signal. A “packaging signal,” “packaging sequence,” or “PSI sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art. One illustrative, but non-limiting PSI is provided by SEQ ID NO:23.

### Rev Responsive Element (RRE).

In certain embodiments the lentiviral vectors described herein comprise a Rev response element (RRE) to enhance nuclear export of unspliced RNA. RREs are well known to those of skill in the art. Illustrative RREs include, but are not limited to RREs such as that located at positions 7622-8459 in the HIV NL4-3 genome (Genbank accession number AF003887) as well as RREs from other strains of HIV or other retroviruses. Such sequences are readily available from Genbank or from the database with URL hiv-web.lanl.gov/content/index. One illustrative, but non-limiting RRE is shown in SEQ ID NO:24).

PolyPurine Tract (cPPT, 3′PPT).

In various embodiments the lentiviral vectors described herein further include a polypurine tract (e.g., central polypurine tract (cPPT), 3′ poplypurine tract (3′PPT)). Insertion of a fragment containing the 3′PPT (see, e.g., SEQ ID NO:26) or the central polypurine tract (cPPT) in lentiviral (e.g., HIV-1) vector constructs is known to enhance transduction efficiency.

### Expression-Stimulating Posttranscriptional Regulatory Element (PRE)

In certain embodiments the lentiviral vectors (LVs) described herein may comprise any of a variety of posttranscriptional regulatory elements (PREs) whose presence within a transcript increases expression of the heterologous nucleic acid (e.g., RAG1) at the protein level. PREs may be particularly useful in certain embodiments, especially those that involve lentiviral constructs with modest promoters.

One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Posttranscriptional regulatory elements that do not rely on splicing events offer the advantage of not being removed during the viral life cycle. Some examples are the posttranscriptional processing element of herpes simplex virus, the posttranscriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit.

The WPRE is characterized and described in U.S. Pat. No. 6,136,597. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7- to 10-fold. Retroviral vectors transfer sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE (see, e.g., SEQ ID NO:25) in a vector results in enhanced expression of transgenes.

### Transduced Host Cells and Methods of Cell Transduction.

The recombinant lentiviral vectors (LV) and resulting virus described herein are capable of transferring a heterologous nucleic acid sequence (e.g., a nucleic acid encoding RAG1 protein) into a mammalian cell. In various embodiments, for delivery to cells, vectors described herein are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

In certain embodiments the vectors are introduced via transfection into a packaging cell line. The packaging cell line produces viral particles that contain the vector genome. Methods for transfection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with or without a dominant selectable marker, such as neomycin, DHFR, Glutamine synthetase, followed by selection in the presence of the appropriate drug and isolation of clones. In certain embodiments the selectable marker gene can be linked physically to the packaging genes in the construct.

Stable cell lines wherein the packaging functions are configured to be expressed by a suitable packaging cell are known (see, e.g., U.S. Pat. No. 5,686,279, which describes packaging cells). In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a lentiviral vector incorporated therein form producer cells. Producer cells are thus cells or cell-lines that can produce or release packaged infectious viral particles carrying the therapeutic gene of interest (e.g., a nucleic acid that encodes a RAG1 protein). These cells can further be anchorage dependent which means that these cells will grow, survive, or maintain function optimally when attached to a surface such as glass or plastic. Some examples of anchorage dependent cell lines used as lentiviral vector packaging cell lines when the vector is replication competent are HeLa or 293 cells and PERC.6 cells.

Accordingly, in certain embodiments, methods are provided of delivering a gene to a cell which is then integrated into the genome of the cell, comprising contacting the cell with a virion containing a lentiviral vector described herein. The cell (e.g., in the form of tissue or an organ) can be contacted (e.g., infected) with the virion ex vivo and then delivered to a subject (e.g., a mammal, animal or human) in which the gene (e.g., a nucleic acid encoding RAG1 protein) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

In certain embodiments, the lentivectors described herein will be particularly useful in the transduction of human hematopoietic progenitor cells or a hematopoietic stem cells, obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ hematopoietic stem and progenitor cells.

### Gene Therapy.

In still other embodiments, methods are provided for transducing a human hematopoietic stem cell. In certain embodiments the methods involve contacting a population of human cells that include hematopoietic stem cells with one of the foregoing lentivectors under conditions to effect the transduction of a human hematopoietic progenitor cell in said population by the vector. The stem cells may be transduced in vivo or in vitro, depending on the ultimate application. Even in the context of human gene therapy, such as gene therapy of human stem cells, one may transduce the stem cell in vivo or, alternatively, transduce in vitro followed by infusion of the transduced stem cell into a human subject. In one aspect of this embodiment, the human stem cell can be removed from a human, e.g., a RAG1 SCID patient, using methods well known to those of skill in the art and transduced as noted above. The transduced stem cells are then reintroduced into the same or a different human.

Stem Cell/Progenitor Cell Gene Therapy.

In various embodiments the lentivectors described herein are particularly useful for the transduction of human hematopoietic progenitor cells or haematopoietic stem cells (HSCs), obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ hematopoietic stem and progenitor cells.

When cells, for instance CD34⁺ cells, dendritic cells, peripheral blood cells or tumor cells are transduced ex vivo, the vector particles are incubated with the cells using a dose generally in the order of between 1 to 50 multiplicities of infection (MOI) which also corresponds to 1×10⁵ to 50×10⁵ transducing units of the viral vector per 10⁵ cells. This can include amounts of vector corresponding to 1, 2, 3, 4, 5, 6, 7, 8, 9, 10, 15, 20, 25, 30, 35, 40, 45, and 50 MOI. Typically, the amount of vector may be expressed in terms of HT-29 transducing units (TU).

In certain embodiments cell-based therapies involve providing stem cells and/or hematopoietic precursors, transduce the cells with the lentivirus encoding, e.g., a nucleic acid encoding a RAG1 protein, and then introduce the transformed cells into a subject in need thereof (e.g., a subject with a mutation in the RAG1 gene).

In certain embodiments the methods involve isolating population of cells, e.g., stem cells from a subject, optionally expand the cells in tissue culture, and administer the lentiviral vector whose presence within a cell results in production of a normal RAG1 protein in the cells in vitro. The cells are then returned to the subject, where, for example, they may provide a population of lymphocytes that produce the RAG1 protein.

In some illustrative, but non-limiting, embodiments, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Where stem cells are to be used, it will be recognized that such cells can be derived from a number of sources including bone marrow (BM), cord blood (CB), mobilized peripheral blood stem cells (mPBSC), and the like. In certain embodiments the use of induced pluripotent stem cells (IPSCs) is contemplated. Methods of isolating hematopoietic stem cells (HSCs), transducing such cells and introducing them into a mammalian subject are well known to those of skill in the art.

Direct Introduction of Vector.

In certain embodiments direct treatment of a subject by direct introduction of the vector(s) described herein is contemplated. The lentiviral compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In various embodiments pharmaceutical compositions can include an LV in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a lentiviral described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a LV calculated to produce the desired therapeutic effect in association with a pharmaceutical carrier.

A unit dose need not be administered as a single injection but may comprise continuous infusion over a set period of time. Unit dose of the LV described herein may conveniently be described in terms of transducing units (T.U.) of lentivector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a LV can include a single treatment or, in many cases, can include a series of treatments.

Illustrative, but non-limiting, doses for administration of gene therapy vectors and methods for determining suitable doses are known in the art. It is furthermore understood that appropriate doses of a LV may depend upon the particular recipient and the mode of administration. The appropriate dose level for any particular subject may depend upon a variety of factors including the age, body weight, general health, gender, and diet of the subject, the time of administration, the route of administration, the rate: of excretion, other administered therapeutic agents, and the like.

In certain embodiments lentiviral gene therapy vectors described herein can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or inhalationally and may be encapsulated or otherwise manipulated to protect them from degradation, enhance uptake into tissues or cells, etc. Pharmaceutical preparations can include a LV in an acceptable diluent, or can comprise a slow release matrix in which a LV is imbedded. Alternatively or additionally, where a vector can be produced intact from recombinant cells, as is the case for retroviral or lentiviral vectors as described herein, a pharmaceutical preparation can include one or more cells which produce vectors. Pharmaceutical compositions comprising a LV described herein can be included in a container, pack, or dispenser, optionally together with instructions for administration.

The foregoing compositions, methods and uses are intended to be illustrative and not limiting. Using the teachings provided herein other variations on the compositions, methods and uses will be readily available to one of skill in the art.

### Example 1

### Development of Lentiviral Vectors for Treatment of Recombination-Activating Gene 1 Severe Combined Immunodeficiency (RAG1 SCID)

The goal of the experiments described below was to develop a novel lentiviral vector driven by endogenous regulatory elements of the native RAG1 gene for the treatment of Recombination-Activating Gene 1 Severe Combined Immunodeficiency (RAG1 SCID). In particular, it was desired to develop a novel clinical lentiviral vector for the treatment of RAG1 SCID. In particular, it was desired to develop a lentiviral vector regulated by endogenous elements of the RAG1 locus for high-level, lineage and temporal specific expression.

A bioinformatic analysis (using publicly available databases: Project Encode, Ensembl, FANTOM, VISTA Enhancer Browser, GeneHancer) was utilized to elucidate the endogenous regulatory elements of the native RAG1 gene. Eighteen putative enhancer elements were identified located within a 400,000 base pair window. It is believed that a subset of the 18 putative enhancer elements are each responsible for RAG1 expression at specific timepoints throughout T and B cell development and that various combinations of these elements can be incorporated into an expression cassette in, for example, a lentiviral vector, to provide vectors that can recapitulate the expression pattern of the native RAG1 gene at therapeutically effective levels.

In order to experimentally identify the critical enhancer elements that regulate the RAG1 gene, each putative enhancer element was cloned upstream of the endogenous RAG1 promoter to drive expression of a reporter (mCitrine) (see, e.g., FIG. 1). A vector was also cloned containing all 18 elements (see, e.g., FIG. 1). Other pre-clinical RAG1 vectors were used as controls.

A duplicate set of the vectors were re-cloned to contain a unique 15 nucleotide barcode in the 3′ untranslated region (3′UTR) to allow for multiplexing (see, e.g., FIG. 2). Each vector has been cloned to contain 2 unique barcodes for a total of 42 new vectors.

The experimental plan involves providing a pooled viral supernatant containing all 42 vectors to transduce CB CD34+ HSCs. Transduced HSCs are transplanted into irradiated NSG neonates and the transduced HSCs containing all 42 vectors will develop into mature T and B cells. Mice will be harvested 16 weeks post transplantation and the different cell stages of T-cell (esp. thymocytes) and B-cell development will be collected. RNA and gDNA will be extracted from each cell population; barcodes in the RNA and gDNA will be amplified by PCR and quantified by next-generation sequencing.

Since each barcode is associated with a known enhancer element, by quantifying the relative abundance of each barcode in a specific cell population, we can elucidate the enhancer elements which are responsible for the high level, lineage and temporal specific expression of RAG through specific stages of T and B cell development.

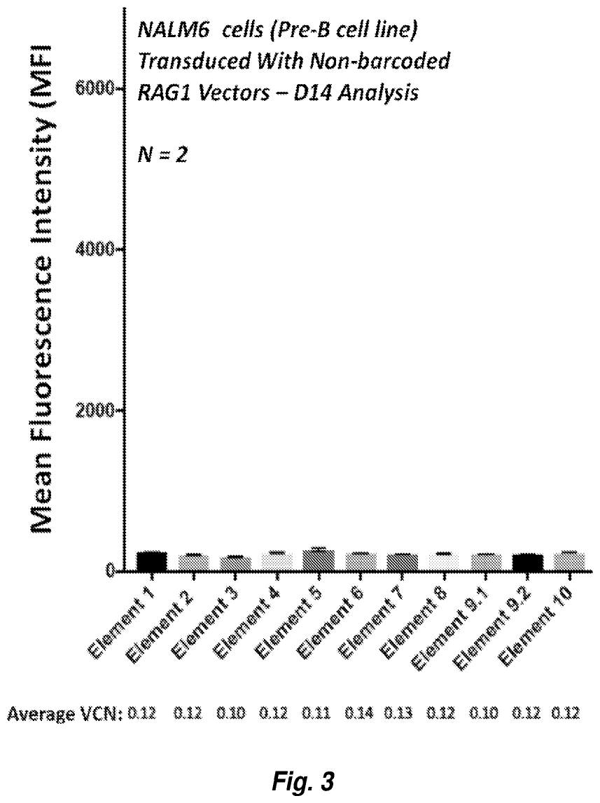

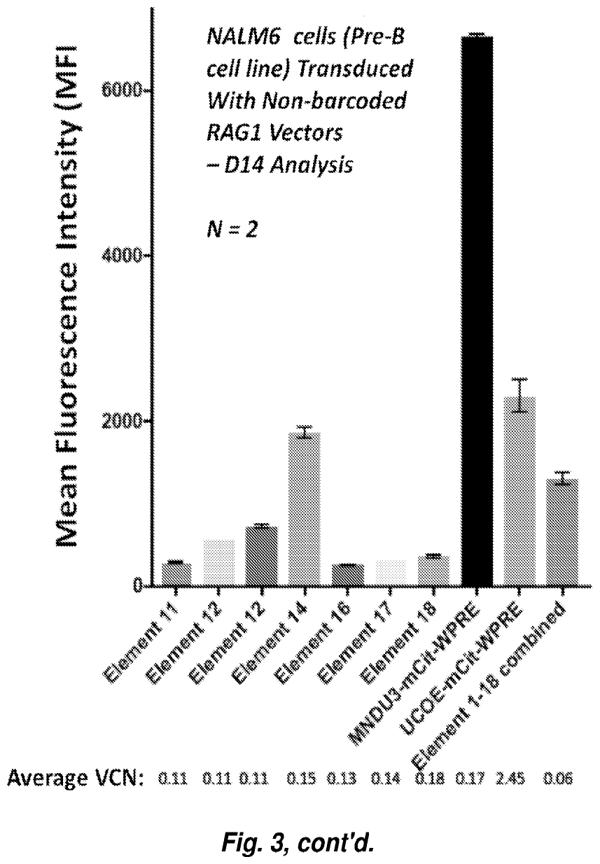

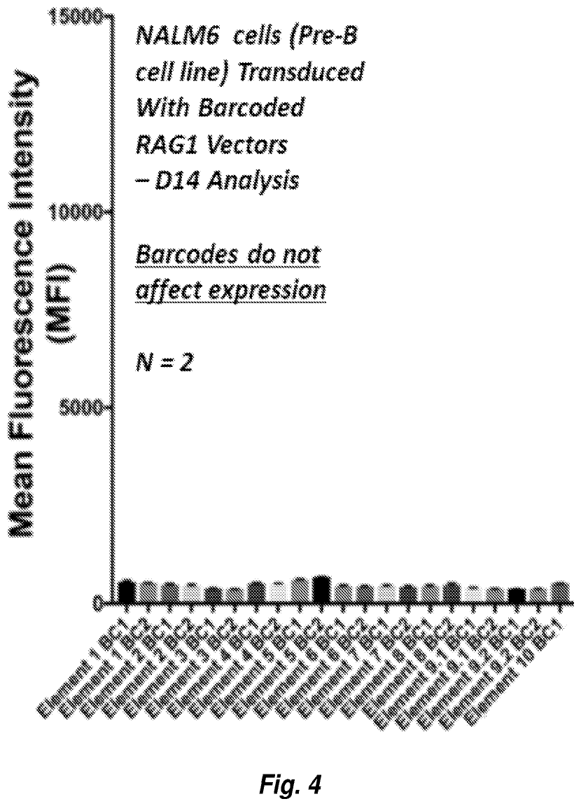

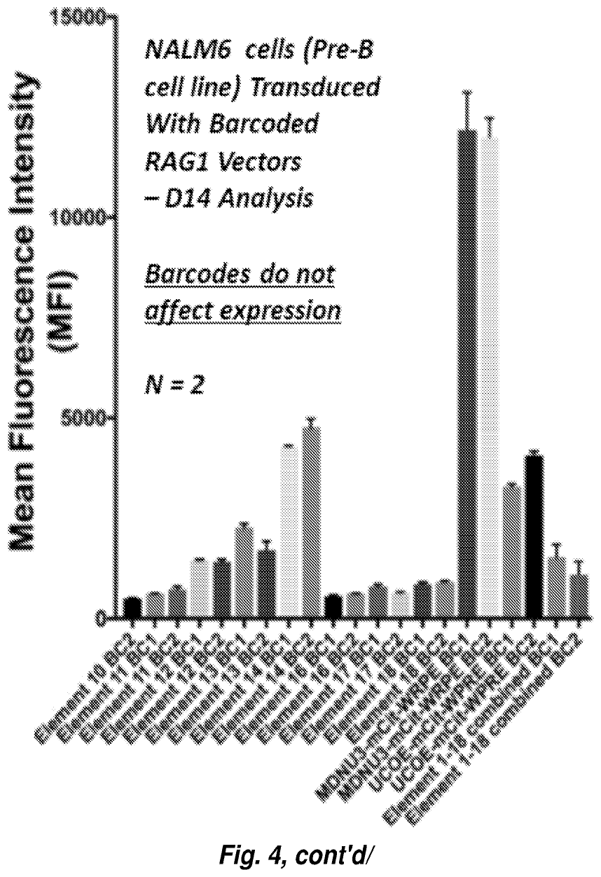

As a proof of concept, NALM6 cells (pre B-cell line) were transduced with non-barcoded RAG1 vectors to validate that the vectors express and to validate known pre-B cell enhancers. Element #14 BRAG is a pre-B cell enhancer needed to advance from the pro-B cell stage to the pre-B cell stage previously defined in the literature. Expression levels of each of the vectors in transduced NALM6 cells are shown in FIG. 3. NALM6 cells (pre B-cell line) were then transduced with barcoded RAG1 vectors to demonstrate that barcodes do not affect expression (see, FIG. 4).

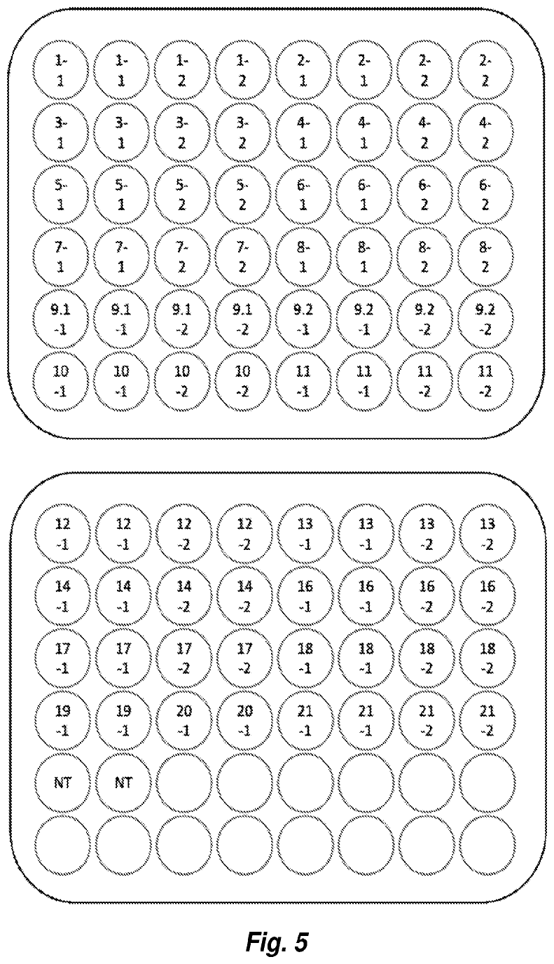

In order to confirm the credibility of multiplexing, all the singleplex transduced barcoded wells are mixed together (see, FIG. 5). RNA and gDNA are extracted from the mixed cell population to demonstrate if we can recapitulate the data of analyzing each well independently by flow cytometry.

Additionally, NALM6 cells were transduced with pooled viral supernatant containing all 42 vectors in increasing vector doses to determine if recombination of barcodes occurs at high vector copy numbers. This was also done to determine if a single multiplexed transduced well can recapitulate the data from 42 singleplex transduced wells.

Multiplexed CB CD34+ HSCs were transduced into NSG neonates to identify lineage and temporal specific enhancers at different stages of T and B cell development. Bone marrow was harvested from femurs and tibias as well as lymph nodes from 21 mice and recovered cells were selected for CD45+ cells. B-cell and T-cell yields were determined.

High cell yields of each B-cell developmental population from NSG mice were observed. Lower but workable yields of T-cell developmental stages (very low CD34+ Thy1, Thy2, Thy3 populations) were also observed.

RNA and gDNA from each population can be collected and the RNA can be cDNA converted and barcodes can be PCR′d out of cDNA and gDNA.

As described below, we performed an in-vivo enhancer screen of the various constructs in:
NOD.Cg-Prkdc^{scid} Il2rg^{tm/Wjl}/SzJ (NSG) mice;
Bone Marrow Liver Thymic (BLT) mice; and
Artificial Thymic Organoids (ATOs).

The in vivo ehancer screen in NSG mice is described above. The in vivo enhancer screen in BLT mice was similar to the NSG screen, but the transduced CD34+ cells are transplanted into BLT mice instead of NSG mice. BLT mice are modified NSG mice transplanted with a human fetal thymus to facilitate human T-cell development.

The enhancer screen in ATOs was similar to the NSG mice but the transduced cells were differentiated in-vitro instead of being transplanted into mice. Briefly, a pool of viral supernatant containing all 42 vectors was used to transduce CB CD34+ HSPCs. The transduced cells were then differentiated into the different stages of T-cell development using the Artificial Thymic Organoid (ATO) system (see, e.g., Seet et al. (2017) Nat. Meth. 14(5): 521-530)J. RNA and gDNA were extracted from each T-cell developmental population and barcodes in the RNA and gDNA were be amplified by PCR and quantified by next-generation sequencing. Since each barcode is associated with a known enhancer element, by quantifying the relative abundance of each barcode in a specific cell population, we can elucidate the enhancer elements which are responsible for the high level, lineage and temporal specific expression of RAG through specific stages of T development.

The results of the enhancer screen throughout the different stages of T cell development are recapitulated across 3 different models—NSG mice, BLT mice and ATOs. Enhancer elements 9 and 12 seem to be the major enhancers for RAG expression in T-cells. Element 9 has homology to a previously identified enhancer element in mouse cells (essential for RAG1 expression at the DP stage—known as the ASE element). Element 12 is a newly identified element. Elements 5, 6, 11, 13, 14, 18 also had enhancer activity in T-cells. The enhancers were most active during the DP stage as expected (during TCRa rearrangement). All enhancer were off at the single positive CD4+/CD8+ stages. Element 9 alone expressed >8-fold higher than UCOE at the DP stage, comparable expression with MNDU3. MNDU3 and UCOE were active at all developmental stages.

Additionally, enhancer elements 14 and 5 seem to be the major enhancers necessary for RAG1 expression in B-cells. Element 14 has homology to a previously defined element in the literature in mouse cells (eRAG). Element 5 is a newly identified element. Expression of elements 5 and 14 peak at the HG1/HG2 stage. Enhancers are off at the NB and MB stage and surprisingly gain expression again at the plasma cell stage. It is possible that a larger element for #5 and #14 can be taken as the illustrated elements express lower than MNDU3—which is under WT levels. 19 (MNDU3) and 20 (UCOE) are active at all stages of B-cell development.

The results from the enhancer screens described above were used to design our lead candidate vectors. In designing our lead candidates, we want to increase expression from the B-cell enhancer elements (elements 5 and 14) since the expression is lower than that of MNDU3 (which is under WT levels). Therefore, we increased the size of the elements in an attempt to increase expression. Without being bound to a particular theory, it is believed that boundaries of the enhancer element were defined too narrowly and can be missing important sequences necessary for complete transcription factor binding. The new boundaries are termed the element 5 and 14 “cores”—which are larger than the original elements. The vectors also contained a novel B-cell enhancer which was missed in the initial screen. The element is adjacent to enhancer element 11 so it is designated “E11 B-cell enhancer”. Additionally, the size of element 9.1 is decreased with the goal of decreasing the size of the vector while maintaining expression. Ten illustrative lead candidate vectors are shown in Table 2.

TABLE 2
Illustrative, but non-limiting vector constructs.

| | | SEQ |
|---|---|---|
| Vector | Description | ID NO |
| 1 | E5 Core, E14 Core, E9.1 Core, E12 OG − RAG1pro-RAG-WPRE (8262 bp) | 37 |
| | (the expanded B cell enhancers E5 and E14 should boost B-cell | |
| | expression. We have shrunk 9.1 to try to retain expression and kept the | |
| | original element 12 fragment) (We hope B-cell expression goes up and | |
| | T-cell expression is maintained) This is the bare minimal using the | |
| | larger key elements. | |
| 2 | E5 Core, E14 Core, E9.1 Core, E12 OG, E11 extra B-cell enhancer − | 38 |
| | RAG1pro-RAG-WPRE (8579 bp) | |
| | (This is expecting that the expanded E5 and E14 elements will boost B- | |
| | cell expression but we would still want an additional boost so we added | |
| | the novel E11 extra b-cell enhancer which we hope will increase | |
| | expression in the B-cell lineage. T cell expression is maintained). | |
| | This is the bare minimal using the larger key elements. Plus the | |
| | additional of a potential novel B-cell enhancer. | |
| 3 | E5 Core, E14 Core, E9.1 OG large, E12 OG − RAG1pro-RAG-WPRE | 39 |
| | (8953 bp) | |
| 4 | E5 Core, E14 Core, E9.1 OG large, E12 OG, E11 extra B-cell enhancer − | 40 |
| | RAG1pro-RAG-WPRE (9270 bp) | |
| 5 | E5 Core, E14 Core, E9.1 Core, E12 OG + 18 right, 11 whole, 13 right − | 41 |
| | RAG1pro-RAG-WPRE (9147 bp) | |
| 6 | E5 Core, E14 Core, E9.1 Core, E12 OG + 18 whole, 11 whole − | 42 |
| | RAG1pro-RAG-WPRE (8966 bp) | |
| 7 | E5 Core, E14 Core, E9.1 Core, E12 OG + 18 whole − RAG1pro-RAG- | 43 |
| | WPRE (8643 bp) | |
| 8 | E5 Core, E14 Core, E9.1 Core, E12 OG, E11 extra B-cell enhancer + 18 | 44 |
| | right, 11 whole, 13 right − RAG1pro-RAG-WPRE (9461 bp) | |
| 9 | E5 Core, E14 Core, E9.1 Core, E12 OG, E11 extra B-cell enhancer + 18 | 45 |
| | whole, 11 whole − RAG1pro-RAG-WPRE (9283 bp) | |
| 10 | E5 Core, E14 Core, E9.1 Core, E12 OG, E11 extra B-cell enhancer + 18 | 46 |
| | whole − RAG1pro-RAG-WPRE (8960 bp) | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCE LISTING

SEQ ID NO: 1 RAG1 Enhancer 1 (TRAF6pro)
GGAGCAGAGGTCAGGGGACAGGGAACGGTGTCCCCGACCTGTGGGAAGCGTGG
GGACAGAGGCTGCGTCCCGACCTGCAGGAAGCGAGGGGCAGGGAACGGTGTCCT
CGACCAGCGGGAAGCGAGAGGACAGGGGCGGCGTCCCCGACCCGTGGGAAGCA
AGCGGGTAGGGGCGGCGTCCCTGACCTGTGAAAAGCGAAGGGACAGGGGAGGC
GTCCCCGACTGGTGGGAAGCGAGGGGACAGGGACTGCGTCCCCGACCTGCCGAG
AGCGAAGGGGCGGGGGGCGGCGTCCCTGACCGCTGGGAAGCGAGGCGGCGGCC
GCCAGGAGGAGGCGCCTGAAGGAGACTCACCGTTCTAGTGCGCGGGGAGGCCGA
ACCAGGAGGGCAGGGCTCCCCCACCAACCGCACGACTCCGCTCAGCCAAGGCGC
TGGTAGAGGACGGACACAGACACTGCGCGCCGAGACGAGGCTGCTTGGACGGCA
AACTCTGGATCCAGTGGGAGCCTTCGCCACCTTCGCTGGCCGCCCGCAGGCCAAG
CCCCAGCTGCGGACGCCACTGCTTCCGCCTTCTCTGCTGGCTGCGGGGAGCGAGG
GGCAGGGAGCGCGCCGGGGAGGAGACAGAGCGGCCGAGTTCGGAGAGAGTGCC
CCCTGGCGGTGATCCTCCAGGCGGACTGTGGCGCGGGCCGGGCGGCGCCCCCAG
CCCCGCAGCCTGGCTTTCTTCCTGCTCGGGTGTCCCACCTGGGAATTCGTTGCACC
GCACAGCCGAACCCGAGTGTGTCAATGGGTGTGTGTTGGAATGAGGATGGCGAC
GAGAAGATCCCTCCCGGAATAACTTAATTTGGTGAGCAGTGGCGACAGTAGTAA
TCCCAAACTGCGCTTTTATAGCACCCCTCTCCAAGCAGGAGAAAACCCCAGCTGG
GCTAGATCGCTTGACCTTCAGGAATTCTTAAACCCCTCCCCAGCTCCACCCTCCTT
GCCCAGGAAGTTTCTGCGATCCATAGAACCCTCTATTATTACCTGCTCTTCCCCTT
CATCCGGTCCAAGGGAGTCAGCCCTTGCCTTAA
SEQ ID NO: 2 RAG Enhancer 2 (Endogenous RAG promoter)
CTAAAGAGCCAGGTGGCAGCTGGAGCTGGGGTCTCCTGGCCCATGATTGGCTGC
CATCATTTGTGGTTAGCCCTCCATGGTGGGGGAGGCTGGGAAGGACAGTGGAAG
CTGATAAACAGCTCAGCAGCATGTTCTGAGAAACAAGAGGGCAAGGAGAGAGC
AGAGAACACACTTTGCCTTCTCTTTGGTATTGAGTAATATCAACCAAATTGCAGA
CATCTCAACACTTTGGCCAGGCAGCCTGCTGAGCAAGGTACCTCAGCCAGC
SEQ ID NO: 3 RAG Enhancer 3 (RAG2-Int1)
CTCAGCCTGAGTGATTATGATCAGAGAGTTGTCCCATTTTGGGTGGGGGGAACAC
CAAAGCCAAGGCCTGCTTGTTTCAAACACCTGTGATGACTGTTATTGAGATACAA
GGCAAGCCATACAGTGGTAATGGATTCCAGATCCAAAGGG
SEQ ID NO: 4 RAG Enhancer 4 (RAG1-Int1)
TTGAGGTTATGTTCTTGAGTCAGGATTCCTGGCCAGGGATTGTGATGATATATTTC
TCTTTCTGAAGTGGTTCATGCAAGAGGTTGTCTGAAGGAAGAGCAAGAATTGTAG
TGTTATTTTGTGGATACTTGAGACTTATAAAAAGGCTTTTT
SEQ ID NO: 5 RAG Enhancer 5 (25 kb upstream of RAG1)
AGGGACCTAGAGGCAGATGTAGACAAATAGCAGGTGAGGAGGAAGGGTGGTGC
TGAGCTGTAAACAACAGCTGCGGGCTGTAAACAATGGAGCTATATTAAGAAAGT
CCTGCATCTGCAGCATTCGGTTCAATCCTGCCACCCACTGGGCGTACTCCTGAAT
TGACTTTCCATTGTCAGCTGATCAGTGACAGATGTGAAATGACTCGTTACACACA
CAGGTCACATTGGGAGGATTCTTAGACGTCATA
SEQ ID NO: 6 RAG Enhancer 6 (Full P. Gfl1ib B1
GGCACCAAACTGTTTTCTACTTGACAGGTTGTTATCATGCTGTATTTGCACCCACC
ATCACAGAGATTGTTTAAAAAAAAAGTTATCTAGGGCATGCCAAATTATATGTAA
AAGTATAGTACAGATTGCAGTGGAAAGATAACGTGATGATTCTGTGATATCTGG
AGAGGAAACATAACCAGTTGGAGATAGTGCCTTCATTTTTGTTTTAAAAACAAAA
GAGAACTCACTTGCGAACTCTTTTTTTTTTTTTCTAAATTTCTTCAGGCCACCATA
GCTGAAGCTCCCTTTACTTCTCACCTGGGTTACTATGGTAGCTCCACACTTTCTTG
TCTCTAGTACCTGAACCACTTTGATCCATTCTATCCATTTGTCTTCTTAGAGCACA
GCTCTAATTGTGACTCCCCAGGATGTAGGTCAAGTACAGAGTTTTTGTCGGGCCC
TCCAAATATACCCCGTCCATGTGTCCCCAAATCTTCCTGGAATAATCGGCGATTC
TTACTCAGGCCTTGTGTGTTCCCACCTTGAGGGCTTTGCTCATGGAATTTTCCCTT
TGTAAAATGACCTCTTCACAGTGCTCACCTGTTGAAATTGTAGCTATCTTTCAAA
GCCCAGCTCAGATGCCTTAGTCCTATGTGAAGCCTTTTGTGATTCTGCCAAACCA
ATTATGATTTTACCATTCTTTGTGTTTCTATCTCTCTTATATCATCTGCCATCGGTG
CCTTCTCTTTTGGTGGTTTGCATGTATGGCTTAACTTCCTAGTTTGTAAACTCTGT
GAGGACAACAAGGACTGAGTCTTAACTGTCTTTGTCACCTTGCATATAGTTGATG
SEQ ID NO: 7 RAG Enhancer 7 (5′PRP5L)
TGTACTGTTAGCAAATTACTTCCCCTCTTGGAACCTCAATTTCATCATCCGTAAAA
TGGGAACAATAATAAAAATACCTTTTGGCCATTAGGGGGAGCTCAAGGATGACT
TATACCCCAAAAGGGGAAGGATTGCTTATGTGGATTCTCAGTTCTAGTCATTGGC
AACGCTAAAGGGCAGAGCTTTCCCCTGTTGTGGGCAAGGGTTTGCACGAGGGGG
ACTGGCCATCCCAGTT
SEQ ID NO: 8 RAG Enhancer 8 (3′ TRAF6)
CTGTGGCTGGGATTCTGCAAACCACATTGCTGCTTTGTCAGCTGGGTCCCTGGTA
GGTTCTGCCAATGGAGGGCGCTAGAGGGAGCCTGGGGGACTGGTGGAGGGAGA
GGAGTTGGGCTCATCCTGTCTTTGTCCACATCACCTAGCATAGACAGCTGGGTCC
AGCAGCAGTGTTATATCCCTTCCAGTAATTTTGGTGGTTGCTTCTCAGCATTCTTG
AGTTTATAGGTGGAGCATACTTGATCCTTGTGTGAGTCCCCAAGAGACACTGGGT
CCTATTTGTGGTTGCTTAGTCAATGACTTCATAGCTTAAAAGAAGTGACCTCGGA
GCCCTTGTTCCTGTATCGTACAATTGCCTAGTTCTTCAGTTACCTTCAGCAGTATG
CATTTCTCACTAAGTCACTCATTAGATTGGCTTTTCATTTCC
SEQ ID NO: 9 RAG Enhancer 9.1 (ASE-Full)
GTGGGCTTTGCCTACAGCACCTGCTTCTGCTTTCCGCATTTACTGATCCACTAAGT
GTAAGTGTGACATATAGAGGTCTGTGTTGGTTGTTGTTTTGTGTTCAAACAGCTG
AACATCTGGCCTCTCATGTGCAGGTGCACAGAACTCAGGAGAAGGGGTTTTAATT
TAAGATAAAAAAAATAAGGATATGTGCTAGTTGCAGATGAGGGTTTGGGTGCTG
GACTTAAGATGCTTCTGTGTTCAAAAACAAGGATATATGAGTCCCTTATATGTCA
GGGACAATGTACAGTGCTGGTCATAATATTCATTTTCTGAAAATGTCTTCACTGA
CATAATGGCTTCTGATTTGTCATCCTGAACAAGTCTATTATATTTTATTTAGTTAT
TGTCCTCAATTTGCAAGGTTCATAAAGAGTTTCACACAGATGGCACCCAAAGTGC
TTATTTCTCATTGTGCTGAAATTAAACTTGAATATGCTATATATCAAATAAATAAT
CCATTCCATGCCATTAAAAGAGTTTAGAATGAGAGATGAAATAAATTTTGCAAA
AAGTGTGCCCACATGTTCATTTTCTCCAAATAGCATAGTCATTACATTTAAAAAC
AATCATTGGTCAAAATAGAAAGTATTAAGAAAATACTAAGTATCAATTGAGGTC
TACCACATGTTCAGCAACAACTTAGGTGAGATGTGGGAGTAGGAAAAAGGAATA
CTTTATAACATCACAGAGAGGCAGATTTTGGCACAGGGAAGAGAAGTCAATGCA
ATACCTCAAGGAACCCAGTGGTGATCTAAGAAAACACTAGTTTGACCCATTGCCT
CAATTAAGTATTTACCTTTCATAGCTAAGAAGAGAAGTAAATAGCCTTTCTTTAT
ACTTTCTAGAAGTTGAGCTCAATGTTCTTTCGGAGAGAGCAGGCTCATCTCCATT
ATGTAGCGCCAATGACAAATACCTGTATATGTCTGTATTTCCTTGGTCTGTCTTGC
TCACCTGTTAAGACACCTTGCTGCACAACAGATGGCACATGCTTATCTTGTGGCA
GTGAAGAGTTGACATCAAAGGCCAAGAAAACCTTGAAACTCATCCTTTTCAGCCC
ACAAGAGCAGCTTCTCTCTACCACAGACGATGAAAGCTGGAGATAACAGGGGCA
TTCCCACCGCTGCACTCTCTCTTGTGGCCAGCTGACAGTTAACACAGGTGGGCGT
GAGGTGCTGCCTACATTTTGAAGGTTGTCTATTGATGGGGAACACCACCTCTCTG
AGCTCAGGCCTCTGCTTTGGCCCGTGGAGCTGCTGCTTCTGAGAAGTAACCAGGT
GGTGAATACCTGCCTGCTTTTCAGAAAATCCGAGGTGGCTGTCAAAACACCCACT
ATCATTCAAAGGCTGTCAGGCAAGGTGGCCGTTTGAGGGGCCACTTTAGTTCTTG
GTTTTTTTCAGAAGTCGAATTGGATTGCCAAACAGCTTGCTCAACTGCCTCTTCCC
ATAAGCTAGATGGGAGGAAGGCTGGGTCTGTGTAACTTGGGGTGCGCACAACTA
AAGTGCATAAAAAGAGAAGGA
SEQ ID NO: 10 RAG Enhancer 9.2 (ASE-core)
GGCTTTGCCTACAGCACCTGCTTCTGCTTTCCGCATTTACTGATCCACTAAGTGTA
AGTGTGACATATAGAGGTCTGTGTTGGTTGTTGTTTTGTGTTCAAACAGCTGAAC
ATCTGGCCTCTCATGTGCAGGTGCACAGAACTCAGGAGAATCTTGCTCACCTGTT
AAGACACCTTGCTGCACAACAGATGGCACATGCTTATCTTGTGGCAGTGAAGAGT
TGACATCAAAGGCCAAGAAAACCTTGAAACTCATCCTTTTCAGCCCACAAGAGC
AGCTTCTCTCTACCACAGACGATGAAAGCTGGAGATAACAGGGGCATTCCCACC
GCTGCACTCTCTCTTGTGGCCAGCTGACAGTTAACACAGGTGGGCGTGAGGTGCT
GTTTGAGGGGCCACTTTAGTTCTTGGTTTTTTTCAGAAGTCGAATTGGATTGCCAA
ACAGCTTGCTCAACTGCCTCTTCCCATAAGCTAGATGGGAGGAAGGCTGGGTCTG
TGTAACTTGGGGTGCGCACAACTAAAGTGCATAAAAAGAG
SEQ ID NO: 11 RAG Enhancer 10 (PRR5L Int1)
ATTACCCACCTCACTATTATGAAGTATAACATACAGCGTGCACAGCACAGTGTCT
GGCCCATTGTAGTTGCTCAATAGCTTTTAGTGTTTGCTTTTATGAGTTTGAGTCAT
CTTTGTGGTGAGGATTACAGACCCTGACCCCTCTACTTGAGGTAGCTAAAGTTTC
CCTCTTGATGATTTAATCTTGGTTGGGGTGAGAAAAAAGCAGGTATCAGCTGTGC
TATTGGGGATTTCCTAACCCTTTATATTATTACATGTTTCATCCTGCCTTGAGGAA
GAAGTTTAAGCTACGCCAATATTCGGGGCT
SEQ ID NO: 12 RAG Enhancer 11 (7.4 kb 5′ RAG2)
TTTTTTAGGTCTACTACTCTTGTGTCTGAAAGTTGCATTTTGTTTGAGTAAGTTCA
TTAAGCAGGGACCTGAAAGTAAACATCAAAGGTGTGCTGAATCAGTGGTGACAA
TTTCATGTATCATATTTCATAATAATGTTCTTTCAGAGAGCACATAACATTAACTA
GCATGGAGCAGTGCATGCTCCCTGTATTTCCTTCTGGGGGCCATGATTGTGAAAG
TGTTGCTTGTGATTTACCAGCAGATATTTATACACATTTACTTATCACAAGACGTT
TTGAGTCTTTCATGTGGAGCTCAAAGCCTTAAAACATAAAAACCAA
SEQ ID NO: 13 RAG Enhancer 12 (TRAF6 3′UTR)
TTGACAATTATTTATTCATTGTAAAATAATCACAGGAACAGCAGCAGTGTAGGTT
TCCCTACCTAGAGGGTGGTATGCAGTGATTCTCAGGCGCTGGTTGGAAGGCACAG
CTGAGGGACACAAACTGCCAGGAAGTAATGTGGTAACTAGCCATGAGCTTGTGG
TACTAATGGTGGCACGGGAAACAAGGTCTCTGCTTGACTTTTATTTTCACTCCAT
AACAAACTCACCAGATCAGGAGCCTAAGGGTGGGTGGGGAGGGAGAAGAGAGA
AAAAAGCAAAGGGAAAGTTCAAAGTGACACTCGCTGGGGCTGAAAACCACACTC
CCCTGCAGATGAGGTCCTTGGCT
SEQ ID NO: 14 RAG Enhancer 13 (P. Gfi1b b2)
CTGAAACCGTTTCACCTTTTATTAGAATAGAGCACTGAGGCATGGCAGTAGCCAC
AGGAAGTCTGCTTTCCTTTGAACATTGATGAAGTTTGAGGAACCCTTTCATCCTTC
AGTTAACTCCTATTTCCAAGTGTCTCCATGCCTATCTTTGGCTTGATTTCTGCCAC
TTGACTTTTTTTGAATTCCTTTATCACTCTTTTACTCTTTTAGTGATCATTGACGTA
GGTTCCTGTACTTGCTGTTGTCAGAAGAAGCAAGTTTCAGTTTTGGAATGTACCC
TGCACGCTTAGTTCTTCTTTGACTATTGTAAGCCATTCTTCTTTGTCTCCTTTCTGG
CTCCCTGCCCCCACCTTTCTGCTCCTTTTTTCTTCTATTCTCTAAAGACCTTTTCTT
TTCATTTACTGCATTAATGCAGGAAGCAAGGCACAGTGGTTAGGAGGATAGGTTC
TAGAGTCAGAAGGCAGTGTTCAGATCCCCACTCTGCTACTTAACCATCTGTAT
SEQ ID NO: 15 RAG Enhancer 14 (eRAG)
AATCCTTGCTGTGTAGCCTTTTGCAGTTATTTTCTGTCATGTGTTAGATAGAGACC
AGAGGGCTTAACATTGTTTTGATACCCTTGCATCTGGCAGATTGCCTGTCAGAGG
AAGTTGGAAACGAACTGACCAGCTTTATGCTTCCAGGTGGCACTGAAGCCACATT
TTCAGTGCCGTTGTTATCAGTCATTTAATTCCCTACTGAATATTGAGTGGTGTTTT
TAGTTCACAGGCCATTTGCAGTGCCTT
SEQ ID NO: 16 RAG Enhancer 16 (8.4 kb 5′RAG2
TATCATTATTTACTGTCTAGCTACTGCAAACACAGTTTATTCAGCTTTGCATTGCT
AAAGCCTGGTGGTGTTACAAGTAAATGTGCATAAGCCATAACCAGTGCTTTTTCG
TGTATCTAATTTTTTCAGGTACAAATAGCTTGAAACAGTT
SEQ ID NO: 17 RAG Enhancer 17 (HS1)
ATGATTTCTTCTTAGCCAGTTCATGGATTCAACAACTTCTGAGACTTTTTCTCATG
GCTTTTTCTGATAGTCAAGAATATAGGCTTTTCTTTGCCCACACGGTGGCACTAGC
TGCTTAGAAAACAGTGTAGCTAAAATCAGAAAGCCCGCAGCAGATCTCCTGAAA
GCATGGAGAGTGAGCTGCAGCCGACGTT
SEQ ID NO: 18 RAG Enhancer 18 (5′ of HS1)
AAAAGTCTATTTTATCTAATATAAGCGTAGTTACTCCTGCTTACTTTTGGTTTCCA
TTTGCATGGAGTATCTTTTTCCATCCTTTCACTTTTCATCTATGTGTGTCTTTGTAG
TTGAAGTGAGTTTCTTGTAGATAGCACATGGTTATGTCCTGTTTGTTTTTGTCCAT
TCCTTCTGTCATTTTATTGTTTTCTGGTTGTTTTGCATTTCTCTTCCTCGTTTCTTTA
TCTCTTACTGTTTATCTTTGTGTGGTTTGCTTGGTGACAAATTTTAGTTACTTTCTC
TTTCGCATCTGTGTATCTGCTCTACTAGTGGGTTCTATACTTTTGTTTGTTCTCCTG
ATGGTAGTTATTGCCCTTTGGCTTTCAGATATAGGGCTCC
SEQ ID NO: 19 RAG1 cDNA
ATGGCAGCCTCTTTCCCACCCACCTTGGGACTCAGTTCTGCCCCAGATGAAATTC
AGCACCCACATATTAAATTTTCAGAATGGAAATTTAAGCTGTTCCGGGTGAGATC
CTTTGAAAAGACACCTGAAGAAGCTCAAAAGGAAAAGAAGGATTCCTTTGAGGG
GAAACCCTCTCTGGAGCAATCTCCAGCAGTCCTGGACAAGGCTGATGGTCAGAA
GCCAGTCCCAACTCAGCCATTGTTAAAAGCCCACCCTAAGTTTTCAAAGAAATTT
CACGACAACGAGAAAGCAAGAGGCAAAGCGATCCATCAAGCCAACCTTCGACAT
CTCTGCCGCATCTGTGGGAATTCTTTTAGAGCTGATGAGCACAACAGGAGATATC
CAGTCCATGGTCCTGTGGATGGTAAAACCCTAGGCCTTTTACGAAAGAAGGAAA
AGAGAGCTACTTCCTGGCCGGACCTCATTGCCAAGGTTTTCCGGATCGATGTGAA
GGCAGATGTTGACTCGATCCACCCCACTGAGTTCTGCCATAACTGCTGGAGCATC
ATGCACAGGAAGTTTAGCAGTGCCCCATGTGAGGTTTACTTCCCGAGGAACGTGA
CCATGGAGTGGCACCCCCACACACCATCCTGTGACATCTGCAACACTGCCCGTCG
GGGACTCAAGAGGAAGAGTCTTCAGCCAAACTTGCAGCTCAGCAAAAAACTCAA
AACTGTGCTTGACCAAGCAAGACAAGCCCGTCAGCACAAGAGAAGAGCTCAGGC
AAGGATCAGCAGCAAGGATGTCATGAAGAAGATCGCCAACTGCAGTAAGATACA
TCTTAGTACCAAGCTCCTTGCAGTGGACTTCCCAGAGCACTTTGTGAAATCCATC
TCCTGCCAGATCTGTGAACACATTCTGGCTGACCCTGTGGAGACCAACTGTAAGC
ATGTCTTTTGCCGGGTCTGCATTCTCAGATGCCTCAAAGTCATGGGCAGCTATTGT
CCCTCTTGCCGATATCCATGCTTCCCTACTGACCTGGAGAGTCCAGTGAAGTCCTT
TCTGAGCGTCTTGAATTCCCTGATGGTGAAATGTCCAGCAAAAGAGTGCAATGAG
GAGGTCAGTTTGGAAAAATATAATCACCACATCTCAAGTCACAAGGAATCAAAA
GAGATTTTTGTGCACATTAATAAAGGGGGCCGGCCCCGCCAACATCTTCTGTCGC
TGACTCGGAGAGCTCAGAAGCACCGGCTGAGGGAGCTCAAGCTGCAAGTCAAAG
CCTTTGCTGACAAAGAAGAAGGTGGAGATGTGAAGTCCGTGTGCATGACCTTGTT
CCTGCTGGCTCTGAGGGCGAGGAATGAGCACAGGCAAGCTGATGAGCTGGAGGC
CATCATGCAGGGAAAGGGCTCTGGCCTGCAGCCAGCTGTTTGCTTGGCCATCCGT
GTCAACACCTTCCTCAGCTGCAGTCAGTACCACAAGATGTACAGGACTGTGAAA
GCCatcacagggagacagatttttcagcctttgcatGCCCTTCGGAATGCTGAGAAGGTACTTCTGCC
AGGCTACCACCACTTTGAGTGGCAGCCACCTCTGAAGAATGTGTCTTCCAGCACT
GATGTTGGCATTATTGATGGGCTGTCTGGACTATCATCCTCTGTGGATGATTACCC
AGTGGACACCATTGCAAAGAGGTTCCGCTATGATTCAGCTTTGGTGTCTGCTTTG
ATGGACATGGAAGAAGACATCTTGGAAGGCATGAGATCCCAAGACCTTGATGAT
TACCTGAATGGCCCCTTCACTGTGGTGGTGAAGGAGTCTTGTGATGGAATGGGAG
ACGTGAGTGAGAAGCATGGGAGTGGGCCTGTAGTTCCAGAAAAGGCAGTCCGTT
TTTCATTCACAATCATGAAAATTACTATTGCCCACAGCTCTCAGAATGTGAAAGT
ATTTGAAGAAGCCAAACCTAACTCTGAACTGTGTTGCAAGCCATTGTGCCTTATG
CTGGCAGATGAGTCTGACCACGAGACGCTGACTGCCATCCTGAGTCCTCTCATTG
CTGAGAGGGAGGCCATGAAGAGCAGTGAATTAATGCTTGAGCTGGGAGGCATTC
TCCGGACTTTCAAGTTCATCTTCAGGGGCACCGGCTATGATGAAAAACTTGTGCG
GGAAGTGGAAGGCCTCGAGGCTTCTGGCTCAGTCTACATTTGTACTCTTTGTGAT
GCCACCCGTCTGGAAGCCTCTCAAAATCTTGTCTTCCACTCTATAACCAGAAGCC
ATGCTGAGAACCTGGAACGTTATGAGGTCTGGCGTTCCAACCCTTACCATGAGTC
TGTGGAAGAACTGCGGGATCGGGTGAAAGGGGTCTCAGCTAAACCTTTCATTGA
GACAGTCCCTTCCATAGATGCACTCCACTGTGACATTGGCAATGCAGCTGAGTTC
TACAAGATCTTCCAGCTAGAGATAGGGGAAGTGTATAAGAATCCCAATGCTTCC
AAAGAGGAAAGGAAAAGGTGGCAGGCCACACTGGACAAGCATCTCCGGAAGAA
GATGAACCTCAAACCAATCATGAGGATGAATGGCAACTTTGCCAGGAAGCTCAT
GACCAAAGAGACTGTGGATGCAGTTTGTGAGTTAATTCCTTCCGAGGAGAGGCA
CGAGGCTCTGAGGGAGCTGATGGATCTTTACCTGAAGATGAAACCAGTATGGCG
ATCATCATGCCCTGCTAAAGAGTGCCCAGAATCCCTCTGCCAGTACAGTTTCAAT
TCACAGCGTTTTGCTGAGCTCCTTTCTACGAAGTTCAAGTATAGGTATGAGGGAA
AAATCACCAATTATTTTCACAAAACCCTGGCCCATGTTCCTGAAATTATTGAGAG
GGATGGCTCCATTGGGGCATGGGCAAGTGAGGGAAATGAGTCTGGTAACAAACT
GTTTAGGCGCTTCCGGAAAATGAATGCCAGGCAGTCCAAATGCTATGAGATGGA
AGATGTCCTGAAACACCACTGGTTGTACACCTCCAAATACCTCCAGAAGTTTATG
AATGCTCATAATGCATTAAAAACCTCTGGGTTTACCATGAACCCTCAGGCAAGCT
TAGGGGACCCATTAGGCATAGAGGACTCTCTGGAAAGCCAAGATTCAATGGAAT
TTTAA
SEQ ID NO: 20 codon optimized RAG1
ATGGCCGCCAGCTTCCCCCCCACCCTGGGCCTGAGCAGCGCCCCCGACGAGATCC
AGCACCCCCACATCAAGTTCAGCGAGTGGAAGTTCAAGCTGTTCCGCGTGCGCA
GCTTCGAGAAGACCCCCGAGGAGGCCCAGAAGGAGAAGAAGGACAGCTTCGAG
GGCAAGCCCAGCCTGGAGCAGAGCCCCGCCGTGCTGGACAAGGCCGACGGCCAG
AAGCCCGTGCCCACCCAGCCCCTGCTGAAGGCCCACCCCAAGTTCAGCAAGAAG
TTCCACGACAACGAGAAGGCCCGCGGCAAGGCCATCCACCAGGCCAACCTGCGC
CACCTGTGCCGCATCTGCGGCAACAGCTTCCGCGCCGACGAGCACAACCGCCGCT
ACCCCGTGCACGGCCCCGTGGACGGCAAGACCCTGGGCCTGCTGCGCAAGAAGG
AGAAGCGCGCCACCAGCTGGCCCGACCTGATCGCCAAGGTGTTCCGCATCGACG
TGAAGGCCGACGTGGACAGCATCCACCCCACCGAGTTCTGCCACAACTGCTGGA
GCATCATGCACCGCAAGTTCAGCAGCGCCCCCTGCGAGGTGTACTTCCCCCGCAA
CGTGACCATGGAGTGGCACCCCCACACCCCCAGCTGCGACATCTGCAACACCGC
CCGCCGCGGCCTGAAGCGCAAGAGCCTGCAGCCCAACCTGCAGCTGAGCAAGAA
GCTGAAGACCGTGCTGGACCAGGCCCGCCAGGCCCGCCAGCACAAGCGCCGCGC
CCAGGCCCGCATCAGCAGCAAGGACGTGATGAAGAAGATCGCCAACTGCAGCAA
GATCCACCTGAGCACCAAGCTGCTGGCCGTGGACTTCCCCGAGCACTTCGTGAAG
AGCATCAGCTGCCAGATCTGCGAGCACATCCTGGCCGACCCCGTGGAGACCAAC
TGCAAGCACGTGTTCTGCCGCGTGTGCATCCTGCGCTGCCTGAAGGTGATGGGCA
GCTACTGCCCCAGCTGCCGCTACCCCTGCTTCCCCACCGACCTGGAGAGCCCCGT
GAAGAGCTTCCTGAGCGTGCTGAACAGCCTGATGGTGAAGTGCCCCGCCAAGGA
GTGCAACGAGGAGGTGAGCCTGGAGAAGTACAACCACCACATCAGCAGCCACAA
GGAGAGCAAGGAGATCTTCGTGCACATCAACAAGGGCGGCCGCCCCCGCCAGCA
CCTGCTGAGCCTGACCCGCCGCGCCCAGAAGCACCGCCTGCGCGAGCTGAAGCT
GCAGGTGAAGGCCTTCGCCGACAAGGAGGAGGGCGGCGACGTGAAGAGCGTGT
GCATGACCCTGTTCCTGCTGGCCCTGCGCGCCCGCAACGAGCACCGCCAGGCCGA
CGAGCTGGAGGCCATCATGCAGGGCAAGGGCAGCGGCCTGCAGCCCGCCGTGTG
CCTGGCCATCCGCGTGAACACCTTCCTGAGCTGCAGCCAGTACCACAAGATGTAC
CGCACCGTGAAGGCCATCACCGGCCGCCAGATCTTCCAGCCCCTGCACGCCCTGC
GCAACGCCGAGAAGGTGCTGCTGCCCGGCTACCACCACTTCGAGTGGCAGCCCC
CCCTGAAGAACGTGAGCAGCAGCACCGACGTGGGCATCATCGACGGCCTGAGCG
GCCTGAGCAGCAGCGTGGACGACTACCCCGTGGACACCATCGCCAAGCGCTTCC
GCTACGACAGCGCCCTGGTGAGCGCCCTGATGGACATGGAGGAGGACATCCTGG
AGGGCATGCGCAGCCAGGACCTGGACGACTACCTGAACGGCCCCTTCACCGTGG
TGGTGAAGGAGAGCTGCGACGGCATGGGCGACGTGAGCGAGAAGCACGGCAGC
GGCCCCGTGGTGCCCGAGAAGGCCGTGCGCTTCAGCTTCACCATCATGAAGATCA
CCATCGCCCACAGCAGCCAGAACGTGAAGGTGTTCGAGGAGGCCAAGCCCAACA
GCGAGCTGTGCTGCAAGCCCCTGTGCCTGATGCTGGCCGACGAGAGCGACCACG
AGACCCTGACCGCCATCCTGAGCCCCCTGATCGCCGAGCGCGAGGCCATGAAGA
GCAGCGAGCTGATGCTGGAGCTGGGCGGCATCCTGCGCACCTTCAAGTTCATCTT
CCGCGGCACCGGCTACGACGAGAAGCTGGTGCGCGAGGTGGAGGGCCTGGAGGC
CAGCGGCAGCGTGTACATCTGCACCCTGTGCGACGCCACCCGCCTGGAGGCCAG
CCAGAACCTGGTGTTCCACAGCATCACCCGCAGCCACGCCGAGAACCTGGAGCG
CTACGAGGTGTGGCGCAGCAACCCCTACCACGAGAGCGTGGAGGAGCTGCGCGA
CCGCGTGAAGGGCGTGAGCGCCAAGCCCTTCATCGAGACCGTGCCCAGCATCGA
CGCCCTGCACTGCGACATCGGCAACGCCGCCGAGTTCTACAAGATCTTCCAGCTG
GAGATCGGCGAGGTGTACAAGAACCCCAACGCCAGCAAGGAGGAGCGCAAGCG
CTGGCAGGCCACCCTGGACAAGCACCTGCGCAAGAAGATGAACCTGAAGCCCAT
CATGCGCATGAACGGCAACTTCGCCCGCAAGCTGATGACCAAGGAGACCGTGGA
CGCCGTGTGCGAGCTGATCCCCAGCGAGGAGCGCCACGAGGCCCTGCGCGAGCT
GATGGACCTGTACCTGAAGATGAAGCCCGTGTGGCGCAGCAGCTGCCCCGCCAA
GGAGTGCCCCGAGAGCCTGTGCCAGTACAGCTTCAACAGCCAGCGCTTCGCCGA
GCTGCTGAGCACCAAGTTCAAGTACCGCTACGAGGGCAAGATCACCAACTACTT
CCACAAGACCCTGGCCCACGTGCCCGAGATCATCGAGCGCGACGGCAGCATCGG
CGCCTGGGCCAGCGAGGGCAACGAGAGCGGCAACAAGCTGTTCCGCCGCTTCCG
CAAGATGAACGCCCGCCAGAGCAAGTGCTACGAGATGGAGGACGTGCTGAAGCA
CCACTGGCTGTACACCAGCAAGTACCTGCAGAAGTTCATGAACGCCCACAACGC
CCTGAAGACCAGCGGCTTCACCATGAACCCCCAGGCCAGCCTGGGCGACCCCCT
GGGCATCGAGGACAGCCTGGAGAGCCAGGACAGCATGGAGTTCTAA
SEQ ID NO: 21 CMV:
AGTTCCGCGTTACATAACTTACGGTAAATGGCCCGCCTGGCTGACCGCCCAACGACCCCCGC
CCATTGACGTCAATAATGACGTATGTTCCCATAGTAACGCCAATAGGGACTTTCCATTGACG
TCAATGGGTGGAGTATTTACGGTAAACTGCCCACTTGGCAGTACATCAAGTGTATCATATGC
CAAGTACGCCCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCTATTACCAT
GGTGATGCGGTTTTGGCAGTACATCAATGGGCGTGGATAGCGGTTTGACTCACGGGGATTTC
CAAGTCTCCACCCCATTGACGTCAATGGGAGTTTGTTTTGGCACCAAAATCAACGGGACTTT
CCAAAATGTCGTAACAACTCCGCCCCATTGACGCAAATGGGCGGTAGGCGTGTACGGTGGGA
GGTCTATATAAGCAGAGCTCGTTTAGTGAACCG
SEQ ID NO: 22 3′R/U5:
GGGTCTCTCTGGTTAGACCAGATCTGAGCCTGGGAGCTCTCTGGCTAACTAGGGAACCCACT
GCTTAAGCCTCAATAAAGCTTGCCTTGAGTGCTTCAAGTAGTGTGTGCCCGTCTGTTGTGTG
ACTCTGGTAACTAGAGATCCCTCAGACCCTTTTAGTCAGTGTGGAAAATCTCTAGC
SEQ ID NO: 23 PSI:
Tcgacgcaggactcggcttgctgaagcgcgcacggcaagaggcgaggggcggcgactggtga
gtacgccaaaaattttgactagcggaggctagaaggagagagatgggtgcgagagcgtcagt
attaagcgggggag
SEQ ID NO: 24 RRE:
Tccttgggttcttgggagcagcaggaagcactatgggcgcagcgtcaatgacgctgacggta
caggccagacaattattgtctggtatagtgcagcagcagaacaatttgctgagggctattga
ggcgcaacagcatctgttgcaactcacagtctggggcatcaagcagctccaggcaagaatcc
tggctgtggaaagatacct
SEQ ID NO: 25 WPRE:
Cccatatttgttctgtttttcttgatttgggtatacatttaaatgttaataaaacaaaatgg
tggggcaatcatttacatttttagggatatgtaattactagttcaggtgtattgccacaaga
caaacatgttaagaaactttcccgttatttacgctctgttcctgttaatcaacctctggatt
acaaaatttgtgaaagattgactgatattcttaactatgttgctccttttacgctgtgtgga
tatgctgctttaatgcctctgtatcatgctattgcttcccgtacggctttcgttttctcctc
cttgtataaatcctggttgctgtctctttatgaggagttgtggcccgttgtccgtcaacgtg
gcgtggtgtgctctgtgtttgctgacgcaacccccactggctggggcattgccaccacctgt
caactcctttctgggactttcgctttccccctcccgatcgccacggcagaactcatcgccgc
ctgccttgcccgctgctggacaggggctaggttgctgggcactgataattccgtggtgttgt
cggggaagggcctgctgccggctctgcggcctcttccgcgtcttcgccttcgccctcagacg
agtcggatctccctttgggccgcctccccgcctgga
SEQ ID NO: 26 3′ PPT:
tttttaaaagaaaaggggggac
SEQ ID NO: 27 3′ delta U3/R/U5
tggaagggctaattcactcccaacgaagacaagatctgctttttgcttgtactgggtctctc
tggttagaccagatctgagcctgggagctctctggctaactagggaacctactgcttaagcc
tcaataaagcttgccttgagtgcttCAAGTAGTGTGTGCCCGTCTGTTGTGTGACTCTGGTA
ACTAGAGATCCCTCAGACCCTTTTAGTCAGTGTGGAAAATCTCTAGC
SEQ ID NO: 28 SV40 ori:
Atcccgcccctaactccgcccagttccgcccattctccgccccatggctgactaattttttt
tatttatgcagaggccgaggccgcctcggcctctgagctattccagaagtagtgaggaggct
tttttggaggcctagg
SEQ ID NO: 29 KANr:
Attgaacaagatggattgcacgcaggttctccggccgcttgggtggagaggctattcggcta
tgactgggcacaacagacaatcggctgctctgatgccgccgtgttccggctgtcagcgcagg
ggcgcccggttctttttgtcaagaccgacctgtccggtgccctgaatgaactgcaagacgag
gcagcgcggctatcgtggctggccacgacgggcgttccttgcgcagctgtgctcgacgttgt
cactgaagcgggaagggactggctgctattgggcgaagtgccggggcaggatctcctgtcat
ctcaccttgctcctgccgagaaagtatccatcatggctgatgcaatgcggcggctgcatacg
cttgatccggctacctgcccattcgaccaccaagcgaaacatcgcatcgagcgagcacgtac
tcggatggaagccggtcttgtcgatcaggatgatctggacgaagagcatcaggggctcgcgc
cagccgaactgttcgccaggctcaaggcgagcatgcccgacggcgaggatctcgtcgtgacc
catggcgatgcctgcttgccgaatatcatggtggaaaatggccgcttttctggattcatcga
ctgtggccggctgggtgtggcggaccgctatcaggacatagcgttggctacccgtgatattg
ctgaagagcttggcggcgaatgggctgaccgcttcctcgtgctttacggtatcgccgctccc
gattcgcagcgcatcgccttctatcgccttcttgacgagttcttctga
SEQ ID NO: 30 COLE1:
agatcaaaggatcttcttgagatcctttttttctgcgcgtaatctgctgcttgcaaacaaaa
aaaccaccgctaccagcggtggtttgtttgccggatcaagagctaccaactctttttccgaa
ggtaactggcttcagcagagcgcagataccaaatactgttcttctagtgtagccgtagttag
gccaccacttcaagaactctgtagcaccgcctacatacctcgctctgctaatcctgttacca
gtggctgctgccagtggcgataagtcgtgtcttaccgggttggactcaagacgatagttacc
ggataaggcgcagcggtcgggctgaacggggggttcgtgcacacagcccagcttggagcgaa
cgacctacaccgaactgagatacctacagcgtgagctatgagaaagcgccacgcttcccgaa
gggagaaaggcggacaggtatccggtaagcggcagggtcggaacaggagagcgcacgaggga
gcttccagggggaaacgcctggtatctttatagtcctgtcgggtttcgccacctctgacttg
agcgtcgatttttgtgatgctcgtcaggggggcggagcctatggaaaaacgccagcaacgcg
SEQ ID NO: 31 Sequence of the larger element 5 B-cell enhancer-“Element 5 Core
agaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatg
SEQ ID NO: 32 Sequence of the larger element 14 B-cell Enhancer-“Element 14 Core”
taaatttccatgagggtgcaaagagaggcttttcccaatctgaagaatgaatttaaaatctgctgtagaaaaataccagagagaaaaatat
agaaagcagctgtccctcttaccaatttcagagcaggaaaggaatttcagtagagcataaaaacagctttctctcataaaagaaaacac
aacaagaaaaataatccttgctgtgtagccttttgcagttattttctgtcatgtgttagatagagaccagagggcttaacattgttttgatacc
cttgcatctggcagattgcctgtcagaggaagttggaaacgaactgaccagctttatgcttccaggtggcactgaagccacattttcagt
gccgttgttatcagtcatttaattccctactgaatattgagtggtgtttttagttcacaggccatttgcagtgcctttctgtttcctttttaacgtt
agccacattaagaaaaaaa
SEQ ID NO: 33 Sequence of “E11 extra B-cell Enhancer”
tctaggcaagtgtctctctctctctccccctctttttcttttagaaaaatacttcgtgataaaaacacaaaagtatgtgaatggtaattgaaaat
gcattgcttccttttagaaacccactaggatttgcataaaccacttggctgtctgataagtcattatctgttctgtcagtccctggggactttta
atattaggtttatatttttatagaaaaggcaagttctaaatttgaagatgggtattgttaaactcttcaataatgacagtttgtgaaactgcatgt
tccaaaagtcctttaaaaagtcaataaggc
SEQ ID NO: 34 Element 9.1 core
gtgggctttgcctacagcacctgcttctgctttccgcatttactgatccactaagtgtaagtgtgacatatagaggtctgtgttggttgttgttt
tgtgttcaaacagctgaacatctggcctctcatgtgcaggtgcacagaactcaggagaaggggttttaatttaagataaaaaaaataagg
atatgtgctagttgcagatgagggtttgggtgctggacttaagatgcttctgtgttcaaaaacaaggagtcctcaatttgcaaggttcataa
agagtttcacacagatggcacccaaagtgcttatttctcattgtgctgaaattaaacttgaatatgctatatatcaaataaatagaagtaaat
agcctttctttatactttctagaagttgagctcaatgttctttcggagagagcaggctcatctccattatgtagcgccaatgacaaatacctgt
atatgtctgtatttccttggtctgtcttgctcacctgttaagacaccttgctgcacaacagatggcacatgcttatcttgtggcagtgaagagt
tgacatcaaaggccaagaaaaccttgaaactcatccttttcagcccacaagagcagcttctctctaccacagacgatgaaagctggaga
taacaggggcattcccaccgctgcactctctcttgtggccagctgacagttaacacaggtgggcgtgaggtgctgcctacattttgaag
gttgtctattgatggggaacaccacctctctgagctcaggcctctgctttggcccgtggagctgctgcttctgagaagtaaccaggtggt
gaatacctgcctgcttttcagaaaatccgaggtggctgtcaa
SEQ ID NO: 35 Element 18 right fragment
cttctgtcattttattgttttctggttgttttgcatttctcttcctcgtttctttatctcttactgtttatctttgtgtggtttgcttggtgacaa
attttagttactttctctttcgcatctgtgtatctgctctactagtgggttctatacttttgtttgttctcctgatggtagttattgccctttggc
tttcagatatagggctcc
SEQ ID NO: 36 Element 13 right fragment
tggcttgatttctgccacttgactttttttgaattcctttatcactcttttactcttttagtgatcattgacgtaggttcctgtacttgctgttgt
cagaagaagcaagtttcagttttggaatgtaccctgcacgcttagttcttctttgactattgtaagccattcttctttgtctcctttctggctccc
tgcccccacctttctgctccttttttcttctattctctaaagaccttttcttttcatttactgcattaatgcaggaagcaaggcacagtggttagg
aggataggttctagagtcagaaggcagtgttcagatccccactctgctacttaaccatctgtat
SEQ ID NO: 37 1) E5 Core, E14 Core, E9.1 Core, E12 OG-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctcgagctaaagagccaggtggcagctggagctggggtctcctggcccatgattggct
gccatcatttgtggttagccctccatggtgggggaggctgggaaggacagtggaagctgataaacagctcagcagcatgttctgagaa
acaagagggcaaggagagagcagagaacacactttgccttctctttggtattgagtaatatcaaccaaattgcagacatctcaacacttt
ggccaggcagcctgctgagcaaggtacctcagccagcatggtgagcaagggcgaggagctgttcaccggggtggtgcccatcctg
gtcgagctggacggcgacgtaaacggccacaagttcagcgtgtccggcgagggcgagggcgatgccacctacggcaagctgacc
ctgaagttcatctgcaccaccggcaagctgcccgtgccctggcccaccctcgtgaccaccttcggctacggcctgatgtgcttcgccc
gctaccccgaccacatgaagcagcacgacttcttcaagtccgccatgcccgaaggctacgtccaggagcgcaccatcttcttcaagg
acgacggcaactacaagacccgcgccgaggtgaagttcgagggcgacaccctggtgaaccgcatcgagctgaagggcatcgactt
caaggaggacggcaacatcctggggcacaagctggagtacaactacaacagccacaacgtctatatcatggccgacaagcagaag
aacggcatcaaggtgaacttcaagatccgccacaacatcgaggacggcagcgtgcagctcgccgaccactaccagcagaacaccc
ccatcggcgacggccccgtgctgctgcccgacaaccactacctgagctaccagtccgccctgagcaaagaccccaacgagaagcg
cgatcacatggtcctgctggagttcgtgaccgccgccgggatcactctcggcatggacgagctgtacaagtgactgcaggaattcga
gcatcttaccgccatttattcccatatttgttctgtttttcttgatttgggtatacatttaaatgttaataaaacaaaatggtggggcaatcattta
catttttagggatatgtaattactagttcaggtgtattgccacaagacaaacatgttaagaaactttcccgttatttacgctctgttcctgttaat
caacctctggattacaaaatttgtgaaagattgactgatattcttaactatgttgctccttttacgctgtgtggatatgctgctttaatgcctctg
tatcatgctattgcttcccgtacggctttcgttttctcctccttgtataaatcctggttgctgtctctttatgaggagttgtggcccgttgtccgtc
aacgtggcgtggtgtgctctgtgtttgctgacgcaacccccactggctggggcattgccaccacctgtcaactcctttctgggactttcg
ctttccccctcccgatcgccacggcagaactcatcgccgcctgccttgcccgctgctggacaggggctaggttgctgggcactgataa
ttccgtggtgttgtcggggaagggcctgctgccggctctgcggcctcttccgcgtcttcgccttcgccctcagacgagtcggatctccct
ttgggccgcctccccgcctggaattcgagctcggtacctttaagaccaatgacttacaaggcagctgtagatcttagccactttttaaaag
aaaaggggggactggaagggctaattcactcccaacgaagacaagatctgctttttgcttgtactgggtctctctggttagaccagatct
gagcctgggagctctctggctaactagggaacctactgcttaagcctcaataaagcttgccttgagtgcttcaagtagtgtgtgcccgtct
gttgtgtgactctggtaactagagatccctcagacccttttagtcagtgtggaaaatctctagcagtagtagttcatgtcatcttattattcag
tatttataacttgcaaagaaatgaatatcagagagtgagaggaacttgtttattgcagcttataatggttacaaataaagcaatagcatcac
aaatttcacaaataaagcatttttttcactgcattctagttgtggtttgtccaaactcatcaatgtatcttatcatgtctggctctagctatcccg
cccctaactccgcccatcccgcccctaactccgcccagttccgcccattctccgccccatggctgactaattttttttatttatgcagaggc
cgaggccgcctcggcctctgagctattccagaagtagtgaggaggcttttttggaggcctagggacgtacccaattcgccctatagtga
gtcgtattacgcgcgctcactggccgtcgttttacaacgtcgtgactgggaaaaccctggcgttacccaacttaatcgccttgcagcaca
tccccctttcgccagctggcgtaatagcgaagaggcccgcaccgatcgcccttcccaacagttgcgcagcctgaatggcgaatggga
cgcgccctgtagcggcgcattaagcgcggcgggtgtggtggttacgcgcagcgtgaccgctacacttgccagcgccctagcgcccg
ctcctttcgctttcttcccttcctttctcgccacgttcgccggctttccccgtcaagctctaaatcgggggctccctttagggttccgatttagt
gctttacggcacctcgaccccaaaaaacttgattagggtgatggttcacgtagtgggccatcgccctgatagacggtttttcgccctttga
cgttggagtccacgttctttaatagtggactcttgttccaaactggaacaacactcaaccctatctcggtctattcttttgatttataagggatt
ttgccgatttcggcctattggttaaaaaatgagctgatttaacaaaaatttaacgcgaattttaacaaaatattaacgcttacaatttaggtgg
cacttttcggggaaatgtgcgcggaacccctatttgtttatttttctaaatacattcaaatatgtatccgctcatgagacaataaccctgataa
atgcttcaataatagcacctagatcaagagacaggatgaggatcgtttcgcatgattgaacaagatggattgcacgcaggttctccggc
cgcttgggtggagaggctattcggctatgactgggcacaacagacaatcggctgctctgatgccgccgtgttccggctgtcagcgcag
gggcgcccggttctttttgtcaagaccgacctgtccggtgccctgaatgaactgcaagacgaggcagcgcggctatcgtggctggcc
acgacgggcgttccttgcgcagctgtgctcgacgttgtcactgaagcgggaagggactggctgctattgggcgaagtgccggggca
ggatctcctgtcatctcaccttgctcctgccgagaaagtatccatcatggctgatgcaatgcggcggctgcatacgcttgatccggctac
ctgcccattcgaccaccaagcgaaacatcgcatcgagcgagcacgtactcggatggaagccggtcttgtcgatcaggatgatctgga
cgaagagcatcaggggctcgcgccagccgaactgttcgccaggctcaaggcgagcatgcccgacggcgaggatctcgtcgtgacc
catggcgatgcctgcttgccgaatatcatggtggaaaatggccgcttttctggattcatcgactgtggccggctgggtgtggcggaccg
ctatcaggacatagcgttggctacccgtgatattgctgaagagcttggcggcgaatgggctgaccgcttcctcgtgctttacggtatcgc
cgctcccgattcgcagcgcatcgccttctatcgccttcttgacgagttcttctgaattattaacgcttacaatttcctgatgcggtattttctcc
ttacgcatctgtgcggtatttcacaccgcatcaggtggcacttttcggggaaatgtgcgcggaacccctatttgtttatttttctaaatacatt
caaatatgtatccgctcatgaccaaaatcccttaacgtgagttttcgttccactgagcgtcagaccccgtagaaaagatcaaaggatcttc
ttgagatcctttttttctgcgcgtaatctgctgcttgcaaacaaaaaaaccaccgctaccagcggtggtttgtttgccggatcaagagctac
caactctttttccgaaggtaactggcttcagcagagcgcagataccaaatactgttcttctagtgtagccgtagttaggccaccacttcaa
gaactctgtagcaccgcctacatacctcgctctgctaatcctgttaccagtggctgctgccagtggcgataagtcgtgtcttaccgggttg
gactcaagacgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacaggtatccggta
agcggcagggtcggaacaggagagcgcacgagggagcttccagggggaaacgcctggtatctttatagtcctgtcgggtttcgcca
cctctgacttgagcgtcgatttttgtgatgctcgtcaggggggcggagcctatggaaaaacgccagcaacgcggcctttttacggttcct
ggccttttgctggccttttgctcacatgttctttcctgcgttatcccctgattctgtggataaccgtattaccgcctttgagtgagctgataccg
ctcgccgcagccgaacgaccgagcgcagcgagtcagtgagcgaggaagcggaagagcgcccaatacgcaaaccgcctctcccc
gcgcgttggccgattcattaatgcagctggcacgacaggtttcccgactggaaagcgggcagtgagcgcaacgcaattaatgtgagtt
agctcactcattaggcaccccaggctttacactttatgcttccggctcgtatgttgtgtggaattgtgagcggataacaatttcacacagga
aacagctatgaccatgattacgccaagcgcgcaattaaccctcactaaagggaacaaaagctggagctgcaagcttggccattgcata
cgttgtatccatatcataatatgtacatttatattggctcatgtccaacattaccgccat
SEQ ID NO: 38 2) E5 Core, E14 Core, E9.1 Core, E12 OG, E11 extra B-cell
enhancer-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctctaggcaagtgtctctctctctctccccctctttttcttttagaaaaatacttcgtgataaa
aacacaaaagtatgtgaatggtaattgaaaatgcattgcttccttttagaaacccactaggatttgcataaaccacttggctgtctgataagt
cattatctgttctgtcagtccctggggacttttaatattaggtttatatttttatagaaaaggcaagttctaaatttgaagatgggtattgttaaa
ctcttcaataatgacagtttgtgaaactgcatgttccaaaagtcctttaaaaagtcaataaggctcgagctaaagagccaggtggcagct
ggagctggggtctcctggcccatgattggctgccatcatttgtggttagccctccatggtgggggaggctgggaaggacagtggaag
ctgataaacagctcagcagcatgttctgagaaacaagagggcaaggagagagcagagaacacactttgccttctctttggtattgagta
atatcaaccaaattgcagacatctcaacactttggccaggcagcctgctgagcaaggtacctcagccagcatggtgagcaagggcga
ggagctgttcaccggggtggtgcccatcctggtcgagctggacggcgacgtaaacggccacaagttcagcgtgtccggcgagggc
gagggcgatgccacctacggcaagctgaccctgaagttcatctgcaccaccggcaagctgcccgtgccctggcccaccctcgtgac
caccttcggctacggcctgatgtgcttcgcccgctaccccgaccacatgaagcagcacgacttcttcaagtccgccatgcccgaaggc
tacgtccaggagcgcaccatcttcttcaaggacgacggcaactacaagacccgcgccgaggtgaagttcgagggcgacaccctggt
gaaccgcatcgagctgaagggcatcgacttcaaggaggacggcaacatcctggggcacaagctggagtacaactacaacagccac
aacgtctatatcatggccgacaagcagaagaacggcatcaaggtgaacttcaagatccgccacaacatcgaggacggcagcgtgca
gctcgccgaccactaccagcagaacacccccatcggcgacggccccgtgctgctgcccgacaaccactacctgagctaccagtccg
ccctgagcaaagaccccaacgagaagcgcgatcacatggtcctgctggagttcgtgaccgccgccgggatcactctcggcatggac
gagctgtacaagtgactgcaggaattcgagcatcttaccgccatttattcccatatttgttctgtttttcttgatttgggtatacatttaaatgtta
ataaaacaaaatggtggggcaatcatttacatttttagggatatgtaattactagttcaggtgtattgccacaagacaaacatgttaagaaa
ctttcccgttatttacgctctgttcctgttaatcaacctctggattacaaaatttgtgaaagattgactgatattcttaactatgttgctcctttta
cgctgtgtggatatgctgctttaatgcctctgtatcatgctattgcttcccgtacggctttcgttttctcctccttgtataaatcctggttgctgtc
tctttatgaggagttgtggcccgttgtccgtcaacgtggcgtggtgtgctctgtgtttgctgacgcaacccccactggctggggcattgcc
accacctgtcaactcctttctgggactttcgctttccccctcccgatcgccacggcagaactcatcgccgcctgccttgcccgctgctgg
acaggggctaggttgctgggcactgataattccgtggtgttgtcggggaagggcctgctgccggctctgcggcctcttccgcgtcttcg
ccttcgccctcagacgagtcggatctccctttgggccgcctccccgcctggaattcgagctcggtacctttaagaccaatgacttacaag
gcagctgtagatcttagccactttttaaaagaaaaggggggactggaagggctaattcactcccaacgaagacaagatctgctttttgct
tgtactgggtctctctggttagaccagatctgagcctgggagctctctggctaactagggaacctactgcttaagcctcaataaagcttgc
cttgagtgcttcaagtagtgtgtgcccgtctgttgtgtgactctggtaactagagatccctcagacccttttagtcagtgtggaaaatctcta
gcagtagtagttcatgtcatcttattattcagtatttataacttgcaaagaaatgaatatcagagagtgagaggaacttgtttattgcagcttat
aatggttacaaataaagcaatagcatcacaaatttcacaaataaagcatttttttcactgcattctagttgtggtttgtccaaactcatcaatgt
atcttatcatgtctggctctagctatcccgcccctaactccgcccatcccgcccctaactccgcccagttccgcccattctccgccccatg
gctgactaattttttttatttatgcagaggccgaggccgcctcggcctctgagctattccagaagtagtgaggaggcttttttggaggccta
gggacgtacccaattcgccctatagtgagtcgtattacgcgcgctcactggccgtcgttttacaacgtcgtgactgggaaaaccctggc
gttacccaacttaatcgccttgcagcacatccccctttcgccagctggcgtaatagcgaagaggcccgcaccgatcgcccttcccaac
agttgcgcagcctgaatggcgaatgggacgcgccctgtagcggcgcattaagcgcggcgggtgtggtggttacgcgcagcgtgac
cgctacacttgccagcgccctagcgcccgctcctttcgctttcttcccttcctttctcgccacgttcgccggctttccccgtcaagctctaa
atcgggggctccctttagggttccgatttagtgctttacggcacctcgaccccaaaaaacttgattagggtgatggttcacgtagtgggc
catcgccctgatagacggtttttcgccctttgacgttggagtccacgttctttaatagtggactcttgttccaaactggaacaacactcaac
cctatctcggtctattcttttgatttataagggattttgccgatttcggcctattggttaaaaaatgagctgatttaacaaaaatttaacgcgaat
tttaacaaaatattaacgcttacaatttaggtggcacttttcggggaaatgtgcgcggaacccctatttgtttatttttctaaatacattcaaat
atgtatccgctcatgagacaataaccctgataaatgcttcaataatagcacctagatcaagagacaggatgaggatcgtttcgcatgatt
gaacaagatggattgcacgcaggttctccggccgcttgggtggagaggctattcggctatgactgggcacaacagacaatcggctgc
tctgatgccgccgtgttccggctgtcagcgcaggggcgcccggttctttttgtcaagaccgacctgtccggtgccctgaatgaactgca
agacgaggcagcgcggctatcgtggctggccacgacgggcgttccttgcgcagctgtgctcgacgttgtcactgaagcgggaaggg
actggctgctattgggcgaagtgccggggcaggatctcctgtcatctcaccttgctcctgccgagaaagtatccatcatggctgatgca
atgcggcggctgcatacgcttgatccggctacctgcccattcgaccaccaagcgaaacatcgcatcgagcgagcacgtactcggatg
gaagccggtcttgtcgatcaggatgatctggacgaagagcatcaggggctcgcgccagccgaactgttcgccaggctcaaggcgag
catgcccgacggcgaggatctcgtcgtgacccatggcgatgcctgcttgccgaatatcatggtggaaaatggccgcttttctggattcat
cgactgtggccggctgggtgtggcggaccgctatcaggacatagcgttggctacccgtgatattgctgaagagcttggcggcgaatg
ggctgaccgcttcctcgtgctttacggtatcgccgctcccgattcgcagcgcatcgccttctatcgccttcttgacgagttcttctgaattat
taacgcttacaatttcctgatgcggtattttctccttacgcatctgtgcggtatttcacaccgcatcaggtggcacttttcggggaaatgtgc
gcggaacccctatttgtttatttttctaaatacattcaaatatgtatccgctcatgaccaaaatcccttaacgtgagttttcgttccactgagcg
tcagaccccgtagaaaagatcaaaggatcttcttgagatcctttttttctgcgcgtaatctgctgcttgcaaacaaaaaaaccaccgctac
cagcggtggtttgtttgccggatcaagagctaccaactctttttccgaaggtaactggcttcagcagagcgcagataccaaatactgttct
tctagtgtagccgtagttaggccaccacttcaagaactctgtagcaccgcctacatacctcgctctgctaatcctgttaccagtggctgct
gccagtggcgataagtcgtgtcttaccgggttggactcaagacgatagttaccggataaggcgcagcggtcgggctgaacgggggg
ttcgtgcacacagcccagcttggagcgaacgacctacaccgaactgagatacctacagcgtgagctatgagaaagcgccacgcttcc
cgaagggagaaaggcggacaggtatccggtaagcggcagggtcggaacaggagagcgcacgagggagcttccagggggaaac
gcctggtatctttatagtcctgtcgggtttcgccacctctgacttgagcgtcgatttttgtgatgctcgtcaggggggcggagcctatgga
aaaacgccagcaacgcggcctttttacggttcctggccttttgctggccttttgctcacatgttctttcctgcgttatcccctgattctgtgga
taaccgtattaccgcctttgagtgagctgataccgctcgccgcagccgaacgaccgagcgcagcgagtcagtgagcgaggaagcgg
aagagcgcccaatacgcaaaccgcctctccccgcgcgttggccgattcattaatgcagctggcacgacaggtttcccgactggaaag
cgggcagtgagcgcaacgcaattaatgtgagttagctcactcattaggcaccccaggctttacactttatgcttccggctcgtatgttgtg
tggaattgtgagcggataacaatttcacacaggaaacagctatgaccatgattacgccaagcgcgcaattaaccctcactaaagggaa
caaaagctggagctgcaagcttggccattgcatacgttgtatccatatcataatatgtacatttatattggctcatgtccaacattaccgcca
t
SEQ ID NO: 39 3) E5 Core. E14 Core. E9.1 OG large. E12 OG-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggatatatgagtcccttatatgtcagggacaa
tgtacagtgctggtcataatattcattttctgaaaatgtcttcactgacataatggcttctgatttgtcatcctgaacaagtctattatattttatt
tagttattgtcctcaatttgcaaggttcataaagagtttcacacagatggcacccaaagtgcttatttctcattgtgctgaaattaaacttgaat
atgctatatatcaaataaataatccattccatgccattaaaagagtttagaatgagagatgaaataaattttgcaaaaagtgtgcccacatgt
tcattttctccaaatagcatagtcattacatttaaaaacaatcattggtcaaaatagaaagtattaagaaaatactaagtatcaattgaggtct
accacatgttcagcaacaacttaggtgagatgtgggagtaggaaaaaggaatactttataacatcacagagaggcagattttggcaca
gggaagagaagtcaatgcaatacctcaaggaacccagtggtgatctaagaaaacactagtttgacccattgcctcaattaagtatttacc
tttcatagctaagaagagaagtaaatagcctttctttatactttctagaagttgagctcaatgttctttcggagagagcaggctcatctccatt
atgtagcgccaatgacaaatacctgtatatgtctgtatttccttggtctgtcttgctcacctgttaagacaccttgctgcacaacagatggca
catgcttatcttgtggcagtgaagagttgacatcaaaggccaagaaaaccttgaaactcatccttttcagcccacaagagcagcttctctc
taccacagacgatgaaagctggagataacaggggcattcccaccgctgcactctctcttgtggccagctgacagttaacacaggtggg
cgtgaggtgctgcctacattttgaaggttgtctattgatggggaacaccacctctctgagctcaggcctctgctttggcccgtggagctgc
tgcttctgagaagtaaccaggtggtgaatacctgcctgcttttcagaaaatccgaggtggctgtcaaaacacccactatcattcaaaggc
tgtcaggcaaggtggccgtttgaggggccactttagttcttggtttttttcagaagtcgaattggattgccaaacagcttgctcaactgcct
cttcccataagctagatgggaggaaggctgggtctgtgtaacttggggtgcgcacaactaaagtgcataaaaagagaaggattgacaa
ttatttattcattgtaaaataatcacaggaacagcagcagtgtaggtttccctacctagagggtggtatgcagtgattctcaggcgctggtt
ggaaggcacagctgagggacacaaactgccaggaagtaatgtggtaactagccatgagcttgtggtactaatggtggcacgggaaa
caaggtctctgcttgacttttattttcactccataacaaactcaccagatcaggagcctaagggtgggtggggagggagaagagagaaa
aaagcaaagggaaagttcaaagtgacactcgctggggctgaaaaccacactcccctgcagatgaggtccttggctctcgagctaaag
agccaggtggcagctggagctggggtctcctggcccatgattggctgccatcatttgtggttagccctccatggtgggggaggctggg
aaggacagtggaagctgataaacagctcagcagcatgttctgagaaacaagagggcaaggagagagcagagaacacactttgcctt
ctctttggtattgagtaatatcaaccaaattgcagacatctcaacactttggccaggcagcctgctgagcaaggtacctcagccagcatg
gtgagcaagggcgaggagctgttcaccggggtggtgcccatcctggtcgagctggacggcgacgtaaacggccacaagttcagcg
tgtccggcgagggcgagggcgatgccacctacggcaagctgaccctgaagttcatctgcaccaccggcaagctgcccgtgccctgg
cccaccctcgtgaccaccttcggctacggcctgatgtgcttcgcccgctaccccgaccacatgaagcagcacgacttcttcaagtccg
ccatgcccgaaggctacgtccaggagcgcaccatcttcttcaaggacgacggcaactacaagacccgcgccgaggtgaagttcgag
ggcgacaccctggtgaaccgcatcgagctgaagggcatcgacttcaaggaggacggcaacatcctggggcacaagctggagtaca
actacaacagccacaacgtctatatcatggccgacaagcagaagaacggcatcaaggtgaacttcaagatccgccacaacatcgag
gacggcagcgtgcagctcgccgaccactaccagcagaacacccccatcggcgacggccccgtgctgctgcccgacaaccactacc
tgagctaccagtccgccctgagcaaagaccccaacgagaagcgcgatcacatggtcctgctggagttcgtgaccgccgccgggatc
actctcggcatggacgagctgtacaagtgactgcaggaattcgagcatcttaccgccatttattcccatatttgttctgtttttcttgatttgg
gtatacatttaaatgttaataaaacaaaatggtggggcaatcatttacatttttagggatatgtaattactagttcaggtgtattgccacaaga
caaacatgttaagaaactttcccgttatttacgctctgttcctgttaatcaacctctggattacaaaatttgtgaaagattgactgatattcttaa
ctatgttgctccttttacgctgtgtggatatgctgctttaatgcctctgtatcatgctattgcttcccgtacggctttcgttttctcctccttgtat
aaatcctggttgctgtctctttatgaggagttgtggcccgttgtccgtcaacgtggcgtggtgtgctctgtgtttgctgacgcaacccccact
ggctggggcattgccaccacctgtcaactcctttctgggactttcgctttccccctcccgatcgccacggcagaactcatcgccgcctgc
cttgcccgctgctggacaggggctaggttgctgggcactgataattccgtggtgttgtcggggaagggcctgctgccggctctgcggc
ctcttccgcgtcttcgccttcgccctcagacgagtcggatctccctttgggccgcctccccgcctggaattcgagctcggtacctttaaga
ccaatgacttacaaggcagctgtagatcttagccactttttaaaagaaaaggggggactggaagggctaattcactcccaacgaagac
aagatctgctttttgcttgtactgggtctctctggttagaccagatctgagcctgggagctctctggctaactagggaacctactgcttaag
cctcaataaagcttgccttgagtgcttcaagtagtgtgtgcccgtctgttgtgtgactctggtaactagagatccctcagacccttttagtca
gtgtggaaaatctctagcagtagtagttcatgtcatcttattattcagtatttataacttgcaaagaaatgaatatcagagagtgagaggaac
ttgtttattgcagcttataatggttacaaataaagcaatagcatcacaaatttcacaaataaagcatttttttcactgcattctagttgtggtttgt
ccaaactcatcaatgtatcttatcatgtctggctctagctatcccgcccctaactccgcccatcccgcccctaactccgcccagttccgcc
cattctccgccccatggctgactaattttttttatttatgcagaggccgaggccgcctcggcctctgagctattccagaagtagtgaggag
gcttttttggaggcctagggacgtacccaattcgccctatagtgagtcgtattacgcgcgctcactggccgtcgttttacaacgtcgtgac
tgggaaaaccctggcgttacccaacttaatcgccttgcagcacatccccctttcgccagctggcgtaatagcgaagaggcccgcaccg
atcgcccttcccaacagttgcgcagcctgaatggcgaatgggacgcgccctgtagcggcgcattaagcgcggcgggtgtggtggtta
cgcgcagcgtgaccgctacacttgccagcgccctagcgcccgctcctttcgctttcttcccttcctttctcgccacgttcgccggctttcc
ccgtcaagctctaaatcgggggctccctttagggttccgatttagtgctttacggcacctcgaccccaaaaaacttgattagggtgatggt
tcacgtagtgggccatcgccctgatagacggtttttcgccctttgacgttggagtccacgttctttaatagtggactcttgttccaaactgga
acaacactcaaccctatctcggtctattcttttgatttataagggattttgccgatttcggcctattggttaaaaaatgagctgatttaacaaaa
atttaacgcgaattttaacaaaatattaacgcttacaatttaggtggcacttttcggggaaatgtgcgcggaacccctatttgtttatttttcta
aatacattcaaatatgtatccgctcatgagacaataaccctgataaatgcttcaataatagcacctagatcaagagacaggatgaggatc
gtttcgcatgattgaacaagatggattgcacgcaggttctccggccgcttgggtggagaggctattcggctatgactgggcacaacag
acaatcggctgctctgatgccgccgtgttccggctgtcagcgcaggggcgcccggttctttttgtcaagaccgacctgtccggtgccct
gaatgaactgcaagacgaggcagcgcggctatcgtggctggccacgacgggcgttccttgcgcagctgtgctcgacgttgtcactga
agcgggaagggactggctgctattgggcgaagtgccggggcaggatctcctgtcatctcaccttgctcctgccgagaaagtatccatc
atggctgatgcaatgcggcggctgcatacgcttgatccggctacctgcccattcgaccaccaagcgaaacatcgcatcgagcgagca
cgtactcggatggaagccggtcttgtcgatcaggatgatctggacgaagagcatcaggggctcgcgccagccgaactgttcgccag
gctcaaggcgagcatgcccgacggcgaggatctcgtcgtgacccatggcgatgcctgcttgccgaatatcatggtggaaaatggccg
cttttctggattcatcgactgtggccggctgggtgtggcggaccgctatcaggacatagcgttggctacccgtgatattgctgaagagct
tggcggcgaatgggctgaccgcttcctcgtgctttacggtatcgccgctcccgattcgcagcgcatcgccttctatcgccttcttgacga
gttcttctgaattattaacgcttacaatttcctgatgcggtattttctccttacgcatctgtgcggtatttcacaccgcatcaggtggcacttttc
ggggaaatgtgcgcggaacccctatttgtttatttttctaaatacattcaaatatgtatccgctcatgaccaaaatcccttaacgtgagttttc
gttccactgagcgtcagaccccgtagaaaagatcaaaggatcttcttgagatcctttttttctgcgcgtaatctgctgcttgcaaacaaaaa
aaccaccgctaccagcggtggtttgtttgccggatcaagagctaccaactctttttccgaaggtaactggcttcagcagagcgcagata
ccaaatactgttcttctagtgtagccgtagttaggccaccacttcaagaactctgtagcaccgcctacatacctcgctctgctaatcctgtt
accagtggctgctgccagtggcgataagtcgtgtcttaccgggttggactcaagacgatagttaccggataaggcgcagcggtcggg
ctgaacggggggttcgtgcacacagcccagcttggagcgaacgacctacaccgaactgagatacctacagcgtgagctatgagaaa
gcgccacgcttcccgaagggagaaaggcggacaggtatccggtaagcggcagggtcggaacaggagagcgcacgagggagctt
ccagggggaaacgcctggtatctttatagtcctgtcgggtttcgccacctctgacttgagcgtcgatttttgtgatgctcgtcaggggggc
ggagcctatggaaaaacgccagcaacgcggcctttttacggttcctggccttttgctggccttttgctcacatgttctttcctgcgttatccc
ctgattctgtggataaccgtattaccgcctttgagtgagctgataccgctcgccgcagccgaacgaccgagcgcagcgagtcagtgag
cgaggaagcggaagagcgcccaatacgcaaaccgcctctccccgcgcgttggccgattcattaatgcagctggcacgacaggtttc
ccgactggaaagcgggcagtgagcgcaacgcaattaatgtgagttagctcactcattaggcaccccaggctttacactttatgcttccg
gctcgtatgttgtgtggaattgtgagcggataacaatttcacacaggaaacagctatgaccatgattacgccaagcgcgcaattaaccct
cactaaagggaacaaaagctggagctgcaagcttggccattgcatacgttgtatccatatcataatatgtacatttatattggctcatgtcc
aacattaccgccat
SEQ ID NO: 40 4) E5 Core, E14 Core, E9.1 OG large, E12 OG, E11 extra B-cell
enhancer-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggatatatgagtcccttatatgtcagggacaa
tgtacagtgctggtcataatattcattttctgaaaatgtcttcactgacataatggcttctgatttgtcatcctgaacaagtctattatattttatt
tagttattgtcctcaatttgcaaggttcataaagagtttcacacagatggcacccaaagtgcttatttctcattgtgctgaaattaaacttgaat
atgctatatatcaaataaataatccattccatgccattaaaagagtttagaatgagagatgaaataaattttgcaaaaagtgtgcccacatgt
tcattttctccaaatagcatagtcattacatttaaaaacaatcattggtcaaaatagaaagtattaagaaaatactaagtatcaattgaggtct
accacatgttcagcaacaacttaggtgagatgtgggagtaggaaaaaggaatactttataacatcacagagaggcagattttggcaca
gggaagagaagtcaatgcaatacctcaaggaacccagtggtgatctaagaaaacactagtttgacccattgcctcaattaagtatttacc
tttcatagctaagaagagaagtaaatagcctttctttatactttctagaagttgagctcaatgttctttcggagagagcaggctcatctccatt
atgtagcgccaatgacaaatacctgtatatgtctgtatttccttggtctgtcttgctcacctgttaagacaccttgctgcacaacagatggca
catgcttatcttgtggcagtgaagagttgacatcaaaggccaagaaaaccttgaaactcatccttttcagcccacaagagcagcttctctc
taccacagacgatgaaagctggagataacaggggcattcccaccgctgcactctctcttgtggccagctgacagttaacacaggtggg
cgtgaggtgctgcctacattttgaaggttgtctattgatggggaacaccacctctctgagctcaggcctctgctttggcccgtggagctgc
tgcttctgagaagtaaccaggtggtgaatacctgcctgcttttcagaaaatccgaggtggctgtcaaaacacccactatcattcaaaggc
tgtcaggcaaggtggccgtttgaggggccactttagttcttggtttttttcagaagtcgaattggattgccaaacagcttgctcaactgcct
cttcccataagctagatgggaggaaggctgggtctgtgtaacttggggtgcgcacaactaaagtgcataaaaagagaaggattgacaa
ttatttattcattgtaaaataatcacaggaacagcagcagtgtaggtttccctacctagagggtggtatgcagtgattctcaggcgctggtt
ggaaggcacagctgagggacacaaactgccaggaagtaatgtggtaactagccatgagcttgtggtactaatggtggcacgggaaa
caaggtctctgcttgacttttattttcactccataacaaactcaccagatcaggagcctaagggtgggtggggagggagaagagagaaa
aaagcaaagggaaagttcaaagtgacactcgctggggctgaaaaccacactcccctgcagatgaggtccttggctctctaggcaagt
gtctctctctctctccccctctttttcttttagaaaaatacttcgtgataaaaacacaaaagtatgtgaatggtaattgaaaatgcattgcttcct
tttagaaacccactaggatttgcataaaccacttggctgtctgataagtcattatctgttctgtcagtccctggggacttttaatattaggttta
tatttttatagaaaaggcaagttctaaatttgaagatgggtattgttaaactcttcaataatgacagtttgtgaaactgcatgttccaaaagtc
ctttaaaaagtcaataaggctcgagctaaagagccaggtggcagctggagctggggtctcctggcccatgattggctgccatcatttgt
ggttagccctccatggtgggggaggctgggaaggacagtggaagctgataaacagctcagcagcatgttctgagaaacaagaggg
caaggagagagcagagaacacactttgccttctctttggtattgagtaatatcaaccaaattgcagacatctcaacactttggccaggca
gcctgctgagcaaggtacctcagccagcatggtgagcaagggcgaggagctgttcaccggggtggtgcccatcctggtcgagctgg
acggcgacgtaaacggccacaagttcagcgtgtccggcgagggcgagggcgatgccacctacggcaagctgaccctgaagttcat
ctgcaccaccggcaagctgcccgtgccctggcccaccctcgtgaccaccttcggctacggcctgatgtgcttcgcccgctaccccga
ccacatgaagcagcacgacttcttcaagtccgccatgcccgaaggctacgtccaggagcgcaccatcttcttcaaggacgacggcaa
ctacaagacccgcgccgaggtgaagttcgagggcgacaccctggtgaaccgcatcgagctgaagggcatcgacttcaaggaggac
ggcaacatcctggggcacaagctggagtacaactacaacagccacaacgtctatatcatggccgacaagcagaagaacggcatcaa
ggtgaacttcaagatccgccacaacatcgaggacggcagcgtgcagctcgccgaccactaccagcagaacacccccatcggcgac
ggccccgtgctgctgcccgacaaccactacctgagctaccagtccgccctgagcaaagaccccaacgagaagcgcgatcacatggt
cctgctggagttcgtgaccgccgccgggatcactctcggcatggacgagctgtacaagtgactgcaggaattcgagcatcttaccgcc
atttattcccatatttgttctgtttttcttgatttgggtatacatttaaatgttaataaaacaaaatggtggggcaatcatttacatttttagggat
atgtaattactagttcaggtgtattgccacaagacaaacatgttaagaaactttcccgttatttacgctctgttcctgttaatcaacctctggatta
caaaatttgtgaaagattgactgatattcttaactatgttgctccttttacgctgtgtggatatgctgctttaatgcctctgtatcatgctattgct
tcccgtacggctttcgttttctcctccttgtataaatcctggttgctgtctctttatgaggagttgtggcccgttgtccgtcaacgtggcgtggt
gtgctctgtgtttgctgacgcaacccccactggctggggcattgccaccacctgtcaactcctttctgggactttcgctttccccctcccg
atcgccacggcagaactcatcgccgcctgccttgcccgctgctggacaggggctaggttgctgggcactgataattccgtggtgttgtc
ggggaagggcctgctgccggctctgcggcctcttccgcgtcttcgccttcgccctcagacgagtcggatctccctttgggccgcctcc
ccgcctggaattcgagctcggtacctttaagaccaatgacttacaaggcagctgtagatcttagccactttttaaaagaaaagggggga
ctggaagggctaattcactcccaacgaagacaagatctgctttttgcttgtactgggtctctctggttagaccagatctgagcctgggagc
tctctggctaactagggaacctactgcttaagcctcaataaagcttgccttgagtgcttcaagtagtgtgtgcccgtctgttgtgtgactctg
gtaactagagatccctcagacccttttagtcagtgtggaaaatctctagcagtagtagttcatgtcatcttattattcagtatttataacttgca
aagaaatgaatatcagagagtgagaggaacttgtttattgcagcttataatggttacaaataaagcaatagcatcacaaatttcacaaata
aagcatttttttcactgcattctagttgtggtttgtccaaactcatcaatgtatcttatcatgtctggctctagctatcccgcccctaactccgcc
catcccgcccctaactccgcccagttccgcccattctccgccccatggctgactaattttttttatttatgcagaggccgaggccgcctcg
gcctctgagctattccagaagtagtgaggaggcttttttggaggcctagggacgtacccaattcgccctatagtgagtcgtattacgcgc
gctcactggccgtcgttttacaacgtcgtgactgggaaaaccctggcgttacccaacttaatcgccttgcagcacatccccctttcgcca
gctggcgtaatagcgaagaggcccgcaccgatcgcccttcccaacagttgcgcagcctgaatggcgaatgggacgcgccctgtagc
ggcgcattaagcgcggcgggtgtggtggttacgcgcagcgtgaccgctacacttgccagcgccctagcgcccgctcctttcgctttctt
cccttcctttctcgccacgttcgccggctttccccgtcaagctctaaatcgggggctccctttagggttccgatttagtgctttacggcacct
cgaccccaaaaaacttgattagggtgatggttcacgtagtgggccatcgccctgatagacggtttttcgccctttgacgttggagtccac
gttctttaatagtggactcttgttccaaactggaacaacactcaaccctatctcggtctattcttttgatttataagggattttgccgatttcggc
ctattggttaaaaaatgagctgatttaacaaaaatttaacgcgaattttaacaaaatattaacgcttacaatttaggtggcacttttcgggga
aatgtgcgcggaacccctatttgtttatttttctaaatacattcaaatatgtatccgctcatgagacaataaccctgataaatgcttcaataata
gcacctagatcaagagacaggatgaggatcgtttcgcatgattgaacaagatggattgcacgcaggttctccggccgcttgggtggag
aggctattcggctatgactgggcacaacagacaatcggctgctctgatgccgccgtgttccggctgtcagcgcaggggcgcccggtt
ctttttgtcaagaccgacctgtccggtgccctgaatgaactgcaagacgaggcagcgcggctatcgtggctggccacgacgggcgtt
ccttgcgcagctgtgctcgacgttgtcactgaagcgggaagggactggctgctattgggcgaagtgccggggcaggatctcctgtcat
ctcaccttgctcctgccgagaaagtatccatcatggctgatgcaatgcggcggctgcatacgcttgatccggctacctgcccattcgac
caccaagcgaaacatcgcatcgagcgagcacgtactcggatggaagccggtcttgtcgatcaggatgatctggacgaagagcatca
ggggctcgcgccagccgaactgttcgccaggctcaaggcgagcatgcccgacggcgaggatctcgtcgtgacccatggcgatgcc
tgcttgccgaatatcatggtggaaaatggccgcttttctggattcatcgactgtggccggctgggtgtggcggaccgctatcaggacata
gcgttggctacccgtgatattgctgaagagcttggcggcgaatgggctgaccgcttcctcgtgctttacggtatcgccgctcccgattcg
cagcgcatcgccttctatcgccttcttgacgagttcttctgaattattaacgcttacaatttcctgatgcggtattttctccttacgcatctgtgc
ggtatttcacaccgcatcaggtggcacttttcggggaaatgtgcgcggaacccctatttgtttatttttctaaatacattcaaatatgtatccg
ctcatgaccaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactctttttccg
aaggtaactggcttcagcagagcgcagataccaaatactgttcttctagtgtagccgtagttaggccaccacttcaagaactctgtagca
ccgcctacatacctcgctctgctaatcctgttaccagtggctgctgccagtggcgataagtcgtgtcttaccgggttggactcaagacga
tagttaccggataaggcgcagcggtcgggctgaacggggggttcgtgcacacagcccagcttggagcgaacgacctacaccgaac
tgagatacctacagcgtgagctatgagaaagcgccacgcttcccgaagggagaaaggcggacaggtatccggtaagcggcagggt
cggaacaggagagcgcacgagggagcttccagggggaaacgcctggtatctttatagtcctgtcgggtttcgccacctctgacttgag
cgtcgatttttgtgatgctcgtcaggggggcggagcctatggaaaaacgccagcaacgcggcctttttacggttcctggccttttgctgg
ccttttgctcacatgttctttcctgcgttatcccctgattctgtggataaccgtattaccgcctttgagtgagctgataccgctcgccgcagc
cgaacgaccgagcgcagcgagtcagtgagcgaggaagcggaagagcgcccaatacgcaaaccgcctctccccgcgcgttggcc
gattcattaatgcagctggcacgacaggtttcccgactggaaagcgggcagtgagcgcaacgcaattaatgtgagttagctcactcatt
aggcaccccaggctttacactttatgcttccggctcgtatgttgtgtggaattgtgagcggataacaatttcacacaggaaacagctatga
ccatgattacgccaagcgcgcaattaaccctcactaaagggaacaaaagctggagctgcaagcttggccattgcatacgttgtatccat
atcataatatgtacatttatattggctcatgtccaacattaccgccat
SEQ ID NO: 41 5) E5 Core, E14 Core, E9.1 Core, E12 OG + 18 right, 11 whole, 13
right-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctcgagcttctgtcattttattgttttctggttgttttgcatttctcttcctcgtttctttatctct
tactgtttatctttgtgtggtttgcttggtgacaaattttagttactttctctttcgcatctgtgtatctgctctactagtgggttctatacttttg
tttgttctcctgatggtagttattgccctttggctttcagatatagggctccttttttaggtctactactcttgtgtctgaaagttgcattttgttt
gagtaagttcattaagcagggacctgaaagtaaacatcaaaggtgtgctgaatcagtggtgacaatttcatgtatcatatttcataataatgttctt
tcagagagcacataacattaactagcatggagcagtgcatgctccctgtatttccttctgggggccatgattgtgaaagtgttgcttgtgattta
ccagcagatatttatacacatttacttatcacaagacgttttgagtctttcatgtggagctcaaagccttaaaacataaaaaccaatggcttg
atttctgccacttgactttttttgaattcctttatcactcttttactcttttagtgatcattgacgtaggttcctgtacttgctgttgtcagaagaa
gcaagtttcagttttggaatgtaccctgcacgcttagttcttctttgactattgtaagccattcttctttgtctcctttctggctccctgcccccac
ctttctgctccttttttcttctattctctaaagaccttttcttttcatttactgcattaatgcaggaagcaaggcacagtggttaggaggataggtt
ctagagtcagaaggcagtgttcagatccccactctgctacttaaccatctgtatgctaaagagccaggtggcagctggagctggggtctcc
tggcccatgattggctgccatcatttgtggttagccctccatggtgggggaggctgggaaggacagtggaagctgataaacagctcag
cagcatgttctgagaaacaagagggcaaggagagagcagagaacacactttgccttctctttggtattgagtaatatcaaccaaattgc
agacatctcaacactttggccaggcagcctgctgagcaaggtacctcagccagcatggtgagcaagggcgaggagctgttcaccgg
ggtggtgcccatcctggtcgagctggacggcgacgtaaacggccacaagttcagcgtgtccggcgagggcgagggcgatgccacc
tacggcaagctgaccctgaagttcatctgcaccaccggcaagctgcccgtgccctggcccaccctcgtgaccaccttcggctacggc
ctgatgtgcttcgcccgctaccccgaccacatgaagcagcacgacttcttcaagtccgccatgcccgaaggctacgtccaggagcgc
accatcttcttcaaggacgacggcaactacaagacccgcgccgaggtgaagttcgagggcgacaccctggtgaaccgcatcgagct
gaagggcatcgacttcaaggaggacggcaacatcctggggcacaagctggagtacaactacaacagccacaacgtctatatcatgg
ccgacaagcagaagaacggcatcaaggtgaacttcaagatccgccacaacatcgaggacggcagcgtgcagctcgccgaccacta
ccagcagaacacccccatcggcgacggccccgtgctgctgcccgacaaccactacctgagctaccagtccgccctgagcaaagac
cccaacgagaagcgcgatcacatggtcctgctggagttcgtgaccgccgccgggatcactctcggcatggacgagctgtacaagtg
actgcaggaattcgagcatcttaccgccatttattcccatatttgttctgtttttcttgatttgggtatacatttaaatgttaataaaacaaaatgg
tggggcaatcatttacatttttagggatatgtaattactagttcaggtgtattgccacaagacaaacatgttaagaaactttcccgttatttac
gctctgttcctgttaatcaacctctggattacaaaatttgtgaaagattgactgatattcttaactatgttgctccttttacgctgtgtggatatg
ctgctttaatgcctctgtatcatgctattgcttcccgtacggctttcgttttctcctccttgtataaatcctggttgctgtctctttatgaggagtt
gtggcccgttgtccgtcaacgtggcgtggtgtgctctgtgtttgctgacgcaacccccactggctggggcattgccaccacctgtcaactc
ctttctgggactttcgctttccccctcccgatcgccacggcagaactcatcgccgcctgccttgcccgctgctggacaggggctaggtt
gctgggcactgataattccgtggtgttgtcggggaagggcctgctgccggctctgcggcctcttccgcgtcttcgccttcgccctcaga
cgagtcggatctccctttgggccgcctccccgcctggaattcgagctcggtacctttaagaccaatgacttacaaggcagctgtagatct
tagccactttttaaaagaaaaggggggactggaagggctaattcactcccaacgaagacaagatctgctttttgcttgtactgggtctctc
tggttagaccagatctgagcctgggagctctctggctaactagggaacctactgcttaagcctcaataaagcttgccttgagtgcttcaa
gtagtgtgtgcccgtctgttgtgtgactctggtaactagagatccctcagacccttttagtcagtgtggaaaatctctagcagtagtagttc
atgtcatcttattattcagtatttataacttgcaaagaaatgaatatcagagagtgagaggaacttgtttattgcagcttataatggttacaaat
aaagcaatagcatcacaaatttcacaaataaagcatttttttcactgcattctagttgtggtttgtccaaactcatcaatgtatcttatcatgtct
ggctctagctatcccgcccctaactccgcccatcccgcccctaactccgcccagttccgcccattctccgccccatggctgactaattttt
tttatttatgcagaggccgaggccgcctcggcctctgagctattccagaagtagtgaggaggcttttttggaggcctagggacgtaccc
aattcgccctatagtgagtcgtattacgcgcgctcactggccgtcgttttacaacgtcgtgactgggaaaaccctggcgttacccaactta
atcgccttgcagcacatccccctttcgccagctggcgtaatagcgaagaggcccgcaccgatcgcccttcccaacagttgcgcagcct
gaatggcgaatgggacgcgccctgtagcggcgcattaagcgcggcgggtgtggtggttacgcgcagcgtgaccgctacacttgcca
gcgccctagcgcccgctcctttcgctttcttcccttcctttctcgccacgttcgccggctttccccgtcaagctctaaatcgggggctccct
ttagggttccgatttagtgctttacggcacctcgaccccaaaaaacttgattagggtgatggttcacgtagtgggccatcgccctgataga
cggtttttcgccctttgacgttggagtccacgttctttaatagtggactcttgttccaaactggaacaacactcaaccctatctcggtctattc
ttttgatttataagggattttgccgatttcggcctattggttaaaaaatgagctgatttaacaaaaatttaacgcgaattttaacaaaatattaa
cgcttacaatttaggtggcacttttcggggaaatgtgcgcggaacccctatttgtttatttttctaaatacattcaaatatgtatccgctcatga
gacaataaccctgataaatgcttcaataatagcacctagatcaagagacaggatgaggatcgtttcgcatgattgaacaagatggattgc
acgcaggttctccggccgcttgggtggagaggctattcggctatgactgggcacaacagacaatcggctgctctgatgccgccgtgtt
ccggctgtcagcgcaggggcgcccggttctttttgtcaagaccgacctgtccggtgccctgaatgaactgcaagacgaggcagcgcg
gctatcgtggctggccacgacgggcgttccttgcgcagctgtgctcgacgttgtcactgaagcgggaagggactggctgctattgggc
gaagtgccggggcaggatctcctgtcatctcaccttgctcctgccgagaaagtatccatcatggctgatgcaatgcggcggctgcatac
gcttgatccggctacctgcccattcgaccaccaagcgaaacatcgcatcgagcgagcacgtactcggatggaagccggtcttgtcgat
caggatgatctggacgaagagcatcaggggctcgcgccagccgaactgttcgccaggctcaaggcgagcatgcccgacggcgag
gatctcgtcgtgacccatggcgatgcctgcttgccgaatatcatggtggaaaatggccgcttttctggattcatcgactgtggccggctg
ggtgtggcggaccgctatcaggacatagcgttggctacccgtgatattgctgaagagcttggcggcgaatgggctgaccgcttcctcg
tgctttacggtatcgccgctcccgattcgcagcgcatcgccttctatcgccttcttgacgagttcttctgaattattaacgcttacaatttcct
gatgcggtattttctccttacgcatctgtgcggtatttcacaccgcatcaggtggcacttttcggggaaatgtgcgcggaacccctatttgt
ttatttttctaaatacattcaaatatgtatccgctcatgaccaaaatcccttaacgtgagttttcgttccactgagcgtcagaccccgtagaaa
agatcaaaggatcttcttgagatcctttttttctgcgcgtaatctgctgcttgcaaacaaaaaaaccaccgctaccagcggtggtttgtttgc
cggatcaagagctaccaactctttttccgaaggtaactggcttcagcagagcgcagataccaaatactgttcttctagtgtagccgtagtt
aggccaccacttcaagaactctgtagcaccgcctacatacctcgctctgctaatcctgttaccagtggctgctgccagtggcgataagtc
gtgtcttaccgggttggactcaagacgatagttaccggataaggcgcagcggtcgggctgaacggggggttcgtgcacacagccca
gcttggagcgaacgacctacaccgaactgagatacctacagcgtgagctatgagaaagcgccacgcttcccgaagggagaaaggc
ggacaggtatccggtaagcggcagggtcggaacaggagagcgcacgagggagcttccagggggaaacgcctggtatctttatagt
cctgtcgggtttcgccacctctgacttgagcgtcgatttttgtgatgctcgtcaggggggcggagcctatggaaaaacgccagcaacgc
ggcctttttacggttcctggccttttgctggccttttgctcacatgttctttcctgcgttatcccctgattctgtggataaccgtattaccgccttt
gagtgagctgataccgctcgccgcagccgaacgaccgagcgcagcgagtcagtgagcgaggaagcggaagagcgcccaatacg
caaaccgcctctccccgcgcgttggccgattcattaatgcagctggcacgacaggtttcccgactggaaagcgggcagtgagcgcaa
cgcaattaatgtgagttagctcactcattaggcaccccaggctttacactttatgcttccggctcgtatgttgtgtggaattgtgagcggata
acaatttcacacaggaaacagctatgaccatgattacgccaagcgcgcaattaaccctcactaaagggaacaaaagctggagctgca
agcttggccattgcatacgttgtatccatatcataatatgtacatttatattggctcatgtccaacattaccgccat
SEQ ID NO: 42 6) e5 core, el4 core, e9.1 core, e12 og + 18 whole, 11 whole-
rag1pro-rag-wpre
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctcgagaaaagtctattttatctaatataagcgtagttactcctgcttacttttggtttccattt
gcatggagtatctttttccatcctttcacttttcatctatgtgtgtctttgtagttgaagtgagtttcttgtagatagcacatggttatgtcctgtt
tgtttttgtccattccttctgtcattttattgttttctggttgttttgcatttctcttcctcgtttctttatctcttactgtttatctttgtgtgg
tttgcttggtgacaaattttagttactttctctttcgcatctgtgtatctgctctactagtgggttctatacttttgtttgttctcctgatggtagt
tattgccctttggctttcagatatagggctccttttttaggtctactactcttgtgtctgaaagttgcattttgtttgagtaagttcattaagcagg
gacctgaaagtaaacatcaaaggtgtgctgaatcagtggtgacaatttcatgtatcatatttcataataatgttctttcagagagcacataacatta
actagcatggagcagtgcatgctccctgtatttccttctgggggccatgattgtgaaagtgttgcttgtgatttaccagcagatatttatacacat
ttacttatcacaagacgttttgagtctttcatgtggagctcaaagccttaaaacataaaaaccaagctaaagagccaggtggcagctggagctgg
ggtctcctggcccatgattggctgccatcatttgtggttagccctccatggtgggggaggctgggaaggacagtggaagctgataaac
agctcagcagcatgttctgagaaacaagagggcaaggagagagcagagaacacactttgccttctctttggtattgagtaatatcaacc
aaattgcagacatctcaacactttggccaggcagcctgctgagcaaggtacctcagccagcatggtgagcaagggcgaggagctgtt
caccggggtggtgcccatcctggtcgagctggacggcgacgtaaacggccacaagttcagcgtgtccggcgagggcgagggcga
tgccacctacggcaagctgaccctgaagttcatctgcaccaccggcaagctgcccgtgccctggcccaccctcgtgaccaccttcgg
ctacggcctgatgtgcttcgcccgctaccccgaccacatgaagcagcacgacttcttcaagtccgccatgcccgaaggctacgtccag
gagcgcaccatcttcttcaaggacgacggcaactacaagacccgcgccgaggtgaagttcgagggcgacaccctggtgaaccgcat
cgagctgaagggcatcgacttcaaggaggacggcaacatcctggggcacaagctggagtacaactacaacagccacaacgtctata
tcatggccgacaagcagaagaacggcatcaaggtgaacttcaagatccgccacaacatcgaggacggcagcgtgcagctcgccga
ccactaccagcagaacacccccatcggcgacggccccgtgctgctgcccgacaaccactacctgagctaccagtccgccctgagca
aagaccccaacgagaagcgcgatcacatggtcctgctggagttcgtgaccgccgccgggatcactctcggcatggacgagctgtac
aagtgactgcaggaattcgagcatcttaccgccatttattcccatatttgttctgtttttcttgatttgggtatacatttaaatgttaataaaacaa
aatggtggggcaatcatttacatttttagggatatgtaattactagttcaggtgtattgccacaagacaaacatgttaagaaactttcccgtt
atttacgctctgttcctgttaatcaacctctggattacaaaatttgtgaaagattgactgatattcttaactatgttgctccttttacgctgtgtgg
atatgctgctttaatgcctctgtatcatgctattgcttcccgtacggctttcgttttctcctccttgtataaatcctggttgctgtctctttatgag
gagttgtggcccgttgtccgtcaacgtggcgtggtgtgctctgtgtttgctgacgcaacccccactggctggggcattgccaccacctgtc
aactcctttctgggactttcgctttccccctcccgatcgccacggcagaactcatcgccgcctgccttgcccgctgctggacaggggct
aggttgctgggcactgataattccgtggtgttgtcggggaagggcctgctgccggctctgcggcctcttccgcgtcttcgccttcgccct
cagacgagtcggatctccctttgggccgcctccccgcctggaattcgagctcggtacctttaagaccaatgacttacaaggcagctgta
gatcttagccactttttaaaagaaaaggggggactggaagggctaattcactcccaacgaagacaagatctgctttttgcttgtactgggt
ctctctggttagaccagatctgagcctgggagctctctggctaactagggaacctactgcttaagcctcaataaagcttgccttgagtgct
tcaagtagtgtgtgcccgtctgttgtgtgactctggtaactagagatccctcagacccttttagtcagtgtggaaaatctctagcagtagta
gttcatgtcatcttattattcagtatttataacttgcaaagaaatgaatatcagagagtgagaggaacttgtttattgcagcttataatggttac
aaataaagcaatagcatcacaaatttcacaaataaagcatttttttcactgcattctagttgtggtttgtccaaactcatcaatgtatcttatcat
gtctggctctagctatcccgcccctaactccgcccatcccgcccctaactccgcccagttccgcccattctccgccccatggctgactaa
ttttttttatttatgcagaggccgaggccgcctcggcctctgagctattccagaagtagtgaggaggcttttttggaggcctagggacgta
cccaattcgccctatagtgagtcgtattacgcgcgctcactggccgtcgttttacaacgtcgtgactgggaaaaccctggcgttacccaa
cttaatcgccttgcagcacatccccctttcgccagctggcgtaatagcgaagaggcccgcaccgatcgcccttcccaacagttgcgca
gcctgaatggcgaatgggacgcgccctgtagcggcgcattaagcgcggcgggtgtggtggttacgcgcagcgtgaccgctacactt
gccagcgccctagcgcccgctcctttcgctttcttcccttcctttctcgccacgttcgccggctttccccgtcaagctctaaatcgggggct
ccctttagggttccgatttagtgctttacggcacctcgaccccaaaaaacttgattagggtgatggttcacgtagtgggccatcgccctga
tagacggtttttcgccctttgacgttggagtccacgttctttaatagtggactcttgttccaaactggaacaacactcaaccctatctcggtct
attcttttgatttataagggattttgccgatttcggcctattggttaaaaaatgagctgatttaacaaaaatttaacgcgaattttaacaaaatat
taacgcttacaatttaggtggcacttttcggggaaatgtgcgcggaacccctatttgtttatttttctaaatacattcaaatatgtatccgctca
tgagacaataaccctgataaatgcttcaataatagcacctagatcaagagacaggatgaggatcgtttcgcatgattgaacaagatggat
tgcacgcaggttctccggccgcttgggtggagaggctattcggctatgactgggcacaacagacaatcggctgctctgatgccgccgt
gttccggctgtcagcgcaggggcgcccggttctttttgtcaagaccgacctgtccggtgccctgaatgaactgcaagacgaggcagc
gcggctatcgtggctggccacgacgggcgttccttgcgcagctgtgctcgacgttgtcactgaagcgggaagggactggctgctattg
ggcgaagtgccggggcaggatctcctgtcatctcaccttgctcctgccgagaaagtatccatcatggctgatgcaatgcggcggctgc
atacgcttgatccggctacctgcccattcgaccaccaagcgaaacatcgcatcgagcgagcacgtactcggatggaagccggtcttgt
cgatcaggatgatctggacgaagagcatcaggggctcgcgccagccgaactgttcgccaggctcaaggcgagcatgcccgacggc
gaggatctcgtcgtgacccatggcgatgcctgcttgccgaatatcatggtggaaaatggccgcttttctggattcatcgactgtggccgg
ctgggtgtggcggaccgctatcaggacatagcgttggctacccgtgatattgctgaagagcttggcggcgaatgggctgaccgcttcc
tcgtgctttacggtatcgccgctcccgattcgcagcgcatcgccttctatcgccttcttgacgagttcttctgaattattaacgcttacaattt
cctgatgcggtattttctccttacgcatctgtgcggtatttcacaccgcatcaggtggcacttttcggggaaatgtgcgcggaacccctatt
tgtttatttttctaaatacattcaaatatgtatccgctcatgaccaaaatcccttaacgtgagttttcgttccactgagcgtcagaccccgtag
aaaagatcaaaggatcttcttgagatcctttttttctgcgcgtaatctgctgcttgcaaacaaaaaaaccaccgctaccagcggtggtttgt
ttgccggatcaagagctaccaactctttttccgaaggtaactggcttcagcagagcgcagataccaaatactgttcttctagtgtagccgt
agttaggccaccacttcaagaactctgtagcaccgcctacatacctcgctctgctaatcctgttaccagtggctgctgccagtggcgata
agtcgtgtcttaccgggttggactcaagacgatagttaccggataaggcgcagcggtcgggctgaacggggggttcgtgcacacagc
ccagcttggagcgaacgacctacaccgaactgagatacctacagcgtgagctatgagaaagcgccacgcttcccgaagggagaaa
ggcggacaggtatccggtaagcggcagggtcggaacaggagagcgcacgagggagcttccagggggaaacgcctggtatctttat
agtcctgtcgggtttcgccacctctgacttgagcgtcgatttttgtgatgctcgtcaggggggcggagcctatggaaaaacgccagcaa
cgcggcctttttacggttcctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagcgcccaat
acgcaaaccgcctctccccgcgcgttggccgattcattaatgcagctggcacgacaggtttcccgactggaaagcgggcagtgagcg
caacgcaattaatgtgagttagctcactcattaggcaccccaggctttacactttatgcttccggctcgtatgttgtgtggaattgtgagcg
gataacaatttcacacaggaaacagctatgaccatgattacgccaagcgcgcaattaaccctcactaaagggaacaaaagctggagct
gcaagcttggccattgcatacgttgtatccatatcataatatgtacatttatattggctcatgtccaacattaccgccat
SEQ ID NO: 43 7) E5 Core, E14 Core, E9.1 Core, E12 OG +18 whole-RAG1pro-
RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctcgagaaaagtctattttatctaatataagcgtagttactcctgcttacttttggtttccattt
gcatggagtatctttttccatcctttcacttttcatctatgtgtgtctttgtagttgaagtgagtttcttgtagatagcacatggttatgtcctgtt
tgtttttgtccattccttctgtcattttattgttttctggttgttttgcatttctcttcctcgtttctttatctcttactgtttatctttgtgtggt
ttgcttggtgacaaattttagttactttctctttcgcatctgtgtatctgctctactagtgggttctatacttttgtttgttctcctgatggtagtt
attgccctttggctttcagatatagggctccgctaaagagccaggtggcagctggagctggggtctcctggcccatgattggctgccatcatttgtg
gttagccctccatggtgggggaggctgggaaggacagtggaagctgataaacagctcagcagcatgttctgagaaacaagagggcaagg
agagagcagagaacacactttgccttctctttggtattgagtaatatcaaccaaattgcagacatctcaacactttggccaggcagcctgc
tgagcaaggtacctcagccagcatggtgagcaagggcgaggagctgttcaccggggtggtgcccatcctggtcgagctggacggc
gacgtaaacggccacaagttcagcgtgtccggcgagggcgagggcgatgccacctacggcaagctgaccctgaagttcatctgcac
caccggcaagctgcccgtgccctggcccaccctcgtgaccaccttcggctacggcctgatgtgcttcgcccgctaccccgaccacat
gaagcagcacgacttcttcaagtccgccatgcccgaaggctacgtccaggagcgcaccatcttcttcaaggacgacggcaactacaa
gacccgcgccgaggtgaagttcgagggcgacaccctggtgaaccgcatcgagctgaagggcatcgacttcaaggaggacggcaa
catcctggggcacaagctggagtacaactacaacagccacaacgtctatatcatggccgacaagcagaagaacggcatcaaggtga
acttcaagatccgccacaacatcgaggacggcagcgtgcagctcgccgaccactaccagcagaacacccccatcggcgacggccc
cgtgctgctgcccgacaaccactacctgagctaccagtccgccctgagcaaagaccccaacgagaagcgcgatcacatggtcctgct
ggagttcgtgaccgccgccgggatcactctcggcatggacgagctgtacaagtgactgcaggaattcgagcatcttaccgccatttatt
cccatatttgttctgtttttcttgatttgggtatacatttaaatgttaataaaacaaaatggtggggcaatcatttacatttttagggatatgtaat
tactagttcaggtgtattgccacaagacaaacatgttaagaaactttcccgttatttacgctctgttcctgttaatcaacctctggattacaaaa
tttgtgaaagattgactgatattcttaactatgttgctccttttacgctgtgtggatatgctgctttaatgcctctgtatcatgctattgcttcccg
tacggctttcgttttctcctccttgtataaatcctggttgctgtctctttatgaggagttgtggcccgttgtccgtcaacgtggcgtggtgtgct
ctgtgtttgctgacgcaacccccactggctggggcattgccaccacctgtcaactcctttctgggactttcgctttccccctcccgatcgc
cacggcagaactcatcgccgcctgccttgcccgctgctggacaggggctaggttgctgggcactgataattccgtggtgttgtcgggg
aagggcctgctgccggctctgcggcctcttccgcgtcttcgccttcgccctcagacgagtcggatctccctttgggccgcctccccgcc
tggaattcgagctcggtacctttaagaccaatgacttacaaggcagctgtagatcttagccactttttaaaagaaaaggggggactggaa
gggctaattcactcccaacgaagacaagatctgctttttgcttgtactgggtctctctggttagaccagatctgagcctgggagctctctg
gctaactagggaacctactgcttaagcctcaataaagcttgccttgagtgcttcaagtagtgtgtgcccgtctgttgtgtgactctggtaac
tagagatccctcagacccttttagtcagtgtggaaaatctctagcagtagtagttcatgtcatcttattattcagtatttataacttgcaaagaa
atgaatatcagagagtgagaggaacttgtttattgcagcttataatggttacaaataaagcaatagcatcacaaatttcacaaataaagcat
ttttttcactgcattctagttgtggtttgtccaaactcatcaatgtatcttatcatgtctggctctagctatcccgcccctaactccgcccatccc
gcccctaactccgcccagttccgcccattctccgccccatggctgactaattttttttatttatgcagaggccgaggccgcctcggcctct
gagctattccagaagtagtgaggaggcttttttggaggcctagggacgtacccaattcgccctatagtgagtcgtattacgcgcgctcac
tggccgtcgttttacaacgtcgtgactgggaaaaccctggcgttacccaacttaatcgccttgcagcacatccccctttcgccagctggc
gtaatagcgaagaggcccgcaccgatcgcccttcccaacagttgcgcagcctgaatggcgaatgggacgcgccctgtagcggcgc
attaagcgcggcgggtgtggtggttacgcgcagcgtgaccgctacacttgccagcgccctagcgcccgctcctttcgctttcttcccttc
ctttctcgccacgttcgccggctttccccgtcaagctctaaatcgggggctccctttagggttccgatttagtgctttacggcacctcgacc
ccaaaaaacttgattagggtgatggttcacgtagtgggccatcgccctgatagacggtttttcgccctttgacgttggagtccacgttcttt
aatagtggactcttgttccaaactggaacaacactcaaccctatctcggtctattcttttgatttataagggattttgccgatttcggcctattg
gttaaaaaatgagctgatttaacaaaaatttaacgcgaattttaacaaaatattaacgcttacaatttaggtggcacttttcggggaaatgtg
cgcggaacccctatttgtttatttttctaaatacattcaaatatgtatccgctcatgagacaataaccctgataaatgcttcaataatagcacc
tagatcaagagacaggatgaggatcgtttcgcatgattgaacaagatggattgcacgcaggttctccggccgcttgggtggagaggct
attcggctatgactgggcacaacagacaatcggctgctctgatgccgccgtgttccggctgtcagcgcaggggcgcccggttctttttg
tcaagaccgacctgtccggtgccctgaatgaactgcaagacgaggcagcgcggctatcgtggctggccacgacgggcgttccttgc
gcagctgtgctcgacgttgtcactgaagcgggaagggactggctgctattgggcgaagtgccggggcaggatctcctgtcatctcac
cttgctcctgccgagaaagtatccatcatggctgatgcaatgcggcggctgcatacgcttgatccggctacctgcccattcgaccacca
agcgaaacatcgcatcgagcgagcacgtactcggatggaagccggtcttgtcgatcaggatgatctggacgaagagcatcaggggc
tcgcgccagccgaactgttcgccaggctcaaggcgagcatgcccgacggcgaggatctcgtcgtgacccatggcgatgcctgcttg
ccgaatatcatggtggaaaatggccgcttttctggattcatcgactgtggccggctgggtgtggcggaccgctatcaggacatagcgtt
ggctacccgtgatattgctgaagagcttggcggcgaatgggctgaccgcttcctcgtgctttacggtatcgccgctcccgattcgcagc
gcatcgccttctatcgccttcttgacgagttcttctgaattattaacgcttacaatttcctgatgcggtattttctccttacgcatctgtgcggta
tttcacaccgcatcaggtggcacttttcggggaaatgtgcgcggaacccctatttgtttatttttctaaatacattcaaatatgtatccgctca
tgaccaaaatcccttaacgtgagttttcgttccactgagcgtcagaccccgtagaaaagatcaaaggatcttcttgagatcctttttttctgc
gcgtaatctgctgcttgcaaacaaaaaaaccaccgctaccagcggtggtttgtttgccggatcaagagctaccaactctttttccgaagg
taactggcttcagcagagcgcagataccaaatactgttcttctagtgtagccgtagttaggccaccacttcaagaactctgtagcaccgc
ctacatacctcgctctgctaatcctgttaccagtggctgctgccagtggcgataagtcgtgtcttaccgggttggactcaagacgatagtt
accggataaggcgcagcggtcgggctgaacggggggttcgtgcacacagcccagcttggagcgaacgacctacaccgaactgag
atacctacagcgtgagctatgagaaagcgccacgcttcccgaagggagaaaggcggacaggtatccggtaagcggcagggtcgga
acaggagagcgcacgagggagcttccagggggaaacgcctggtatctttatagtcctgtcgggtttcgccacctctgacttgagcgtc
gatttttgtgatgctcgtcaggggggcggagcctatggaaaaacgccagcaacgcggcctttttacggttcctggccttttgctggccttt
tgctcacatgttctttcctgcgttatcccctgattctgtggataaccgtattaccgcctttgagtgagctgataccgctcgccgcagccgaa
cgaccgagcgcagcgagtcagtgagcgaggaagcggaagagcgcccaatacgcaaaccgcctctccccgcgcgttggccgattc
attaatgcagctggcacgacaggtttcccgactggaaagcgggcagtgagcgcaacgcaattaatgtgagttagctcactcattaggc
accccaggctttacactttatgcttccggctcgtatgttgtgtggaattgtgagcggataacaatttcacacaggaaacagctatgaccat
gattacgccaagcgcgcaattaaccctcactaaagggaacaaaagctggagctgcaagcttggccattgcatacgttgtatccatatca
taatatgtacatttatattggctcatgtccaacattaccgccat
SEQ ID NO: 44 8) E5 Core, E14 Core, E9.1 Core, E12 OG, E11 extra B-cell
enhancer + 18 right, 11 whole, 13 right-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctctaggcaagtgtctctctctctctccccctctttttcttttagaaaaatacttcgtgataaa
aacacaaaagtatgtgaatggtaattgaaaatgcattgcttccttttagaaacccactaggatttgcataaaccacttggctgtctgataagt
cattatctgttctgtcagtccctggggacttttaatattaggtttatatttttatagaaaaggcaagttctaaatttgaagatgggtattgttaaa
ctcttcaataatgacagtttgtgaaactgcatgttccaaaagtcctttaaaaagtcaataaggcgcttctgtcattttattgttttctggttgtttt
gcatttctcttcctcgtttctttatctcttactgtttatctttgtgtggtttgcttggtgacaaattttagttactttctctttcgcatctgtgtat
ctgctctactagtgggttctatacttttgtttgttctcctgatggtagttattgccctttggctttcagatatagggctccttttttaggtctacta
ctcttgtgtctgaaagttgcattttgtttgagtaagttcattaagcagggacctgaaagtaaacatcaaaggtgtgctgaatcagtggtgacaattt
catgtatcatatttcataataatgttctttcagagagcacataacattaactagcatggagcagtgcatgctccctgtatttccttctgggggcca
tgattgtgaaagtgttgcttgtgatttaccagcagatatttatacacatttacttatcacaagacgttttgagtctttcatgtggagctcaaagc
cttaaaacataaaaaccaatggcttgatttctgccacttgactttttttgaattcctttatcactcttttactcttttagtgatcattgacgtaggt
tcctgtacttgctgttgtcagaagaagcaagtttcagttttggaatgtaccctgcacgcttagttcttctttgactattgtaagccattcttctttg
tctcctttctggctccctgcccccacctttctgctccttttttcttctattctctaaagaccttttcttttcatttactgcattaatgcaggaagca
aggcacagtggttaggaggataggttctagagtcagaaggcagtgttcagatccccactctgctacttaaccatctgtatgctaaagagc
caggtggcagctggagctggggtctcctggcccatgattggctgccatcatttgtggttagccctccatggtgggggaggctgggaag
gacagtggaagctgataaacagctcagcagcatgttctgagaaacaagagggcaaggagagagcagagaacacactttgccttctct
ttggtattgagtaatatcaaccaaattgcagacatctcaacactttggccaggcagcctgctgagcaaggtacctcagccagcatggtg
agcaagggcgaggagctgttcaccggggtggtgcccatcctggtcgagctggacggcgacgtaaacggccacaagttcagcgtgt
ccggcgagggcgagggcgatgccacctacggcaagctgaccctgaagttcatctgcaccaccggcaagctgcccgtgccctggcc
caccctcgtgaccaccttcggctacggcctgatgtgcttcgcccgctaccccgaccacatgaagcagcacgacttcttcaagtccgcc
atgcccgaaggctacgtccaggagcgcaccatcttcttcaaggacgacggcaactacaagacccgcgccgaggtgaagttcgagg
gcgacaccctggtgaaccgcatcgagctgaagggcatcgacttcaaggaggacggcaacatcctggggcacaagctggagtacaa
ctacaacagccacaacgtctatatcatggccgacaagcagaagaacggcatcaaggtgaacttcaagatccgccacaacatcgagg
acggcagcgtgcagctcgccgaccactaccagcagaacacccccatcggcgacggccccgtgctgctgcccgacaaccactacct
gagctaccagtccgccctgagcaaagaccccaacgagaagcgcgatcacatggtcctgctggagttcgtgaccgccgccgggatca
ctctcggcatggacgagctgtacaagtgactgcaggaattcgagcatcttaccgccatttattcccatatttgttctgtttttcttgatttgggt
atacatttaaatgttaataaaacaaaatggtggggcaatcatttacatttttagggatatgtaattactagttcaggtgtattgccacaagaca
aacatgttaagaaactttcccgttatttacgctctgttcctgttaatcaacctctggattacaaaatttgtgaaagattgactgatattcttaact
atgttgctccttttacgctgtgtggatatgctgctttaatgcctctgtatcatgctattgcttcccgtacggctttcgttttctcctccttgtataa
atcctggttgctgtctctttatgaggagttgtggcccgttgtccgtcaacgtggcgtggtgtgctctgtgtttgctgacgcaacccccactgg
ctggggcattgccaccacctgtcaactcctttctgggactttcgctttccccctcccgatcgccacggcagaactcatcgccgcctgcctt
gcccgctgctggacaggggctaggttgctgggcactgataattccgtggtgttgtcggggaagggcctgctgccggctctgcggcct
cttccgcgtcttcgccttcgccctcagacgagtcggatctccctttgggccgcctccccgcctggaattcgagctcggtacctttaagac
caatgacttacaaggcagctgtagatcttagccactttttaaaagaaaaggggggactggaagggctaattcactcccaacgaagaca
agatctgctttttgcttgtactgggtctctctggttagaccagatctgagcctgggagctctctggctaactagggaacctactgcttaagc
ctcaataaagcttgccttgagtgcttcaagtagtgtgtgcccgtctgttgtgtgactctggtaactagagatccctcagacccttttagtcag
tgtggaaaatctctagcagtagtagttcatgtcatcttattattcagtatttataacttgcaaagaaatgaatatcagagagtgagaggaact
tgtttattgcagcttataatggttacaaataaagcaatagcatcacaaatttcacaaataaagcatttttttcactgcattctagttgtggtttgt
ccaaactcatcaatgtatcttatcatgtctggctctagctatcccgcccctaactccgcccatcccgcccctaactccgcccagttccgcc
cattctccgccccatggctgactaattttttttatttatgcagaggccgaggccgcctcggcctctgagctattccagaagtagtgaggag
gcttttttggaggcctagggacgtacccaattcgccctatagtgagtcgtattacgcgcgctcactggccgtcgttttacaacgtcgtgac
tgggaaaaccctggcgttacccaacttaatcgccttgcagcacatccccctttcgccagctggcgtaatagcgaagaggcccgcaccg
atcgcccttcccaacagttgcgcagcctgaatggcgaatgggacgcgccctgtagcggcgcattaagcgcggcgggtgtggtggtta
cgcgcagcgtgaccgctacacttgccagcgccctagcgcccgctcctttcgctttcttcccttcctttctcgccacgttcgccggctttcc
ccgtcaagctctaaatcgggggctccctttagggttccgatttagtgctttacggcacctcgaccccaaaaaacttgattagggtgatggt
tcacgtagtgggccatcgccctgatagacggtttttcgccctttgacgttggagtccacgttctttaatagtggactcttgttccaaactgga
acaacactcaaccctatctcggtctattcttttgatttataagggattttgccgatttcggcctattggttaaaaaatgagctgatttaacaaaa
atttaacgcgaattttaacaaaatattaacgcttacaatttaggtggcacttttcggggaaatgtgcgcggaacccctatttgtttatttttcta
aatacattcaaatatgtatccgctcatgagacaataaccctgataaatgcttcaataatagcacctagatcaagagacaggatgaggatc
gtttcgcatgattgaacaagatggattgcacgcaggttctccggccgcttgggtggagaggctattcggctatgactgggcacaacag
acaatcggctgctctgatgccgccgtgttccggctgtcagcgcaggggcgcccggttctttttgtcaagaccgacctgtccggtgccct
gaatgaactgcaagacgaggcagcgcggctatcgtggctggccacgacgggcgttccttgcgcagctgtgctcgacgttgtcactga
agcgggaagggactggctgctattgggcgaagtgccggggcaggatctcctgtcatctcaccttgctcctgccgagaaagtatccatc
atggctgatgcaatgcggcggctgcatacgcttgatccggctacctgcccattcgaccaccaagcgaaacatcgcatcgagcgagca
cgtactcggatggaagccggtcttgtcgatcaggatgatctggacgaagagcatcaggggctcgcgccagccgaactgttcgccag
gctcaaggcgagcatgcccgacggcgaggatctcgtcgtgacccatggcgatgcctgcttgccgaatatcatggtggaaaatggccg
cttttctggattcatcgactgtggccggctgggtgtggcggaccgctatcaggacatagcgttggctacccgtgatattgctgaagagct
tggcggcgaatgggctgaccgcttcctcgtgctttacggtatcgccgctcccgattcgcagcgcatcgccttctatcgccttcttgacga
gttcttctgaattattaacgcttacaatttcctgatgcggtattttctccttacgcatctgtgcggtatttcacaccgcatcaggtggcacttttc
ggggaaatgtgcgcggaacccctatttgtttatttttctaaatacattcaaatatgtatccgctcatgaccaaaatcccttaacgtgagttttc
gttccactgagcgtcagaccccgtagaaaagatcaaaggatcttcttgagatcctttttttctgcgcgtaatctgctgcttgcaaacaaaaa
aaccaccgctaccagcggtggtttgtttgccggatcaagagctaccaactctttttccgaaggtaactggcttcagcagagcgcagata
ccaaatactgttcttctagtgtagccgtagttaggccaccacttcaagaactctgtagcaccgcctacatacctcgctctgctaatcctgtt
accagtggctgctgccagtggcgataagtcgtgtcttaccgggttggactcaagacgatagttaccggataaggcgcagcggtcggg
ctgaacggggggttcgtgcacacagcccagcttggagcgaacgacctacaccgaactgagatacctacagcgtgagctatgagaaa
gcgccacgcttcccgaagggagaaaggcggacaggtatccggtaagcggcagggtcggaacaggagagcgcacgagggagctt
ccagggggaaacgcctggtatctttatagtcctgtcgggtttcgccacctctgacttgagcgtcgatttttgtgatgctcgtcaggggggc
ggagcctatggaaaaacgccagcaacgcggcctttttacggttcctggccttttgctggccttttgctcacatgttctttcctgcgttatccc
ctgattctgtggataaccgtattaccgcctttgagtgagctgataccgctcgccgcagccgaacgaccgagcgcagcgagtcagtgag
cgaggaagcggaagagcgcccaatacgcaaaccgcctctccccgcgcgttggccgattcattaatgcagctggcacgacaggtttc
ccgactggaaagcgggcagtgagcgcaacgcaattaatgtgagttagctcactcattaggcaccccaggctttacactttatgcttccg
gctcgtatgttgtgtggaattgtgagcggataacaatttcacacaggaaacagctatgaccatgattacgccaagcgcgcaattaaccct
cactaaagggaacaaaagctggagctgcaagcttggccattgcatacgttgtatccatatcataatatgtacatttatattggctcatgtcc
aacattaccgccat
SEQ ID NO: 45 9) E5 Core, E14 Core, E9.1 Core, E12 OG, E11 extra B-cell
enhancer + 18 whole, 11 whole-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctcgagtctaggcaagtgtctctctctctctccccctctttttcttttagaaaaatacttcgtg
ataaaaacacaaaagtatgtgaatggtaattgaaaatgcattgcttccttttagaaacccactaggatttgcataaaccacttggctgtctg
ataagtcattatctgttctgtcagtccctggggacttttaatattaggtttatatttttatagaaaaggcaagttctaaatttgaagatgggtatt
gttaaactcttcaataatgacagtttgtgaaactgcatgttccaaaagtcctttaaaaagtcaataaggcgaaaagtctattttatctaatata
agcgtagttactcctgcttacttttggtttccatttgcatggagtatctttttccatcctttcacttttcatctatgtgtgtctttgtagttgaagt
gagtttcttgtagatagcacatggttatgtcctgtttgtttttgtccattccttctgtcattttattgttttctggttgttttgcatttctcttcct
cgtttctttatctcttactgtttatctttgtgtggtttgcttggtgacaaattttagttactttctctttcgcatctgtgtatctgctctactagtg
ggttctatacttttgtttgttctcctgatggtagttattgccctttggctttcagatatagggctccttttttaggtctactactcttgtgtctgaa
agttgcattttgtttgagtaagttcattaagcagggacctgaaagtaaacatcaaaggtgtgctgaatcagtggtgacaatttcatgtatcatattt
cataataatgttctttcagagagcacataacattaactagcatggagcagtgcatgctccctgtatttccttctgggggccatgattgtgaaagtgt
tgcttgtgatttaccagcagatatttatacacatttacttatcacaagacgttttgagtctttcatgtggagctcaaagccttaaaacataaaaacc
aagctaaagagccaggtggcagctggagctggggtctcctggcccatgattggctgccatcatttgtggttagccctccatggtggggga
ggctgggaaggacagtggaagctgataaacagctcagcagcatgttctgagaaacaagagggcaaggagagagcagagaacaca
ctttgccttctctttggtattgagtaatatcaaccaaattgcagacatctcaacactttggccaggcagcctgctgagcaaggtacctcagc
cagcatggtgagcaagggcgaggagctgttcaccggggtggtgcccatcctggtcgagctggacggcgacgtaaacggccacaag
ttcagcgtgtccggcgagggcgagggcgatgccacctacggcaagctgaccctgaagttcatctgcaccaccggcaagctgcccgt
gccctggcccaccctcgtgaccaccttcggctacggcctgatgtgcttcgcccgctaccccgaccacatgaagcagcacgacttcttc
aagtccgccatgcccgaaggctacgtccaggagcgcaccatcttcttcaaggacgacggcaactacaagacccgcgccgaggtga
agttcgagggcgacaccctggtgaaccgcatcgagctgaagggcatcgacttcaaggaggacggcaacatcctggggcacaagct
ggagtacaactacaacagccacaacgtctatatcatggccgacaagcagaagaacggcatcaaggtgaacttcaagatccgccaca
acatcgaggacggcagcgtgcagctcgccgaccactaccagcagaacacccccatcggcgacggccccgtgctgctgcccgaca
accactacctgagctaccagtccgccctgagcaaagaccccaacgagaagcgcgatcacatggtcctgctggagttcgtgaccgcc
gccgggatcactctcggcatggacgagctgtacaagtgactgcaggaattcgagcatcttaccgccatttattcccatatttgttctgttttt
cttgatttgggtatacatttaaatgttaataaaacaaaatggtggggcaatcatttacatttttagggatatgtaattactagttcaggtgtattg
ccacaagacaaacatgttaagaaactttcccgttatttacgctctgttcctgttaatcaacctctggattacaaaatttgtgaaagattgactg
atattcttaactatgttgctccttttacgctgtgtggatatgctgctttaatgcctctgtatcatgctattgcttcccgtacggctttcgttttctc
ctccttgtataaatcctggttgctgtctctttatgaggagttgtggcccgttgtccgtcaacgtggcgtggtgtgctctgtgtttgctgacgcaa
cccccactggctggggcattgccaccacctgtcaactcctttctgggactttcgctttccccctcccgatcgccacggcagaactcatcg
ccgcctgccttgcccgctgctggacaggggctaggttgctgggcactgataattccgtggtgttgtcggggaagggcctgctgccgg
ctctgcggcctcttccgcgtcttcgccttcgccctcagacgagtcggatctccctttgggccgcctccccgcctggaattcgagctcggt
acctttaagaccaatgacttacaaggcagctgtagatcttagccactttttaaaagaaaaggggggactggaagggctaattcactccca
acgaagacaagatctgctttttgcttgtactgggtctctctggttagaccagatctgagcctgggagctctctggctaactagggaaccta
ctgcttaagcctcaataaagcttgccttgagtgcttcaagtagtgtgtgcccgtctgttgtgtgactctggtaactagagatccctcagacc
cttttagtcagtgtggaaaatctctagcagtagtagttcatgtcatcttattattcagtatttataacttgcaaagaaatgaatatcagagagtg
agaggaacttgtttattgcagcttataatggttacaaataaagcaatagcatcacaaatttcacaaataaagcatttttttcactgcattctagt
tgtggtttgtccaaactcatcaatgtatcttatcatgtctggctctagctatcccgcccctaactccgcccatcccgcccctaactccgccc
agttccgcccattctccgccccatggctgactaattttttttatttatgcagaggccgaggccgcctcggcctctgagctattccagaagta
gtgaggaggcttttttggaggcctagggacgtacccaattcgccctatagtgagtcgtattacgcgcgctcactggccgtcgttttacaa
cgtcgtgactgggaaaaccctggcgttacccaacttaatcgccttgcagcacatccccctttcgccagctggcgtaatagcgaagagg
cccgcaccgatcgcccttcccaacagttgcgcagcctgaatggcgaatgggacgcgccctgtagcggcgcattaagcgcggcgggt
gtggtggttacgcgcagcgtgaccgctacacttgccagcgccctagcgcccgctcctttcgctttcttcccttcctttctcgccacgttcg
ccggctttccccgtcaagctctaaatcgggggctccctttagggttccgatttagtgctttacggcacctcgaccccaaaaaacttgatta
gggtgatggttcacgtagtgggccatcgccctgatagacggtttttcgccctttgacgttggagtccacgttctttaatagtggactcttgtt
ccaaactggaacaacactcaaccctatctcggtctattcttttgatttataagggattttgccgatttcggcctattggttaaaaaatgagctg
atttaacaaaaatttaacgcgaattttaacaaaatattaacgcttacaatttaggtggcacttttcggggaaatgtgcgcggaacccctattt
gtttatttttctaaatacattcaaatatgtatccgctcatgagacaataaccctgataaatgcttcaataatagcacctagatcaagagacag
gatgaggatcgtttcgcatgattgaacaagatggattgcacgcaggttctccggccgcttgggtggagaggctattcggctatgactgg
gcacaacagacaatcggctgctctgatgccgccgtgttccggctgtcagcgcaggggcgcccggttctttttgtcaagaccgacctgt
ccggtgccctgaatgaactgcaagacgaggcagcgcggctatcgtggctggccacgacgggcgttccttgcgcagctgtgctcgac
gttgtcactgaagcgggaagggactggctgctattgggcgaagtgccggggcaggatctcctgtcatctcaccttgctcctgccgaga
aagtatccatcatggctgatgcaatgcggcggctgcatacgcttgatccggctacctgcccattcgaccaccaagcgaaacatcgcat
cgagcgagcacgtactcggatggaagccggtcttgtcgatcaggatgatctggacgaagagcatcaggggctcgcgccagccgaa
ctgttcgccaggctcaaggcgagcatgcccgacggcgaggatctcgtcgtgacccatggcgatgcctgcttgccgaatatcatggtg
gaaaatggccgcttttctggattcatcgactgtggccggctgggtgtggcggaccgctatcaggacatagcgttggctacccgtgatatt
gctgaagagcttggcggcgaatgggctgaccgcttcctcgtgctttacggtatcgccgctcccgattcgcagcgcatcgccttctatcg
ccttcttgacgagttcttctgaattattaacgcttacaatttcctgatgcggtattttctccttacgcatctgtgcggtatttcacaccgcatcag
gtggcacttttcggggaaatgtgcgcggaacccctatttgtttatttttctaaatacattcaaatatgtatccgctcatgaccaaaatccctta
acgtgagttttcgttccactgagcgtcagaccccgtagaaaagatcaaaggatcttcttgagatcctttttttctgcgcgtaatctgctgctt
gcaaacaaaaaaaccaccgctaccagcggtggtttgtttgccggatcaagagctaccaactctttttccgaaggtaactggcttcagca
gagcgcagataccaaatactgttcttctagtgtagccgtagttaggccaccacttcaagaactctgtagcaccgcctacatacctcgctct
gctaatcctgttaccagtggctgctgccagtggcgataagtcgtgtcttaccgggttggactcaagacgatagttaccggataaggcgc
agcggtcgggctgaacggggggttcgtgcacacagcccagcttggagcgaacgacctacaccgaactgagatacctacagcgtga
gctatgagaaagcgccacgcttcccgaagggagaaaggcggacaggtatccggtaagcggcagggtcggaacaggagagcgca
cgagggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttcttt
cctgcgttatcccctgattctgtggataaccgtattaccgcctttgagtgagctgataccgctcgccgcagccgaacgaccgagcgcag
cgagtcagtgagcgaggaagcggaagagcgcccaatacgcaaaccgcctctccccgcgcgttggccgattcattaatgcagctggc
acgacaggtttcccgactggaaagcgggcagtgagcgcaacgcaattaatgtgagttagctcactcattaggcaccccaggctttaca
ctttatgcttccggctcgtatgttgtgtggaattgtgagcggataacaatttcacacaggaaacagctatgaccatgattacgccaagcgc
gcaattaaccctcactaaagggaacaaaagctggagctgcaagcttggccattgcatacgttgtatccatatcataatatgtacatttatat
tggctcatgtccaacattaccgccat
SEQ ID NO: 46 10) E5 Core, E14 Core, E9.1 Core, E12 OG, E11 extra B-cell
enhancer + 18 whole-RAG1pro-RAG-WPRE
cttgacattgattattgactagttattaatagtaatcaattacggggtcattagttcatagcccatatatggagttccgcgttacataacttacg
gtaaatggcccgcctggctgaccgcccaacgacccccgcccattgacgtcaataatgacgtatgttcccatagtaacgccaataggga
ctttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctacgtattag
tcatcgctattaccatggtgatgcggttttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatggg
cggtaggcgtgtacggtgggaggtctatataagcagagctcgtttagtgaaccggggtctctctggttagaccagatctgagcctggg
agctctctggctaactagggaacccactgcttaagcctcaataaagcttgccttgagtgcttcaagtagtgtgtgcccgtctgttgtgtga
ctctggtaactagagatccctcagacccttttagtcagtgtggaaaatctctagcagtggcgcccgaacagggacttgaaagcgaaag
ggaaaccagaggagctctctcgacgcaggactcggcttgctgaagcgcgcacggcaagaggcgaggggcggcgactggtgagta
cgccaaaaattttgactagcggaggctagaaggagagagatgggtgcgagagcgtcagtattaagcgggggagaattagatcgcga
tgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaag
aacttagatcattatataatacagtagcaaccctctattgtgtgcatcaaaggatagagataaaagacaccaaggaagctttagacaaga
tagaggaagagcaaaacaaaagtaagaccaccgcacagcaagcggccgctgatcttcagacctggaggaggagatatgagggac
aattggagaagtgaattatataaatataaagtagtaaaaattgaaccattaggagtagcacccaccaaggcaaagagaagagtggtgc
agagagaaaaaagagcagtgggaataggagctttgttccttgggttcttgggagcagcaggaagcactatgggcgcagcgtcaatga
cgctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctggg
gatttggggttgctctggaaaactcatttgcaccactgctgtgccttggaatgctagttggagtaataaatctctggaacagatttggaatc
acacgacctggatggagtgggacagagaaattaacaattacacaagcttaatacactccttaattgaagaatcgcaaaaccagcaaga
aaagaatgaacaagaattattggaattagataaatgggcaagtttgtggaattggtttaacataacaaattggctgtggtatataaaattatt
cataatgatagtaggaggcttggtaggtttaagaatagtttttgctgtactttctatagtgaatagagttaggcagggatattcaccattatcg
tttcagacccacctcccaaccccgaggggacccgacaggcccgaaggaatagaagaagaaggtggagagagagacagagacag
atccattcgattagtgaacggatctcgacggtatcggttaacttttaaaagaaaaggggggattggggggtacagtgcaggggaaaga
atagtagacataatagcaacagacatacaaactaaagaattacaaaaacaaattacaaaaattcaaaattttatcgatcacgagactagc
cagaaaagagggattggggccctctcccaacagacatgagtatttaggagtccccagttccggtttccacatgacatgatatgatggtct
gttgttttcctcagggacctagaggcagatgtagacaaatagcaggtgaggaggaagggtggtgctgagctgtaaacaacagctgcg
ggctgtaaacaatggagctatattaagaaagtcctgcatctgcagcattcggttcaatcctgccacccactgggcgtactcctgaattga
ctttccattgtcagctgatcagtgacagatgtgaaatgactcgttacacacacaggtcacattgggaggattcttagacgtcatattctttg
gtgcttcacaaagtcttactgtggcacatgaaatgatttcagatggtgcacaagatgatttcacatgtaaatttccatgagggtgcaaaga
gaggcttttcccaatctgaagaatgaatttaaaatctgctgtagaaaaataccagagagaaaaatatagaaagcagctgtccctcttacc
aatttcagagcaggaaaggaatttcagtagagcataaaaacagctttctctcataaaagaaaacacaacaagaaaaataatccttgctgt
gtagccttttgcagttattttctgtcatgtgttagatagagaccagagggcttaacattgttttgatacccttgcatctggcagattgcctgtca
gaggaagttggaaacgaactgaccagctttatgcttccaggtggcactgaagccacattttcagtgccgttgttatcagtcatttaattccc
tactgaatattgagtggtgtttttagttcacaggccatttgcagtgcctttctgtttcctttttaacgttagccacattaagaaaaaaagtgggc
tttgcctacagcacctgcttctgctttccgcatttactgatccactaagtgtaagtgtgacatatagaggtctgtgttggttgttgttttgtgttc
aaacagctgaacatctggcctctcatgtgcaggtgcacagaactcaggagaaggggttttaatttaagataaaaaaaataaggatatgt
gctagttgcagatgagggtttgggtgctggacttaagatgcttctgtgttcaaaaacaaggagtcctcaatttgcaaggttcataaagagt
ttcacacagatggcacccaaagtgcttatttctcattgtgctgaaattaaacttgaatatgctatatatcaaataaatagaagtaaatagcctt
tctttatactttctagaagttgagctcaatgttctttcggagagagcaggctcatctccattatgtagcgccaatgacaaatacctgtatatgt
ctgtatttccttggtctgtcttgctcacctgttaagacaccttgctgcacaacagatggcacatgcttatcttgtggcagtgaagagttgaca
tcaaaggccaagaaaaccttgaaactcatccttttcagcccacaagagcagcttctctctaccacagacgatgaaagctggagataaca
ggggcattcccaccgctgcactctctcttgtggccagctgacagttaacacaggtgggcgtgaggtgctgcctacattttgaaggttgtc
tattgatggggaacaccacctctctgagctcaggcctctgctttggcccgtggagctgctgcttctgagaagtaaccaggtggtgaatac
ctgcctgcttttcagaaaatccgaggtggctgtcaattgacaattatttattcattgtaaaataatcacaggaacagcagcagtgtaggtttc
cctacctagagggtggtatgcagtgattctcaggcgctggttggaaggcacagctgagggacacaaactgccaggaagtaatgtggt
aactagccatgagcttgtggtactaatggtggcacgggaaacaaggtctctgcttgacttttattttcactccataacaaactcaccagatc
aggagcctaagggtgggtggggagggagaagagagaaaaaagcaaagggaaagttcaaagtgacactcgctggggctgaaaac
cacactcccctgcagatgaggtccttggctctcgagtctaggcaagtgtctctctctctctccccctctttttcttttagaaaaatacttcgtg
ataaaaacacaaaagtatgtgaatggtaattgaaaatgcattgcttccttttagaaacccactaggatttgcataaaccacttggctgtctg
ataagtcattatctgttctgtcagtccctggggacttttaatattaggtttatatttttatagaaaaggcaagttctaaatttgaagatgggtatt
gttaaactcttcaataatgacagtttgtgaaactgcatgttccaaaagtcctttaaaaagtcaataaggcgaaaagtctattttatctaatata
agcgtagttactcctgcttacttttggtttccatttgcatggagtatctttttccatcctttcacttttcatctatgtgtgtctttgtagttgaagt
gagtttcttgtagatagcacatggttatgtcctgtttgtttttgtccattccttctgtcattttattgttttctggttgttttgcatttctcttcct
cgtttctttatctcttactgtttatctttgtgtggtttgcttggtgacaaattttagttactttctctttcgcatctgtgtatctgctctactagtg
ggttctatacttttgtttgttctcctgatggtagttattgccctttggctttcagatatagggctccttttttaggtctactactcttgtgtctga
aagttgcattttgtttgagtaagttcattaagcagggacctgaaagtaaacatcaaaggtgtgctgaatcagtggtgacaatttcatgtatcatatt
tcataataatgttctttcagagagcacataacattaactagcatggagcagtgcatgctccctgtatttccttctgggggccatgattgtgaaagtg
ttgcttgtgatttaccagcagatatttatacacatttacttatcacaagacgttttgagtctttcatgtggagctcaaagccttaaaacataaaaac
caagctaaagagccaggtggcagctggagctggggtctcctggcccatgattggctgccatcatttgtggttagccctccatggtggggga
ggctgggaaggacagtggaagctgataaacagctcagcagcatgttctgagaaacaagagggcaaggagagagcagagaacaca
ctttgccttctctttggtattgagtaatatcaaccaaattgcagacatctcaacactttggccaggcagcctgctgagcaaggtacctcagc
cagcatggtgagcaagggcgaggagctgttcaccggggtggtgcccatcctggtcgagctggacggcgacgtaaacggccacaag
ttcagcgtgtccggcgagggcgagggcgatgccacctacggcaagctgaccctgaagttcatctgcaccaccggcaagctgcccgt
gccctggcccaccctcgtgaccaccttcggctacggcctgatgtgcttcgcccgctaccccgaccacatgaagcagcacgacttcttc
aagtccgccatgcccgaaggctacgtccaggagcgcaccatcttcttcaaggacgacggcaactacaagacccgcgccgaggtga
agttcgagggcgacaccctggtgaaccgcatcgagctgaagggcatcgacttcaaggaggacggcaacatcctggggcacaagct
ggagtacaactacaacagccacaacgtctatatcatggccgacaagcagaagaacggcatcaaggtgaacttcaagatccgccaca
acatcgaggacggcagcgtgcagctcgccgaccactaccagcagaacacccccatcggcgacggccccgtgctgctgcccgaca
accactacctgagctaccagtccgccctgagcaaagaccccaacgagaagcgcgatcacatggtcctgctggagttcgtgaccgcc
gccgggatcactctcggcatggacgagctgtacaagtgactgcaggaattcgagcatcttaccgccatttattcccatatttgttctgttttt
cttgatttgggtatacatttaaatgttaataaaacaaaatggtggggcaatcatttacatttttagggatatgtaattactagttcaggtgtattg
ccacaagacaaacatgttaagaaactttcccgttatttacgctctgttcctgttaatcaacctctggattacaaaatttgtgaaagattgactg
atattcttaactatgttgctccttttacgctgtgtggatatgctgctttaatgcctctgtatcatgctattgcttcccgtacggctttcgttttctc
ctccttgtataaatcctggttgctgtctctttatgaggagttgtggcccgttgtccgtcaacgtggcgtggtgtgctctgtgtttgctgacgcaa
cccccactggctggggcattgccaccacctgtcaactcctttctgggactttcgctttccccctcccgatcgccacggcagaactcatcg
ccgcctgccttgcccgctgctggacaggggctaggttgctgggcactgataattccgtggtgttgtcggggaagggcctgctgccgg
ctctgcggcctcttccgcgtcttcgccttcgccctcagacgagtcggatctccctttgggccgcctccccgcctggaattcgagctcggt
acctttaagaccaatgacttacaaggcagctgtagatcttagccactttttaaaagaaaaggggggactggaagggctaattcactccca
acgaagacaagatctgctttttgcttgtactgggtctctctggttagaccagatctgagcctgggagctctctggctaactagggaaccta
ctgcttaagcctcaataaagcttgccttgagtgcttcaagtagtgtgtgcccgtctgttgtgtgactctggtaactagagatccctcagacc
cttttagtcagtgtggaaaatctctagcagtagtagttcatgtcatcttattattcagtatttataacttgcaaagaaatgaatatcagagagtg
agaggaacttgtttattgcagcttataatggttacaaataaagcaatagcatcacaaatttcacaaataaagcatttttttcactgcattctagt
tgtggtttgtccaaactcatcaatgtatcttatcatgtctggctctagctatcccgcccctaactccgcccatcccgcccctaactccgccc
agttccgcccattctccgccccatggctgactaattttttttatttatgcagaggccgaggccgcctcggcctctgagctattccagaagta
gtgaggaggcttttttggaggcctagggacgtacccaattcgccctatagtgagtcgtattacgcgcgctcactggccgtcgttttacaa
cgtcgtgactgggaaaaccctggcgttacccaacttaatcgccttgcagcacatccccctttcgccagctggcgtaatagcgaagagg
cccgcaccgatcgcccttcccaacagttgcgcagcctgaatggcgaatgggacgcgccctgtagcggcgcattaagcgcggcgggt
gtggtggttacgcgcagcgtgaccgctacacttgccagcgccctagcgcccgctcctttcgctttcttcccttcctttctcgccacgttcg
ccggctttccccgtcaagctctaaatcgggggctccctttagggttccgatttagtgctttacggcacctcgaccccaaaaaacttgatta
gggtgatggttcacgtagtgggccatcgccctgatagacggtttttcgccctttgacgttggagtccacgttctttaatagtggactcttgtt
ccaaactggaacaacactcaaccctatctcggtctattcttttgatttataagggattttgccgatttcggcctattggttaaaaaatgagctg
atttaacaaaaatttaacgcgaattttaacaaaatattaacgcttacaatttaggtggcacttttcggggaaatgtgcgcggaacccctattt
gtttatttttctaaatacattcaaatatgtatccgctcatgagacaataaccctgataaatgcttcaataatagcacctagatcaagagacag
gatgaggatcgtttcgcatgattgaacaagatggattgcacgcaggttctccggccgcttgggtggagaggctattcggctatgactgg
gcacaacagacaatcggctgctctgatgccgccgtgttccggctgtcagcgcaggggcgcccggttctttttgtcaagaccgacctgt
ccggtgccctgaatgaactgcaagacgaggcagcgcggctatcgtggctggccacgacgggcgttccttgcgcagctgtgctcgac
gttgtcactgaagcgggaagggactggctgctattgggcgaagtgccggggcaggatctcctgtcatctcaccttgctcctgccgaga
aagtatccatcatggctgatgcaatgcggcggctgcatacgcttgatccggctacctgcccattcgaccaccaagcgaaacatcgcat
cgagcgagcacgtactcggatggaagccggtcttgtcgatcaggatgatctggacgaagagcatcaggggctcgcgccagccgaa
ctgttcgccaggctcaaggcgagcatgcccgacggcgaggatctcgtcgtgacccatggcgatgcctgcttgccgaatatcatggtg
gaaaatggccgcttttctggattcatcgactgtggccggctgggtgtggcggaccgctatcaggacatagcgttggctacccgtgatatt
gctgaagagcttggcggcgaatgggctgaccgcttcctcgtgctttacggtatcgccgctcccgattcgcagcgcatcgccttctatcg
ccttcttgacgagttcttctgaattattaacgcttacaatttcctgatgcggtattttctccttacgcatctgtgcggtatttcacaccgcatcag
gtggcacttttcggggaaatgtgcgcggaacccctatttgtttatttttctaaatacattcaaatatgtatccgctcatgaccaaaatccctta
acgtgagttttcgttccactgagcgtcagaccccgtagaaaagatcaaaggatcttcttgagatcctttttttctgcgcgtaatctgctgctt
gcaaacaaaaaaaccaccgctaccagcggtggtttgtttgccggatcaagagctaccaactctttttccgaaggtaactggcttcagca
gagcgcagataccaaatactgttcttctagtgtagccgtagttaggccaccacttcaagaactctgtagcaccgcctacatacctcgctct
gctaatcctgttaccagtggctgctgccagtggcgataagtcgtgtcttaccgggttggactcaagacgatagttaccggataaggcgc
agcggtcgggctgaacggggggttcgtgcacacagcccagcttggagcgaacgacctacaccgaactgagatacctacagcgtga
gctatgagaaagcgccacgcttcccgaagggagaaaggcggacaggtatccggtaagcggcagggtcggaacaggagagcgca
cgagggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttcttt
cctgcgttatcccctgattctgtggataaccgtattaccgcctttgagtgagctgataccgctcgccgcagccgaacgaccgagcgcag
cgagtcagtgagcgaggaagcggaagagcgcccaatacgcaaaccgcctctccccgcgcgttggccgattcattaatgcagctggc
acgacaggtttcccgactggaaagcgggcagtgagcgcaacgcaattaatgtgagttagctcactcattaggcaccccaggctttaca
ctttatgcttccggctcgtatgttgtgtggaattgtgagcggataacaatttcacacaggaaacagctatgaccatgattacgccaagcgc
gcaattaaccctcactaaagggaacaaaagctggagctgcaagcttggccattgcatacgttgtatccatatcataatatgtacatttatat
tggctcatgtccaacattaccgccat

What is claimed is:

1. A recombinant lentiviral vector (LV) for the treatment of Recombination-Activating Gene 1 (RAG1) Severe Combined Immunodeficiency, said vector comprising:
an expression cassette comprising:
a nucleic acid encoding an effective fragment of the endogenous promoter of the RAG1 gene; and
a nucleic acid that encodes the RAG1 protein operably linked to said effective fragment of the endogenous promoter of the RAG1 gene.

2. The vector of claim 1, wherein the sequence of said effective fragment of the endogenous promoter of the RAG1 gene comprises or consists of the sequence of RAGpro (SEQ ID NO:2).

3. The vector of claim 2, wherein the sequence of said effective fragment of the endogenous promoter of the RAG1 gene consists of the sequence of RAGpro (SEQ ID NO:2).

4. The vector according to any one of claims 1-3, wherein said expression cassette comprises a RAG1 enhancer element 1 (SEQ ID NO: 1) or an effective fragment thereof.

5. The vector of claim 4, wherein the sequence of said RAG1 enhancer element consists the sequence of SEQ ID NO: 1 or an effective fragment thereof.

6. The vector according to any one of claims 1-5, wherein said expression cassette comprises a RAG1 enhancer element 3 (SEQ ID NO:3) or an effective fragment thereof.

7. The vector of claim 6, wherein the sequence of said RAG1 enhancer element 3 consists of the sequence of SEQ ID NO:3 or an effective fragment thereof.

8. The vector according to any one of claims 1-7, wherein said expression cassette comprises a RAG1 enhancer element 4 (SEQ ID NO:4) or an effective fragment thereof.

9. The vector of claim 8, wherein the sequence of said RAG1 enhancer element 4 consists of the sequence of SEQ ID NO:4 or an effective fragment thereof.

10. The vector according to any one of claims 1-9, wherein said expression cassette comprises a RAG1 enhancer element 5 (SEQ ID NO:5) or an effective fragment thereof.

11. The vector of claim 10, wherein the sequence of said RAG1 enhancer element 5 consists of the sequence of SEQ ID NO:5 or an effective fragment thereof.

12. The vector of claim 10, wherein the sequence of said RAG1 enhancer element 5 consists of the sequence of SEQ ID NO:31 or an effective fragment thereof.

13. The vector according to any one of claims 1-12, wherein said expression cassette comprises a RAG1 enhancer element 6 (SEQ ID NO:6) or an effective fragment thereof.

14. The vector of claim 13, wherein the sequence of said RAG1 enhancer element 6 consists of the sequence of SEQ ID NO:6 or an effective fragment thereof.

15. The vector according to any one of claims 1-14, wherein said expression cassette comprises a RAG1 enhancer element 7 (SEQ ID NO:7) or an effective fragment thereof.

16. The vector of claim 15, wherein the sequence of said RAG1 enhancer element 7 consists of the sequence of SEQ ID NO:7 or an effective fragment thereof.

17. The vector according to any one of claims 1-16, wherein said expression cassette comprises a RAG1 enhancer element 8 (SEQ ID NO:8) or an effective fragment thereof.

18. The vector of claim 17, wherein the sequence of said RAG1 enhancer element 8 consists of the sequence of SEQ ID NO:8 or an effective fragment thereof.

19. The vector according to any one of claims 1-18, wherein said expression cassette comprises a RAG1 enhancer element 9.1 (SEQ ID NO:9) or an effective fragment thereof.

20. The vector of claim 19, wherein the sequence of said RAG1 enhancer element 9.1 consists of the sequence of SEQ ID NO:9 or an effective fragment thereof.

21. The vector of claim 19, wherein the sequence of said RAG1 enhancer element 9.1 consists of enhancer element 9.1 core sequence of SEQ ID NO:34 or an effective fragment thereof.

22. The vector according to any one of claims 1-21, wherein said expression cassette comprises a RAG1 enhancer element 9.2 (SEQ ID NO:10) or an effective fragment thereof.

23. The vector of claim 22, wherein the sequence of said RAG1 enhancer element 9.2 consists of the sequence of SEQ ID NO:10 or an effective fragment thereof.

24. The vector according to any one of claims 1-23, wherein said expression cassette comprises a RAG1 enhancer element 10 (SEQ ID NO:11) or an effective fragment thereof.

25. The vector of claim 24, wherein the sequence of said RAG1 enhancer element 10 consists of the sequence of SEQ ID NO:11 or an effective fragment thereof.

26. The vector according to any one of claims 1-25, wherein said expression cassette comprises or consists of a RAG1 enhancer element 11 extra (SEQ ID NO:33) or an effective fragment thereof.

27. The vector according to any one of claims 1-25, wherein said expression cassette comprises a RAG1 enhancer element 11 (SEQ ID NO:12) or an effective fragment thereof.

28. The vector of claim 27, wherein the sequence of said RAG1 enhancer element 11 consists of the sequence of SEQ ID NO:12 or an effective fragment thereof.

29. The vector according to any one of claims 1-28, wherein said expression cassette comprises a RAG1 enhancer element 12 (SEQ ID NO:13) or an effective fragment thereof.

30. The vector of claim 29, wherein the sequence of said RAG1 enhancer element 12 consists of the sequence of SEQ ID NO:13 or an effective fragment thereof.

31. The vector according to any one of claims 1-30, wherein said expression cassette comprises a RAG1 enhancer element 13 (SEQ ID NO:14) or an effective fragment thereof.

32. The vector of claim 31, wherein the sequence of said RAG1 enhancer element 13 consists of the sequence of SEQ ID NO:14 or an effective fragment thereof.

33. The vector of claim 31, wherein the sequence of said RAG1 enhancer element 13 consists of the sequence of SEQ ID NO:36 or an effective fragment thereof.

34. The vector according to any one of claims 1-33, wherein said expression cassette comprises a RAG1 enhancer element 14 (SEQ ID NO:15) or an effective fragment thereof.

35. The vector of claim 34, wherein said expression cassette comprises or consists of an enlarged RAG1 enhancer element 14 core (SEQ ID NO:32) or an effective fragment thereof.

36. The vector of claim 34, wherein the sequence of said RAG1 enhancer element 14 consists of the sequence of SEQ ID NO:15 or an effective fragment thereof.

37. The vector according to any one of claims 1-36, wherein said expression cassette comprises a RAG1 enhancer element 16 (SEQ ID NO:16) or an effective fragment thereof.

38. The vector of claim 37, wherein the sequence of said RAG1 enhancer element 16 consists of the sequence of SEQ ID NO:16 or an effective fragment thereof.

39. The vector according to any one of claims 1-38, wherein said expression cassette comprises a RAG1 enhancer element 17 (SEQ ID NO:17) or an effective fragment thereof.

40. The vector of claim 39, wherein the sequence of said RAG1 enhancer element 17 consists of the sequence of SEQ ID NO:17 or an effective fragment thereof.

41. The vector according to any one of claims 1-40, wherein said expression cassette comprises a RAG1 enhancer element 18 (SEQ ID NO:18) or an effective fragment thereof.

42. The vector of claim 41, wherein the sequence of said RAG1 enhancer element 18 consists of the sequence of SEQ ID NO:18 or an effective fragment thereof.

43. The vector of claim 41, wherein the sequence of said RAG1 enhancer element 18 consists of the sequence of SEQ ID NO:35 or an effective fragment thereof.

44. The vector according to any one of claims 1-43, wherein said nucleic acid that encodes a RAG1 Protein is a RAG1 cDNA or a codon-optimized RAG1 gene or cDNA.

45. The vector of claim 44, wherein said nucleic acid that encodes a nucleic acid that encodes RAG1 protein is a RAG1 cDNA (SEQ ID NO:19).

46. The vector of claim 44, wherein said nucleic acid that encodes a nucleic acid that encodes RAG1 protein is a codon optimized RAG1.

47. The vector of claim 46, wherein the sequence of said nucleic acid that encodes RAG1 protein is a codon optimized RAG1 selected from the group consisting of jCAT codon optimized RAG1, GeneArt optimized RAG1, and IDT optimized RAG1.

48. The vector according to any one of claims 1-47, wherein said vector comprises a ψ region vector genome packaging signal.

49. The vector according to any one of claims 1-48, wherein said vector comprise a 5′ LTR comprising a CMV enhancer/promoter.

50. The vector according to any one of claims 1-49, wherein said vector comprises a Rev Responsive Element (RRE).

51. The vector according to any one of claims 1-50, wherein said vector comprises a central polypurine tract.

52. The vector according to any one of claims 1-51, wherein said vector comprises a post-translational regulatory element.

53. The vector of claim 52, wherein the posttranscriptional regulatory element is modified Woodchuck Post-transcriptional Regulatory Element (WPRE).

54. The vector according to any one of claims 1-53, wherein said vector is incapable of reconstituting a wild-type lentivirus through recombination.

55. The vector according to any one of claims 1-54, wherein said vector shows high expression in T-cells.

56. The vector according to any one of claims 1-55, wherein said vector shows high expression in B-cells.

57. The vector of claim 1, wherein said vector comprises the vector elements selected from the group consisting of:
1) E5 Core, E14 Core, E9.1 Core, E12 OG—RAG1pro-RAG-WPRE;
2) E5 Core, E14 Core, E9.1 Core, E12 OG, E11 extra B-cell enhancer—RAG1pro-RAG-WPRE;
3) E5 Core, E14 Core, E9.1 OG large, E12 OG—RAG1pro-RAG-WPRE;
4) E5 Core, E14 Core, E9.1 OG large, E12 OG, E11 extra B-cell enhancer—RAG1pro-RAG-WPRE;
5) E5 Core, E14 Core, E9.1 Core, E12 OG+18 right, 11 whole, 13 right—RAG1pro-RAG-WPRE;
6) E5 Core, E14 Core, E9.1 Core, E12 OG+18 whole, 11 whole—RAG1pro-RAG-WPRE;
7) E5 Core, E14 Core, E9.1 Core, E12 OG+18 whole—RAG1pro-RAG-WPRE;
8) E5 Core, E14 Core, E9.1 Core, E12 OG, E11 extra B-cell enhancer+18 right, 11 whole, 13 right—RAG1pro-RAG-WPRE;
9) E5 Core, E14 Core, E9.1 Core, E12 OG, E11 extra B-cell enhancer+18 whole, 11 whole—RAG1pro-RAG-WPRE; and
10) E5 Core, E14 Core, E9.1 Core, E12 OG, E11 extra B-cell enhancer+18 whole—RAG1pro-RAG-WPRE.

58. The vector of claim 57, wherein said vector comprises the vector elements E5 Core, E14 Core, E9.1 Core, E12 OG—RAG1pro-RAG-WPRE.

59. The vector of claim 58, wherein said vector comprises the nucleotide sequence of SEQ ID NO:37.

60. The vector of claim 57, wherein said vector comprises the vector elements E5 Core, E14 Core, E9.1 Core, E12 OG, E11 extra B-cell enhancer—RAG1pro-RAG-WPRE.

61. The vector of claim 58, wherein said vector comprises the nucleotide sequence of SEQ ID NO:38.

62. The vector of claim 57, wherein said vector comprises the vector elements E5 Core, E14 Core, E9.1 OG large, E12 OG—RAG1pro-RAG-WPRE.

63. The vector of claim 58, wherein said vector comprises the nucleotide sequence of SEQ ID NO:39.

64. The vector of claim 57, wherein said vector comprises the vector elements E5 Core, E14 Core, E9.1 OG large, E12 OG, E11 extra B-cell enhancer—RAG1pro-RAG-WPRE.

65. The vector of claim 58, wherein said vector comprises the nucleotide sequence of SEQ ID NO:40.

66. The vector of claim 57, wherein said vector comprises the vector elements E5 Core, E14 Core, E9.1 Core, E12 OG+18 right, 11 whole, 13 right—RAG1pro-RAG-WPRE.

67. The vector of claim 58, wherein said vector comprises the nucleotide sequence of SEQ ID NO:41.

68. The vector of claim 57, wherein said vector comprises the vector elements E5 Core, E14 Core, E9.1 Core, E12 OG+18 whole, 11 whole—RAG1pro-RAG-WPRE.

69. The vector of claim 58, wherein said vector comprises the nucleotide sequence of SEQ ID NO:42.

70. The vector of claim 57, wherein said vector comprises the vector elements E5 Core, E14 Core, E9.1 Core, E12 OG+18 whole—RAG1pro-RAG-WPRE.

71. The vector of claim 58, wherein said vector comprises the nucleotide sequence of SEQ ID NO:43.

72. The vector of claim 57, wherein said vector comprises the vector elements E5 Core, E14 Core, E9.1 Core, E12 OG, E11 extra B-cell enhancer+18 right, 11 whole, 13 right—RAG1pro-RAG-WPRE.

73. The vector of claim 58, wherein said vector comprises the nucleotide sequence of SEQ ID NO:44.

74. The vector of claim 57, wherein said vector comprises the vector elements E5 Core, E14 Core, E9.1 Core, E12 OG, E11 extra B-cell enhancer+18 whole, 11 whole—RAG1pro-RAG-WPRE.

75. The vector of claim 58, wherein said vector comprises the nucleotide sequence of SEQ ID NO:45.

76. The vector of claim 57, wherein said vector comprises the vector elements E5 Core, E14 Core, E9.1 Core, E12 OG, E11 extra B-cell enhancer+18 whole—RAG1pro-RAG-WPRE.

77. The vector of claim 58, wherein said vector comprises the nucleotide sequence of SEQ ID NO:46.

78. A host cell transduced with a vector according to any one of claims 1-77.

79. The host cell of claim 78, wherein the cell is a stem cell.

80. The host cell of claim 79, wherein said cell is a stem cell derived from bone marrow, and/or from umbilical cord blood, and/or from peripheral blood.

81. The host cell of claim 78, wherein the cell is a human hematopoietic progenitor cell.

82. The host cell of claim 81, wherein the human hematopoietic progenitor cell is a CD34+ cell.

83. A method of treating Recombination-Activating Gene 1 (RAG1) Severe Combined Immunodeficiency (RAG1 SCID), in a subject, said method comprising:
transducing a stem cell and/or progenitor cell from said subject with a vector according to any one of claims 1-77; and
transplanting said transduced cell or cells derived therefrom into said subject where said cells or derivatives therefrom express said RAG1 protein.

84. The method of claim 83, wherein the cell is a stem cell.

85. The method of claim 83, wherein said cell is a stem cell derived from bone marrow.

86. The method of claim 83, wherein the cell is a human hematopoietic stem and progenitor cell.

87. The method of claim 86, wherein the human hematopoietic progenitor cell is a CD34⁺ cell.

88. A recombinant nucleic acid comprising one or more of the following:
an effective fragment of the endogenous promoter of the RAG1 gene that comprises or consists of the sequence of RAGpro (SEQ ID NO:2); and/or
a RAG1 enhancer element 1 (SEQ ID NO: 1) or an effective fragment thereof; and/or
a RAG1 enhancer element 3 (SEQ ID NO:3) or an effective fragment thereof; and/or
a RAG1 enhancer element 4 (SEQ ID NO:4) or an effective fragment thereof; and/or
an enlarged RAG1 enhancer element 5 core (SEQ ID NO:31) or an effective fragment thereof a RAG1 enhancer element 5 (SEQ ID NO:5) or an effective fragment thereof; and/or
a RAG1 enhancer element 6 (SEQ ID NO:6) or an effective fragment thereof a RAG1 enhancer element 7 (SEQ ID NO:78) or an effective fragment thereof; and/or
a RAG1 enhancer element 8 (SEQ ID NO:8) or an effective fragment thereof; and/or
a RAG1 enhancer element 9.1 (SEQ ID NO:9) or an effective fragment thereof; and/or
a RAG1 enhancer element 9.1 core sequence (SEQ ID NO:34) or an effective fragment thereof; and/or
a RAG1 enhancer element 9.2 (SEQ ID NO:10) or an effective fragment thereof; and/or
a RAG1 enhancer element 10 (SEQ ID NO:11) or an effective fragment thereof; and/or
a RAG1 enhancer element 11 extra (SEQ ID NO:33) or an effective fragment thereof; and/or
a RAG1 enhancer element 11 (SEQ ID NO:12) or an effective fragment thereof; and/or
a RAG1 enhancer element 12 (SEQ ID NO:13) or an effective fragment thereof; and/or
a RAG1 enhancer element 13 (SEQ ID NO:14) or an effective fragment thereof; and/or
an enlarged RAG1 enhancer element 14 core (SEQ ID NO:32) or an effective fragment thereof; and/or
a RAG1 enhancer element 14 (SEQ ID NO:15) or an effective fragment thereof; and/or
a RAG1 enhancer element 16 (SEQ ID NO:16) or an effective fragment thereof; and/or
a RAG1 enhancer element 17 (SEQ ID NO:17) or an effective fragment thereof; and/or
a RAG1 enhancer element 18 (SEQ ID NO:18) or an effective fragment thereof.

89. The nucleic acid of claim 88, wherein said nucleic acid comprises a sequence consisting of an effective fragment of the endogenous promoter of the RAG1 gene (SEQ ID NO:2).

90. The nucleic acid according to any one of claims 88-89, wherein said nucleic acid comprises a sequence consisting of a RAG1 enhancer element 1 (SEQ ID NO: 1) or an effective fragment thereof.

91. The nucleic acid according to any one of claims 88-90, wherein said nucleic acid comprises a sequence consisting of a RAG1 enhancer element 3 (SEQ ID NO:3) or an effective fragment thereof.

92. The nucleic acid according to any one of claims 88-91, wherein said nucleic acid comprises a sequence consisting of a RAG1 enhancer element 4 (SEQ ID NO:4) or an effective fragment thereof.

93. The nucleic acid according to any one of claims 88-92, wherein said nucleic acid comprises a an enlarged RAG1 enhancer element 5 core (SEQ ID NO:31) or an effective fragment thereof.

94. The nucleic acid according to any one of claims 88-92, wherein said nucleic acid comprises a sequence consisting of a RAG1 enhancer element 5 (SEQ ID NO:5) or an effective fragment thereof.

95. The nucleic acid according to any one of claims 88-94, wherein said nucleic acid comprises a sequence consisting of a RAG1 enhancer element 6 (SEQ ID NO:6) or an effective fragment thereof.

96. The nucleic acid according to any one of claims 88-95, wherein said nucleic acid comprises a sequence consisting of a RAG1 enhancer element 7 (SEQ ID NO:78) or an effective fragment thereof.

97. The nucleic acid according to any one of claims 88-96, wherein said nucleic acid comprises a sequence consisting of a RAG1 enhancer element 8 (SEQ ID NO:8) or an effective fragment thereof.

98. The nucleic acid according to any one of claims 88-97, wherein said nucleic acid comprises a sequence consisting of a RAG1 enhancer element 9.1 (SEQ ID NO:9) or an effective fragment thereof.

99. The nucleic acid according to any one of claims 88-98, wherein said nucleic acid comprises a sequence consisting of a RAG1 enhancer element 9.2 (SEQ ID NO:10) or an effective fragment thereof.

100. The nucleic acid according to any one of claims 88-99, wherein said nucleic acid comprises a sequence consisting of a RAG1 enhancer element 10 (SEQ ID NO:11) or an effective fragment thereof.

101. The nucleic acid according to any one of claims 88-100, wherein said nucleic acid comprises a sequence consisting of a RAG1 enhancer element 11 (SEQ ID NO:12) or an effective fragment thereof.

102. The nucleic acid according to any one of claims 88-101, wherein said nucleic acid comprises a sequence consisting of a RAG1 enhancer element 12 (SEQ ID NO:13) or an effective fragment thereof.

103. The nucleic acid according to any one of claims 88-102, wherein said nucleic acid comprises a sequence consisting of a RAG1 enhancer element 13 (SEQ ID NO:14) or an effective fragment thereof.

104. The nucleic acid according to any one of claims 88-103, wherein said nucleic acid comprises a sequence consisting of an enlarged RAG1 enhancer element 14 core (SEQ ID NO:32) or an effective fragment thereof.

105. The nucleic acid according to any one of claims 88-103, wherein said nucleic acid comprises a sequence consisting of a RAG1 enhancer element 14 (SEQ ID NO:15) or an effective fragment thereof.

106. The nucleic acid according to any one of claims 88-105, wherein said nucleic acid comprises a sequence consisting of a RAG1 enhancer element 16 (SEQ ID NO:16) or an effective fragment thereof.

107. The nucleic acid according to any one of claims 88-106, wherein said nucleic acid comprises a sequence consisting of a RAG1 enhancer element 17 (SEQ ID NO:17) or an effective fragment thereof.

108. The nucleic acid according to any one of claims 88-107, wherein said nucleic acid comprises a sequence consisting of a RAG1 enhancer element 18 (SEQ ID NO:18) or an effective fragment thereof.

109. The nucleic acid according to any one of claims 88-108, wherein said nucleic acid comprises an expression cassette.

110. The nucleic acid of claim 109, wherein said expression cassette comprises a nucleic acid that encodes a RAG1 protein.

111. The nucleic acid of claim 110, wherein said nucleic acid that encodes a RAG1 protein comprises a RAG1 cDNA.

112. The nucleic acid of claim 110, wherein said nucleic acid that encodes a RAG1 protein comprise a codon-optimized RAG1 cDNA.

113. The nucleic acid according to any one of claims 110-112, wherein said nucleic acid that encodes a RAG1 protein is operably linked to an endogenous RAG1 promoter or an effective fragment thereof.

114. The nucleic acid according to any one of claims 110-113, wherein said nucleic acid comprises a lentiviral vector according to any one of claims 1-77.

115. The nucleic acid according to any one of claims 88-114, wherein said nucleic acid does not comprise the endogenous RAG1 gene.


SEQUENCE LISTING (46 sequences)

SEQ ID NO: 1 - DNA, 1063 bp, Artificial
  RAG1 enhancer element
ggagcagaggtcaggggacagggaacggtgtccccgacctgtgggaagcgtggggacagaggctgcgtcccgacctgcaggaagcgaggggcagggaacggtgtcctcgaccagcgggaagcgagaggacaggggcggcgtccccgacccgtgggaagcaagcgggtaggggcggcgtccctgacctgtgaaaagcgaagggacaggggaggcgtccccgactggtgggaagcgaggggacagggactgcgtccccgacctgccgagagcgaaggggcggggggcggcgtccctgaccgctgggaagcgaggcggcggccgccaggaggaggcgcctgaaggagactcaccgttctagtgcgcggggaggccgaaccaggagggcagggctcccccaccaaccgcacgactccgctcagccaaggcgctggtagaggacggacacagacactgcgcgccgagacgaggctgcttggacggcaaactctggatccagtgggagccttcgccaccttcgctggccgcccgcaggccaagccccagctgcggacgccactgcttccgccttctctgctggctgcggggagcgaggggcagggagcgcgccggggaggagacagagcggccgagttcggagagagtgccccctggcggtgatcctccaggcggactgtggcgcgggccgggcggcgcccccagccccgcagcctggctttcttcctgctcgggtgtcccacctgggaattcgttgcaccgcacagccgaacccgagtgtgtcaatgggtgtgtgttggaatgaggatggcgacgagaagatccctcccggaataacttaatttggtgagcagtggcgacagtagtaatcccaaactgcgcttttatagcacccctctccaagcaggagaaaaccccagctgggctagatcgcttgaccttcaggaattcttaaacccctccccagctccaccctccttgcccaggaagtttctgcgatccatagaaccctctattattacctgctcttccccttcatccggtccaagggagtcagcccttgccttaa

SEQ ID NO: 2 - DNA, 267 bp, Artificial
  RAG1 promoter
ctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagc

SEQ ID NO: 3 - DNA, 150 bp, Artificial
  RAG1 enhancer element
ctcagcctgagtgattatgatcagagagttgtcccattttgggtggggggaacaccaaagccaaggcctgcttgtttcaaacacctgtgatgactgttattgagatacaaggcaagccatacagtggtaatggattccagatccaaaggg

SEQ ID NO: 4 - DNA, 152 bp, Artificial
  RAG1 enhancer element
ttgaggttatgttcttgagtcaggattcctggccagggattgtgatgatatatttctctttctgaagtggttcatgcaagaggttgtctgaaggaagagcaagaattgtagtgttattttgtggatacttgagacttataaaaaggcttttt

SEQ ID NO: 5 - DNA, 250 bp, Artificial
  RAG1 enhancer element
agggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcata

SEQ ID NO: 6 - DNA, 832 bp, Artificial
  RAG1 enhancer element
ggcaccaaactgttttctacttgacaggttgttatcatgctgtatttgcacccaccatcacagagattgtttaaaaaaaaagttatctagggcatgccaaattatatgtaaaagtatagtacagattgcagtggaaagataacgtgatgattctgtgatatctggagaggaaacataaccagttggagatagtgccttcatttttgttttaaaaacaaaagagaactcacttgcgaactctttttttttttttctaaatttcttcaggccaccatagctgaagctccctttacttctcacctgggttactatggtagctccacactttcttgtctctagtacctgaaccactttgatccattctatccatttgtcttcttagagcacagctctaattgtgactccccaggatgtaggtcaagtacagagtttttgtcgggccctccaaatataccccgtccatgtgtccccaaatcttcctggaataatcggcgattcttactcaggccttgtgtgttcccaccttgagggctttgctcatggaattttccctttgtaaaatgacctcttcacagtgctcacctgttgaaattgtagctatctttcaaagcccagctcagatgccttagtcctatgtgaagccttttgtgattctgccaaaccaattatgattttaccattctttgtgtttctatctctcttatatcatctgccatcggtgccttctcttttggtggtttgcatgtatggcttaacttcctagtttgtaaactctgtgaggacaacaaggactgagtcttaactgtctttgtcaccttgcatatagttgatg

SEQ ID NO: 7 - DNA, 235 bp, Artificial
  RAG1 enhancer element
tgtactgttagcaaattacttcccctcttggaacctcaatttcatcatccgtaaaatgggaacaataataaaaataccttttggccattagggggagctcaaggatgacttataccccaaaaggggaaggattgcttatgtggattctcagttctagtcattggcaacgctaaagggcagagctttcccctgttgtgggcaagggtttgcacgagggggactggccatcccagtt

SEQ ID NO: 8 - DNA, 427 bp, Artificial
  RAG1 enhancer element
ctgtggctgggattctgcaaaccacattgctgctttgtcagctgggtccctggtaggttctgccaatggagggcgctagagggagcctgggggactggtggagggagaggagttgggctcatcctgtctttgtccacatcacctagcatagacagctgggtccagcagcagtgttatatcccttccagtaattttggtggttgcttctcagcattcttgagtttataggtggagcatacttgatccttgtgtgagtccccaagagacactgggtcctatttgtggttgcttagtcaatgacttcatagcttaaaagaagtgacctcggagcccttgttcctgtatcgtacaattgcctagttcttcagttaccttcagcagtatgcatttctcactaagtcactcattagattggcttttcatttcc

SEQ ID NO: 9 - DNA, 1559 bp, Artificial
  RAG1 enhancer element
gtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggatatatgagtcccttatatgtcagggacaatgtacagtgctggtcataatattcattttctgaaaatgtcttcactgacataatggcttctgatttgtcatcctgaacaagtctattatattttatttagttattgtcctcaatttgcaaggttcataaagagtttcacacagatggcacccaaagtgcttatttctcattgtgctgaaattaaacttgaatatgctatatatcaaataaataatccattccatgccattaaaagagtttagaatgagagatgaaataaattttgcaaaaagtgtgcccacatgttcattttctccaaatagcatagtcattacatttaaaaacaatcattggtcaaaatagaaagtattaagaaaatactaagtatcaattgaggtctaccacatgttcagcaacaacttaggtgagatgtgggagtaggaaaaaggaatactttataacatcacagagaggcagattttggcacagggaagagaagtcaatgcaatacctcaaggaacccagtggtgatctaagaaaacactagtttgacccattgcctcaattaagtatttacctttcatagctaagaagagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaaaacacccactatcattcaaaggctgtcaggcaaggtggccgtttgaggggccactttagttcttggtttttttcagaagtcgaattggattgccaaacagcttgctcaactgcctcttcccataagctagatgggaggaaggctgggtctgtgtaacttggggtgcgcacaactaaagtgcataaaaagagaagga

SEQ ID NO: 10 - DNA, 535 bp, Artificial
  RAG1 enhancer element
ggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaatcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgtttgaggggccactttagttcttggtttttttcagaagtcgaattggattgccaaacagcttgctcaactgcctcttcccataagctagatgggaggaaggctgggtctgtgtaacttggggtgcgcacaactaaagtgcataaaaagag

SEQ ID NO: 11 - DNA, 307 bp, Artificial
  RAG1 enhancer element
attacccacctcactattatgaagtataacatacagcgtgcacagcacagtgtctggcccattgtagttgctcaatagcttttagtgtttgcttttatgagtttgagtcatctttgtggtgaggattacagaccctgacccctctacttgaggtagctaaagtttccctcttgatgatttaatcttggttggggtgagaaaaaagcaggtatcagctgtgctattggggatttcctaaccctttatattattacatgtttcatcctgccttgaggaagaagtttaagctacgccaatattcggggct

SEQ ID NO: 12 - DNA, 323 bp, Artificial
  RAG1 enhancer element
ttttttaggtctactactcttgtgtctgaaagttgcattttgtttgagtaagttcattaagcagggacctgaaagtaaacatcaaaggtgtgctgaatcagtggtgacaatttcatgtatcatatttcataataatgttctttcagagagcacataacattaactagcatggagcagtgcatgctccctgtatttccttctgggggccatgattgtgaaagtgttgcttgtgatttaccagcagatatttatacacatttacttatcacaagacgttttgagtctttcatgtggagctcaaagccttaaaacataaaaaccaa

SEQ ID NO: 13 - DNA, 349 bp, Artificial
  RAG1 enhancer element
ttgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggct

SEQ ID NO: 14 - DNA, 501 bp, Artificial
  RAG1 enhancer element
ctgaaaccgtttcaccttttattagaatagagcactgaggcatggcagtagccacaggaagtctgctttcctttgaacattgatgaagtttgaggaaccctttcatccttcagttaactcctatttccaagtgtctccatgcctatctttggcttgatttctgccacttgactttttttgaattcctttatcactcttttactcttttagtgatcattgacgtaggttcctgtacttgctgttgtcagaagaagcaagtttcagttttggaatgtaccctgcacgcttagttcttctttgactattgtaagccattcttctttgtctcctttctggctccctgcccccacctttctgctccttttttcttctattctctaaagaccttttcttttcatttactgcattaatgcaggaagcaaggcacagtggttaggaggataggttctagagtcagaaggcagtgttcagatccccactctgctacttaaccatctgtat

SEQ ID NO: 15 - DNA, 249 bp, Artificial
  RAG1 enhancer element
aatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctt

SEQ ID NO: 16 - DNA, 151 bp, Artificial
  RAG1 enhancer element
tatcattatttactgtctagctactgcaaacacagtttattcagctttgcattgctaaagcctggtggtgttacaagtaaatgtgcataagccataaccagtgctttttcgtgtatctaattttttcaggtacaaatagcttgaaacagtt

SEQ ID NO: 17 - DNA, 194 bp, Artificial
  RAG1 enhancer element
atgatttcttcttagccagttcatggattcaacaacttctgagactttttctcatggctttttctgatagtcaagaatataggcttttctttgcccacacggtggcactagctgcttagaaaacagtgtagctaaaatcagaaagcccgcagcagatctcctgaaagcatggagagtgagctgcagccgacgtt

SEQ ID NO: 18 - DNA, 381 bp, Artificial
  RAG1 enhancer element
aaaagtctattttatctaatataagcgtagttactcctgcttacttttggtttccatttgcatggagtatctttttccatcctttcacttttcatctatgtgtgtctttgtagttgaagtgagtttcttgtagatagcacatggttatgtcctgtttgtttttgtccattccttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctcc

SEQ ID NO: 19 - DNA, 3132 bp, Artificial
  RAG1 cDNA
atggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaa

SEQ ID NO: 20 - DNA, 3132 bp, Artificial
  Codon optimized RAG1 DNA
atggccgccagcttcccccccaccctgggcctgagcagcgcccccgacgagatccagcacccccacatcaagttcagcgagtggaagttcaagctgttccgcgtgcgcagcttcgagaagacccccgaggaggcccagaaggagaagaaggacagcttcgagggcaagcccagcctggagcagagccccgccgtgctggacaaggccgacggccagaagcccgtgcccacccagcccctgctgaaggcccaccccaagttcagcaagaagttccacgacaacgagaaggcccgcggcaaggccatccaccaggccaacctgcgccacctgtgccgcatctgcggcaacagcttccgcgccgacgagcacaaccgccgctaccccgtgcacggccccgtggacggcaagaccctgggcctgctgcgcaagaaggagaagcgcgccaccagctggcccgacctgatcgccaaggtgttccgcatcgacgtgaaggccgacgtggacagcatccaccccaccgagttctgccacaactgctggagcatcatgcaccgcaagttcagcagcgccccctgcgaggtgtacttcccccgcaacgtgaccatggagtggcacccccacacccccagctgcgacatctgcaacaccgcccgccgcggcctgaagcgcaagagcctgcagcccaacctgcagctgagcaagaagctgaagaccgtgctggaccaggcccgccaggcccgccagcacaagcgccgcgcccaggcccgcatcagcagcaaggacgtgatgaagaagatcgccaactgcagcaagatccacctgagcaccaagctgctggccgtggacttccccgagcacttcgtgaagagcatcagctgccagatctgcgagcacatcctggccgaccccgtggagaccaactgcaagcacgtgttctgccgcgtgtgcatcctgcgctgcctgaaggtgatgggcagctactgccccagctgccgctacccctgcttccccaccgacctggagagccccgtgaagagcttcctgagcgtgctgaacagcctgatggtgaagtgccccgccaaggagtgcaacgaggaggtgagcctggagaagtacaaccaccacatcagcagccacaaggagagcaaggagatcttcgtgcacatcaacaagggcggccgcccccgccagcacctgctgagcctgacccgccgcgcccagaagcaccgcctgcgcgagctgaagctgcaggtgaaggccttcgccgacaaggaggagggcggcgacgtgaagagcgtgtgcatgaccctgttcctgctggccctgcgcgcccgcaacgagcaccgccaggccgacgagctggaggccatcatgcagggcaagggcagcggcctgcagcccgccgtgtgcctggccatccgcgtgaacaccttcctgagctgcagccagtaccacaagatgtaccgcaccgtgaaggccatcaccggccgccagatcttccagcccctgcacgccctgcgcaacgccgagaaggtgctgctgcccggctaccaccacttcgagtggcagccccccctgaagaacgtgagcagcagcaccgacgtgggcatcatcgacggcctgagcggcctgagcagcagcgtggacgactaccccgtggacaccatcgccaagcgcttccgctacgacagcgccctggtgagcgccctgatggacatggaggaggacatcctggagggcatgcgcagccaggacctggacgactacctgaacggccccttcaccgtggtggtgaaggagagctgcgacggcatgggcgacgtgagcgagaagcacggcagcggccccgtggtgcccgagaaggccgtgcgcttcagcttcaccatcatgaagatcaccatcgcccacagcagccagaacgtgaaggtgttcgaggaggccaagcccaacagcgagctgtgctgcaagcccctgtgcctgatgctggccgacgagagcgaccacgagaccctgaccgccatcctgagccccctgatcgccgagcgcgaggccatgaagagcagcgagctgatgctggagctgggcggcatcctgcgcaccttcaagttcatcttccgcggcaccggctacgacgagaagctggtgcgcgaggtggagggcctggaggccagcggcagcgtgtacatctgcaccctgtgcgacgccacccgcctggaggccagccagaacctggtgttccacagcatcacccgcagccacgccgagaacctggagcgctacgaggtgtggcgcagcaacccctaccacgagagcgtggaggagctgcgcgaccgcgtgaagggcgtgagcgccaagcccttcatcgagaccgtgcccagcatcgacgccctgcactgcgacatcggcaacgccgccgagttctacaagatcttccagctggagatcggcgaggtgtacaagaaccccaacgccagcaaggaggagcgcaagcgctggcaggccaccctggacaagcacctgcgcaagaagatgaacctgaagcccatcatgcgcatgaacggcaacttcgcccgcaagctgatgaccaaggagaccgtggacgccgtgtgcgagctgatccccagcgaggagcgccacgaggccctgcgcgagctgatggacctgtacctgaagatgaagcccgtgtggcgcagcagctgccccgccaaggagtgccccgagagcctgtgccagtacagcttcaacagccagcgcttcgccgagctgctgagcaccaagttcaagtaccgctacgagggcaagatcaccaactacttccacaagaccctggcccacgtgcccgagatcatcgagcgcgacggcagcatcggcgcctgggccagcgagggcaacgagagcggcaacaagctgttccgccgcttccgcaagatgaacgcccgccagagcaagtgctacgagatggaggacgtgctgaagcaccactggctgtacaccagcaagtacctgcagaagttcatgaacgcccacaacgccctgaagaccagcggcttcaccatgaacccccaggccagcctgggcgaccccctgggcatcgaggacagcctggagagccaggacagcatggagttctaa

SEQ ID NO: 21 - DNA, 529 bp, Artificial
  CMV promoter
agttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 22 - DNA, 180 bp, Artificial
  Component of lentiviral vector
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagc

SEQ ID NO: 23 - DNA, 138 bp, Artificial
  Packaging signal in lentiviral vector
tcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggag

SEQ ID NO: 24 - DNA, 205 bp, Artificial
  RRE in lentiviral vector
tccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacct

SEQ ID NO: 25 - DNA, 656 bp, Artificial
  WPRE in lentiviral vector
cccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctgga

SEQ ID NO: 26 - DNA, 22 bp, Artificial
  PPT in lentiviral vector
tttttaaaagaaaaggggggac

SEQ ID NO: 27 - DNA, 233 bp, Artificial
  Component of lentiviral vector
tggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagc

SEQ ID NO: 28 - DNA, 140 bp, Artificial
  SV40 origin of replication
atcccgcccctaactccgcccagttccgcccattctccgccccatggctgactaattttttttatttatgcagaggccgaggccgcctcggcctctgagctattccagaagtagtgaggaggcttttttggaggcctagg

SEQ ID NO: 29 - DNA, 792 bp, Artificial
  Drug resistance gene
attgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 30 - DNA, 620 bp, Artificial
  Drug resistance gene
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcg

SEQ ID NO: 31 - DNA, 425 bp, Artificial
  Component of lentiviral vector
agaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatg

SEQ ID NO: 32 - DNA, 480 bp, Artificial
  Component of lentiviral vector
taaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaa

SEQ ID NO: 33 - DNA, 316 bp, Artificial
  Component of lentiviral vector
tctaggcaagtgtctctctctctctccccctctttttcttttagaaaaatacttcgtgataaaaacacaaaagtatgtgaatggtaattgaaaatgcattgcttccttttagaaacccactaggatttgcataaaccacttggctgtctgataagtcattatctgttctgtcagtccctggggacttttaatattaggtttatatttttatagaaaaggcaagttctaaatttgaagatgggtattgttaaactcttcaataatgacagtttgtgaaactgcatgttccaaaagtcctttaaaaagtcaataaggc

SEQ ID NO: 34 - DNA, 868 bp, Artificial
  Component of lentiviral vector
gtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaa

SEQ ID NO: 35 - DNA, 210 bp, Artificial
  Component of lentiviral vector
cttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctcc

SEQ ID NO: 36 - DNA, 352 bp, Artificial
  Component of lentiviral vector
tggcttgatttctgccacttgactttttttgaattcctttatcactcttttactcttttagtgatcattgacgtaggttcctgtacttgctgttgtcagaagaagcaagtttcagttttggaatgtaccctgcacgcttagttcttctttgactattgtaagccattcttctttgtctcctttctggctccctgcccccacctttctgctccttttttcttctattctctaaagaccttttcttttcatttactgcattaatgcaggaagcaaggcacagtggttaggaggataggttctagagtcagaaggcagtgttcagatccccactctgctacttaaccatctgtat

SEQ ID NO: 37 - DNA, 9760 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctcgagctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 38 - DNA, 10076 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctctaggcaagtgtctctctctctctccccctctttttcttttagaaaaatacttcgtgataaaaacacaaaagtatgtgaatggtaattgaaaatgcattgcttccttttagaaacccactaggatttgcataaaccacttggctgtctgataagtcattatctgttctgtcagtccctggggacttttaatattaggtttatatttttatagaaaaggcaagttctaaatttgaagatgggtattgttaaactcttcaataatgacagtttgtgaaactgcatgttccaaaagtcctttaaaaagtcaataaggctcgagctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 39 - DNA, 10451 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggatatatgagtcccttatatgtcagggacaatgtacagtgctggtcataatattcattttctgaaaatgtcttcactgacataatggcttctgatttgtcatcctgaacaagtctattatattttatttagttattgtcctcaatttgcaaggttcataaagagtttcacacagatggcacccaaagtgcttatttctcattgtgctgaaattaaacttgaatatgctatatatcaaataaataatccattccatgccattaaaagagtttagaatgagagatgaaataaattttgcaaaaagtgtgcccacatgttcattttctccaaatagcatagtcattacatttaaaaacaatcattggtcaaaatagaaagtattaagaaaatactaagtatcaattgaggtctaccacatgttcagcaacaacttaggtgagatgtgggagtaggaaaaaggaatactttataacatcacagagaggcagattttggcacagggaagagaagtcaatgcaatacctcaaggaacccagtggtgatctaagaaaacactagtttgacccattgcctcaattaagtatttacctttcatagctaagaagagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaaaacacccactatcattcaaaggctgtcaggcaaggtggccgtttgaggggccactttagttcttggtttttttcagaagtcgaattggattgccaaacagcttgctcaactgcctcttcccataagctagatgggaggaaggctgggtctgtgtaacttggggtgcgcacaactaaagtgcataaaaagagaaggattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctcgagctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 40 - DNA, 10767 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggatatatgagtcccttatatgtcagggacaatgtacagtgctggtcataatattcattttctgaaaatgtcttcactgacataatggcttctgatttgtcatcctgaacaagtctattatattttatttagttattgtcctcaatttgcaaggttcataaagagtttcacacagatggcacccaaagtgcttatttctcattgtgctgaaattaaacttgaatatgctatatatcaaataaataatccattccatgccattaaaagagtttagaatgagagatgaaataaattttgcaaaaagtgtgcccacatgttcattttctccaaatagcatagtcattacatttaaaaacaatcattggtcaaaatagaaagtattaagaaaatactaagtatcaattgaggtctaccacatgttcagcaacaacttaggtgagatgtgggagtaggaaaaaggaatactttataacatcacagagaggcagattttggcacagggaagagaagtcaatgcaatacctcaaggaacccagtggtgatctaagaaaacactagtttgacccattgcctcaattaagtatttacctttcatagctaagaagagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaaaacacccactatcattcaaaggctgtcaggcaaggtggccgtttgaggggccactttagttcttggtttttttcagaagtcgaattggattgccaaacagcttgctcaactgcctcttcccataagctagatgggaggaaggctgggtctgtgtaacttggggtgcgcacaactaaagtgcataaaaagagaaggattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctctaggcaagtgtctctctctctctccccctctttttcttttagaaaaatacttcgtgataaaaacacaaaagtatgtgaatggtaattgaaaatgcattgcttccttttagaaacccactaggatttgcataaaccacttggctgtctgataagtcattatctgttctgtcagtccctggggacttttaatattaggtttatatttttatagaaaaggcaagttctaaatttgaagatgggtattgttaaactcttcaataatgacagtttgtgaaactgcatgttccaaaagtcctttaaaaagtcaataaggctcgagctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 41 - DNA, 10646 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctcgagcttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctccttttttaggtctactactcttgtgtctgaaagttgcattttgtttgagtaagttcattaagcagggacctgaaagtaaacatcaaaggtgtgctgaatcagtggtgacaatttcatgtatcatatttcataataatgttctttcagagagcacataacattaactagcatggagcagtgcatgctccctgtatttccttctgggggccatgattgtgaaagtgttgcttgtgatttaccagcagatatttatacacatttacttatcacaagacgttttgagtctttcatgtggagctcaaagccttaaaacataaaaaccaatggcttgatttctgccacttgactttttttgaattcctttatcactcttttactcttttagtgatcattgacgtaggttcctgtacttgctgttgtcagaagaagcaagtttcagttttggaatgtaccctgcacgcttagttcttctttgactattgtaagccattcttctttgtctcctttctggctccctgcccccacctttctgctccttttttcttctattctctaaagaccttttcttttcatttactgcattaatgcaggaagcaaggcacagtggttaggaggataggttctagagtcagaaggcagtgttcagatccccactctgctacttaaccatctgtatgctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 42 - DNA, 10465 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctcgagaaaagtctattttatctaatataagcgtagttactcctgcttacttttggtttccatttgcatggagtatctttttccatcctttcacttttcatctatgtgtgtctttgtagttgaagtgagtttcttgtagatagcacatggttatgtcctgtttgtttttgtccattccttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctccttttttaggtctactactcttgtgtctgaaagttgcattttgtttgagtaagttcattaagcagggacctgaaagtaaacatcaaaggtgtgctgaatcagtggtgacaatttcatgtatcatatttcataataatgttctttcagagagcacataacattaactagcatggagcagtgcatgctccctgtatttccttctgggggccatgattgtgaaagtgttgcttgtgatttaccagcagatatttatacacatttacttatcacaagacgttttgagtctttcatgtggagctcaaagccttaaaacataaaaaccaagctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 43 - DNA, 10142 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctcgagaaaagtctattttatctaatataagcgtagttactcctgcttacttttggtttccatttgcatggagtatctttttccatcctttcacttttcatctatgtgtgtctttgtagttgaagtgagtttcttgtagatagcacatggttatgtcctgtttgtttttgtccattccttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctccgctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 44 - DNA, 10958 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctctaggcaagtgtctctctctctctccccctctttttcttttagaaaaatacttcgtgataaaaacacaaaagtatgtgaatggtaattgaaaatgcattgcttccttttagaaacccactaggatttgcataaaccacttggctgtctgataagtcattatctgttctgtcagtccctggggacttttaatattaggtttatatttttatagaaaaggcaagttctaaatttgaagatgggtattgttaaactcttcaataatgacagtttgtgaaactgcatgttccaaaagtcctttaaaaagtcaataaggcgcttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctccttttttaggtctactactcttgtgtctgaaagttgcattttgtttgagtaagttcattaagcagggacctgaaagtaaacatcaaaggtgtgctgaatcagtggtgacaatttcatgtatcatatttcataataatgttctttcagagagcacataacattaactagcatggagcagtgcatgctccctgtatttccttctgggggccatgattgtgaaagtgttgcttgtgatttaccagcagatatttatacacatttacttatcacaagacgttttgagtctttcatgtggagctcaaagccttaaaacataaaaaccaatggcttgatttctgccacttgactttttttgaattcctttatcactcttttactcttttagtgatcattgacgtaggttcctgtacttgctgttgtcagaagaagcaagtttcagttttggaatgtaccctgcacgcttagttcttctttgactattgtaagccattcttctttgtctcctttctggctccctgcccccacctttctgctccttttttcttctattctctaaagaccttttcttttcatttactgcattaatgcaggaagcaaggcacagtggttaggaggataggttctagagtcagaaggcagtgttcagatccccactctgctacttaaccatctgtatgctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 45 - DNA, 10782 bp, Artificial
  Lentiviral vector
cttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagccagaaaagagggattggggccctctcccaacagacatgagtatttaggagtccccagttccggtttccacatgacatgatatgatggtctgttgttttcctcagggacctagaggcagatgtagacaaatagcaggtgaggaggaagggtggtgctgagctgtaaacaacagctgcgggctgtaaacaatggagctatattaagaaagtcctgcatctgcagcattcggttcaatcctgccacccactgggcgtactcctgaattgactttccattgtcagctgatcagtgacagatgtgaaatgactcgttacacacacaggtcacattgggaggattcttagacgtcatattctttggtgcttcacaaagtcttactgtggcacatgaaatgatttcagatggtgcacaagatgatttcacatgtaaatttccatgagggtgcaaagagaggcttttcccaatctgaagaatgaatttaaaatctgctgtagaaaaataccagagagaaaaatatagaaagcagctgtccctcttaccaatttcagagcaggaaaggaatttcagtagagcataaaaacagctttctctcataaaagaaaacacaacaagaaaaataatccttgctgtgtagccttttgcagttattttctgtcatgtgttagatagagaccagagggcttaacattgttttgatacccttgcatctggcagattgcctgtcagaggaagttggaaacgaactgaccagctttatgcttccaggtggcactgaagccacattttcagtgccgttgttatcagtcatttaattccctactgaatattgagtggtgtttttagttcacaggccatttgcagtgcctttctgtttcctttttaacgttagccacattaagaaaaaaagtgggctttgcctacagcacctgcttctgctttccgcatttactgatccactaagtgtaagtgtgacatatagaggtctgtgttggttgttgttttgtgttcaaacagctgaacatctggcctctcatgtgcaggtgcacagaactcaggagaaggggttttaatttaagataaaaaaaataaggatatgtgctagttgcagatgagggtttgggtgctggacttaagatgcttctgtgttcaaaaacaaggagtcctcaatttgcaaggttcataaagagtttcacacagatggcacccaaagtgcttatttctcattgtgctgaaattaaacttgaatatgctatatatcaaataaatagaagtaaatagcctttctttatactttctagaagttgagctcaatgttctttcggagagagcaggctcatctccattatgtagcgccaatgacaaatacctgtatatgtctgtatttccttggtctgtcttgctcacctgttaagacaccttgctgcacaacagatggcacatgcttatcttgtggcagtgaagagttgacatcaaaggccaagaaaaccttgaaactcatccttttcagcccacaagagcagcttctctctaccacagacgatgaaagctggagataacaggggcattcccaccgctgcactctctcttgtggccagctgacagttaacacaggtgggcgtgaggtgctgcctacattttgaaggttgtctattgatggggaacaccacctctctgagctcaggcctctgctttggcccgtggagctgctgcttctgagaagtaaccaggtggtgaatacctgcctgcttttcagaaaatccgaggtggctgtcaattgacaattatttattcattgtaaaataatcacaggaacagcagcagtgtaggtttccctacctagagggtggtatgcagtgattctcaggcgctggttggaaggcacagctgagggacacaaactgccaggaagtaatgtggtaactagccatgagcttgtggtactaatggtggcacgggaaacaaggtctctgcttgacttttattttcactccataacaaactcaccagatcaggagcctaagggtgggtggggagggagaagagagaaaaaagcaaagggaaagttcaaagtgacactcgctggggctgaaaaccacactcccctgcagatgaggtccttggctctcgagtctaggcaagtgtctctctctctctccccctctttttcttttagaaaaatacttcgtgataaaaacacaaaagtatgtgaatggtaattgaaaatgcattgcttccttttagaaacccactaggatttgcataaaccacttggctgtctgataagtcattatctgttctgtcagtccctggggacttttaatattaggtttatatttttatagaaaaggcaagttctaaatttgaagatgggtattgttaaactcttcaataatgacagtttgtgaaactgcatgttccaaaagtcctttaaaaagtcaataaggcgaaaagtctattttatctaatataagcgtagttactcctgcttacttttggtttccatttgcatggagtatctttttccatcctttcacttttcatctatgtgtgtctttgtagttgaagtgagtttcttgtagatagcacatggttatgtcctgtttgtttttgtccattccttctgtcattttattgttttctggttgttttgcatttctcttcctcgtttctttatctcttactgtttatctttgtgtggtttgcttggtgacaaattttagttactttctctttcgcatctgtgtatctgctctactagtgggttctatacttttgtttgttctcctgatggtagttattgccctttggctttcagatatagggctccttttttaggtctactactcttgtgtctgaaagttgcattttgtttgagtaagttcattaagcagggacctgaaagtaaacatcaaaggtgtgctgaatcagtggtgacaatttcatgtatcatatttcataataatgttctttcagagagcacataacattaactagcatggagcagtgcatgctccctgtatttccttctgggggccatgattgtgaaagtgttgcttgtgatttaccagcagatatttatacacatttacttatcacaagacgttttgagtctttcatgtggagctcaaagccttaaaacataaaaaccaagctaaagagccaggtggcagctggagctggggtctcctggcccatgattggctgccatcatttgtggttagccctccatggtgggggaggctgggaaggacagtggaagctgataaacagctcagcagcatgttctgagaaacaagagggcaaggagagagcagagaacacactttgccttctctttggtattgagtaatatcaaccaaattgcagacatctcaacactttggccaggcagcctgctgagcaaggtacctcagccagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccat

SEQ ID NO: 46 - DNA, 10782 bp, Artificial; identical to SEQ ID NO: 45
  Lentiviral vector